(19) United States
(12) Patent Application Publication
(10) Pub. No.: US 2017/0005274 A1
(43) Pub. Date: Jan. 5, 2017

(54) COMPOUND, ORGANIC ELECTROLUMINESCENT DEVICE MATERIAL, ORGANIC ELECTROLUMINESCENT DEVICE, AND ELECTRONIC EQUIPMENT

(71) Applicant: IDEMITSU KOSAN CO., LTD., Tokyo (JP)

(72) Inventors: Yu KUDO, Chiba-shi, Chiba (JP); Kei YOSHIDA, Chiba-shi, Chiba (JP); Masahiro KAWAMURA, Chiba-shi, Chiba (JP); Yumiko MIZUKI, Basel (CH); Hirokatsu ITO, Ichihara-shi, Chiba (JP); Tasuku HAKETA, Chiba-shi, Chiba (JP); Tomoharu HAYAMA, Utsunomiya-shi, Tochigi (JP)

(73) Assignee: IDEMITSU KOSAN CO., LTD., Tokyo (JP)

(21) Appl. No.: 15/107,300

(22) PCT Filed: Aug. 14, 2015
(86) PCT No.: PCT/JP2015/072975

§ 371 Date: Jun. 22, 2016

(30) Foreign Application Priority Data
Aug. 15, 2014 (JP) 2014-165453

(51) Int. Cl. H01L 51/00; C07D 491/048; C09K 11/02; C09K 11/06

(52) U.S. Cl. CPC H01L 51/0071; C09K 11/06; C07D 491/048; H01L 51/0067; H01L 51/0054; H01L 51/0072; H01L 51/0052; H01L 51/0073; H01L 51/0074; C09K 11/025; H01L 51/0085; H01L 51/5012

(57) ABSTRACT

Disclosed are a compound having, as a main skeleton, a polycyclic structure represented by the following general formula (1), a material for organic electroluminescence devices including the compound, an organic electroluminescence device using the compound, and electronic equipment having the organic electroluminescence device mounted thereon. A compound capable of realizing an organic EL device with high emission efficiency, a material for organic electroluminescence devices including the compound, an organic electroluminescence device using the compound, and electronic equipment having the organic electroluminescence device mounted thereon, are provided.

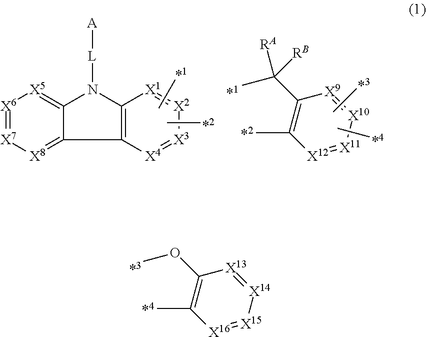

In the formula (1), A, L, X¹ to X¹⁶, R, R^{A}, and R^{B} are those defined in the specification.

### TECHNICAL FIELD

The present invention relates to a compound, a material for organic electroluminescence devices, an organic electroluminescence device, and an electronic equipment.

### BACKGROUND ART

An organic electroluminescence device (hereinafter referred to as “organic EL device”) which is provided with an organic thin film layer including a light emitting layer between an anode and a cathode and from which emission is obtained from exciton energy generated by recombination of holes and electrons as injected in the light emitting layer is known.

The organic EL device is expected as a light emitting device which is excellent in high emission efficiency, high image quality, and low power consumption as well as design properties of a thin model while employing advantages as a self-light emitting device. It is known to form the light emitting layer into a host/dopant light emitting layer by doping a host with a light emitting material as a dopant.

In the host/dopant light emitting layer, excitons can be efficiently generated from charges injected on the host. Then, emission with high efficiency can be obtained from the dopant by moving the energy of generated excitons into the dopant.

In recent years, for the purpose of achieving a performance improvement of organic EL devices, further researches regarding the host/dopant system have also been made, and searches on suitable host materials and other materials for organic EL devices are continued.

### CITATION LIST

### Patent Literature

PTL 1: KR 10-2013-0052485A
PTL 2: WO 2010/136109A

### SUMMARY OF INVENTION

### Technical Problem

An object of the present invention is to provide a compound capable of realizing an organic EL device with high emission efficiency. In addition, another object is to provide a material for organic EL devices including the compound, an organic EL device including the compound, and electronic equipment having the organic EL device mounted thereon.

### Solution to Problem

The present inventors made extensive and intensive investigations. As a result, it has been found that a compound having, as a main skeleton, a polycyclic structure represented by the following general formula (1) can solve the aforementioned problem.

Specifically, according to an embodiment of the present invention, the following [1] to [4] are provided.

[1] A compound represented by the following general formula (1):

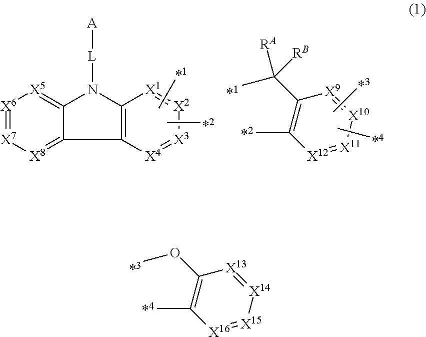

In the general formula (1),

A is a substituted or unsubstituted aryl group having 6 to 50 ring carbon atoms or a substituted or unsubstituted heteroaryl group having 3 to 50 ring atoms;

L is a single bond, a substituted or unsubstituted arylene group having 6 to 60 ring carbon atoms, or a substituted or unsubstituted heteroarylene group having 3 to 50 ring atoms;

two selected from X¹ to X⁴ are a carbon atom bonding to *¹ or *², respectively, and the other two of X¹ to X⁴ are each independently C(R) or a nitrogen atom;

two selected from X⁹ to X¹² are a carbon atom bonding to *³ or *⁴, respectively, and the other two of X⁹ to X¹² are each independently C(R) or a nitrogen atom;

X⁵ to X⁸ and X¹³ to X¹⁶ are each independently C(R) or a nitrogen atom,

Rs are each independently a hydrogen atom or a substituent, plural Rs may be the same as or different from every other R, and two selected from plural Rs may be bonded to each other to form a ring; and

R^{A} and R^{B} are each independently a hydrogen atom, a substituted or unsubstituted alkyl group having 1 to 20 carbon atoms, a substituted or unsubstituted aryl group having 6 to 50 ring carbon atoms, a substituted or unsubstituted heteroaryl group having 3 to 50 ring atoms, a halogen atom, a mono-substituted, di-substituted, or tri-substituted silyl group having a substituent selected from an alkyl group having 1 to 50 carbon atoms and an aryl group having 6 to 50 ring carbon atoms, a substituted or unsubstituted alkoxy group having 1 to 20 carbon atoms, a substituted or unsubstituted aryloxy group having 6 to 50 ring carbon atoms, or a cyano group, R^{A} and R^{B} may be the same as or different from each other, and R^{A} and R^{B} may be bonded to each other to form a ring.

[2] A material for organic electroluminescence devices including the compound according to the above W.
[3] An organic electroluminescence device including a cathode, an anode, and one or more organic thin film layers between the cathode and the anode, wherein

the one or more organic thin film layers include a light emitting layer, and at least one of the one or more organic thin film layers is a layer containing the compound according to the above [1].

[4] Electronic equipment including the organic electroluminescence device according to the above [3] mounted thereon.

### Advantageous Effects of Invention

In accordance with the present invention, a compound capable of realizing an organic EL device with high emission efficiency can be provided.

### BRIEF DESCRIPTION OF DRAWING

FIG. 1 is a view showing a schematic configuration of an organic EL device according to an embodiment of the present invention.

### DESCRIPTION OF EMBODIMENTS

In the present specification, the term of “XX to YY carbon atoms” referred to by “a substituted or unsubstituted group ZZ having XX to YY carbon atoms” is the number of carbon atoms in the case where the group ZZ is unsubstituted and does not include any carbon atom in the substituent in the case where the group ZZ is substituted. Here, “YY” is larger than “XX”, and each of “XX” and “YY” means an integer of 1 or more.

In addition, in the present specification, the term of “XX to YY atoms” referred to by “a substituted or unsubstituted group ZZ having XX to YY atoms” is the number of atoms in the case where the group ZZ is unsubstituted and does not include any atom in the substituent in the case where the group ZZ is substituted. Here, “YY” is larger than “XX”, and each of “XX” and “YY” represents an integer of 1 or more.

The number of “ring carbon atoms” referred to in the present specification means the number of the carbon atoms included in the atoms which are members forming the ring itself of a compound in which a series of atoms is bonded to form a ring (for example, a monocyclic compound, a fused ring compound, a crosslinked compound, a carbocyclic compound, and a heterocyclic compound). In the case where the ring is substituted with a substituent, the carbon atom in the substituent is not included in the ring carbon atom. The same applies to the number of “ring carbon atom” described below, unless otherwise noted. For example, a benzene ring has 6 ring carbon atoms, a naphthalene ring has 10 ring carbon atoms, a pyridinyl group has 5 ring carbon atoms, and a furanyl group has 4 ring carbon atoms. In the case where a benzene ring or a naphthalene ring has, for example, an alkyl group as a substituent, the carbon atom in the alkyl group is not counted as the ring carbon atom of the benzene or naphthalene ring. In the case where, for example, a fluorene ring is bonded as a substituent to a fluorene ring (inclusive of a spirofluorene ring), the carbon atom in the fluorene ring as the substituent is not counted as the ring carbon atom of the fluorene ring.

In addition, the number of “ring atom” referred to in the present specification means the number of the atoms which are members forming the ring itself (for example, a monocyclic ring, a fused ring, and a ring assembly) of a compound in which a series of atoms is bonded to form the ring (for example, a monocyclic compound, a fused ring compound, a crosslinked compound, a carbocyclic compound, and a heterocyclic compound). The atom not forming the ring (for example, hydrogen atom(s) for saturating the valence of the atom which forms the ring) and the atom in a substituent, in the case where the ring is substituted with a substituent, are not counted as the ring atom. The same applies to the number of “ring atoms” described below, unless otherwise noted. For example, a pyridine ring has 6 ring atoms, a quinazoline ring has 10 ring atoms, and a furan ring has 5 ring atoms. The hydrogen atom on the ring carbon atom of a pyridine ring or a quinazoline ring and the atom in a substituent are not counted as the ring atom. In the case of a fluorene ring to which a fluorene ring is bonded as a substituent to a fluorene ring (inclusive of a spirofluorene ring), the atom in the fluorene ring as the substituent is not counted as the ring atom of the fluorene ring.

In addition, the definition of “hydrogen atom” as referred to in the present specification includes isotopes different in the neutron numbers, namely, light hydrogen (protium), heavy hydrogen (deuterium), and tritium.

Each of the terms of “heteroaryl group” and “heteroarylene group” as referred to in the present specification means a group having at least one hetero atom as a ring atom. The hetero atom is preferably at least one selected from a nitrogen atom, an oxygen atom, a sulfur atom, a phosphorus atom, a silicon atom, and a selenium atom, and more preferably at least one selected from a nitrogen atom, an oxygen atom, and a sulfur atom.

In the present specification, the “substituted or unsubstituted carbazolyl group” represents any of the following carbazolyl groups:

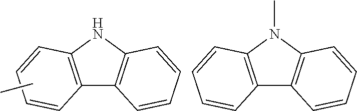

and substituted carbazolyl groups each further having an optional substituent on the aforementioned groups.

It is to be noted that in the substituted carbazolyl groups, optional substituents may be bonded to each other to form a fused ring, and the substituted carbazolyl group may contain a hetero atom, such as a nitrogen atom, an oxygen atom, a silicon atom, a selenium atom, etc. A bonding position may be any of the 1-position to 9-position. Specific examples of such substituted carbazolyl groups include the following groups.

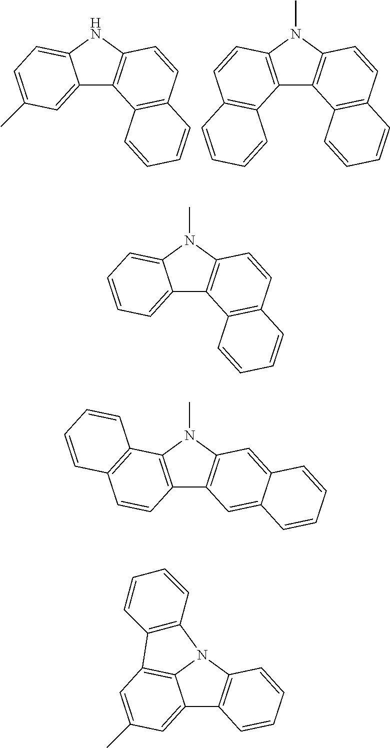

In the present specification, the “substituted or unsubstituted dibenzofuranyl group” and “substituted or unsubstituted dibenzothiophenyl group” represent any of the following dibenzofuranyl group and dibenzothiophenyl group:

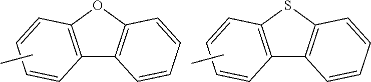

and substituted dibenzofuranyl groups and substituted dibenzothiophenyl groups each further having an optional substituent on the aforementioned groups.

It is to be noted that in the substituted dibenzofuranyl groups and substituted dibenzothiophenyl groups, optional substituents may be bonded to each other to form a fused ring, and the substituted dibenzofuranyl groups and substituted dibenzothiophenyl groups may contain a hetero atom, such as a nitrogen atom, an oxygen atom, a silicon atom, a selenium atom, etc. A bonding position may be any of the 1-position to 8-position.

Specific examples of such substituted dibenzofuranyl groups and substituted dibenzothiophenyl groups include the following groups.

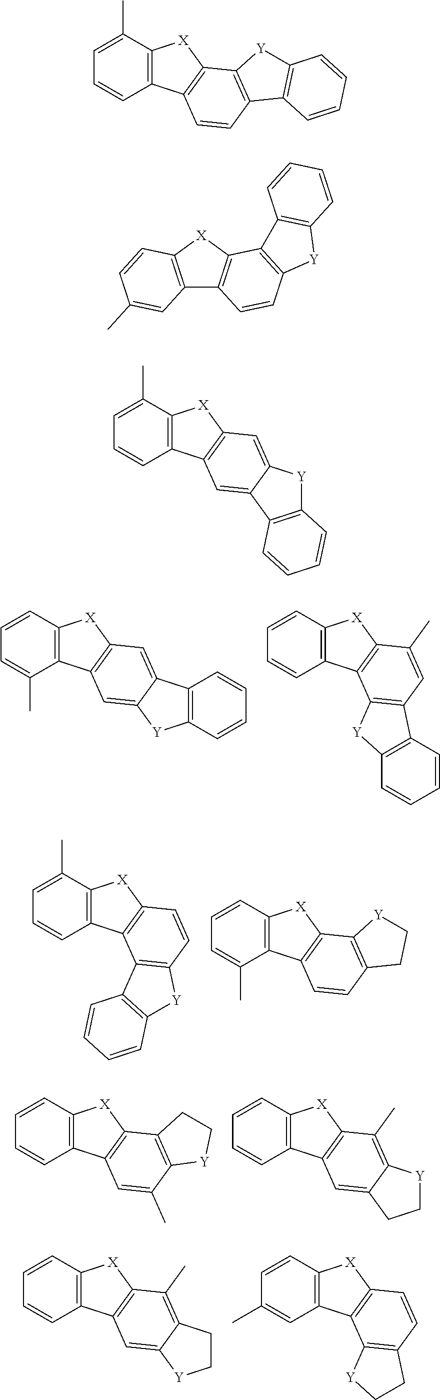

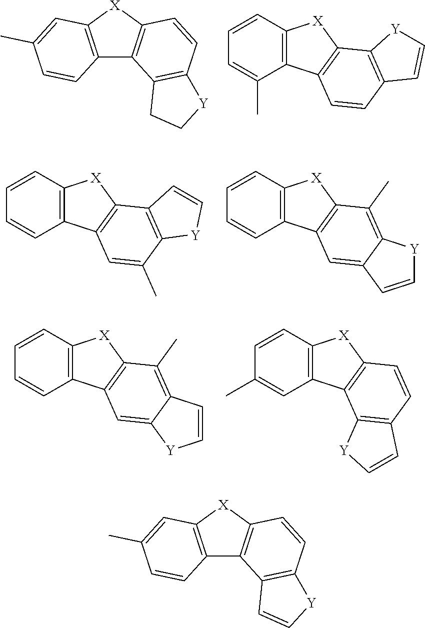

In the foregoing formulae, X represents an oxygen atom or a sulfur atom; and Y represents an oxygen atom, a sulfur atom, NH, NR^{a} (R^{a} is a substituent), CH₂, or CR^{b}₂ (R^{b} is a substituent).

The “substituent” or the substituent referred to by the description of “substituted or unsubstituted” is preferably a group selected from the group consisting of an alkyl group having 1 to 50 carbon atoms (preferably 1 to 18 carbon atoms, and more preferably 1 to 8 carbon atoms); a cycloalkyl group having 3 to 50 ring carbon atoms (preferably 3 to 10 ring carbon atoms, more preferably 3 to 8 ring carbon atoms, and still more preferably 5 or 6 ring carbon atoms); an aryl group having 6 to 50 ring carbon atoms (preferably 6 to 25 ring carbon atoms, and more preferably 6 to 18 ring carbon atoms); an aralkyl group having 7 to 51 carbon atoms (preferably 7 to 30 carbon atoms, and more preferably 7 to 20 carbon atoms) having an aryl group having 6 to 50 ring carbon atoms (preferably 6 to 25 ring carbon atoms, and more preferably 6 to 18 ring carbon atoms); an amino group; a mono-substituted or di-substituted amino group having a substituent selected from an alkyl group having 1 to 50 carbon atoms (preferably 1 to 18 carbon atoms, and more preferably 1 to 8 carbon atoms) and an aryl group having 6 to 50 ring carbon atoms (preferably 6 to 25 ring carbon atoms, and more preferably 6 to 18 ring carbon atoms); an alkoxy group having an alkyl group having 1 to 50 carbon atoms (preferably 1 to 18 carbon atoms, and more preferably 1 to 8 carbon atoms); an aryloxy group having an aryl group having 6 to 50 ring carbon atoms (preferably 6 to 25 ring carbon atoms, and more preferably 6 to 18 ring carbon atoms); a mono-substituted, di-substituted, or tri-substituted silyl group having a substituent selected from an alkyl group having 1 to 50 carbon atoms (preferably 1 to 18 carbon atoms, and more preferably 1 to 8 carbon atoms) and an aryl group having 6 to 50 ring carbon atoms (preferably 6 to 25 ring carbon atoms, and more preferably 6 to 18 ring carbon atoms); a heteroaryl group having 5 to 50 ring atoms (preferably 5 to 24 ring atoms, and more preferably 5 to 13 ring atoms); a haloalkyl group having 1 to 50 carbon atoms (preferably 1 to 18 carbon atoms, and more preferably 1 to 8 carbon atoms); a halogen atom (e.g., a fluorine atom, a chlorine atom, a bromine atom, and an iodine atom); a cyano group; a nitro group; a sulfonyl group having a substituent selected from an alkyl group having 1 to 50 carbon atoms (preferably 1 to 18 carbon atoms, and more preferably 1 to 8 carbon atoms) and an aryl group having 6 to 50 ring carbon atoms (preferably 6 to 25 ring carbon atoms, and more preferably 6 to 18 ring carbon atoms); a di-substituted phosphoryl group having a substituent selected from an alkyl group having 1 to 50 carbon atoms (preferably 1 to 18 carbon atoms, and more preferably 1 to 8 carbon atoms) and an aryl group having 6 to 50 ring carbon atoms (preferably 6 to 25 ring carbon atoms, and more preferably 6 to 18 ring carbon atoms); an alkylsulfonyloxy group; an arylsulfonyloxy group; an alkylcarbonyloxy group; an arylcarbonyloxy group; a boron-containing group; a zinc-containing group; a tin-containing group; a silicon-containing group; a magnesium-containing group; a lithium-containing group; a hydroxyl group; an alkyl-substituted or aryl-substituted carbonyl group; a carboxyl group; a vinyl group; a (meth)acryloyl group; an epoxy group; and an oxetanyl group.

Such a substituent may be further substituted with an optional substituent as described above. A plurality of such a substituent may be bonded to each other to form a ring.

The “unsubstituted” referred to by the description of “substituted or unsubstituted” means that the group is not substituted with such a substituent, and a hydrogen atom is bonded thereto.

Among the aforementioned substituents, a substituted or unsubstituted alkyl group having 1 to 50 carbon atoms (preferably 1 to 18 carbon atoms, and more preferably 1 to 8 carbon atoms); a substituted or unsubstituted cycloalkyl group having 3 to 50 ring carbon atoms (preferably 3 to 10 ring carbon atoms, more preferably 3 to 8 ring carbon atoms, and still more preferably 5 or 6 ring carbon atoms); a substituted or unsubstituted aryl group having 6 to 50 ring carbon atoms (preferably 6 to 25 ring carbon atoms, and more preferably 6 to 18 ring carbon atoms); a mono-substituted or di-substituted amino group having a substituent selected from a substituted or unsubstituted alkyl group having 1 to 50 carbon atoms (preferably 1 to 18 carbon atoms, and more preferably 1 to 8 carbon atoms) and a substituted or unsubstituted aryl group having 6 to 50 ring carbon atoms (preferably 6 to 25 ring carbon atoms, and more preferably 6 to 18 ring carbon atoms); a substituted or unsubstituted heteroaryl group having 5 to 50 ring atoms (preferably 5 to 24 ring atoms, and more preferably 5 to 13 ring atoms); a halogen atom; and a cyano group are more preferred.

In the present specification, those which are defined as being preferred can be selected arbitrarily, and it may be said that a combination thereof is a more preferred embodiment.

### [Compound]

In the embodiment of the present invention, a compound represented by the following general formula (1) (hereinafter also referred to as “compound (1)”) is provided. The compound is useful as a material for organic electroluminescence devices.

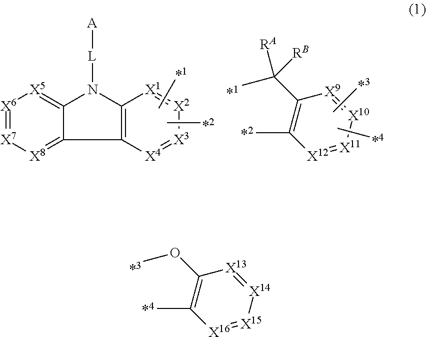

### <Re: A in the General Formula (1)>

In the general formula (1), A is a substituted or unsubstituted aryl group having 6 to 50 ring carbon atoms or a substituted or unsubstituted heteroaryl group having 3 to 50 ring atoms.

In the embodiment of the present invention, A is preferably a substituted or unsubstituted aryl group having 6 to 24 ring carbon atoms or a substituted or unsubstituted heteroaryl group having 3 to 24 ring atoms and containing a nitrogen atom, an oxygen atom, or a sulfur atom.

In the general formula (1), the number of ring carbon atoms of the aryl group represented by A is 6 to 50, preferably 6 to 24, and more preferably 6 to 18.

Examples of the aryl group represented by A in the general formula (1) include a phenyl group, a naphthyl group, a naphthylphenyl group, a biphenylyl group, a terphenylyl group, an acenaphthelenyl group, an anthryl group, a benzanthryl group, an aceanthryl group, a phenanthryl group, a benzophenanthryl group, a phenalenyl group, a fluorenyl group, a 9,9′-spirobifluorenyl group, a benzofluorenyl group, a dibenzofluorenyl group, a picenyl group, a pentacenyl group, a pyrenyl group, a chrysenyl group, a benzochrysenyl group, an s-indacenyl group, an as-indacenyl group, a fluoranthenyl group, a benzofluoranthenyl group, a tetracenyl group, a triphenylenyl group, a benzotriphenylenyl group, a perylenyl group, a coronyl group, a dibenzanthryl group, and the like.

Of these, A is preferably a substituted or unsubstituted fused aryl group and preferably a substituted or unsubstituted fused aryl group having 10 to 24 ring carbon atoms (preferably 10 to 18 ring carbon atoms).

Examples of the fused aryl group which may be selected as A include a naphthyl group, a naphthylphenyl group, an acenaphthylenyl group, an anthryl group, a benzanthryl group, an aceanthryl group, a phenanthryl group, a benzophenanthryl group, a phenalenyl group, a fluorenyl group, a 9,9′-spirobifluorenyl group, a benzofluorenyl group, a dibenzofluorenyl group, a picenyl group, a pentacenyl group, a pyrenyl group, a chrysenyl group, a benzochrysenyl group, an s-indacenyl group, an as-indacenyl group, a fluoranthenyl group, a benzofluoranthenyl group, a tetracenyl group, a triphenylenyl group, a benzotriphenylenyl group, a perylenyl group, a coronyl group, a dibenzanthryl group, and the like.

In the embodiment of the present invention, the fused aryl group which may be selected as A is more preferably a monovalent residual group of a compound represented by any one of the following general formulae (a1-1) to (a1-6).

It is to be noted that the carbon atoms bonding to the hydrogen atoms in the following general formulae (a1-1) to (a1-6) may be substituted with the aforementioned substituents in place of the hydrogen atoms.

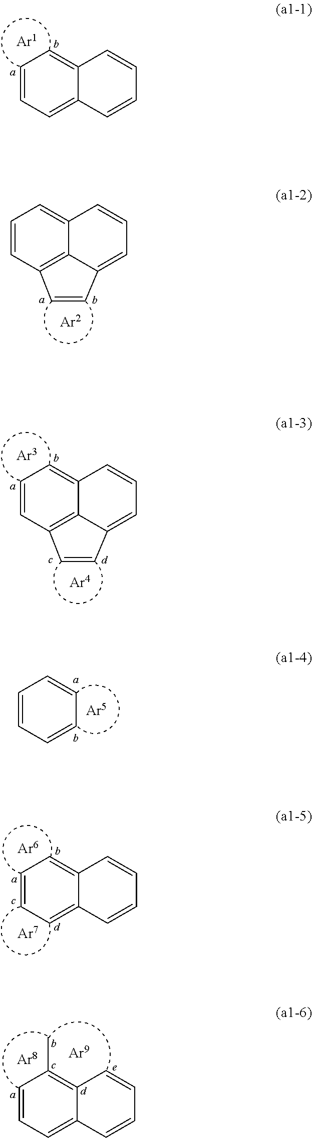

In the general formulae (a1-1) to (a1-5), each of Ar¹ to Ar⁷ independently forms a ring structure having 6 to 18 ring carbon atoms (preferably 6 to 12 ring carbon atoms) together with two carbon atoms expressed by a and b, or c and d, in each of the formulae. It is to be noted that the two carbon atoms expressed by a and b, or c and d, are also counted in the aforementioned ring carbon atom number.

In the general formula (a1-6), Ar⁸ forms a ring structure having 6 to 18 ring carbon atoms (preferably 6 to 12 ring carbon atoms) together with three carbon atoms expressed by a, b, and c in the formula, and Ar⁹ forms a ring structure having 6 to 18 ring carbon atoms together with four carbon atoms expressed by b, c, d, and e in the formula. It is to be noted that the three carbon atoms expressed by a, b, and c, or the four carbon atoms expressed by b, c, d, and e, are also counted in the aforementioned ring carbon atom number.

Examples of the ring structures represented by Ar¹ to Ar⁹ include a benzene structure, a naphthalene structure, an anthracene structure, a phenanthrene structure, a fluorene structure, an indane structure, a trindene structure, a chrysene structure, a naphthacene structure, a triphenylene structure, and the like.

Examples of the monovalent residual group of the compound represented by the general formula (a1-1) include monovalent residual groups of the following compounds. It is to be noted that the carbon atoms bonding to the hydrogen atoms in the structures of these compounds may be substituted with the aforementioned substituents in place of the hydrogen atoms.

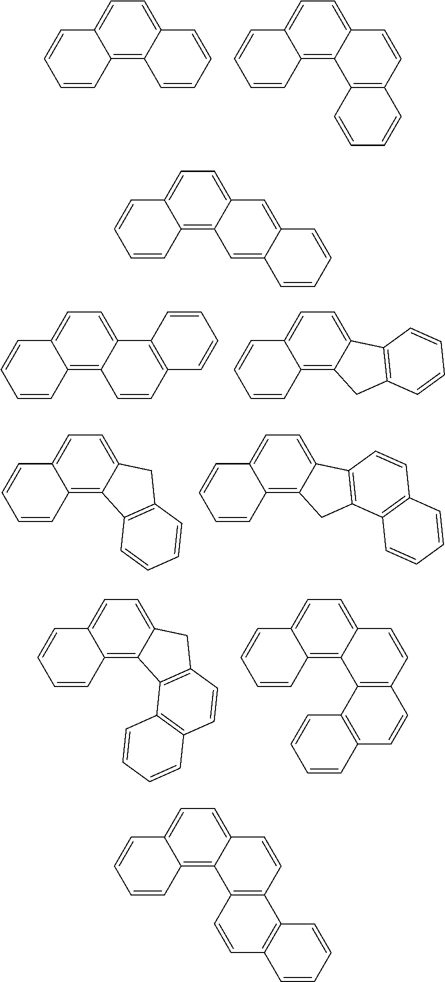

Examples of the monovalent residual group of the compound represented by the general formula (a1-2) include monovalent residual groups of the following compounds. It is to be noted that the carbon atoms bonding to the hydrogen atoms in the structures of these compounds may be substituted with the aforementioned substituents in place of the hydrogen atoms.

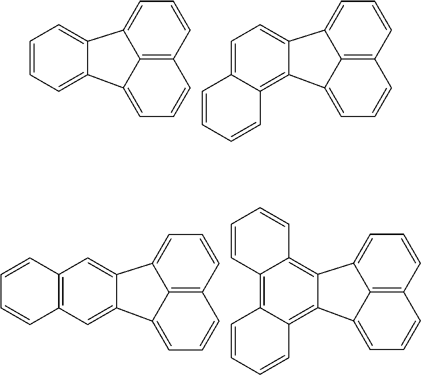

Examples of the monovalent residual group of the compound represented by the general formula (a1-3) include monovalent residual groups of the following compounds. It is to be noted that the carbon atoms bonding to the hydrogen atoms in the structures of these compounds may be substituted with the aforementioned substituents in place of the hydrogen atoms.

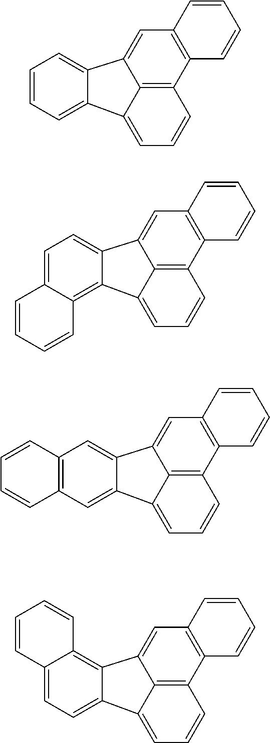

Examples of the monovalent residual group of the compound represented by the general formula (a1-4) include monovalent residual groups of the following compounds. It is to be noted that the carbon atoms bonding to the hydrogen atoms in the structures of these compounds may be substituted with the aforementioned substituents in place of the hydrogen atoms.

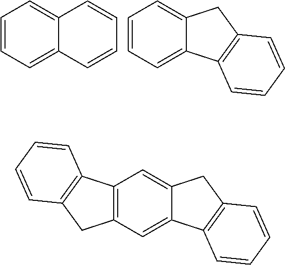

Examples of the monovalent residual group of the compound represented by the general formula (a1-5) include monovalent residual groups of the following compounds. It is to be noted that the carbon atoms bonding to the hydrogen atoms in the structures of these compounds may be substituted with the aforementioned substituents in place of the hydrogen atoms.

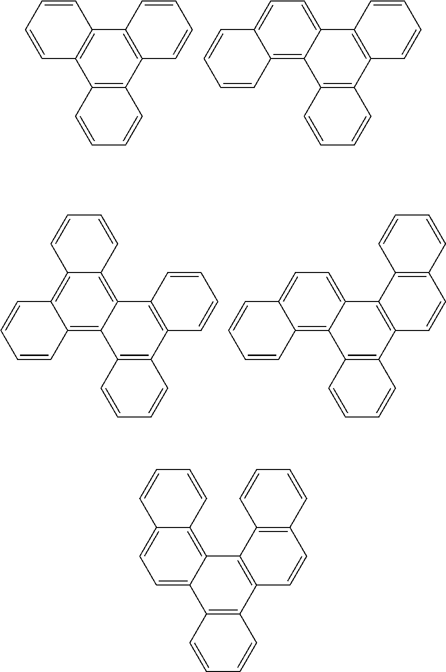

Examples of the monovalent residual group of the compound represented by the general formula (a1-6) include monovalent residual groups of the following compounds. It is to be noted that the carbon atoms bonding to the hydrogen atoms in the structures of these compounds may be substituted with the aforementioned substituents in place of the hydrogen atoms.

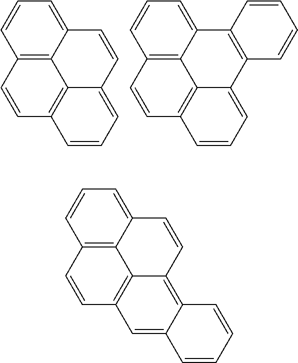

In the general formula (1), the number of ring atoms of the heteroaryl group represented by A is 3 to 50, preferably 6 to 20, and more preferably 6 to 14.

The heteroaryl group is preferably any one of a monocyclic ring, a fused ring constituted of two rings, and a fused ring constituted of three rings.

The number of hetero atoms contained in the heteroaryl group is preferably 1 to 3, and more preferably 2 or 3. In particular, in the case where the heteroaryl group is a monocyclic ring, the number of hetero atoms contained therein is preferably 2 or 3, and more preferably 3, and in the case where the heteroaryl group is a fused ring constituted of two rings or three rings, the number of hetero atoms contained therein is preferably 2.

It is to be noted that examples of the hetero atom of the heteroaryl group include a nitrogen atom, an oxygen atom, a sulfur atom, a phosphorus atom, a silicon atom, a selenium atom, and the like. Of these, a nitrogen atom, an oxygen atom, and a sulfur atom are preferred, with a nitrogen atom being more preferred.

Examples of the heteroaryl group represented by A in the general formula (1) include a monovalent residual group of a nitrogen-containing heterocyclic compound, such as pyrrole, pyridine, imidazopyridine, pyrazole, triazole, tetrazole, indole, isoindole, carbazole, etc.; a monovalent residual group of an oxygen-containing heterocyclic compound, such as furan, benzofuran, isobenzofuran, dibenzofuran, oxazole, oxadiazole, benzoxazole, benzonaphthofuran, dinaphthofuran, etc.; a monovalent residual group of a sulfur-containing heterocyclic compound, such as thiophene, benzothiophene, dibenzothiophene, thiazole, thiadiazole, benzothiazole, benzonaphthothiophene, dinaphthothiophene, etc.; and the like.

The heteroaryl group represented by A is especially preferably a nitrogen-containing heteroaryl group. Specific examples thereof include monovalent residual groups of compounds selected from pyrrole, pyridine, pyridazine, imidazopyridine, pyrimidine, pyrazine, triazine, imidazole, pyrazole, oxadiazole, thiadiazole, triazole, tetrazole, indole, isoindole, indolizine, quinolidine, quinoline, isoquinoline, naphthyridine, cinnoline, phthalazine, quinazoline, benzo[f]quinazoline, benzo[h]quinazoline, quinoxaline, benzoimidazole, indazole, carbazole, biscarbazole, phenanthridine, acridine, phenanthroline, phenazine, azatriphenylene, diazatriphenylene, hexaazatriphenylene, azacarbazole, azadibenzofuran, azadibenzothiophene, and dinaphtho[2′,3′:2,3:2′,3′:6,7]carbazole; and the like.

Among those described above, residual groups of compounds selected from the following group are preferred as the nitrogen-containing heteroaryl group.

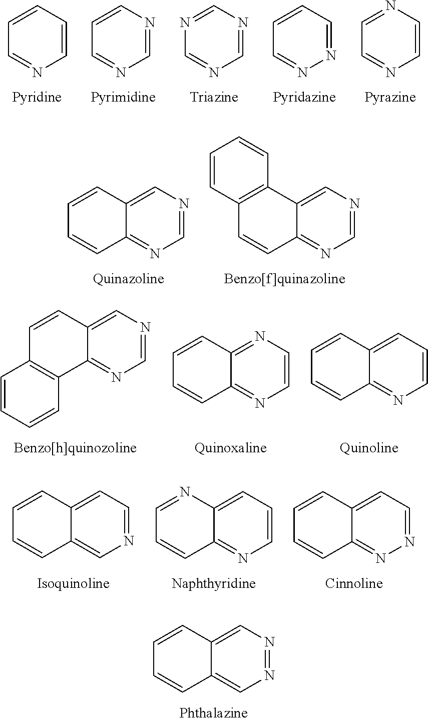

In the embodiment of the present invention, A is preferably a monovalent residual group of a compound represented by the following general formula (a2).

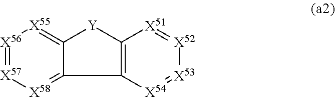

In the general formula (a2), X⁵¹ to X⁵⁸ are each independently C(R) or a nitrogen atom. Rs are each independently a hydrogen atom or a substituent, and two selected from plural Rs may be bonded to each other to form a ring.

Y is an oxygen atom, a sulfur atom, —NR^{c}, or —C(R^{d})(R^{e})—, and preferably an oxygen atom, a sulfur atom, or —C(R^{d})(R^{e})—. R^{c}, R^{d}, and R^{e} are each independently a hydrogen atom or a substituent, and R^{d} and R^{e} may be bonded to each other to form a ring. Examples of the substituent include those described above.

Furthermore, in the embodiment of the present invention, A is more preferably a monovalent residual group of a compound represented by the following general formula (a2-1).

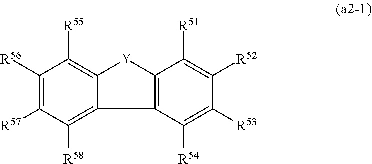

In the general formula (a2-1), Y is the same as that described regarding the general formula (a2).

R⁵¹ to R⁵⁸ are each independently a hydrogen atom or a substituent, and two selected from R⁵¹ to R⁵⁸ may be bonded to each other to form a ring. Examples of the substituent include those described above.

In the embodiment of the present invention, A is preferably a monovalent residual group of a compound represented by the following general formula (a3).

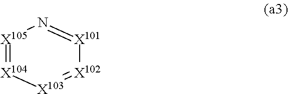

In the general formula (a3), X¹⁰¹ to X¹⁰⁵ are each independently C(R^{Y}) or a nitrogen atom.

R^{Y} is a hydrogen atom or a substituent, and plural R^{Y}s may be the same as or different from every other R^{Y}, and two selected from plural R^{Y}s may be bonded to each other to form a ring. Examples of the substituent include those described above.

Furthermore, in the embodiment of the present invention, A is more preferably a monovalent residual group of a compound represented by the following general formula (a3-1).

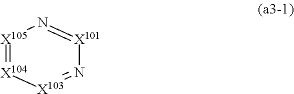

In the general formula (a3-1), X¹⁰¹ and X¹⁰³ to X¹⁰⁵ are each independently C(R^{Y}) or a nitrogen atom.

R^{Y} is a hydrogen atom or a substituent, and plural R^{Y}s may be the same as or different from every other R^{Y}. Two selected from plural R^{Y}s may be bonded to each other to form a ring. Examples of the substituent include those described above.

Furthermore, in the embodiment of the present invention, A is more preferably a monovalent residual group of a compound represented by the following general formula (a3-1-i).

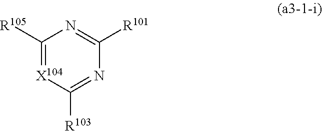

In the general formula (a3-1-i), X¹⁰⁴ is C(R¹⁰⁴) or a nitrogen atom.

R¹⁰¹ and R¹⁰³ to R¹⁰⁵ are each independently a hydrogen atom or a substituent, and two selected from R¹⁰³ to R¹⁰⁵ may be bonded to each other to form a ring. Examples of the substituent include those described above.

In addition, in the embodiment of the present invention, A is preferably a monovalent residual group of a compound represented by the following general formula (a3-2).

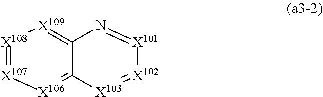

In the general formula (a3-2), X¹⁰¹ to X¹⁰³ and X¹⁰⁶ to X¹⁰⁹ are each independently C(R^{Y}) or a nitrogen atom.

R^{Y} is a hydrogen atom or a substituent, and plural R^{Y}s may be the same as or different from every other R^{Y}. Two selected from plural R^{Y}s may be bonded to each other to form a ring. Examples of the substituent include those described above.

Furthermore, in the embodiment of the present invention, A is more preferably a monovalent residual group of a compound represented by the following general formula (a3-3).

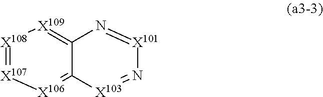

In the general formula (a3-3), X¹⁰¹, X¹⁰³, and X¹⁰⁶ to X¹⁰⁹ are each independently C(R^{Y}) or a nitrogen atom.

R^{Y} is a hydrogen atom or a substituent, and plural R^{Y}s may be the same as or different from every other R^{Y}. Two selected from plural R^{Y}s may be bonded to each other to form a ring. Examples of the substituent include those described above.

Furthermore, in the embodiment of the present invention, A is more preferably a monovalent residual group of a compound represented by the following general formula (a3-3-i).

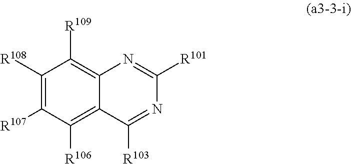

In the general formula (a3-3-i), R¹⁰¹, R¹⁰³, and R¹⁰⁶ to R¹⁰⁹ are each independently a hydrogen atom or a substituent, and two selected from R¹⁰³ and R¹⁰⁶ to R¹⁰⁹ may be bonded to each other to form a ring. Examples of the substituent include those described above.

### <R^{e}: L in the General Formula (1)>

In the general formula (1), L is a single bond, a substituted or unsubstituted arylene group having 6 to 60 ring carbon atoms, or a substituted or unsubstituted heteroarylene group having 3 to 50 ring atoms.

It is to be noted that the substituent which each of the arylene group and the heteroarylene group may have is the same as that described above.

In the embodiment of the present invention, the number of ring carbon atoms of the arylene group represented by L is 6 to 60, preferably 6 to 24, more preferably 6 to 18, still more preferably 6 to 12, and especially preferably 6 to 10.

Examples of the arylene group include a phenylene group, a naphthylene group, a biphenylene group, a terphenylene group, an acenaphthylene group, an anthrylene group, a benzanthrylene group, an aceanthrylene group, a phenanthrylene group, a benzophenanthrylene group, a phenalenylene group, a fluorenylene group, a spirobifluorenylene group, a benzofluorenylene group, a dibenzofluorenylene group, a picenylene group, a pentacenylene group, a pyrenylene group, a chrysenylene group, a benzochrysenylene group, an s-indacenylene group, an as-indacenylene group, a fluoranthenylene group, a benzofluoranthenylene group, a tetracenylene group, a triphenylenylene group, a benzotriphenylenylene group, a perylenylene group, a coronylene group, a dibenzanthrylene group, and the like. The arylene group may also be a connecting group of a combination of two or more selected from these divalent groups.

Of these arylene groups, divalent connecting groups selected from a phenylene group, a naphthylene group, a biphenylene group, a terphenylene group, an acenaphthylene group, a phenanthrylene group, a benzophenanthrylene group, a phenalenylene group, a fluorenylene group, a spirobifluorenylene group, a benzofluorenylene group, a dibenzofluorenylene group, a picenylene group, a chrysenylene group, a benzochrysenylene group, a fluoranthenylene group, a benzofluoranthenylene group, a triphenylenylene group, a benzotriphenylenylene group, a perylenylene group, and a coronylene group are preferred.

In the embodiment of the present invention, L is preferably a group represented by any one of the following general formulae (i) to (iii) or the like.

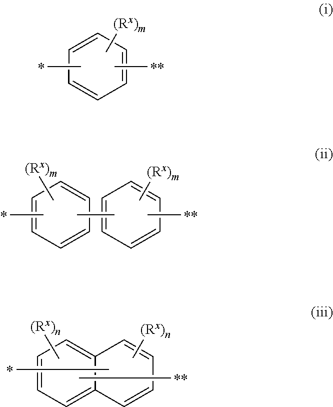

In the formulae (i) to (iii), R^{X}s are each independently a hydrogen atom or a substituent, and in the case where plural R^{X}s are present, plural R^{X}s may be the same as or different from every other R^{X}, and two selected from plural R^{X}s may be bonded to each other to form a ring structure. Examples of the substituent include those described above. It is to be noted that R^{X} represents a substituent of each of the benzene rings in the formulae (i) to (iii) and bonds to the carbon atom of each of the benzene rings.

Each m is independently an integer of 0 to 4, preferably an integer of 0 to 2, more preferably an integer of 0 to 1, and still more preferably 0.

Each n is independently an integer of 0 to 3, preferably an integer of 0 to 2, more preferably an integer of 0 to 1, and still more preferably 0.

Each of * and ** expresses a bonding position to the nitrogen atom or A in the general formula (1). Namely, one of * and ** expresses a bonding position to the nitrogen atom in the general formula (1), and the other expresses a bonding position to A in the general formula (1).

It is to be noted that examples of the group in the case where two selected from plural R^{X}s in the formula (i) or (ii) are bonded to each other to form a ring structure include groups represented by the following formulae. These groups are also included in the group represented by the formula (i) or (ii).

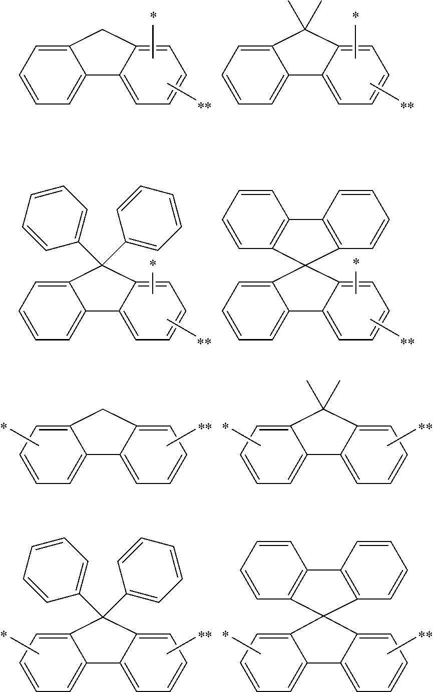

In the foregoing formulae, each of * and ** expresses a bonding position to the nitrogen atom or A in the general formula (1). The carbon atoms at other bonding positions in the foregoing formulae may be bonded to the aforementioned substituents.

Specific examples of the arylene group represented by L include groups represented by the following formulae.

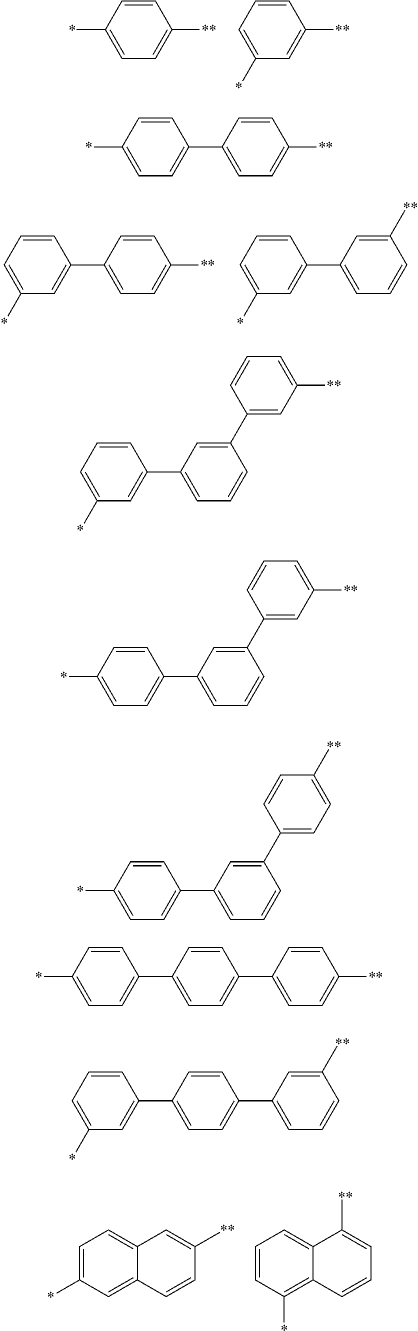

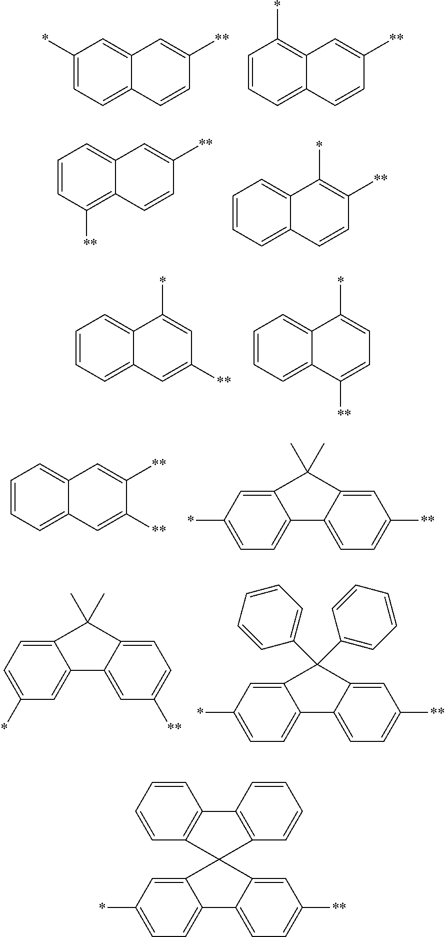

In the foregoing formulae, each of * and ** expresses a bonding position to the nitrogen atom or A in the general formula (1). The carbon atoms at other bonding positions in the foregoing formulae may be bonded to the aforementioned substituents.

In the general formula (1), the number of ring atoms of the heteroarylene group represented by L is 3 to 50, preferably 3 to 18, more preferably 3 to 13, and especially preferably 3 to 10.

Examples of the heteroarylene group represented by L in the general formula (1) include a divalent residual group of a nitrogen-containing heterocyclic compound, such as pyrrole, pyridine, imidazopyridine, pyrazole, triazole, tetrazole, indole, isoindole, carbazole, etc.; a divalent residual group of an oxygen-containing heterocyclic compound, such as furan, benzofuran, isobenzofuran, dibenzofuran, oxazole, oxadiazole, benzoxazole, benzonaphthofuran, dinaphthofuran, etc.; a divalent residual group of a sulfur-containing heterocyclic compound, such as thiophene, benzothiophene, dibenzothiophene, thiazole, thiadiazole, benzothiazole, benzonaphthothiophene, dinaphthothiophene, etc.; and the like. The heteroarylene group may also be a connecting group of a combination of two or more selected from these divalent groups.

Of these heteroarylene groups, a divalent residual group of a nitrogen-containing heterocyclic compound, such as pyridine, indole, carbazole, benzocarbazole, etc.; a divalent residual group of an oxygen-containing heterocyclic compound, such as dibenzofuran, benzonaphthofuran, etc.; a divalent residual group of a sulfur-containing heterocyclic compound, such as dibenzothiophene, benzonaphthothiophene, etc.; and the like are preferred.

As the embodiment of the present invention, a group represented by any one of the following general formulae (iv) to (vii) is preferred.

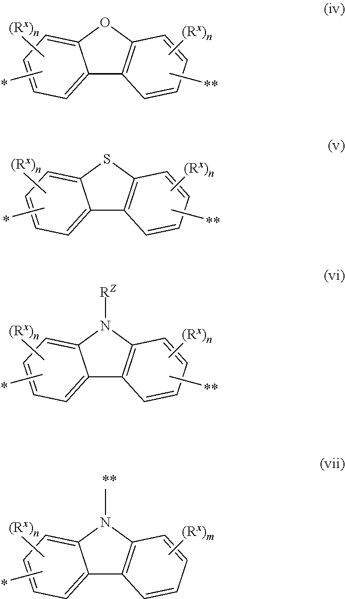

In the formulae (iv) to (vii), R^{X}s are each independently a hydrogen atom or a substituent, and in the case where plural R^{X}s are present, plural R^{X}s may be the same as or different from every other R^{X}, and two selected from plural R^{X}s may be bonded to each other to form a ring structure. Examples of the substituent include those described above. It is to be noted that R^{X} represents a substituent of each of the benzene rings in the formulae (iv) to (vii) and bonds to the carbon atom of each of the benzene rings.

In the formula (vi), R^{Z} is a hydrogen atom or a substituent.

Each m is independently an integer of 0 to 4, preferably an integer of 0 to 2, more preferably an integer of 0 to 1, and still more preferably 0.

Each n is independently an integer of 0 to 3, preferably an integer of 0 to 2, more preferably an integer of 0 to 1, and still more preferably 0.

Each of * and ** expresses a bonding position to the nitrogen atom or A in the general formula (1). Namely, one of * and ** expresses a bonding position to the nitrogen atom in the general formula (1), and the other expresses a bonding position to A in the general formula (1).

<R^{e}: X¹ to X¹⁶ in the general formula (1)>

In the general formula (1), two selected from X¹ to X⁴ are a carbon atom bonding to *¹ or *², respectively, and the other two of X¹ to X⁴ are each independently C(R) or a nitrogen atom, and preferably C(R).

Two selected from X⁹ to X¹² are a carbon atom bonding to *³ or *⁴, respectively, and the other two of X⁹ to X¹² are each independently C(R) or a nitrogen atom, and preferably C(R).

X⁵ to X⁸ and X¹³ to X¹⁶ are each independently C(R) or a nitrogen atom.

In the embodiment of the present invention, it is preferred that a pair selected from X¹ and X², X² and X³, and X³ and X⁴ is a carbon atom bonding to *¹ or *²; and that a pair selected from X⁹ and X¹⁰, X¹⁰ and X¹¹, and X¹¹ and X¹² is a carbon atom bonding to *³ or *⁴.

In the general formula (1), X¹ to X¹⁶ which do not take part in the formation of a prescribed ring structure represented by the general formula (1) are each independently C(R) or a nitrogen atom, and in the embodiment of the present invention, all of such X¹ to X¹⁶ are preferably CO.

Here, R is a hydrogen atom or a substituent, and plural Rs may be the same as or different from every other R. Two selected from plural Rs may be bonded to each other to form a ring. Examples of the substituent include those described above.

<R^{A} and R^{B} in the General Formula (1)>

In the general formula (1), R^{A} and R^{B} are each independently a hydrogen atom, a substituted or unsubstituted alkyl group having 1 to 20 carbon atoms, a substituted or unsubstituted aryl group having 6 to 50 ring carbon atoms, a substituted or unsubstituted heteroaryl group having 3 to 50 ring atoms, a halogen atom, a mono-substituted, di-substituted, or tri-substituted silyl group having a substituent selected from an alkyl group having 1 to 50 carbon atoms and an aryl group having 6 to 50 ring carbon atoms, a substituted or unsubstituted alkoxy group having 1 to 20 carbon atoms, a substituted or unsubstituted aryloxy group having 6 to 50 ring carbon atoms, or a cyano group. R^{A} and R^{B} may be the same as or different from each other. R^{A} and R^{B} may be bonded to each other to form a ring.

It is to be noted that the substituent which such a group may have is the same as that described above.

The number of carbon atoms of the alkyl group which may be selected as R^{A} and R^{B} is 1 to 20, preferably 1 to 18, and more preferably 1 to 8.

Examples of the alkyl group include a methyl group, an ethyl group, an n-propyl group, an isopropyl group, an n-butyl group, an isobutyl group, an s-butyl group, a t-butyl group, a pentyl group, a hexyl group, a heptyl group, an octyl group, a nonyl group, a decyl group, an undecyl group, a dodecyl group, a tridecyl group, a tetradecyl group, an octadecyl group, a tetracosanyl group, a tetracontanyl group, and the like. Of these, a methyl group, an ethyl group, an n-propyl group, an isopropyl group, an n-butyl group, an isobutyl group, an s-butyl group, a t-butyl group, a pentyl group, a hexyl group, a heptyl group, an octyl group, a nonyl group, a decyl group, an undecyl group, a dodecyl group, a tridecyl group, a tetradecyl group, and an octadecyl group are preferred, with a methyl group, an ethyl group, an n-propyl group, an isopropyl group, an n-butyl group, an isobutyl group, an s-butyl group, a t-butyl group, a pentyl group, a hexyl group, a heptyl group, and an octyl group being more preferred. In the case where an isomeric group is present in each of the above-described groups, the isomeric group is also included therein.

The number of ring carbon atoms of the aryl group which may be selected as R^{A} and R^{B} is 6 to 50, preferably 6 to 18, more preferably 6 to 13, still more preferably 6 to 12, and especially preferably 6 to 10. The aryl group may be any of a non-fused aryl group, a fused aryl group, and a combination thereof.

Examples of the aryl group include a phenyl group, a biphenylyl group, a terphenylyl group, a quaterphenylyl group, a quinquephenylyl group, a naphthyl group (e.g., a 1-naphthyl group and a 2-naphthyl group), an acenaphthelenyl group, an anthryl group, a benzanthryl group, an aceanthryl group, a phenanthryl group, a benzophenanthryl group, a phenalenyl group, a fluorenyl group (inclusive of a 9,9-dimethylfluorenyl group, a 9,9-diphenylfluorenyl group, and a 9,9′-spirobifluorenyl group), a benzofluorenyl group, a dibenzofluorenyl group, a picenyl group, a pentacenyl group, a pyrenyl group, a chrysenyl group, a benzochrysenyl group, a fluoranthenyl group, a benzofluoranthenyl group, a tetracenyl group, a perylenyl group, a coronyl group, a dibenzanthryl group, a naphthylphenyl group, an s-indacenyl group, an as-indacenyl group, a triphenylenyl group, a benzotriphenylenyl group, and the like. In the case where an isomeric group is present in each of the above-described groups, the isomeric group is also included therein.

More specifically, aryl groups selected from the following group are preferred as the aryl group which may be selected as R^{A} and R^{B} are preferred.

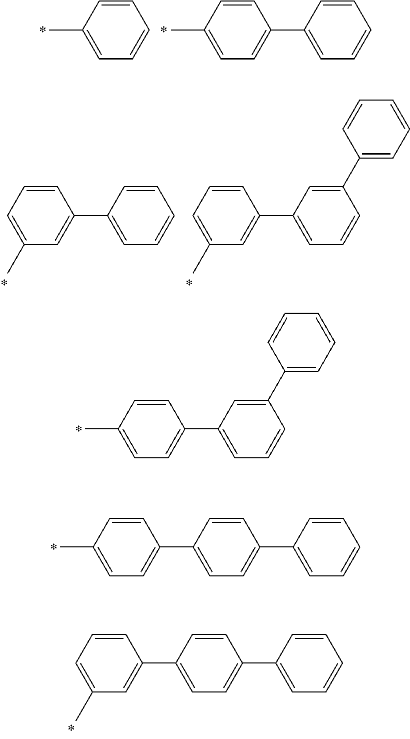

In the foregoing formulae, * expresses a bonding position. The carbon atoms at a position other than the bonding position in each of the foregoing formulae may be bonded to the substituent.

The number of ring atoms of the heteroaryl group which may be selected as R^{A} and R^{B} is 3 to 50, preferably 5 to 20, more preferably 5 to 14, and still more preferably 5 to 10.

The heteroaryl group contains at least one, preferably 1 to 5, more preferably 1 to 3, and more preferably 1 to 2 hetero groups which are the same as or different from each other.

Examples of the heteroaryl group include a pyrrolyl group, a furyl group, a thienyl group, a pyridyl group, a pyridazinyl group, a pyrimidinyl group, a pyrazinyl group, a triazinyl group, an imidazolyl group, an oxazolyl group, a thiazolyl group, a pyrazolyl group, an isooxazolyl group, an isothiazolyl group, an oxadiazolyl group, a thiadiazolyl group, a triazolyl group, a tetrazolyl group, an indolyl group, an isoindolyl group, a benzofuranyl group, an isobenzofuranyl group, a benzothiophenyl group, an isobenzothiophenyl group, an indolizinyl group, a quinolizinyl group, a quinolyl group, an isoquinolyl group, a cinnolyl group, a phthalazinyl group, a quinazolinyl group, a quinoxalinyl group, a benzimidazolyl group, a benzoxazolyl group, a benzothiazolyl group, an indazolyl group, a benzisoxazolyl group, a benzisothiazolyl group, a dibenzofuranyl group, a dibenzothiophenyl group, a carbazolyl group, a biscarbazolyl group, a phenanthridinyl group, an acridinyl group, a phenanthrolinyl group, a phenazinyl group, a phenothiazinyl group, a phenoxazinyl group, an azatriphenylenyl group, a diazatriphenylenyl group, a xanthenyl group, an azacarbazolyl group, an azadibenzofuranyl group, an azadibenzothiophenyl group, a benzofuranobenzothiophenyl group, a benzothienobenzothiophenyl group, a dibenzofuranonaphthyl group, a dibenzothienonaphthyl group, a dinaphthothienothiophenyl group, and a dinaphtho[2′,3′:2,3:2′,3′:6,7]carbazolyl group.

Examples of the halogen atom which may be selected as R^{A} and R^{B} include a fluorine atom, a chlorine atom, a bromine atom, an iodine atom, and the like.

Examples of the mono-substituted, di-substituted, or tri-substituted silyl group having a substituent selected from an alkyl group having 1 to 50 carbon atoms and an aryl group having 6 to 50 ring carbon atoms, which may be selected as R^{A} and R^{B}, include a trialkylsilyl group, a triarylsilyl group, a monoalkyldiarylsilyl group, and a dialkylmonoarylsilyl group. Specific examples thereof include a trimethylsilyl group, a triethylsilyl group, a tributylsilyl group, a trioctylsilyl group, a triisobutylsilyl group, a dimethylethylsilyl group, a dimethylisopropylsilyl group, a dimethylpropylsilyl group, a dimethylbutylsilyl group, a dimethyl-tertiary-butylsilyl group, a diethylisopropylsilyl group, a phenyldimethylsilyl group, a diphenylmethylsilyl group, a diphenyl-tertiary-butyl group, a triphenylsilyl group, and the like. Of these, a trimethylsilyl group, a triethylsilyl group, and a tributylsilyl group are preferred.

The alkoxy group having 1 to 20 carbon atoms, which may be selected as R^{A} and R^{B}, is a group represented by —OR′, and R′ represents the aforementioned alkyl group having 1 to 20 carbon atoms.

Specific examples of the alkoxy group include a methoxy group, an ethoxy group, a propoxy group, a butoxy group, a pentyloxy group, a hexyloxy group, and the like.

The aryloxy group having 6 to 50 ring carbon atoms, which may be selected as R^{A} and R^{B}, is a group represented by —OR″, and R″ represents the aforementioned aryl group having 6 to 50 carbon atoms.

Specific examples of the aryloxy group include a phenoxy group, a biphenyloxy group, a 1-naphthyloxy group, a 2-naphthyloxy group, a p-tertphenyl-4-yloxy group, a p-tolyloxy group, and the like.

In the compounds in the embodiment of the present invention, in the case where the aforementioned A, R, R^{A}, R^{B}, R¹ to R¹⁶, R^{a}, R^{b}, R^{X}, R^{Y}, and R^{Z} are a substituted or unsubstituted aryl group, the aryl group is preferably a monovalent residual group of a compound selected from the following [Group A] or [Group B].

### [Group A]

A group consisting of phenanthrene, triphenylene, benzimidazole, indole, carbazole, benzofuran, dibenzofuran, benzothiophene, and dibenzothiophene.

### [Group B]

A group consisting of chrysene, picene, fluoranthene, benzophenanthrene, quinoxaline, quinazoline, naphthylidine, phthalazine, phenanthroline, naphthalene, quinoline, and isoquinoline.

It is to be noted that the monovalent residual group of the compound selected from the aforementioned Group A or Group B may have the aforementioned substituent in place of the hydrogen atom bonding to the carbon atom which the residual group has.

### Compound of the Embodiment of the Present Invention

The compound of the embodiment represented by the general formula (1) is preferably a compound represented by the following general formula (2) (hereinafter also referred to as “compound (2)”).

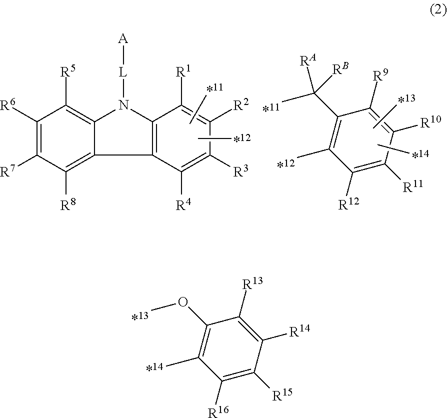

In the general formula (2), A, L, R^{A}, and R^{B} are the same as those described regarding the general formula (1).

Two groups selected from R¹ to R⁴ split off, the carbon atoms bonded to the subject groups bond to *¹¹ or *¹², and the other two of R¹ to R⁴ are each independently a hydrogen atom or a substituent.

Two groups selected from R⁹ to R¹² split off, the carbon atoms bonded to the subject groups bond to *¹³ or *¹⁴, and the other two of R⁹ to R¹² are each independently a hydrogen atom or a substituent.

R⁵ to R⁸ and R¹³ to R¹⁶ are each independently a hydrogen atom or a substituent and may be the same as or different from each other.

It is to be noted that two selected from R¹ to R¹⁶ which do not take part in the bonding to *¹¹ to *¹⁴ may be bonded to each other to form a ring.

It is to be noted that in the embodiment of the present invention, it is preferred that in the general formula (2), two groups of a pair selected from R¹ and R², R² and R³, and R³ and R⁴ split off, the carbon atoms bonded to the subject groups bond to *¹¹ or *¹²; and that two groups of a pair selected from R⁹ and R¹⁰, R¹⁰ and R¹¹, and R¹¹ and R¹² split off, the carbon atoms bonded to the subject groups bond to *¹³ or *¹⁴.

The compound of the embodiment represented by the general formula (1) is more preferably a compound represented by the following general formula (3) (hereinafter also referred to as “compound (3)”).

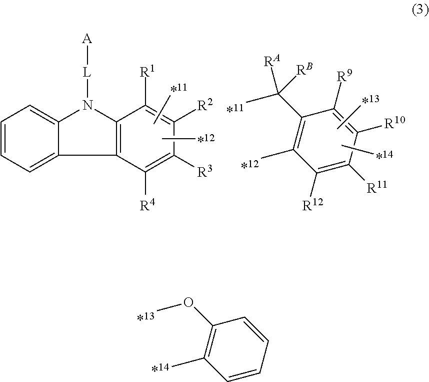

In the general formula (3), A, L, R^{A}, and R^{B} are the same as those described regarding the general formula (1).

Two groups selected from R¹ to R⁴ split off, the carbon atoms bonded to the subject groups bond to *¹¹ or *¹², and the other two of R¹ to R⁴ are a hydrogen atom.

Two groups selected from R⁹ to R¹² split off, the carbon atoms bonded to the subject groups bond to *¹³ or *¹⁴, and the other two of R⁹ to R¹² are a hydrogen atom.

It is to be noted that in the embodiment of the present invention, it is preferred that in the general formula (3), two groups of a pair selected from R¹ and R², R² and R³, and R³ and R⁴ split off, the carbon atoms bonded to the subject groups bond to *¹¹ or *¹²; and that two groups of a pair selected from R⁹ and R¹⁰, R¹⁰ and R¹¹, and R¹¹ and R¹² split off, the oxygen atom bonded to the subject groups bond to *¹³ or *¹⁴.

The compound of the embodiment of the compound (2) is preferably a compound represented by the following general formula (4) (hereinafter also referred to as “compound (4)”).

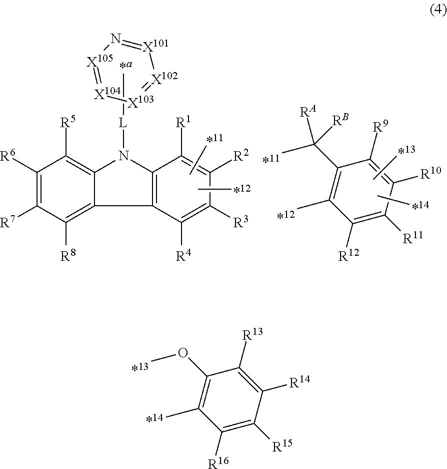

In the general formula (4), L, R^{A}, and R^{B} are the same as those described regarding the general formula (1), and R¹ to R¹⁶ and *¹¹ to *¹⁴ are the same as those described regarding the general formula (2).

X¹⁰¹ to X¹⁰⁵ are each independently a carbon atom bonding to *^{a}, C(R^{Y}), or a nitrogen atom. R^{Y} is a hydrogen atom or a substituent, and plural R^{Y}s may be the same as or different from every other R^{Y}. Two selected from plural R^{Y}s may be bonded to each other to form a ring. Examples of the substituent include those described above.

The compound of another embodiment of the compound (2) is more preferably a compound represented by the following general formula (5) (hereinafter also referred to as “compound (5)”).

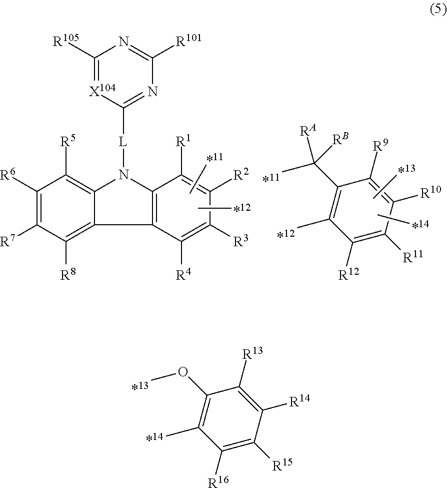

In the general formula (5), L, R^{A}, and R^{B} are the same as those described regarding the general formula (1), and R¹ to R¹⁶ and *¹¹ to *¹⁴ are the same as those described regarding the general formula (2).

X¹⁰⁴ is C(R¹⁰⁴) or a nitrogen atom. R¹⁰¹, R¹⁰⁴, and R¹⁰⁵ are each independently a hydrogen atom or a substituent, and R¹⁰⁴ and R¹⁰⁵ may be bonded to each other to form a ring. Examples of the substituent include those described above.

In the embodiment of the present invention, the compound represented by the general formula (2) (compound (2)) is preferably any one of compounds represented by the following general formulae (2-1) to (2-36) (hereinafter also referred to as “compounds (2-1) to (2-36)”).

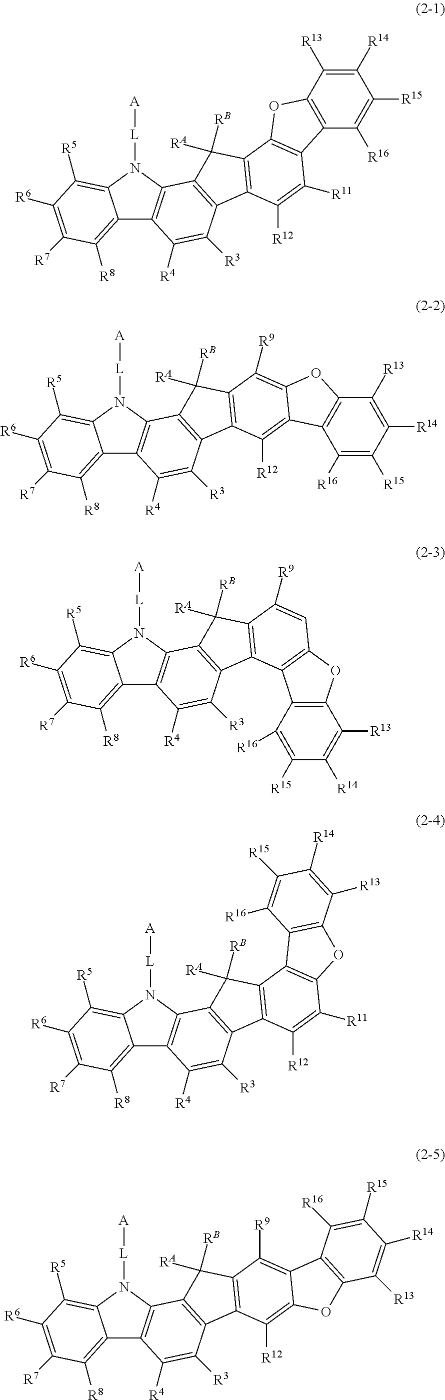

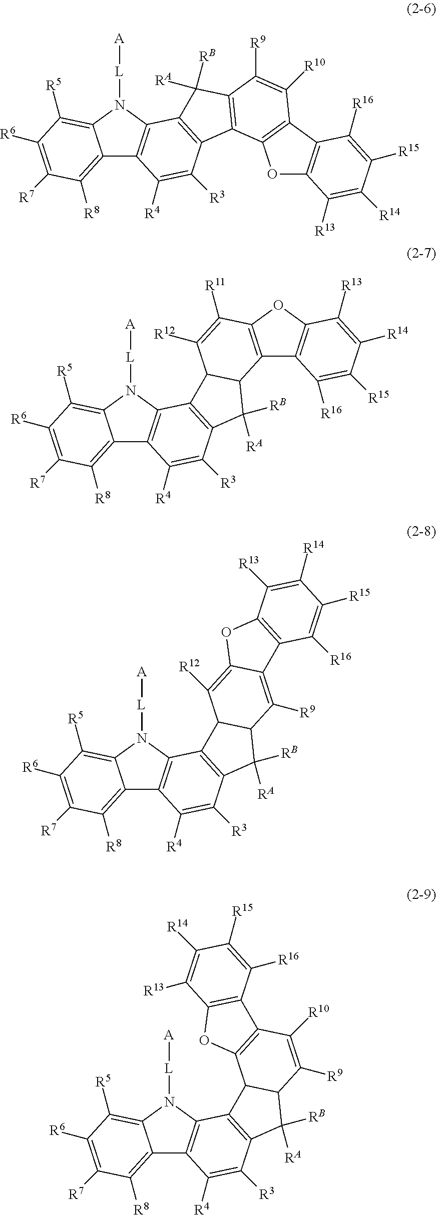

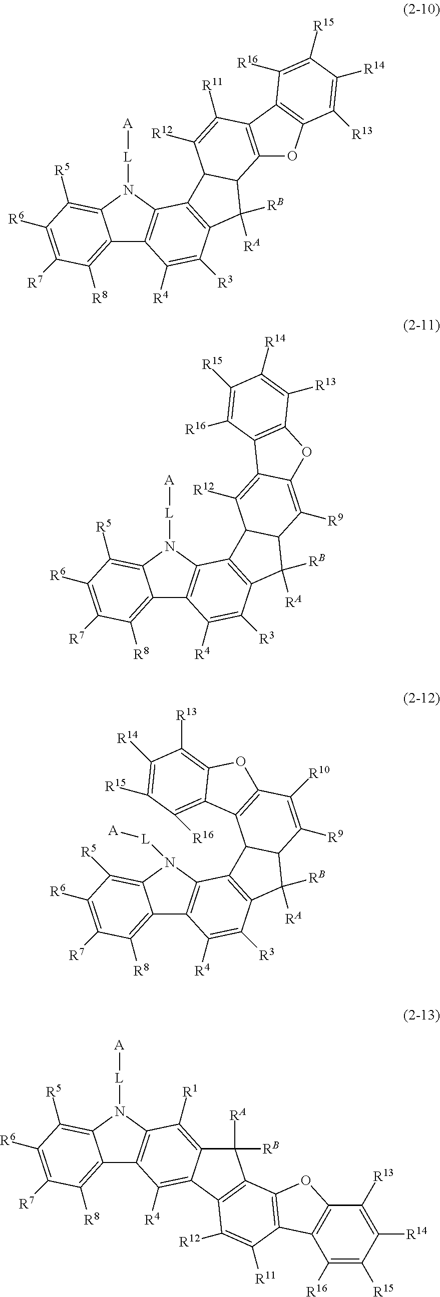

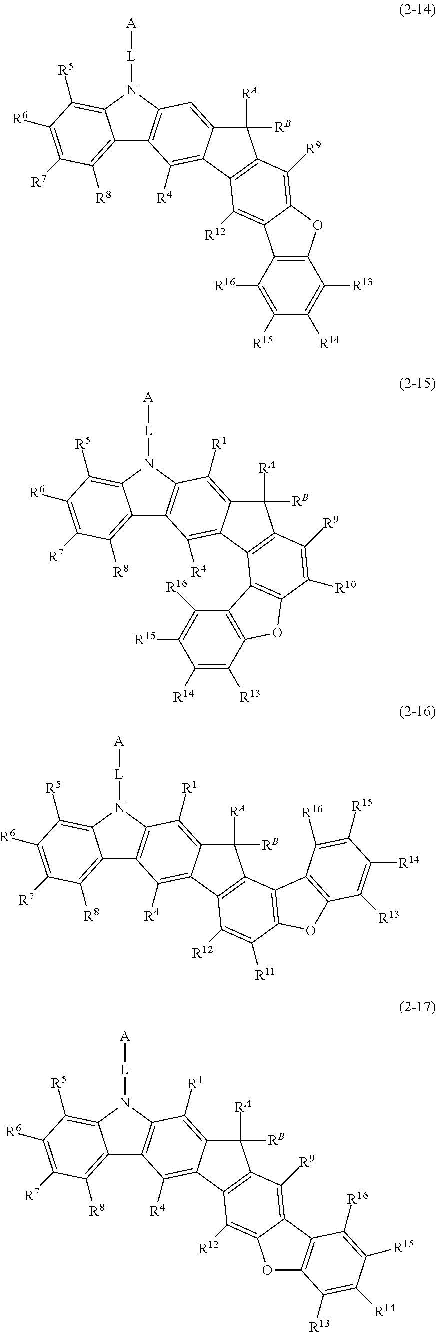

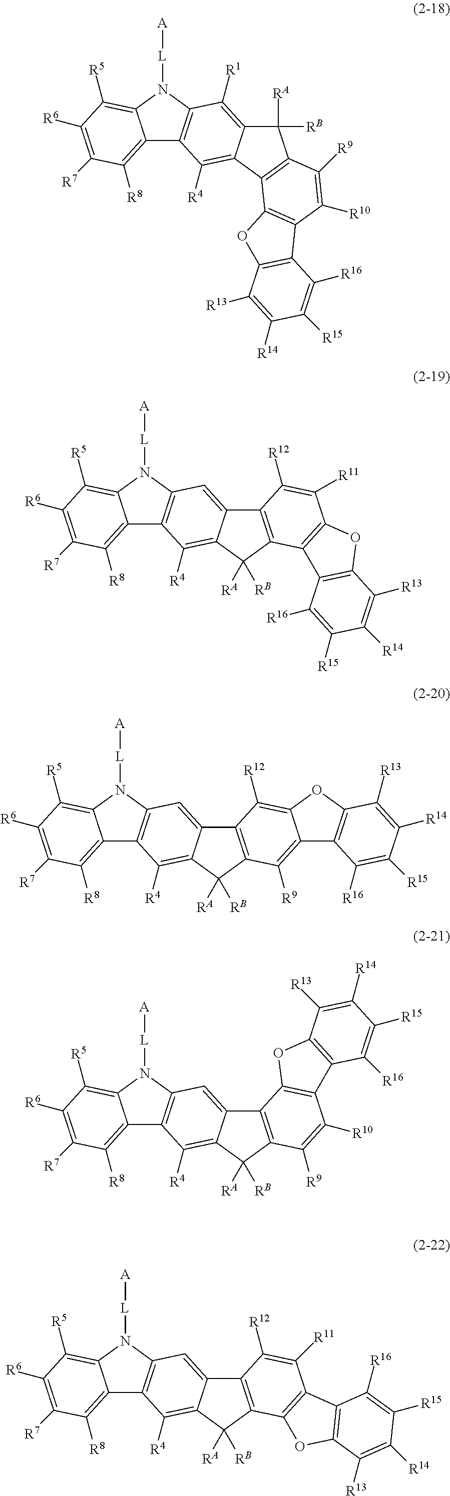

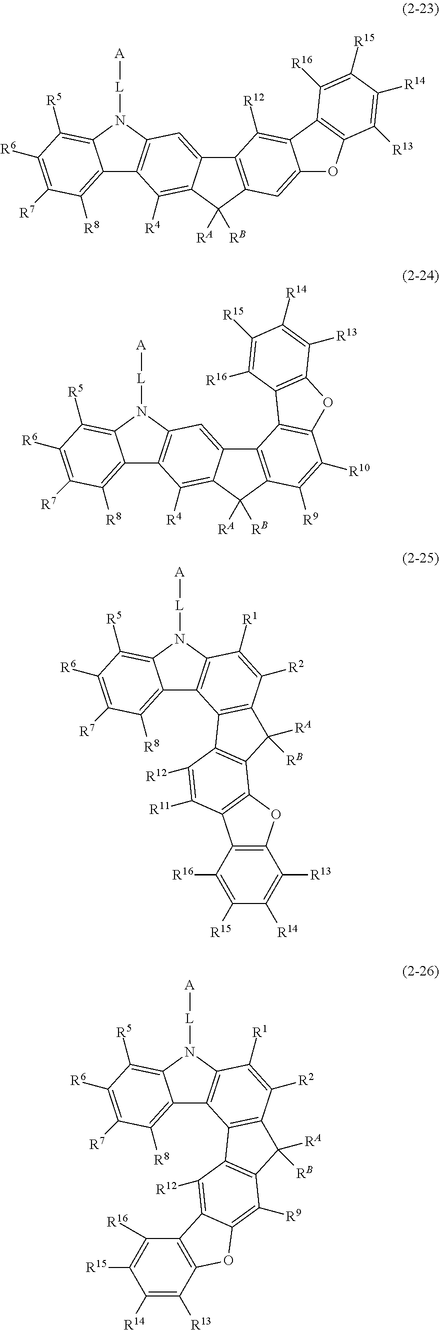

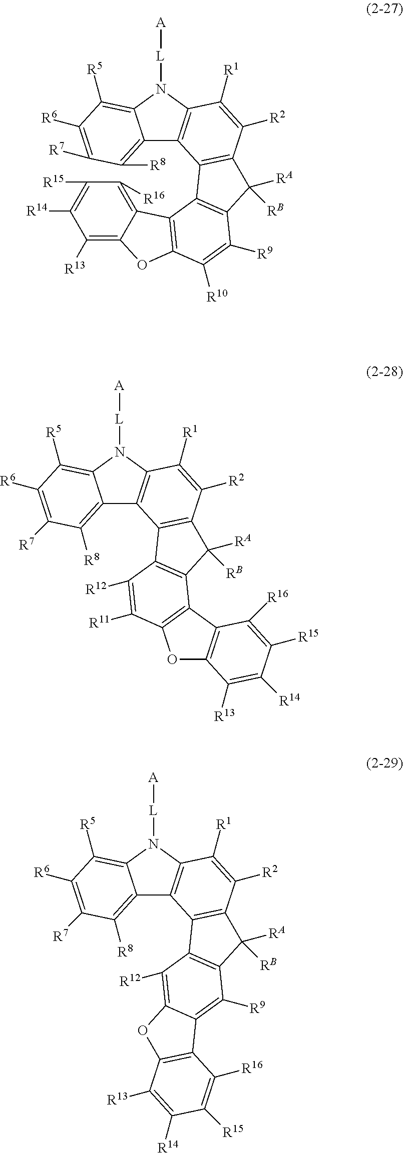

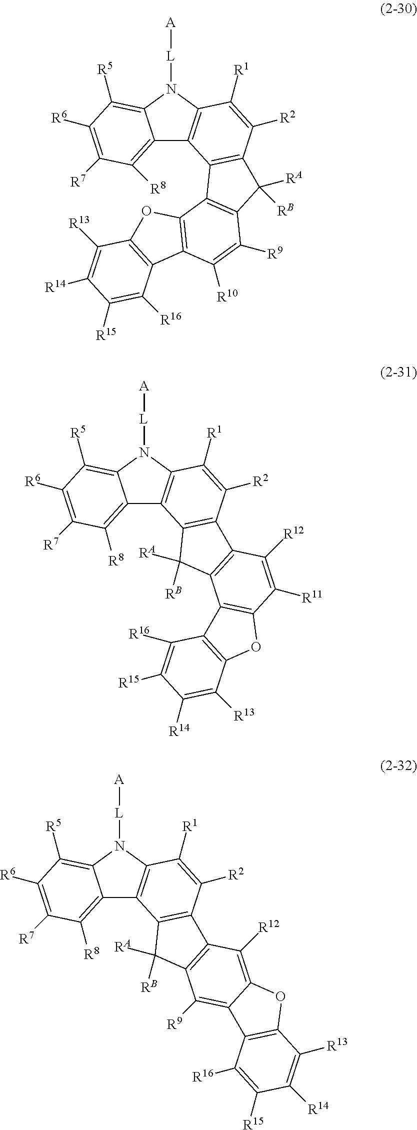

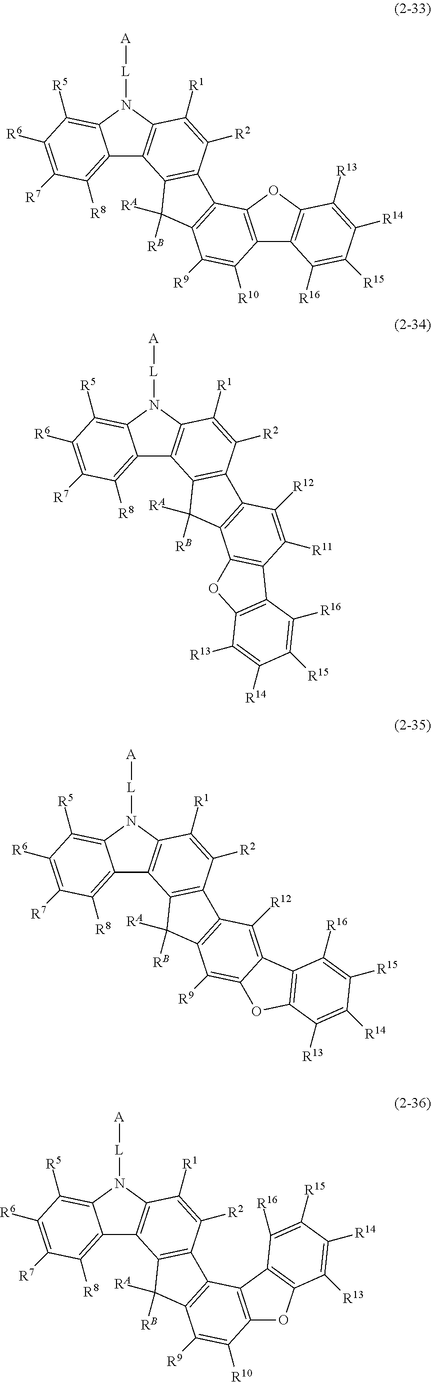

In the general formulae (2-1) to (2-36), A, L, R^{A}, and R^{B} are the same as those described regarding the general formula (1), and R¹ to R¹⁶ and *¹¹ to *¹⁴ are the same as those described regarding the general formula (2).

The compound of the embodiment of the present invention is more preferably a compound represented by any one of the general formulae (2-2), (2-3), (2-4), (2-6), (2-7), (2-8), (2-9), (2-10), (2-11), (2-12), (2-13), (2-14), (2-15), (2-16), (2-17), (2-18), (2-19), (2-21), (2-23), (2-24), (2-25), (2-26), (2-27), (2-28), (2-29), (2-30), (2-31), (2-32), (2-33), (2-34), (2-35), and (2-36).

Furthermore, the compound of the embodiment of the present invention is still more preferably a compound represented by any one of the general formulae (2-7), (2-9), (2-11), (2-12), (2-14), (2-15), (2-16), (2-18), (2-26), (2-27), (2-28), (2-30), (2-31), (2-33), (2-35), and (2-36).

Furthermore, the compound of the embodiment of the present invention is yet still more preferably a compound represented by any one of the general formulae (2-7), (2-9), (2-11), (2-14), (2-16), (2-18), (2-31), (2-33), and (2-35).

Specific examples of the compound of the embodiment of the present invention are hereunder described, but it should not be construed that the present invention is limited thereto.

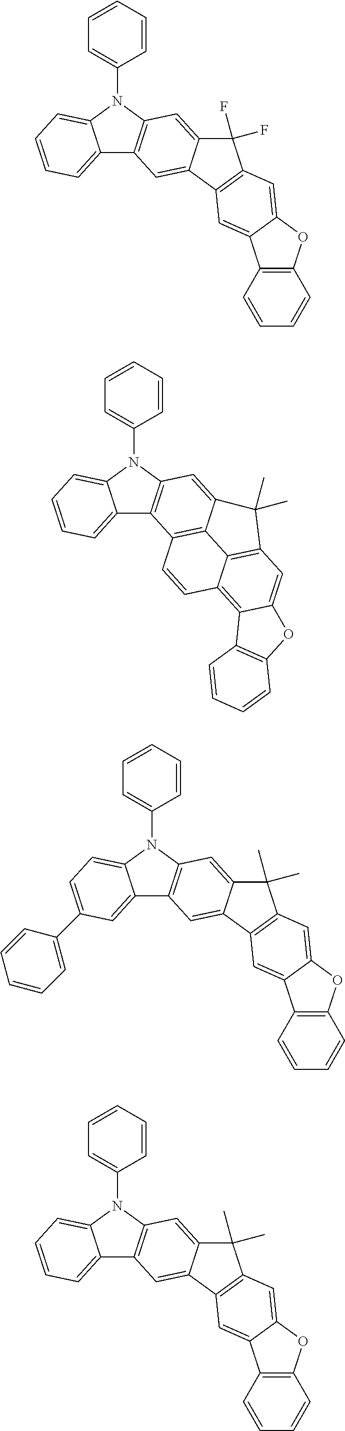 [SMILES: FC1(F)c2cc3oc4ccccc4c3cc2-c2cc3c4ccccc4n(-c4ccccc4)c3cc21 ; CC1(C)c2cc3oc4ccccc4c3c3ccc4c(c1cc1c4c4ccccc4n1-c1ccccc1)c23 ; CC1(C)c2cc3oc4ccccc4c3cc2-c2cc3c4cc(-c5ccccc5)ccc4n(-c4ccccc4)c3cc21 ; CC1(C)c2cc3oc4ccccc4c3cc2-c2cc3c4ccccc4n(-c4ccccc4)c3cc21]

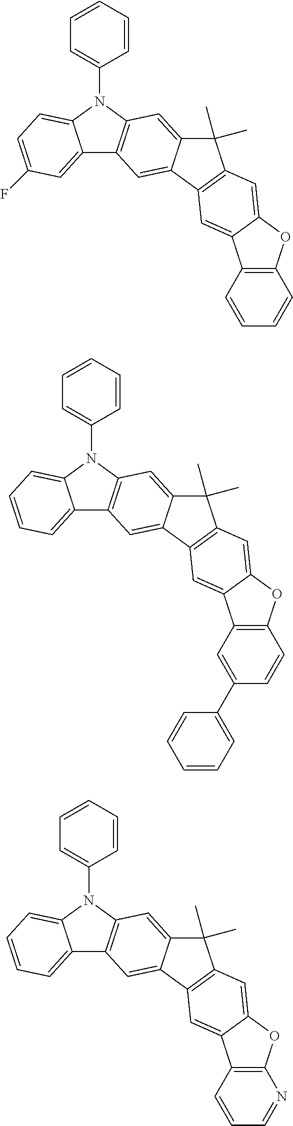 [SMILES: CC1(C)c2cc3oc4ccccc4c3cc2-c2cc3c4cc(F)ccc4n(-c4ccccc4)c3cc21 ; CC1(C)c2cc3oc4ccc(-c5ccccc5)cc4c3cc2-c2cc3c4ccccc4n(-c4ccccc4)c3cc21 ; CC1(C)c2cc3oc4ncccc4c3cc2-c2cc3c4ccccc4n(-c4ccccc4)c3cc21]

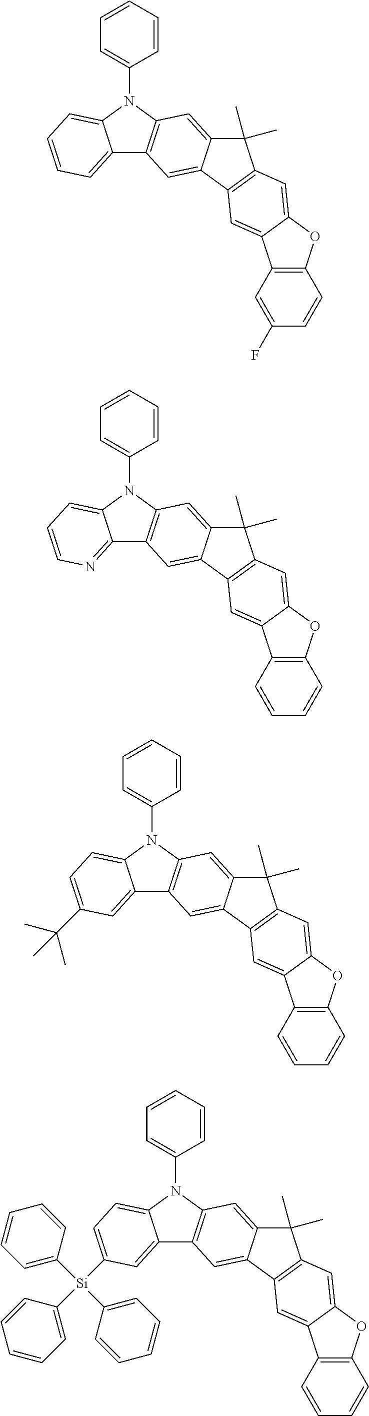 [SMILES: CC1(C)c2cc3oc4ccc(F)cc4c3cc2-c2cc3c4ccccc4n(-c4ccccc4)c3cc21 ; CC1(C)c2cc3oc4ccccc4c3cc2-c2cc3c4ncccc4n(-c4ccccc4)c3cc21 ; CC(C)(C)c1ccc2c(c1)c1cc3c(cc1n2-c1ccccc1)C(C)(C)c1cc2oc4ccccc4c2cc1-3 ; CC1(C)c2cc3oc4ccccc4c3cc2-c2cc3c4cc([Si](c5ccccc5)(c5ccccc5)c5ccccc5)ccc4n(-c4ccccc4)c3cc21]

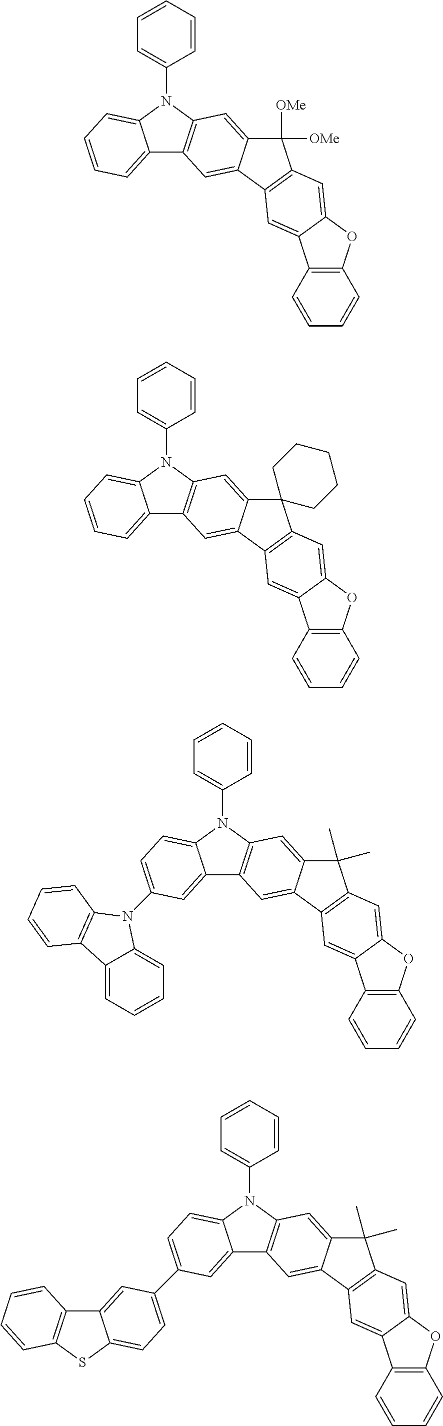 [SMILES: COC1(OC)c2cc3oc4ccccc4c3cc2-c2cc3c4ccccc4n(-c4ccccc4)c3cc21 ; c1ccc(-n2c3ccccc3c3cc4c(cc32)C2(CCCCC2)c2cc3oc5ccccc5c3cc2-4)cc1 ; CC1(C)c2cc3oc4ccccc4c3cc2-c2cc3c4cc(-n5c6ccccc6c6ccccc65)ccc4n(-c4ccccc4)c3cc21 ; CC1(C)c2cc3oc4ccccc4c3cc2-c2cc3c4cc(-c5ccc6sc7ccccc7c6c5)ccc4n(-c4ccccc4)c3cc21]

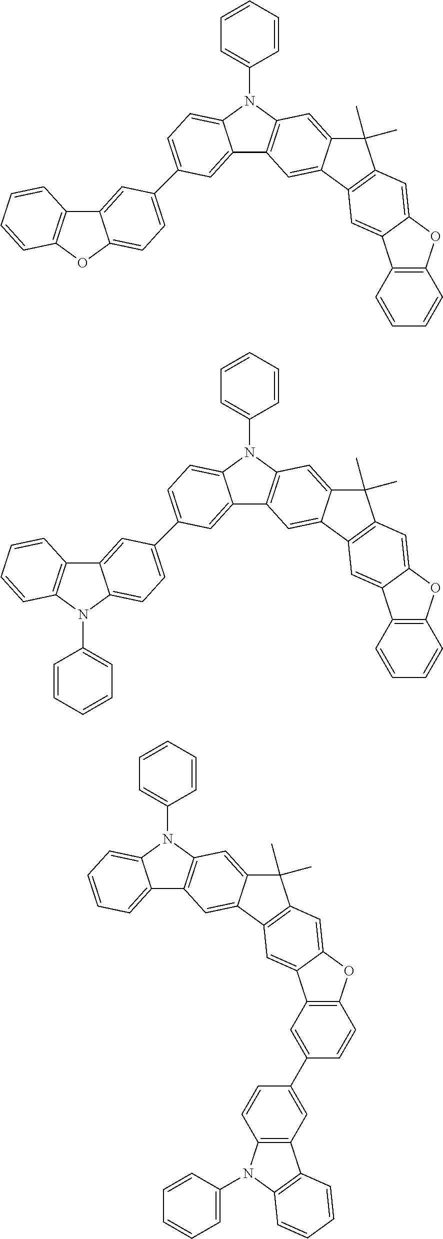 [SMILES: CC1(C)c2cc3oc4ccccc4c3cc2-c2cc3c4cc(-c5ccc6oc7ccccc7c6c5)ccc4n(-c4ccccc4)c3cc21 ; CC1(C)c2cc3oc4ccccc4c3cc2-c2cc3c4cc(-c5ccc6c(c5)c5ccccc5n6-c5ccccc5)ccc4n(-c4ccccc4)c3cc21 ; CC1(C)c2cc3oc4ccc(-c5ccc6c(c5)c5ccccc5n6-c5ccccc5)cc4c3cc2-c2cc3c4ccccc4n(-c4ccccc4)c3cc21]

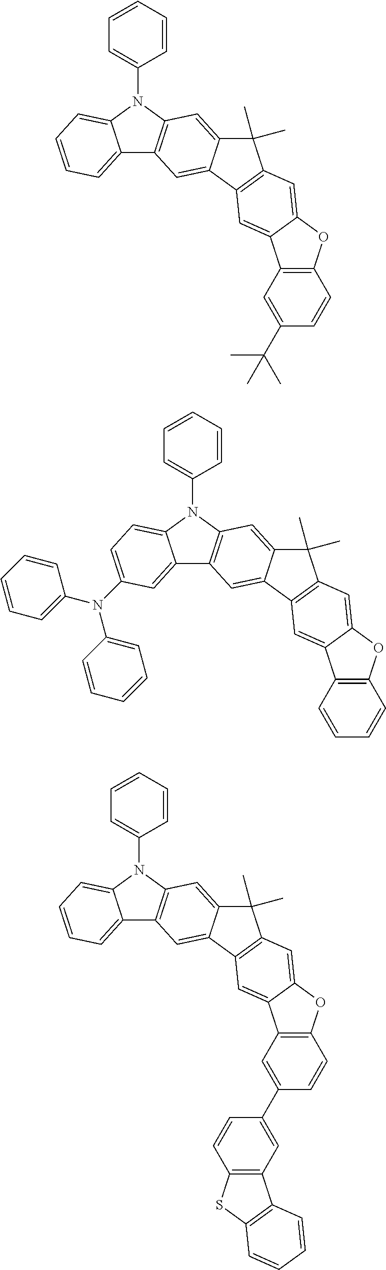 [SMILES: CC(C)(C)c1ccc2oc3cc4c(cc3c2c1)-c1cc2c3ccccc3n(-c3ccccc3)c2cc1C4(C)C ; CC1(C)c2cc3oc4ccccc4c3cc2-c2cc3c4cc(N(c5ccccc5)c5ccccc5)ccc4n(-c4ccccc4)c3cc21 ; CC1(C)c2cc3oc4ccc(-c5ccc6sc7ccccc7c6c5)cc4c3cc2-c2cc3c4ccccc4n(-c4ccccc4)c3cc21]

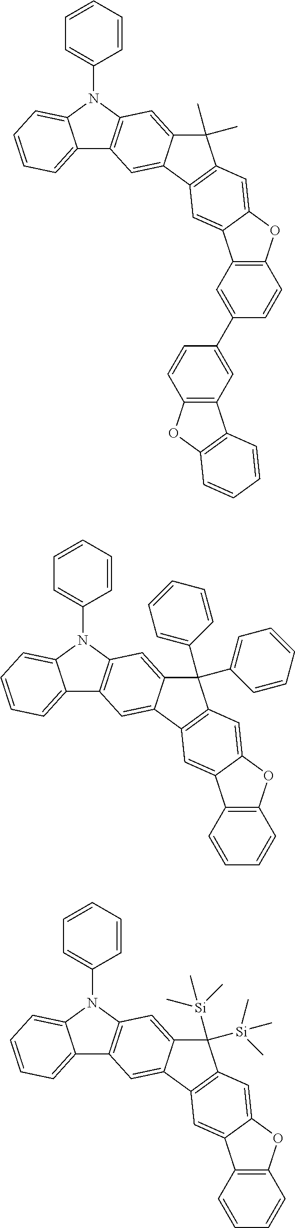 [SMILES: CC1(C)c2cc3oc4ccc(-c5ccc6oc7ccccc7c6c5)cc4c3cc2-c2cc3c4ccccc4n(-c4ccccc4)c3cc21 ; c1ccc(-n2c3ccccc3c3cc4c(cc32)C(c2ccccc2)(c2ccccc2)c2cc3oc5ccccc5c3cc2-4)cc1 ; C[Si](C)(C)C1([Si](C)(C)C)c2cc3oc4ccccc4c3cc2-c2cc3c4ccccc4n(-c4ccccc4)c3cc21]

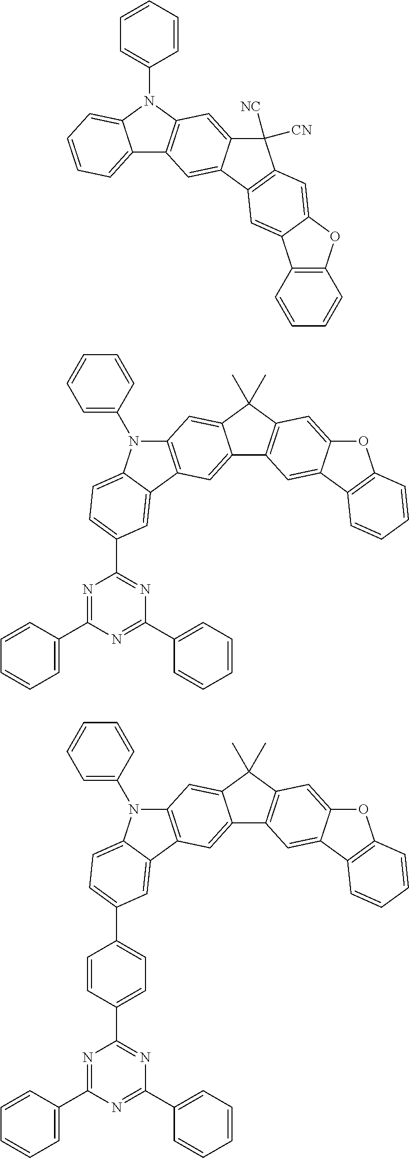 [SMILES: N#CC1(C#N)c2cc3oc4ccccc4c3cc2-c2cc3c4ccccc4n(-c4ccccc4)c3cc21 ; CC1(C)c2cc3oc4ccccc4c3cc2-c2cc3c4cc(-c5nc(-c6ccccc6)nc(-c6ccccc6)n5)ccc4n(-c4ccccc4)c3cc21 ; CC1(C)c2cc3oc4ccccc4c3cc2-c2cc3c4cc(-c5ccc(-c6nc(-c7ccccc7)nc(-c7ccccc7)n6)cc5)ccc4n(-c4ccccc4)c3cc21]

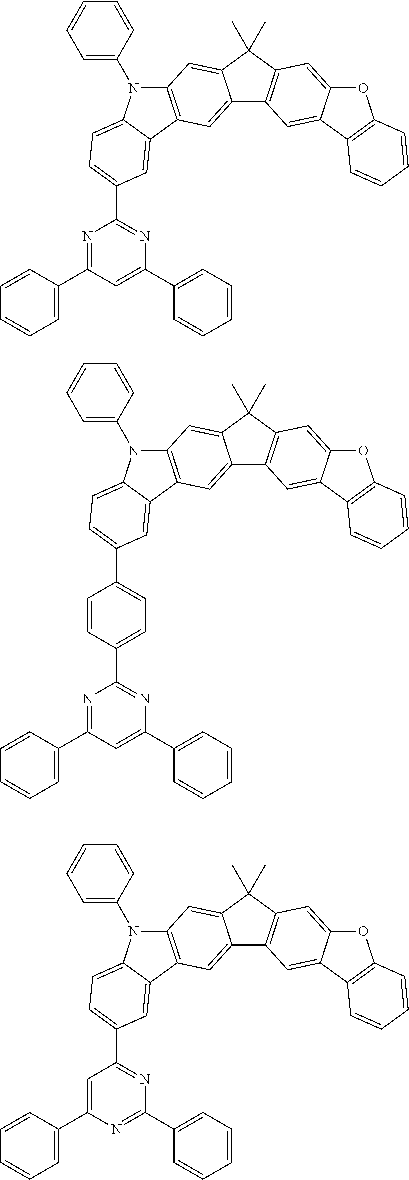 [SMILES: CC1(C)c2cc3oc4ccccc4c3cc2-c2cc3c4cc(-c5nc(-c6ccccc6)cc(-c6ccccc6)n5)ccc4n(-c4ccccc4)c3cc21 ; CC1(C)c2cc3oc4ccccc4c3cc2-c2cc3c4cc(-c5ccc(-c6nc(-c7ccccc7)cc(-c7ccccc7)n6)cc5)ccc4n(-c4ccccc4)c3cc21 ; CC1(C)c2cc3oc4ccccc4c3cc2-c2cc3c4cc(-c5cc(-c6ccccc6)nc(-c6ccccc6)n5)ccc4n(-c4ccccc4)c3cc21]

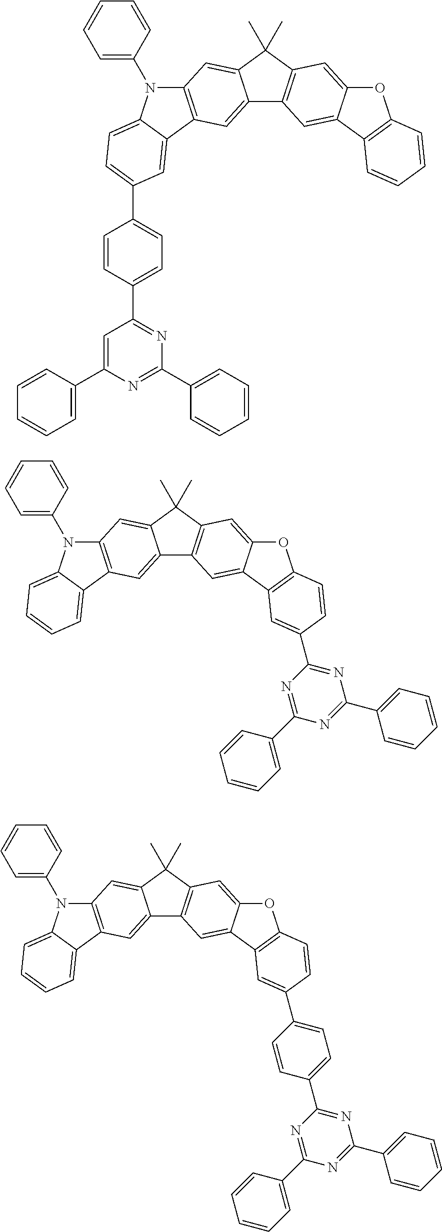

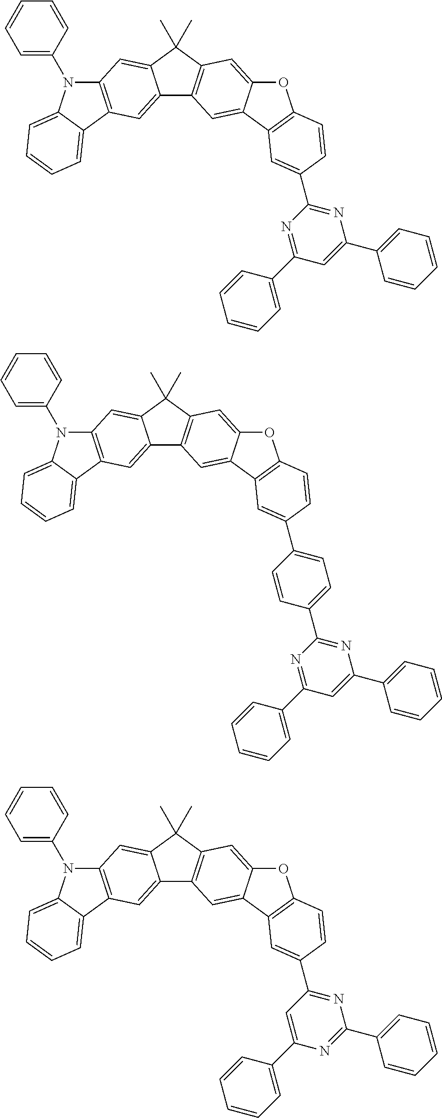 [SMILES: CC1(C)c2cc3oc4ccc(-c5nc(-c6ccccc6)cc(-c6ccccc6)n5)cc4c3cc2-c2cc3c4ccccc4n(-c4ccccc4)c3cc21 ; CC1(C)c2cc3oc4ccc(-c5ccc(-c6nc(-c7ccccc7)cc(-c7ccccc7)n6)cc5)cc4c3cc2-c2cc3c4ccccc4n(-c4ccccc4)c3cc21 ; CC1(C)c2cc3oc4ccc(-c5cc(-c6ccccc6)nc(-c6ccccc6)n5)cc4c3cc2-c2cc3c4ccccc4n(-c4ccccc4)c3cc21]

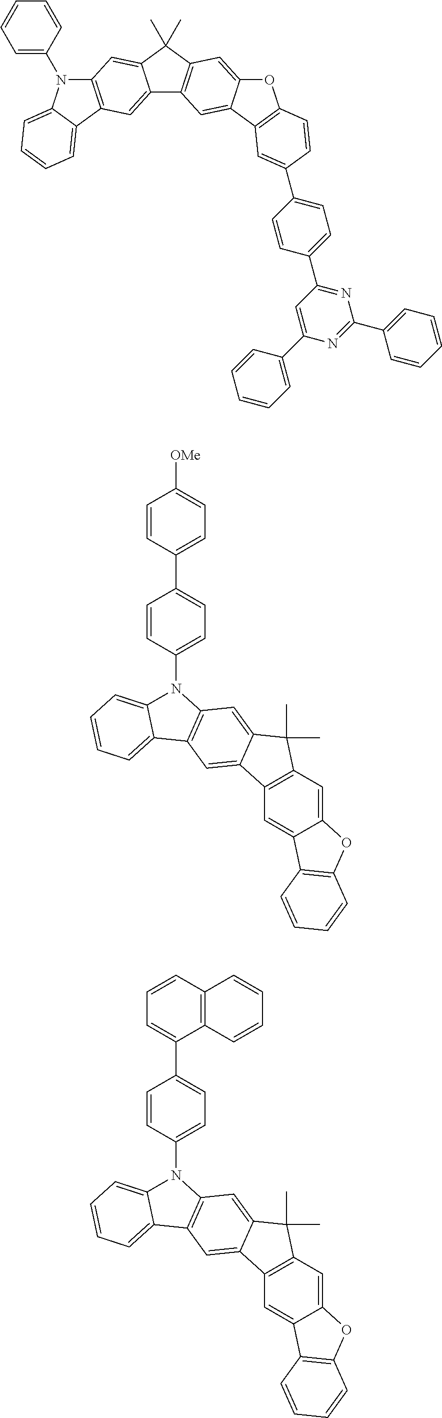 [SMILES: CC1(C)c2cc3oc4ccc(-c5ccc(-c6cc(-c7ccccc7)nc(-c7ccccc7)n6)cc5)cc4c3cc2-c2cc3c4ccccc4n(-c4ccccc4)c3cc21 ; COc1ccc(-c2ccc(-n3c4ccccc4c4cc5c(cc43)C(C)(C)c3cc4oc6ccccc6c4cc3-5)cc2)cc1 ; CC1(C)c2cc3oc4ccccc4c3cc2-c2cc3c4ccccc4n(-c4ccc(-c5cccc6ccccc56)cc4)c3cc21]

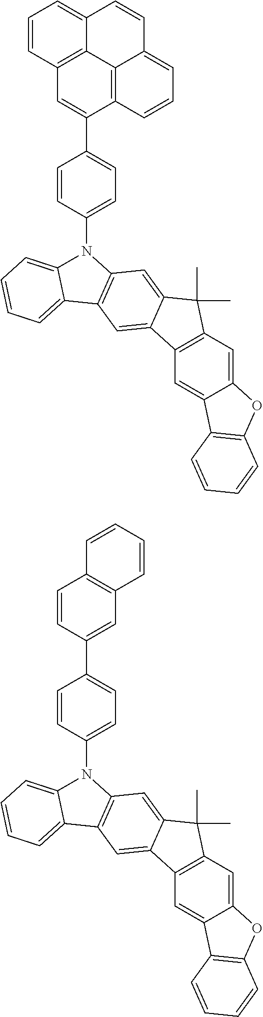 [SMILES: CC1(C)c2cc3oc4ccccc4c3cc2-c2cc3c4ccccc4n(-c4ccc(-c5cc6cccc7ccc8cccc5c8c76)cc4)c3cc21 ; CC1(C)c2cc3oc4ccccc4c3cc2-c2cc3c4ccccc4n(-c4ccc(-c5ccc6ccccc6c5)cc4)c3cc21]

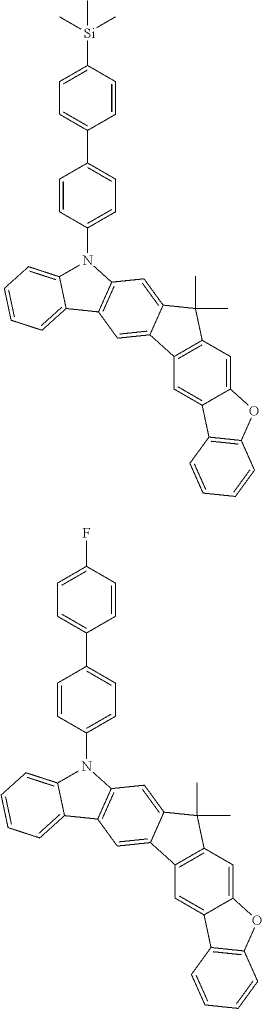 [SMILES: CC1(C)c2cc3oc4ccccc4c3cc2-c2cc3c4ccccc4n(-c4ccc(-c5ccc([Si](C)(C)C)cc5)cc4)c3cc21 ; CC1(C)c2cc3oc4ccccc4c3cc2-c2cc3c4ccccc4n(-c4ccc(-c5ccc(F)cc5)cc4)c3cc21]

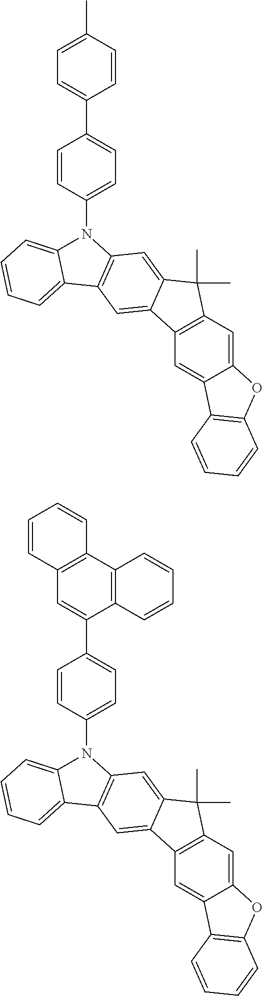 [SMILES: Cc1ccc(-c2ccc(-n3c4ccccc4c4cc5c(cc43)C(C)(C)c3cc4oc6ccccc6c4cc3-5)cc2)cc1 ; CC1(C)c2cc3oc4ccccc4c3cc2-c2cc3c4ccccc4n(-c4ccc(-c5cc6ccccc6c6ccccc56)cc4)c3cc21]

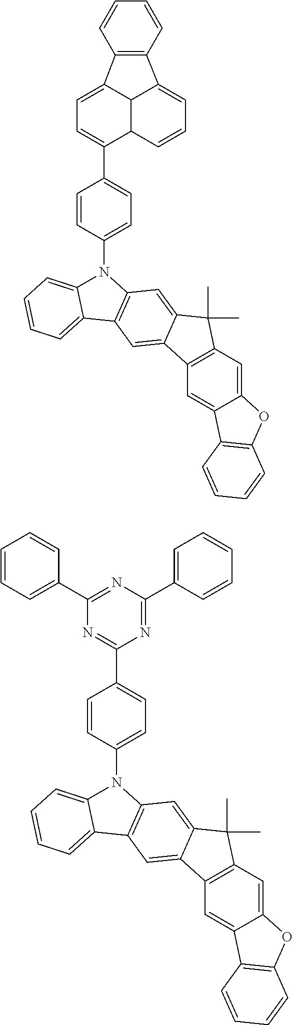

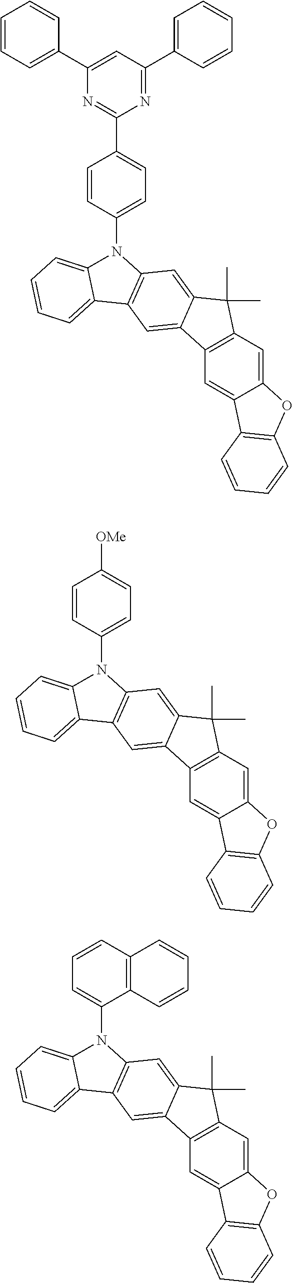 [SMILES: CC1(C)c2cc3oc4ccccc4c3cc2-c2cc3c4ccccc4n(-c4ccc(-c5nc(-c6ccccc6)cc(-c6ccccc6)n5)cc4)c3cc21 ; COc1ccc(-n2c3ccccc3c3cc4c(cc32)C(C)(C)c2cc3oc5ccccc5c3cc2-4)cc1 ; CC1(C)c2cc3oc4ccccc4c3cc2-c2cc3c4ccccc4n(-c4cccc5ccccc45)c3cc21]

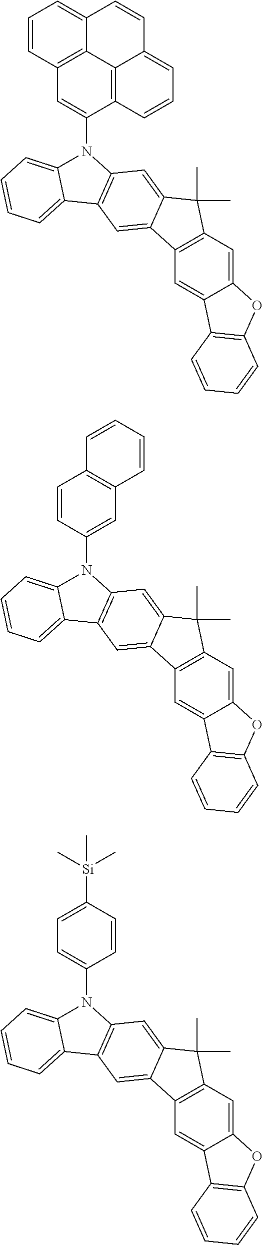 [SMILES: CC1(C)c2cc3oc4ccccc4c3cc2-c2cc3c4ccccc4n(-c4cc5cccc6ccc7cccc4c7c65)c3cc21 ; CC1(C)c2cc3oc4ccccc4c3cc2-c2cc3c4ccccc4n(-c4ccc5ccccc5c4)c3cc21 ; CC1(C)c2cc3oc4ccccc4c3cc2-c2cc3c4ccccc4n(-c4ccc([Si](C)(C)C)cc4)c3cc21]

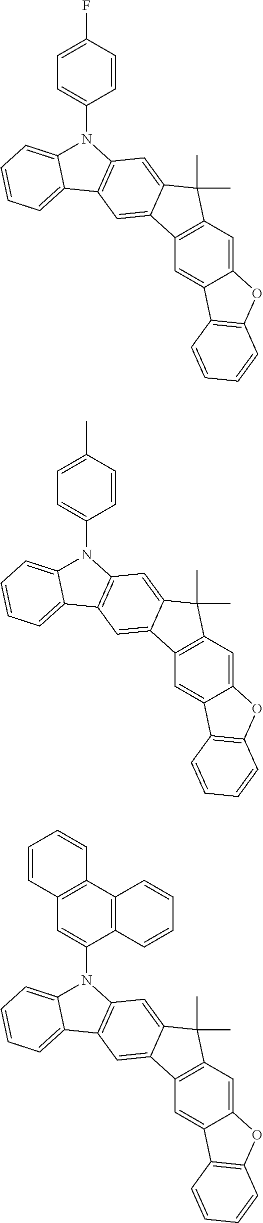 [SMILES: CC1(C)c2cc3oc4ccccc4c3cc2-c2cc3c4ccccc4n(-c4ccc(F)cc4)c3cc21 ; Cc1ccc(-n2c3ccccc3c3cc4c(cc32)C(C)(C)c2cc3oc5ccccc5c3cc2-4)cc1 ; CC1(C)c2cc3oc4ccccc4c3cc2-c2cc3c4ccccc4n(-c4cc5ccccc5c5ccccc45)c3cc21]

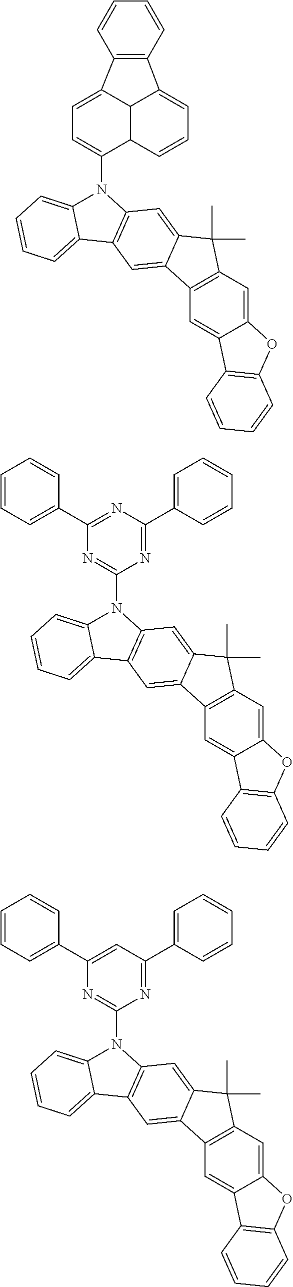

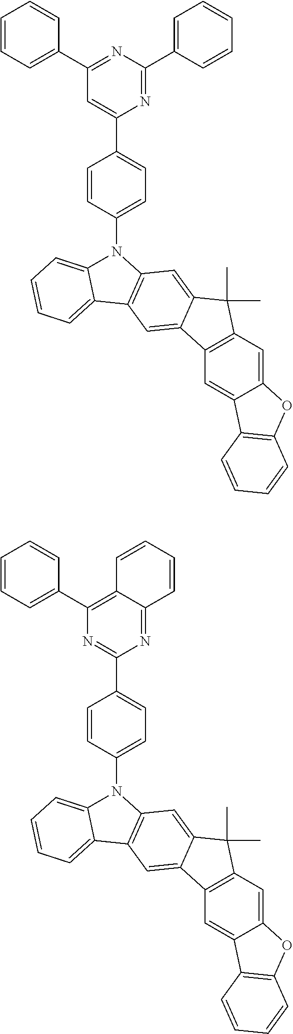 [SMILES: CC1(C)c2cc3oc4ccccc4c3cc2-c2cc3c4ccccc4n(-c4ccc(-c5cc(-c6ccccc6)nc(-c6ccccc6)n5)cc4)c3cc21 ; CC1(C)c2cc3oc4ccccc4c3cc2-c2cc3c4ccccc4n(-c4ccc(-c5nc(-c6ccccc6)c6ccccc6n5)cc4)c3cc21]

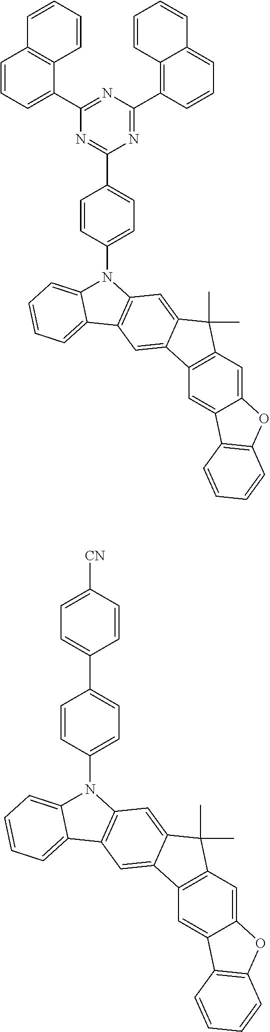 [SMILES: CC1(C)c2cc3oc4ccccc4c3cc2-c2cc3c4ccccc4n(-c4ccc(-c5nc(-c6cccc7ccccc67)nc(-c6cccc7ccccc67)n5)cc4)c3cc21 ; CC1(C)c2cc3oc4ccccc4c3cc2-c2cc3c4ccccc4n(-c4ccc(-c5ccc(C#N)cc5)cc4)c3cc21]

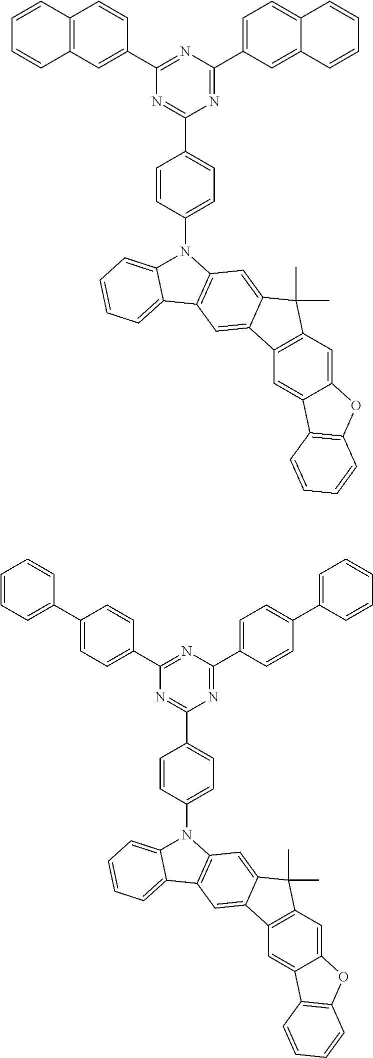 [SMILES: CC1(C)c2cc3oc4ccccc4c3cc2-c2cc3c4ccccc4n(-c4ccc(-c5nc(-c6ccc7ccccc7c6)nc(-c6ccc7ccccc7c6)n5)cc4)c3cc21 ; CC1(C)c2cc3oc4ccccc4c3cc2-c2cc3c4ccccc4n(-c4ccc(-c5nc(-c6ccc(-c7ccccc7)cc6)nc(-c6ccc(-c7ccccc7)cc6)n5)cc4)c3cc21]

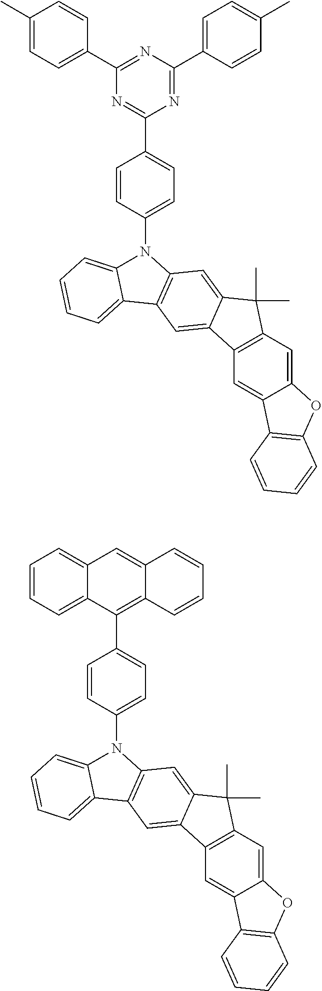 [SMILES: Cc1ccc(-c2nc(-c3ccc(C)cc3)nc(-c3ccc(-n4c5ccccc5c5cc6c(cc54)C(C)(C)c4cc5oc7ccccc7c5cc4-6)cc3)n2)cc1 ; CC1(C)c2cc3oc4ccccc4c3cc2-c2cc3c4ccccc4n(-c4ccc(-c5c6ccccc6cc6ccccc56)cc4)c3cc21]

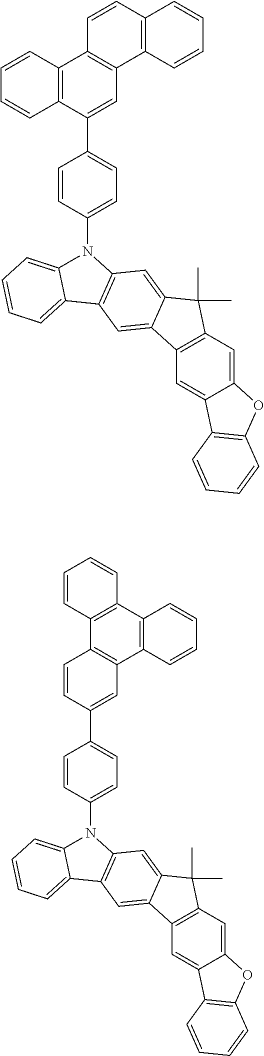 [SMILES: CC1(C)c2cc3oc4ccccc4c3cc2-c2cc3c4ccccc4n(-c4ccc(-c5cc6c7ccccc7ccc6c6ccccc56)cc4)c3cc21 ; CC1(C)c2cc3oc4ccccc4c3cc2-c2cc3c4ccccc4n(-c4ccc(-c5ccc6c7ccccc7c7ccccc7c6c5)cc4)c3cc21]

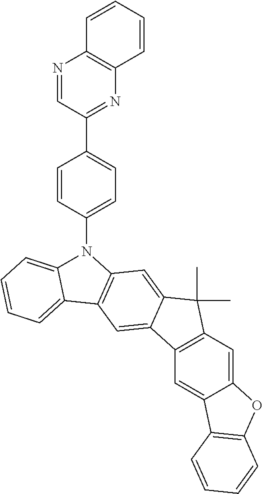 [SMILES: CC1(C)c2cc3oc4ccccc4c3cc2-c2cc3c4ccccc4n(-c4ccc(-c5cnc6ccccc6n5)cc4)c3cc21]

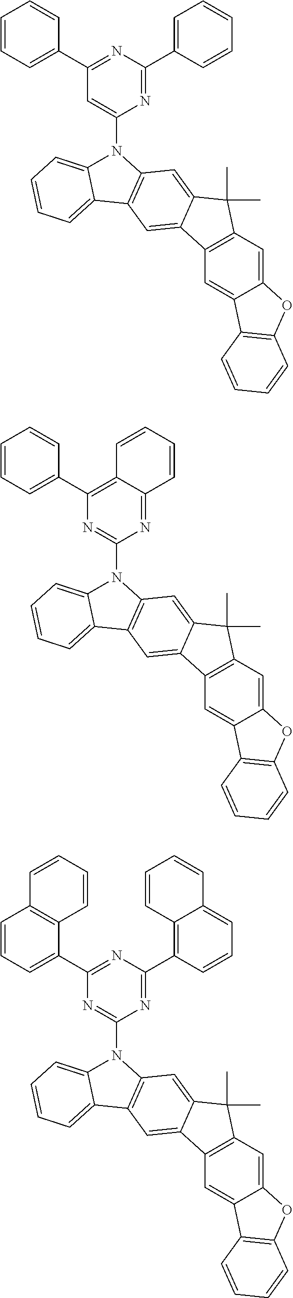 [SMILES: CC1(C)c2cc3oc4ccccc4c3cc2-c2cc3c4ccccc4n(-c4cc(-c5ccccc5)nc(-c5ccccc5)n4)c3cc21 ; CC1(C)c2cc3oc4ccccc4c3cc2-c2cc3c4ccccc4n(-c4nc(-c5ccccc5)c5ccccc5n4)c3cc21 ; CC1(C)c2cc3oc4ccccc4c3cc2-c2cc3c4ccccc4n(-c4nc(-c5cccc6ccccc56)nc(-c5cccc6ccccc56)n4)c3cc21]

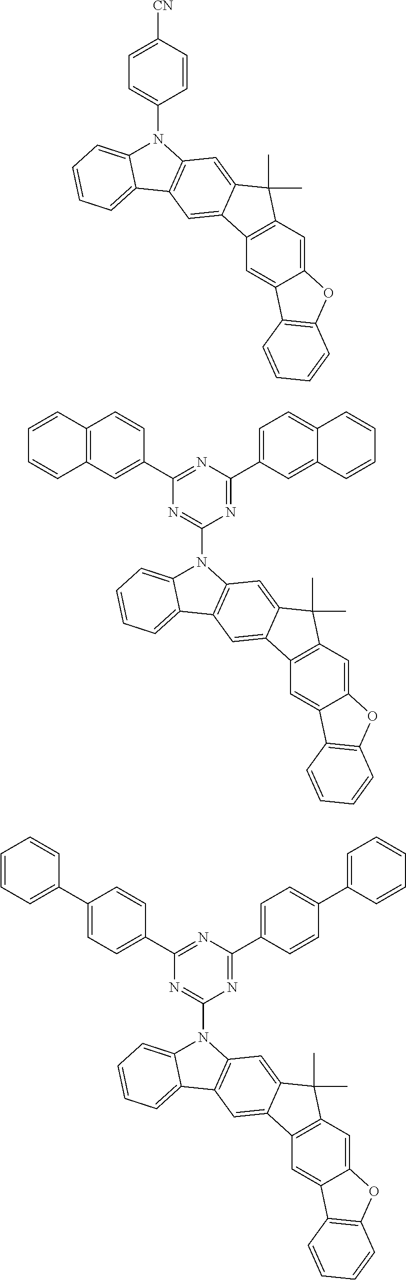

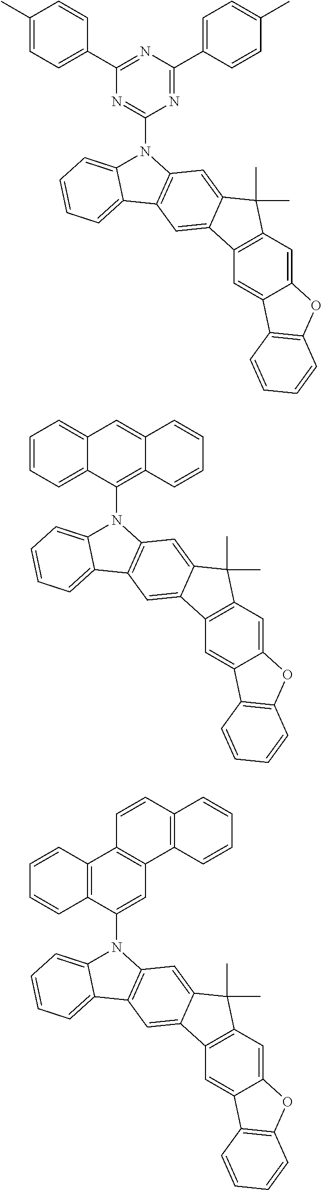 [SMILES: Cc1ccc(-c2nc(-c3ccc(C)cc3)nc(-n3c4ccccc4c4cc5c(cc43)C(C)(C)c3cc4oc6ccccc6c4cc3-5)n2)cc1 ; CC1(C)c2cc3oc4ccccc4c3cc2-c2cc3c4ccccc4n(-c4c5ccccc5cc5ccccc45)c3cc21 ; CC1(C)c2cc3oc4ccccc4c3cc2-c2cc3c4ccccc4n(-c4cc5c6ccccc6ccc5c5ccccc45)c3cc21]

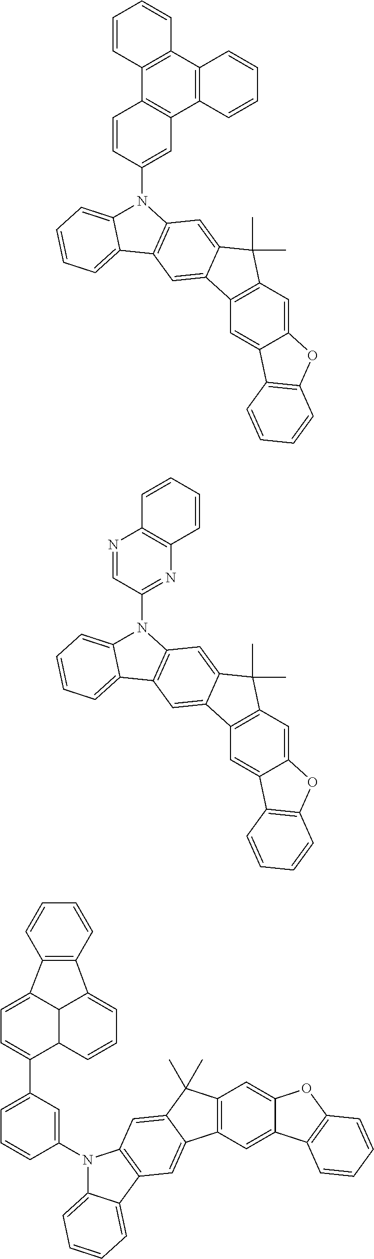 [SMILES: CC1(C)c2cc3oc4ccccc4c3cc2-c2cc3c4ccccc4n(-c4ccc5c6ccccc6c6ccccc6c5c4)c3cc21 ; CC1(C)c2cc3oc4ccccc4c3cc2-c2cc3c4ccccc4n(-c4cnc5ccccc5n4)c3cc21 ; CC1(C)c2cc3oc4ccccc4c3cc2-c2cc3c4ccccc4n(-c4cccc(C5=CC=C6c7ccccc7C7=CC=CC5C76)c4)c3cc21]

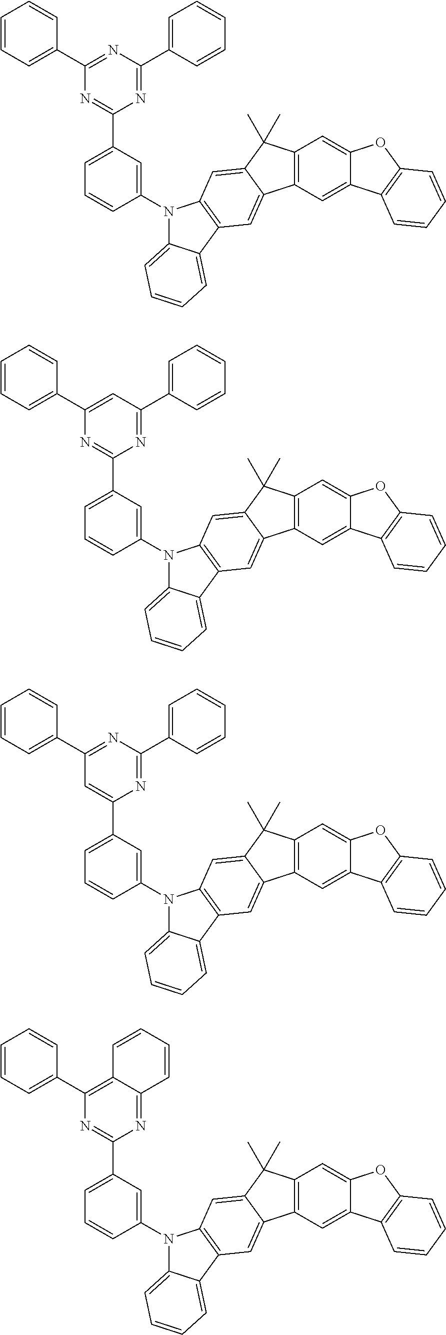

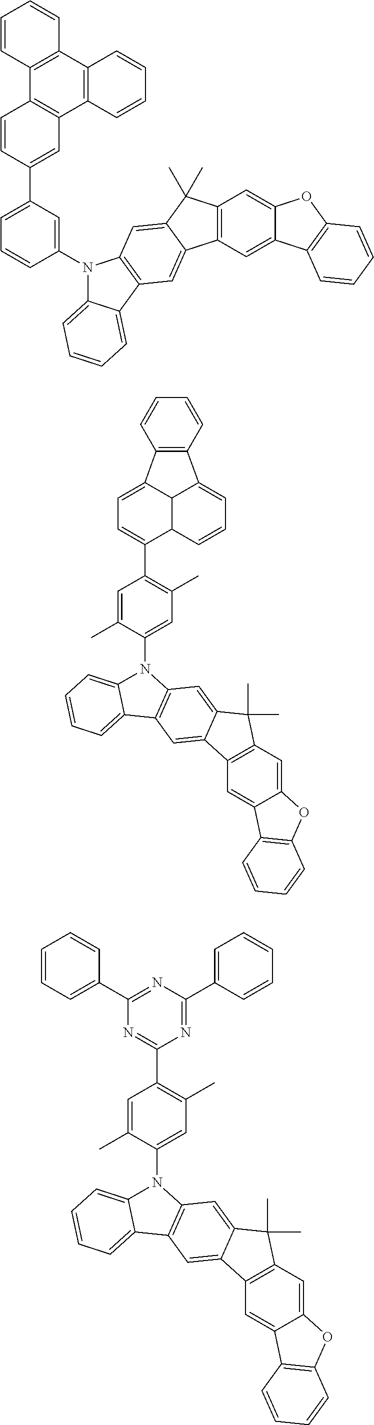

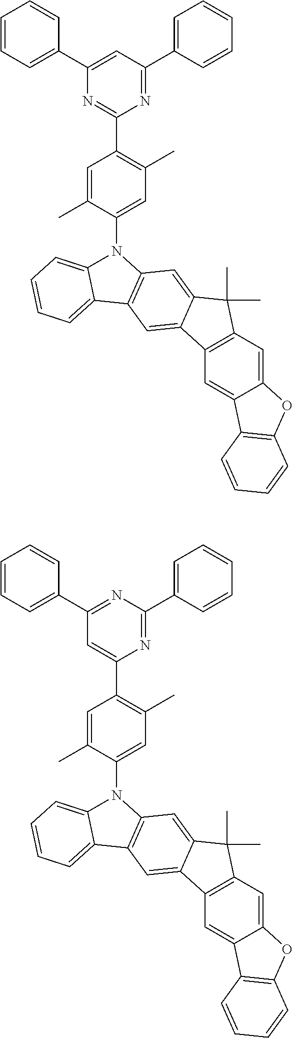 [SMILES: Cc1cc(-n2c3ccccc3c3cc4c(cc32)C(C)(C)c2cc3oc5ccccc5c3cc2-4)c(C)cc1-c1nc(-c2ccccc2)cc(-c2ccccc2)n1 ; Cc1cc(-n2c3ccccc3c3cc4c(cc32)C(C)(C)c2cc3oc5ccccc5c3cc2-4)c(C)cc1-c1cc(-c2ccccc2)nc(-c2ccccc2)n1]

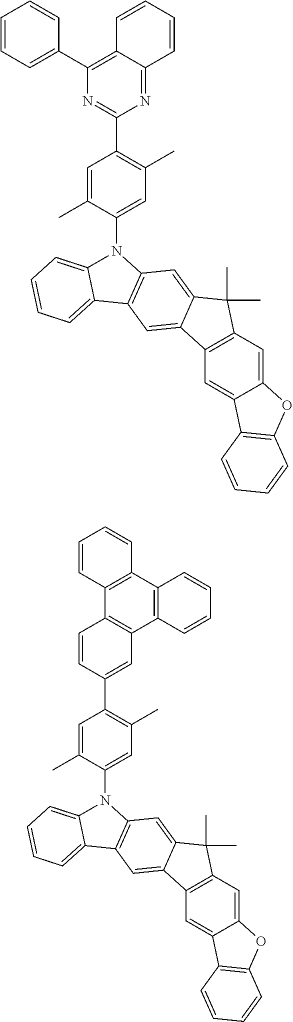 [SMILES: Cc1cc(-n2c3ccccc3c3cc4c(cc32)C(C)(C)c2cc3oc5ccccc5c3cc2-4)c(C)cc1-c1nc(-c2ccccc2)c2ccccc2n1 ; Cc1cc(-n2c3ccccc3c3cc4c(cc32)C(C)(C)c2cc3oc5ccccc5c3cc2-4)c(C)cc1-c1ccc2c3ccccc3c3ccccc3c2c1]

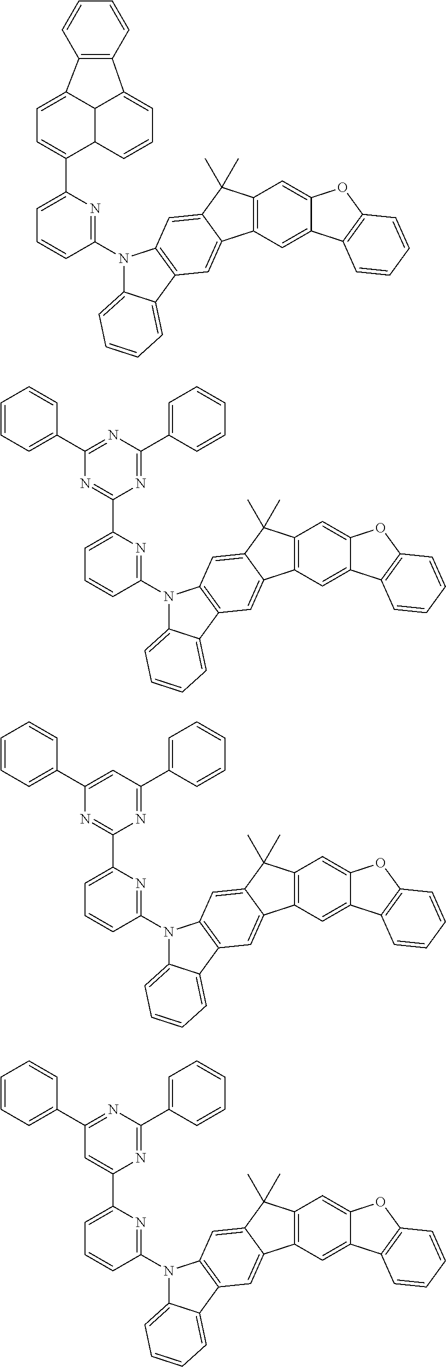

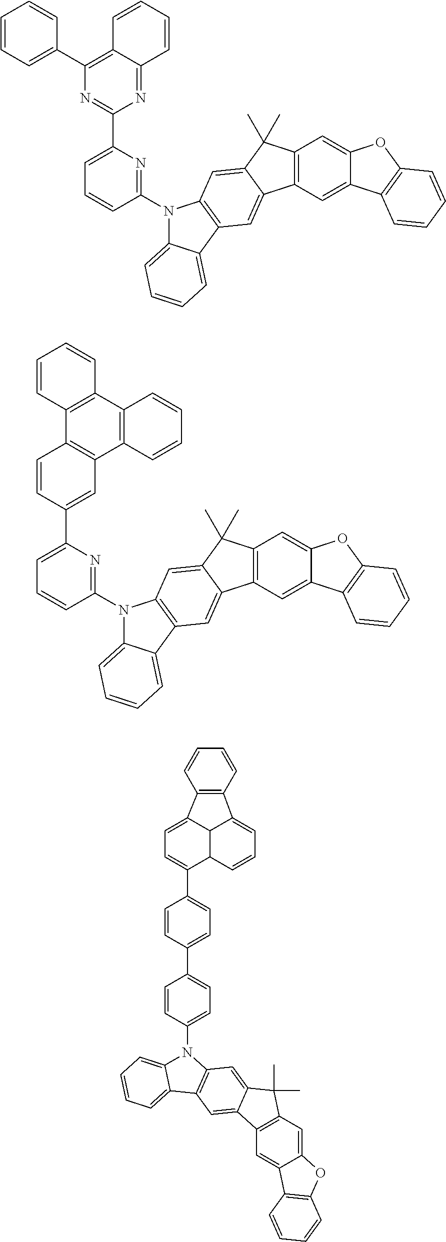 [SMILES: CC1(C)c2cc3oc4ccccc4c3cc2-c2cc3c4ccccc4n(-c4cccc(-c5nc(-c6ccccc6)c6ccccc6n5)n4)c3cc21 ; CC1(C)c2cc3oc4ccccc4c3cc2-c2cc3c4ccccc4n(-c4cccc(-c5ccc6c7ccccc7c7ccccc7c6c5)n4)c3cc21 ; CC1(C)c2cc3oc4ccccc4c3cc2-c2cc3c4ccccc4n(-c4ccc(-c5ccc(C6=CC=C7c8ccccc8C8=CC=CC6C87)cc5)cc4)c3cc21]

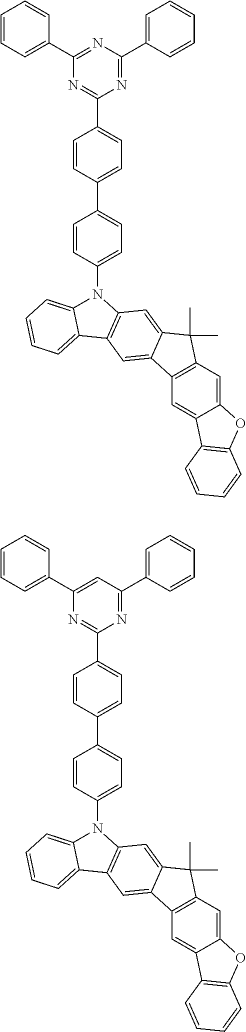 [SMILES: CC1(C)c2cc3oc4ccccc4c3cc2-c2cc3c4ccccc4n(-c4ccc(-c5ccc(-c6nc(-c7ccccc7)nc(-c7ccccc7)n6)cc5)cc4)c3cc21 ; CC1(C)c2cc3oc4ccccc4c3cc2-c2cc3c4ccccc4n(-c4ccc(-c5ccc(-c6nc(-c7ccccc7)cc(-c7ccccc7)n6)cc5)cc4)c3cc21]

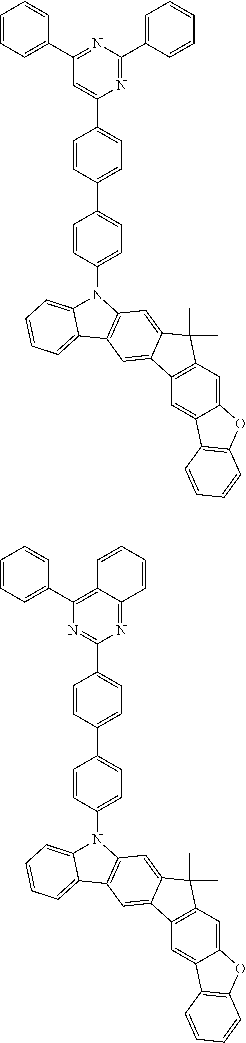 [SMILES: CC1(C)c2cc3oc4ccccc4c3cc2-c2cc3c4ccccc4n(-c4ccc(-c5ccc(-c6cc(-c7ccccc7)nc(-c7ccccc7)n6)cc5)cc4)c3cc21 ; CC1(C)c2cc3oc4ccccc4c3cc2-c2cc3c4ccccc4n(-c4ccc(-c5ccc(-c6nc(-c7ccccc7)c7ccccc7n6)cc5)cc4)c3cc21]

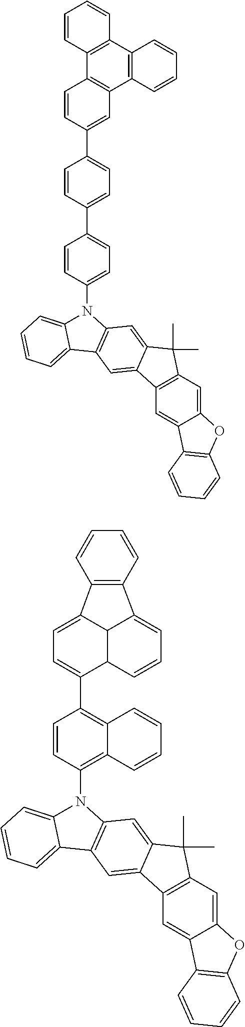 [SMILES: CC1(C)c2cc3oc4ccccc4c3cc2-c2cc3c4ccccc4n(-c4ccc(-c5ccc(-c6ccc7c8ccccc8c8ccccc8c7c6)cc5)cc4)c3cc21 ; CC1(C)c2cc3oc4ccccc4c3cc2-c2cc3c4ccccc4n(-c4ccc(C5=CC=C6c7ccccc7C7=CC=CC5C76)c5ccccc45)c3cc21]

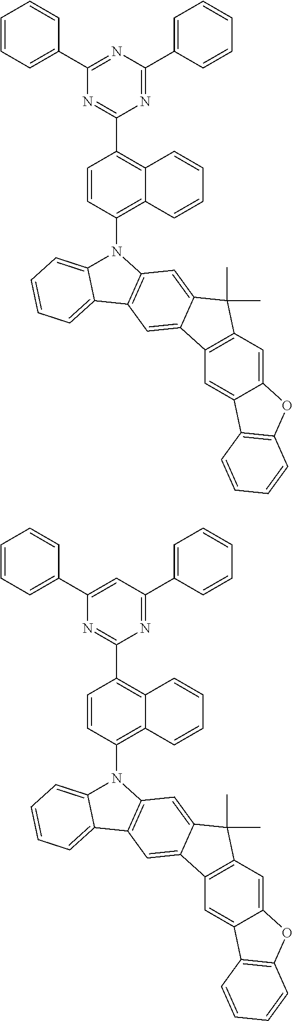 [SMILES: CC1(C)c2cc3oc4ccccc4c3cc2-c2cc3c4ccccc4n(-c4ccc(-c5nc(-c6ccccc6)nc(-c6ccccc6)n5)c5ccccc45)c3cc21 ; CC1(C)c2cc3oc4ccccc4c3cc2-c2cc3c4ccccc4n(-c4ccc(-c5nc(-c6ccccc6)cc(-c6ccccc6)n5)c5ccccc45)c3cc21]

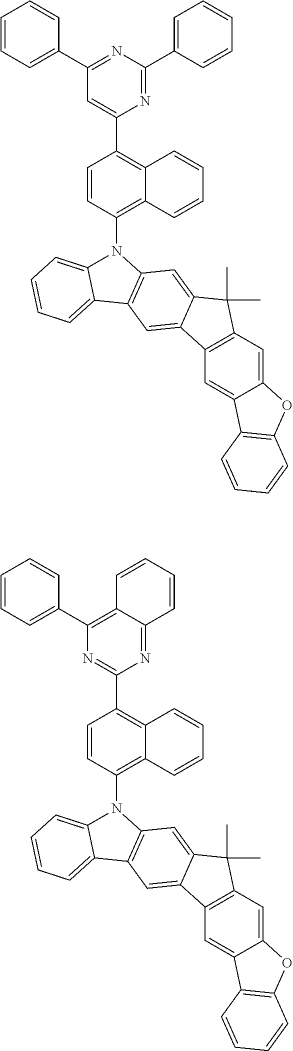 [SMILES: CC1(C)c2cc3oc4ccccc4c3cc2-c2cc3c4ccccc4n(-c4ccc(-c5cc(-c6ccccc6)nc(-c6ccccc6)n5)c5ccccc45)c3cc21 ; CC1(C)c2cc3oc4ccccc4c3cc2-c2cc3c4ccccc4n(-c4ccc(-c5nc(-c6ccccc6)c6ccccc6n5)c5ccccc45)c3cc21]

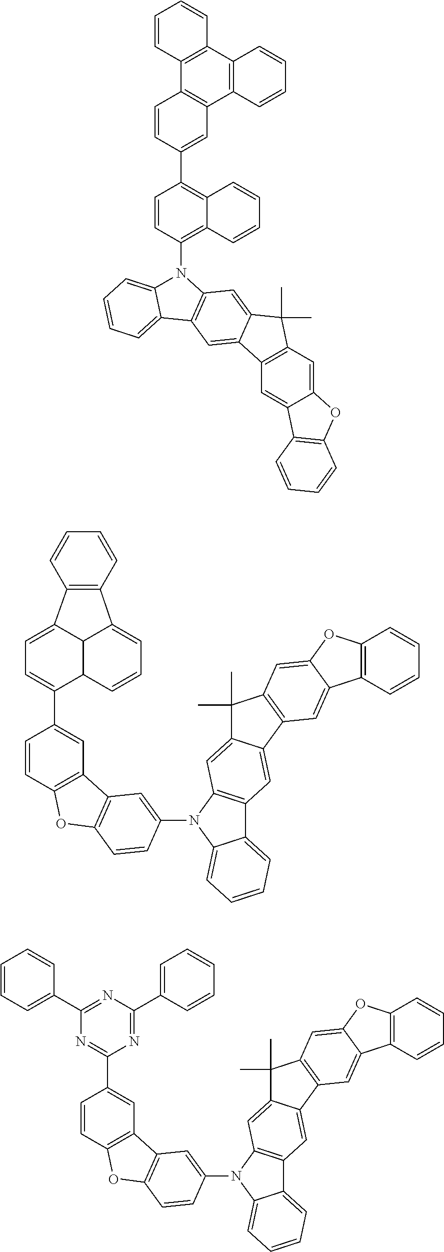 [SMILES: CC1(C)c2cc3oc4ccccc4c3cc2-c2cc3c4ccccc4n(-c4ccc(-c5ccc6c7ccccc7c7ccccc7c6c5)c5ccccc45)c3cc21 ; CC1(C)c2cc3oc4ccccc4c3cc2-c2cc3c4ccccc4n(-c4ccc5oc6ccc(C7=CC=C8c9ccccc9C9=CC=CC7C98)cc6c5c4)c3cc21 ; CC1(C)c2cc3oc4ccccc4c3cc2-c2cc3c4ccccc4n(-c4ccc5oc6ccc(-c7nc(-c8ccccc8)nc(-c8ccccc8)n7)cc6c5c4)c3cc21]

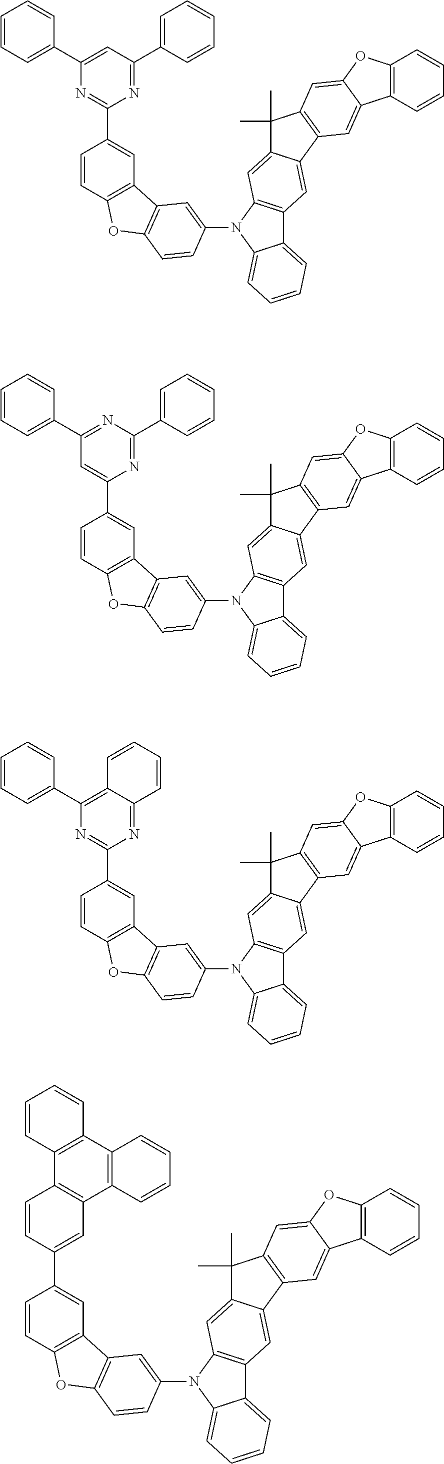 [SMILES: CC1(C)c2cc3oc4ccccc4c3cc2-c2cc3c4ccccc4n(-c4ccc5oc6ccc(-c7nc(-c8ccccc8)cc(-c8ccccc8)n7)cc6c5c4)c3cc21 ; CC1(C)c2cc3oc4ccccc4c3cc2-c2cc3c4ccccc4n(-c4ccc5oc6ccc(-c7cc(-c8ccccc8)nc(-c8ccccc8)n7)cc6c5c4)c3cc21 ; CC1(C)c2cc3oc4ccccc4c3cc2-c2cc3c4ccccc4n(-c4ccc5oc6ccc(-c7nc(-c8ccccc8)c8ccccc8n7)cc6c5c4)c3cc21 ; CC1(C)c2cc3oc4ccccc4c3cc2-c2cc3c4ccccc4n(-c4ccc5oc6ccc(-c7ccc8c9ccccc9c9ccccc9c8c7)cc6c5c4)c3cc21]

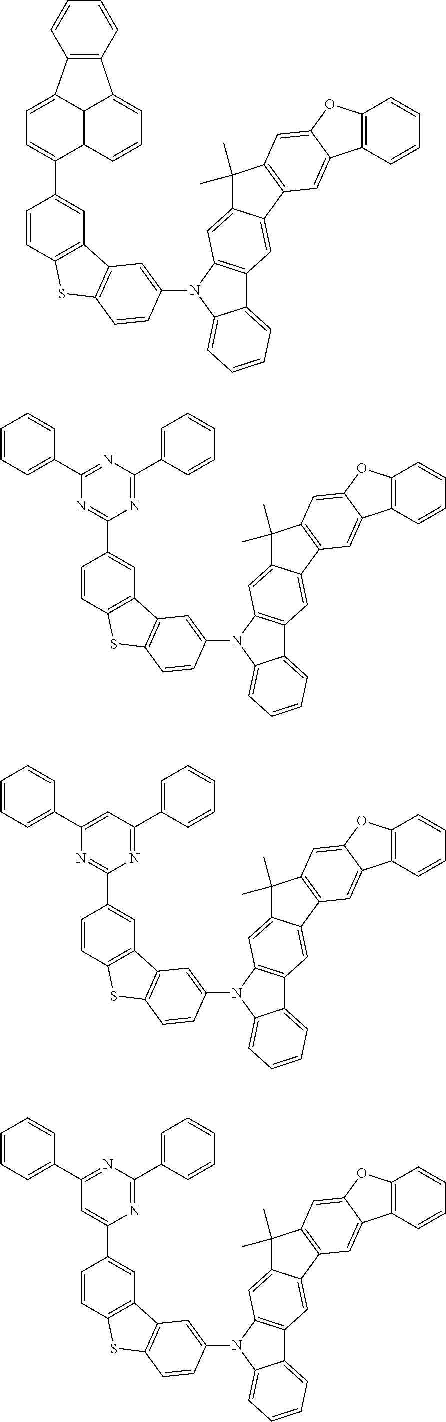 [SMILES: CC1(C)c2cc3oc4ccccc4c3cc2-c2cc3c4ccccc4n(-c4ccc5sc6ccc(C7=CC=C8c9ccccc9C9=CC=CC7C98)cc6c5c4)c3cc21 ; CC1(C)c2cc3oc4ccccc4c3cc2-c2cc3c4ccccc4n(-c4ccc5sc6ccc(-c7nc(-c8ccccc8)nc(-c8ccccc8)n7)cc6c5c4)c3cc21 ; CC1(C)c2cc3oc4ccccc4c3cc2-c2cc3c4ccccc4n(-c4ccc5sc6ccc(-c7nc(-c8ccccc8)cc(-c8ccccc8)n7)cc6c5c4)c3cc21 ; CC1(C)c2cc3oc4ccccc4c3cc2-c2cc3c4ccccc4n(-c4ccc5sc6ccc(-c7cc(-c8ccccc8)nc(-c8ccccc8)n7)cc6c5c4)c3cc21]

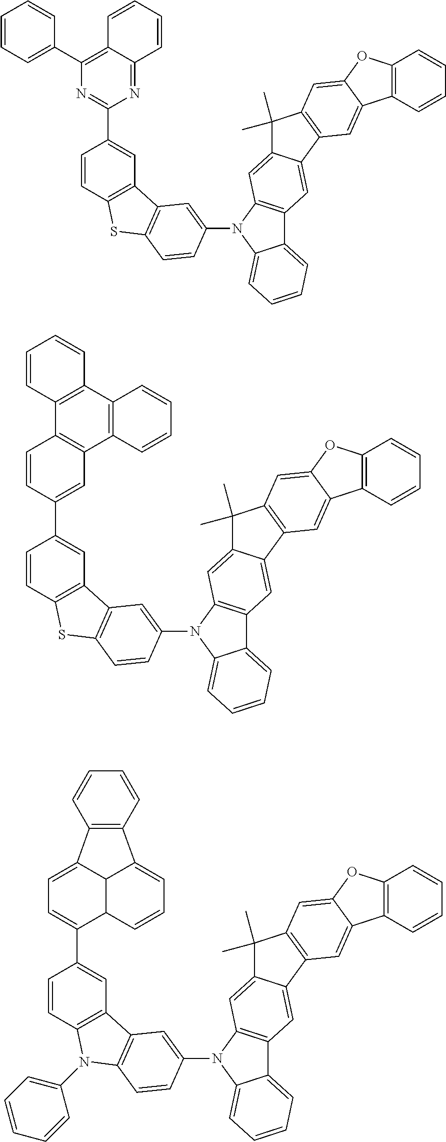 [SMILES: CC1(C)c2cc3oc4ccccc4c3cc2-c2cc3c4ccccc4n(-c4ccc5sc6ccc(-c7nc(-c8ccccc8)c8ccccc8n7)cc6c5c4)c3cc21 ; CC1(C)c2cc3oc4ccccc4c3cc2-c2cc3c4ccccc4n(-c4ccc5sc6ccc(-c7ccc8c9ccccc9c9ccccc9c8c7)cc6c5c4)c3cc21 ; CC1(C)c2cc3oc4ccccc4c3cc2-c2cc3c4ccccc4n(-c4ccc5c(c4)c4cc(C6=CC=C7c8ccccc8C8=CC=CC6C87)ccc4n5-c4ccccc4)c3cc21]

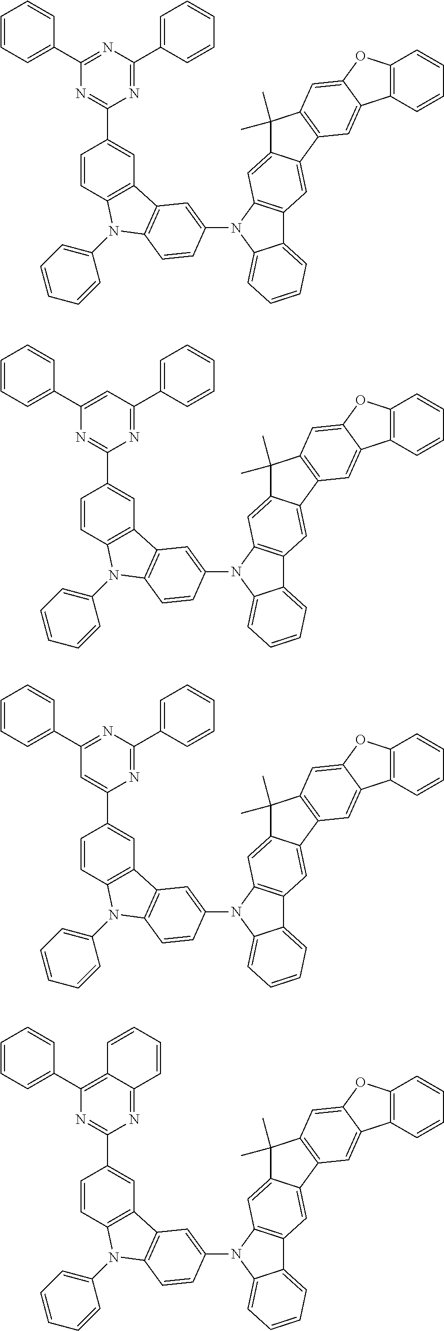 [SMILES: CC1(C)c2cc3oc4ccccc4c3cc2-c2cc3c4ccccc4n(-c4ccc5c(c4)c4cc(-c6nc(-c7ccccc7)nc(-c7ccccc7)n6)ccc4n5-c4ccccc4)c3cc21 ; CC1(C)c2cc3oc4ccccc4c3cc2-c2cc3c4ccccc4n(-c4ccc5c(c4)c4cc(-c6nc(-c7ccccc7)cc(-c7ccccc7)n6)ccc4n5-c4ccccc4)c3cc21 ; CC1(C)c2cc3oc4ccccc4c3cc2-c2cc3c4ccccc4n(-c4ccc5c(c4)c4cc(-c6cc(-c7ccccc7)nc(-c7ccccc7)n6)ccc4n5-c4ccccc4)c3cc21 ; CC1(C)c2cc3oc4ccccc4c3cc2-c2cc3c4ccccc4n(-c4ccc5c(c4)c4cc(-c6nc(-c7ccccc7)c7ccccc7n6)ccc4n5-c4ccccc4)c3cc21]

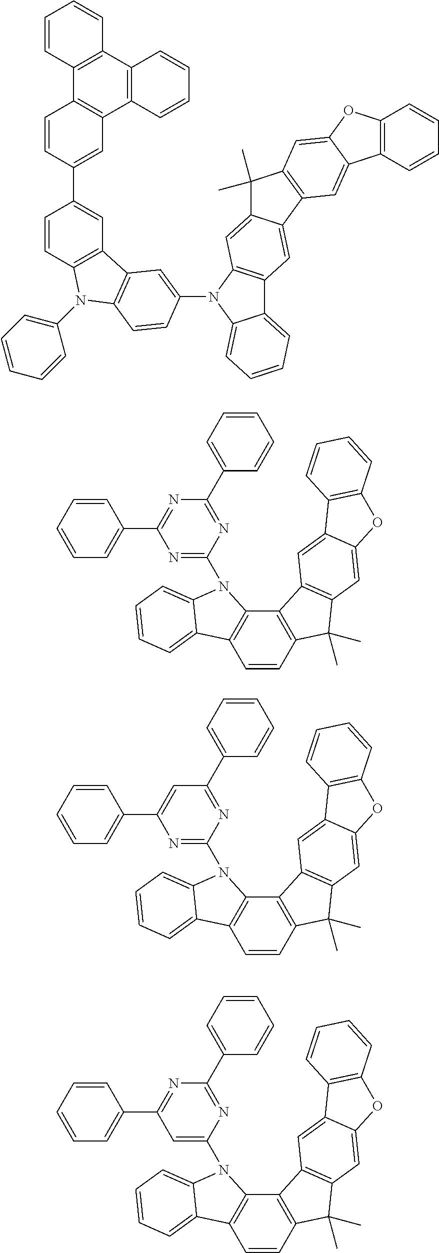

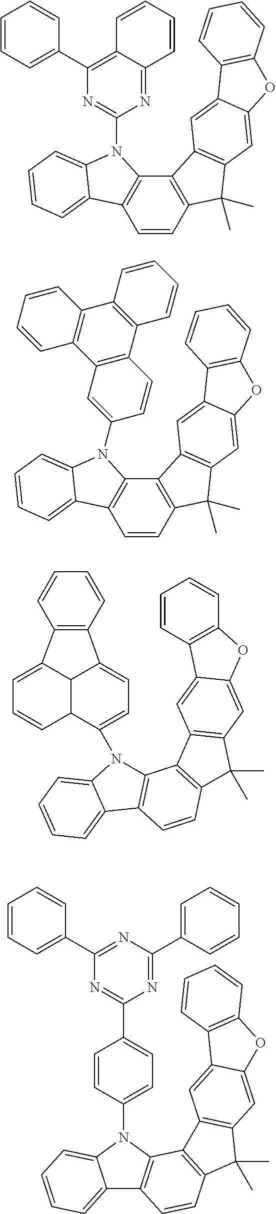

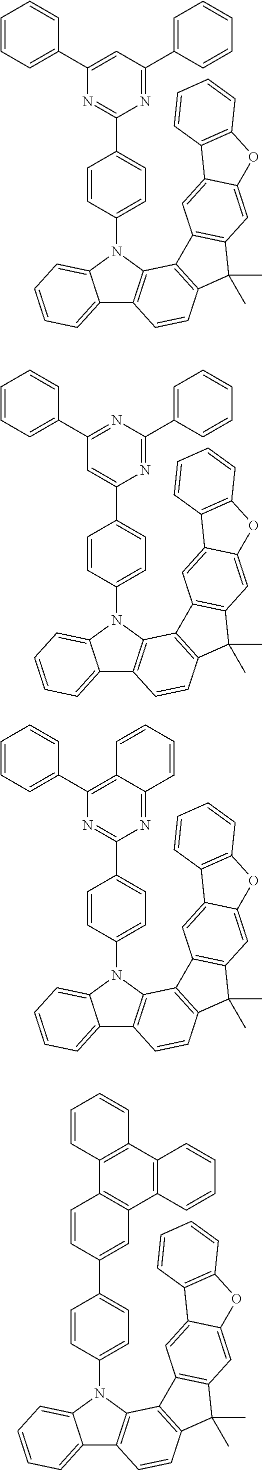

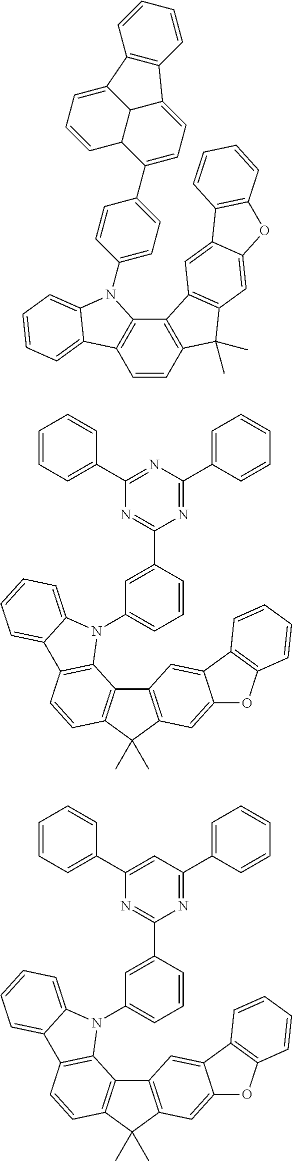

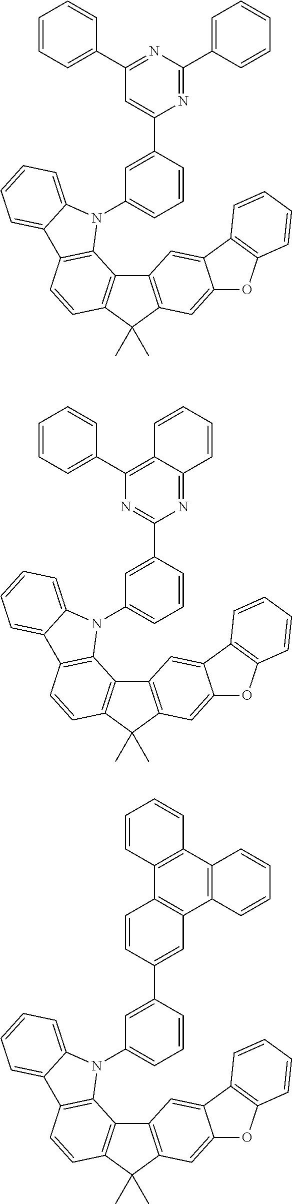

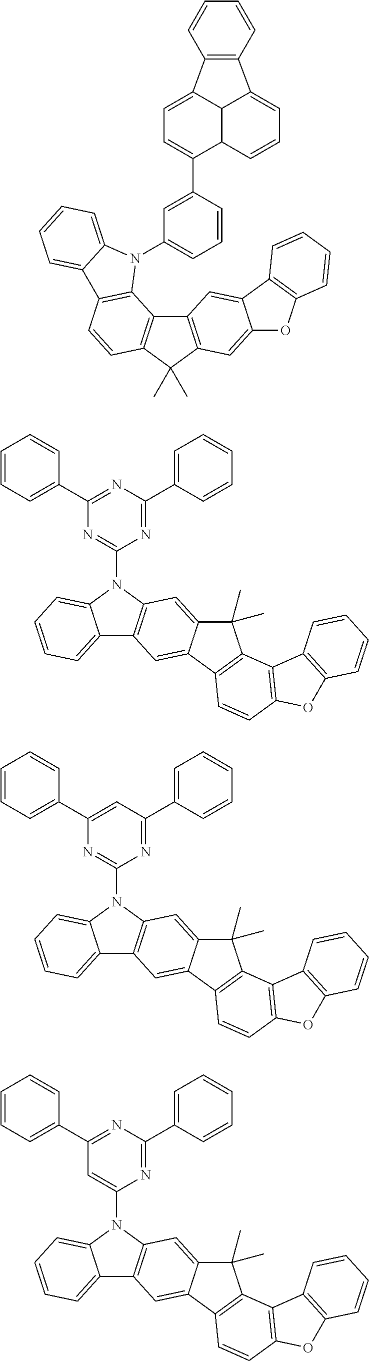

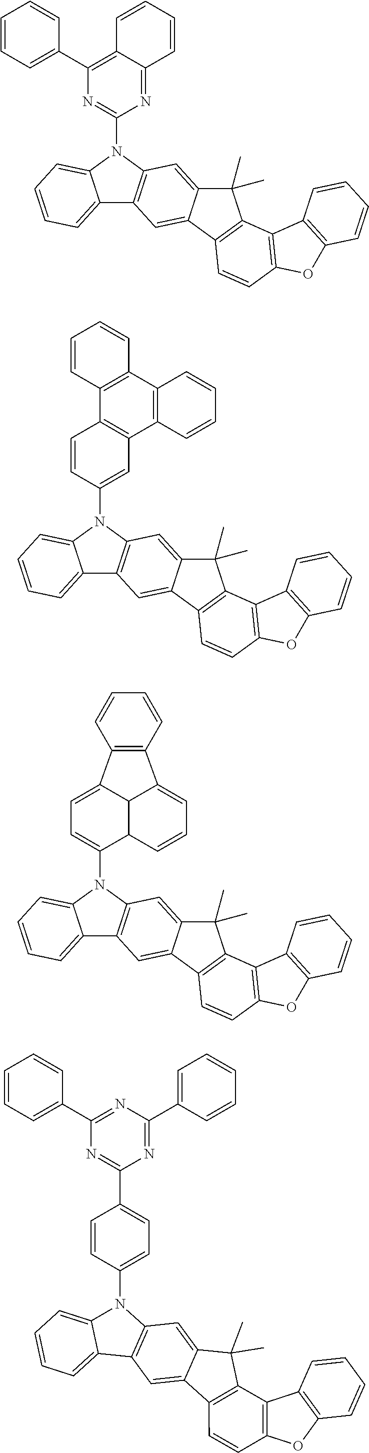

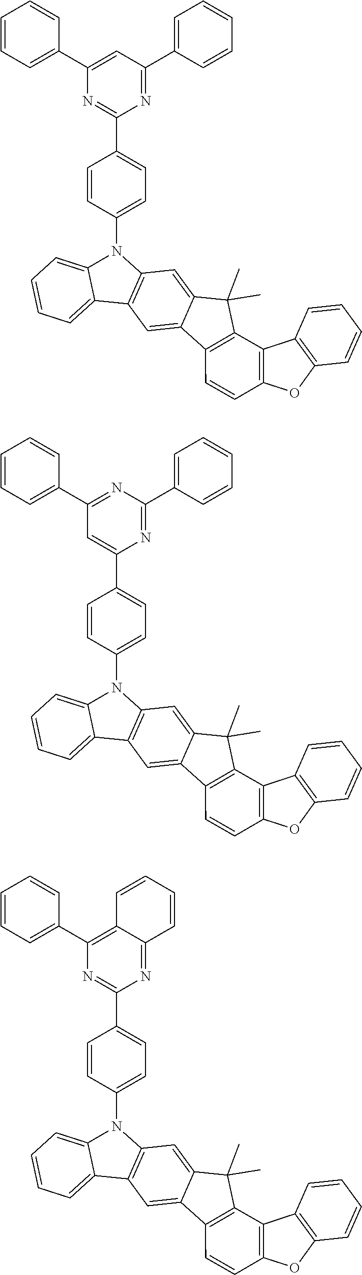 [SMILES: CC1(C)c2cc3c(cc2-c2ccc4oc5ccccc5c4c21)c1ccccc1n3-c1ccc(-c2nc(-c3ccccc3)cc(-c3ccccc3)n2)cc1 ; CC1(C)c2cc3c(cc2-c2ccc4oc5ccccc5c4c21)c1ccccc1n3-c1ccc(-c2cc(-c3ccccc3)nc(-c3ccccc3)n2)cc1 ; CC1(C)c2cc3c(cc2-c2ccc4oc5ccccc5c4c21)c1ccccc1n3-c1ccc(-c2nc(-c3ccccc3)c3ccccc3n2)cc1]

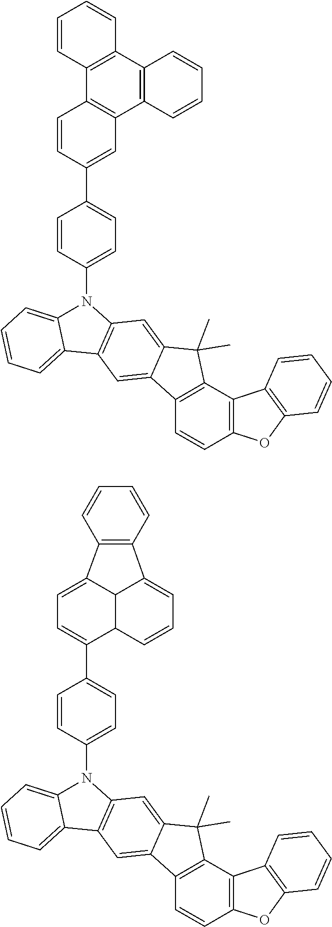 [SMILES: CC1(C)c2cc3c(cc2-c2ccc4oc5ccccc5c4c21)c1ccccc1n3-c1ccc(-c2ccc3c4ccccc4c4ccccc4c3c2)cc1 ; CC1(C)c2cc3c(cc2-c2ccc4oc5ccccc5c4c21)c1ccccc1n3-c1ccc(C2=CC=C3c4ccccc4C4=CC=CC2C43)cc1]

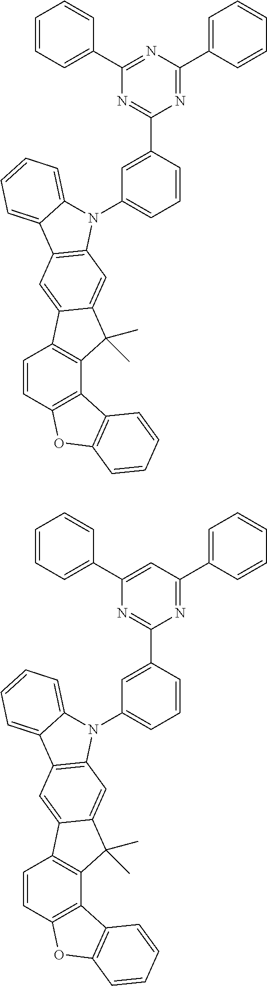 [SMILES: CC1(C)c2cc3c(cc2-c2ccc4oc5ccccc5c4c21)c1ccccc1n3-c1cccc(-c2nc(-c3ccccc3)nc(-c3ccccc3)n2)c1 ; CC1(C)c2cc3c(cc2-c2ccc4oc5ccccc5c4c21)c1ccccc1n3-c1cccc(-c2nc(-c3ccccc3)cc(-c3ccccc3)n2)c1]

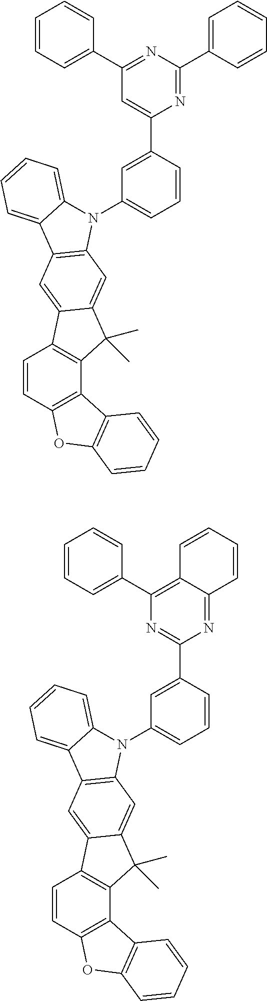 [SMILES: CC1(C)c2cc3c(cc2-c2ccc4oc5ccccc5c4c21)c1ccccc1n3-c1cccc(-c2cc(-c3ccccc3)nc(-c3ccccc3)n2)c1 ; CC1(C)c2cc3c(cc2-c2ccc4oc5ccccc5c4c21)c1ccccc1n3-c1cccc(-c2nc(-c3ccccc3)c3ccccc3n2)c1]

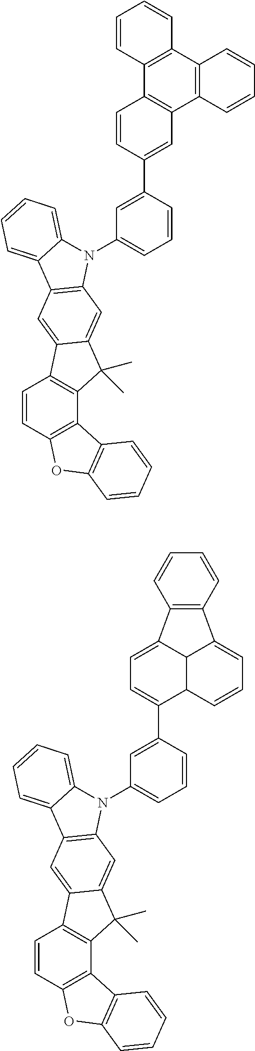 [SMILES: CC1(C)c2cc3c(cc2-c2ccc4oc5ccccc5c4c21)c1ccccc1n3-c1cccc(-c2ccc3c4ccccc4c4ccccc4c3c2)c1 ; CC1(C)c2cc3c(cc2-c2ccc4oc5ccccc5c4c21)c1ccccc1n3-c1cccc(C2=CC=C3c4ccccc4C4=CC=CC2C43)c1]

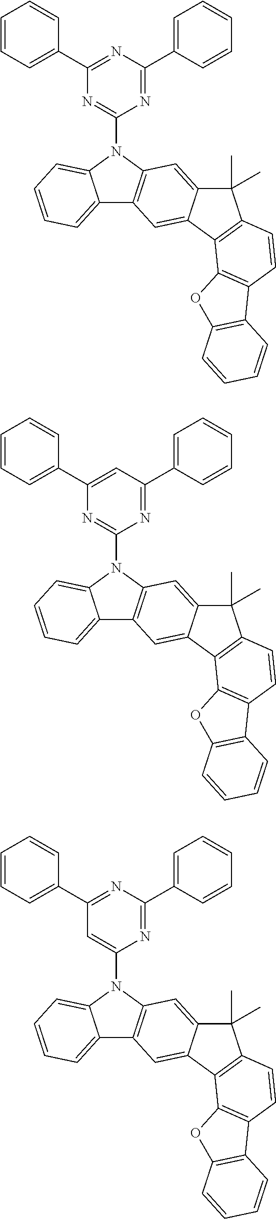

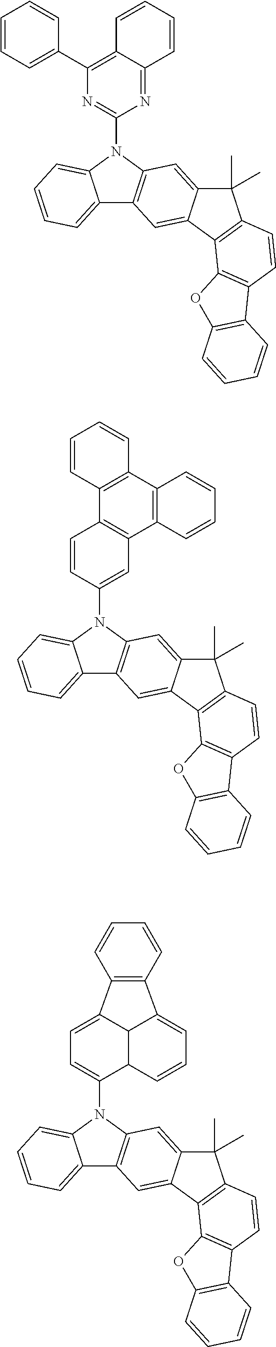 [SMILES: CC1(C)c2cc3c(cc2-c2c1ccc1c2oc2ccccc21)c1ccccc1n3-c1nc(-c2ccccc2)c2ccccc2n1 ; CC1(C)c2cc3c(cc2-c2c1ccc1c2oc2ccccc21)c1ccccc1n3-c1ccc2c3ccccc3c3ccccc3c2c1 ; CC1(C)c2cc3c(cc2-c2c1ccc1c2oc2ccccc21)c1ccccc1n3C1=CC=C2c3ccccc3C3=CC=CC1C32]

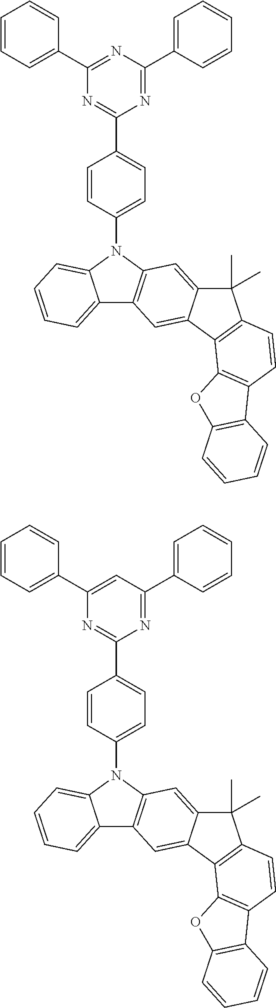 [SMILES: CC1(C)c2cc3c(cc2-c2c1ccc1c2oc2ccccc21)c1ccccc1n3-c1ccc(-c2nc(-c3ccccc3)nc(-c3ccccc3)n2)cc1 ; CC1(C)c2cc3c(cc2-c2c1ccc1c2oc2ccccc21)c1ccccc1n3-c1ccc(-c2nc(-c3ccccc3)cc(-c3ccccc3)n2)cc1]

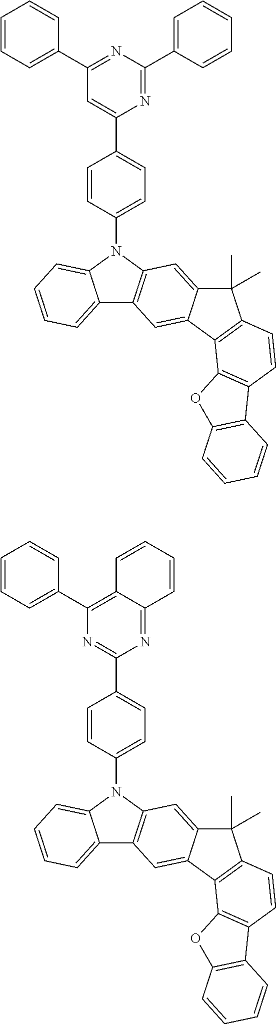 [SMILES: CC1(C)c2cc3c(cc2-c2c1ccc1c2oc2ccccc21)c1ccccc1n3-c1ccc(-c2cc(-c3ccccc3)nc(-c3ccccc3)n2)cc1 ; CC1(C)c2cc3c(cc2-c2c1ccc1c2oc2ccccc21)c1ccccc1n3-c1ccc(-c2nc(-c3ccccc3)c3ccccc3n2)cc1]

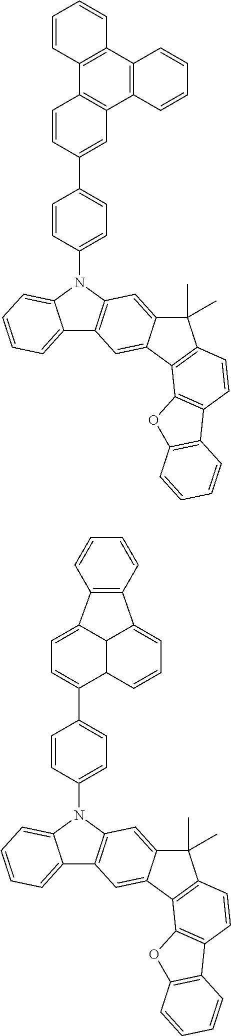 [SMILES: CC1(C)c2cc3c(cc2-c2c1ccc1c2oc2ccccc21)c1ccccc1n3-c1ccc(-c2ccc3c4ccccc4c4ccccc4c3c2)cc1 ; CC1(C)c2cc3c(cc2-c2c1ccc1c2oc2ccccc21)c1ccccc1n3-c1ccc(C2=CC=C3c4ccccc4C4=CC=CC2C43)cc1]

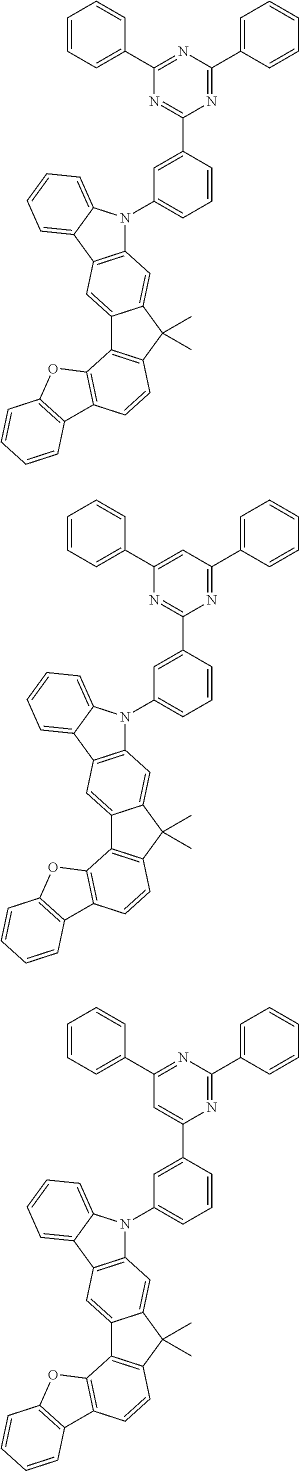 [SMILES: CC1(C)c2cc3c(cc2-c2c1ccc1c2oc2ccccc21)c1ccccc1n3-c1cccc(-c2nc(-c3ccccc3)nc(-c3ccccc3)n2)c1 ; CC1(C)c2cc3c(cc2-c2c1ccc1c2oc2ccccc21)c1ccccc1n3-c1cccc(-c2nc(-c3ccccc3)cc(-c3ccccc3)n2)c1 ; CC1(C)c2cc3c(cc2-c2c1ccc1c2oc2ccccc21)c1ccccc1n3-c1cccc(-c2cc(-c3ccccc3)nc(-c3ccccc3)n2)c1]

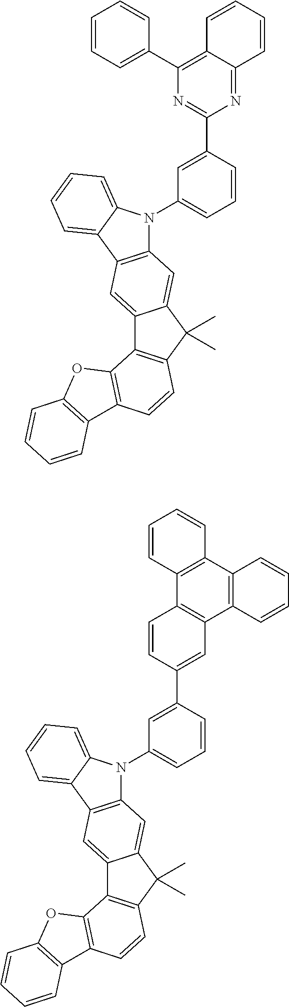 [SMILES: CC1(C)c2cc3c(cc2-c2c1ccc1c2oc2ccccc21)c1ccccc1n3-c1cccc(-c2nc(-c3ccccc3)c3ccccc3n2)c1 ; CC1(C)c2cc3c(cc2-c2c1ccc1c2oc2ccccc21)c1ccccc1n3-c1cccc(-c2ccc3c4ccccc4c4ccccc4c3c2)c1]

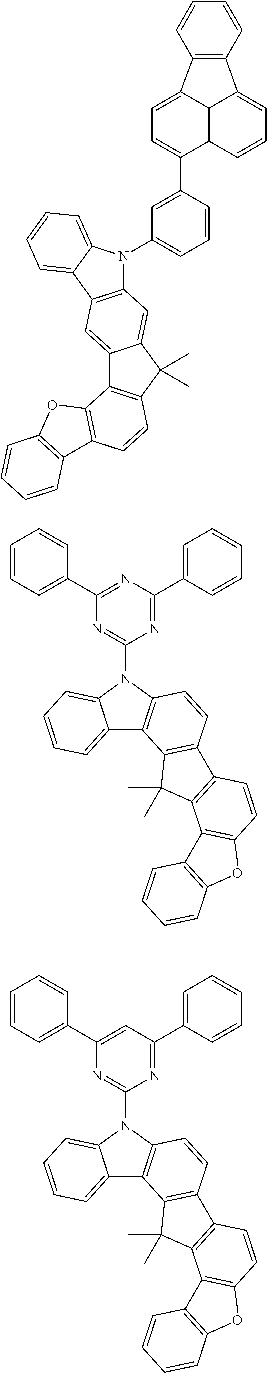

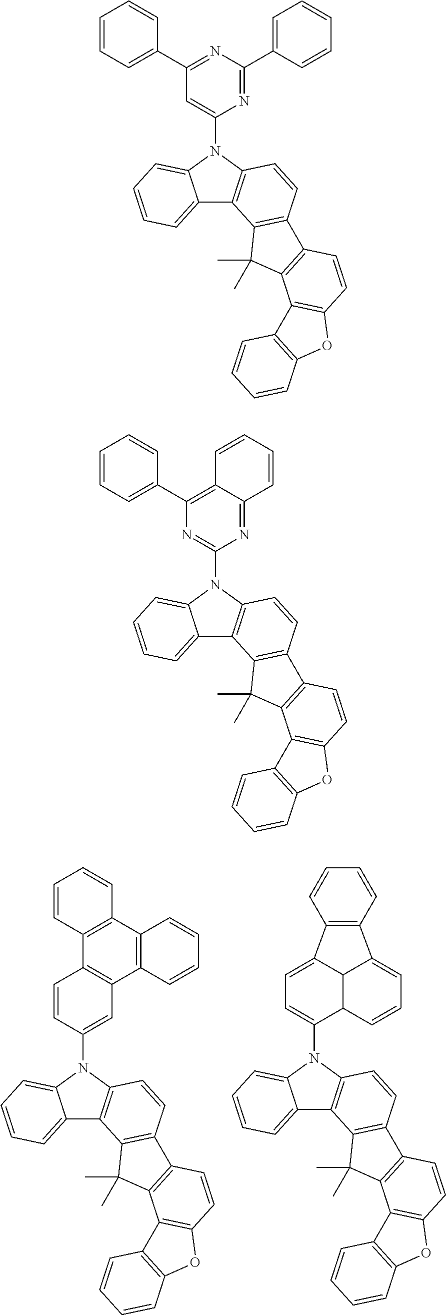

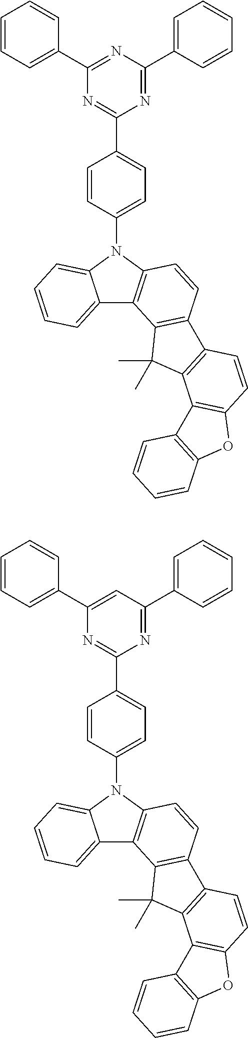 [SMILES: CC1(C)c2c(ccc3oc4ccccc4c23)-c2ccc3c(c21)c1ccccc1n3-c1ccc(-c2nc(-c3ccccc3)nc(-c3ccccc3)n2)cc1 ; CC1(C)c2c(ccc3oc4ccccc4c23)-c2ccc3c(c21)c1ccccc1n3-c1ccc(-c2nc(-c3ccccc3)cc(-c3ccccc3)n2)cc1]

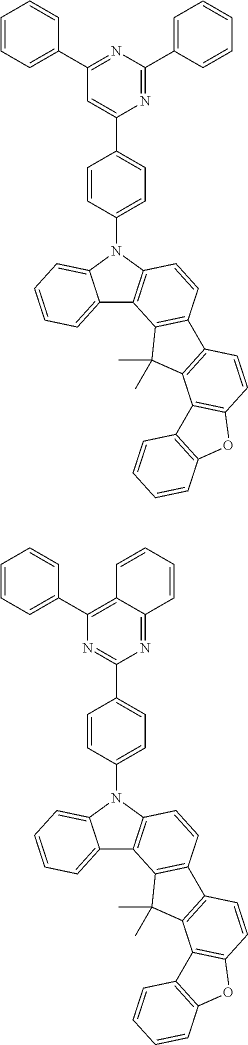 [SMILES: CC1(C)c2c(ccc3oc4ccccc4c23)-c2ccc3c(c21)c1ccccc1n3-c1ccc(-c2cc(-c3ccccc3)nc(-c3ccccc3)n2)cc1 ; CC1(C)c2c(ccc3oc4ccccc4c23)-c2ccc3c(c21)c1ccccc1n3-c1ccc(-c2nc(-c3ccccc3)c3ccccc3n2)cc1]

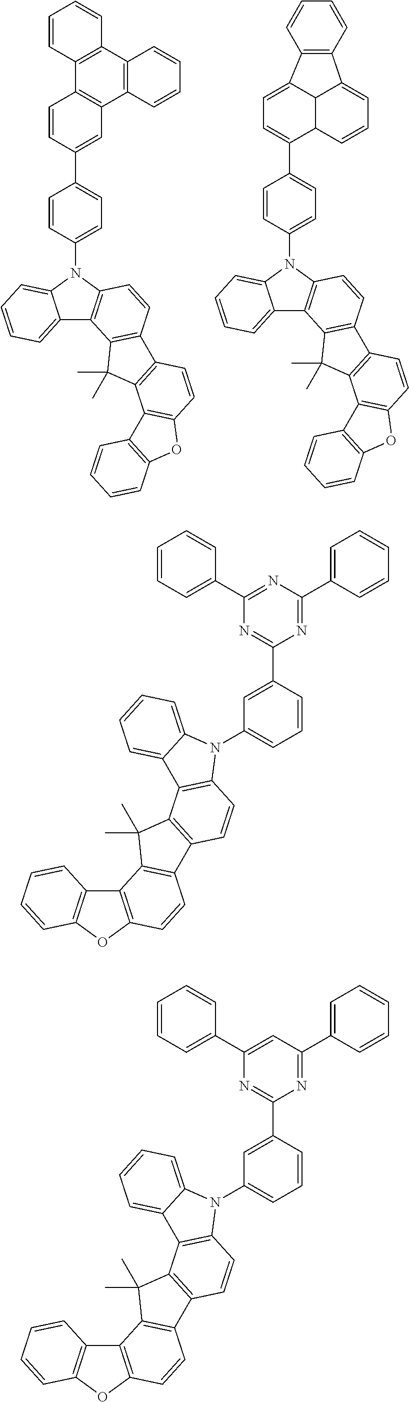 [SMILES: CC1(C)c2c(ccc3oc4ccccc4c23)-c2ccc3c(c21)c1ccccc1n3-c1ccc(-c2ccc3c4ccccc4c4ccccc4c3c2)cc1 ; CC1(C)c2c(ccc3oc4ccccc4c23)-c2ccc3c(c21)c1ccccc1n3-c1ccc(C2=CC=C3c4ccccc4C4=CC=CC2C43)cc1 ; CC1(C)c2c(ccc3oc4ccccc4c23)-c2ccc3c(c21)c1ccccc1n3-c1cccc(-c2nc(-c3ccccc3)nc(-c3ccccc3)n2)c1 ; CC1(C)c2c(ccc3oc4ccccc4c23)-c2ccc3c(c21)c1ccccc1n3-c1cccc(-c2nc(-c3ccccc3)cc(-c3ccccc3)n2)c1]

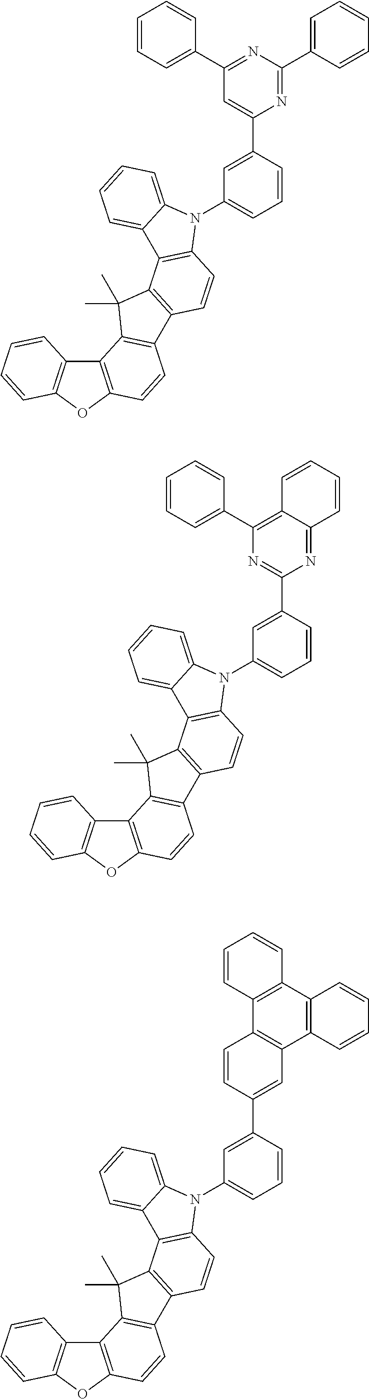 [SMILES: CC1(C)c2c(ccc3oc4ccccc4c23)-c2ccc3c(c21)c1ccccc1n3-c1cccc(-c2cc(-c3ccccc3)nc(-c3ccccc3)n2)c1 ; CC1(C)c2c(ccc3oc4ccccc4c23)-c2ccc3c(c21)c1ccccc1n3-c1cccc(-c2nc(-c3ccccc3)c3ccccc3n2)c1 ; CC1(C)c2c(ccc3oc4ccccc4c23)-c2ccc3c(c21)c1ccccc1n3-c1cccc(-c2ccc3c4ccccc4c4ccccc4c3c2)c1]

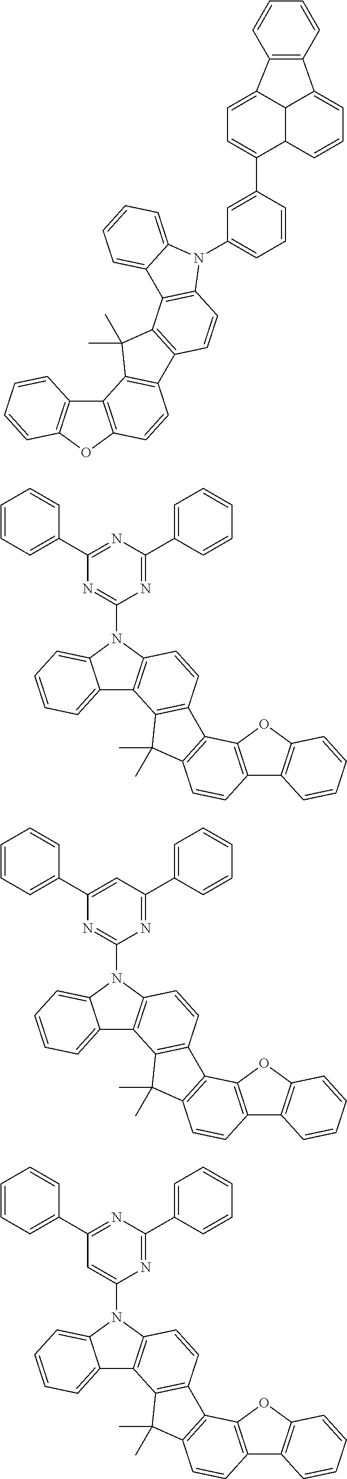

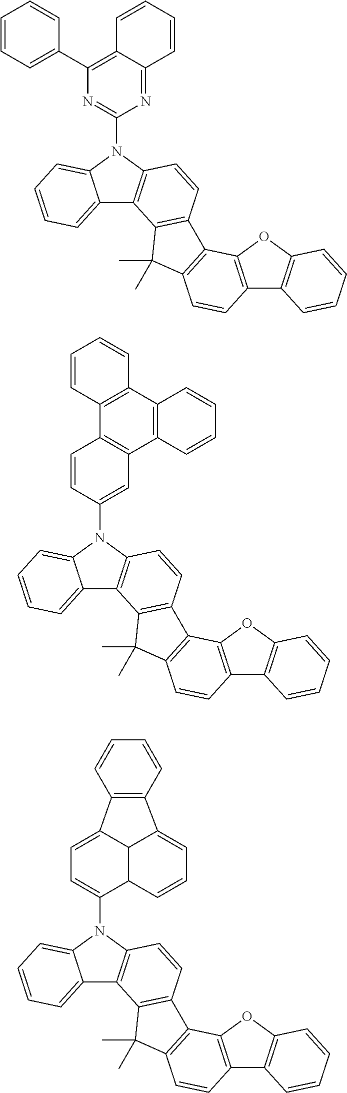 [SMILES: CC1(C)c2ccc3c(oc4ccccc43)c2-c2ccc3c(c21)c1ccccc1n3-c1nc(-c2ccccc2)c2ccccc2n1 ; CC1(C)c2ccc3c(oc4ccccc43)c2-c2ccc3c(c21)c1ccccc1n3-c1ccc2c3ccccc3c3ccccc3c2c1 ; CC1(C)c2ccc3c(oc4ccccc43)c2-c2ccc3c(c21)c1ccccc1n3C1=CC=C2c3ccccc3C3=CC=CC1C32]

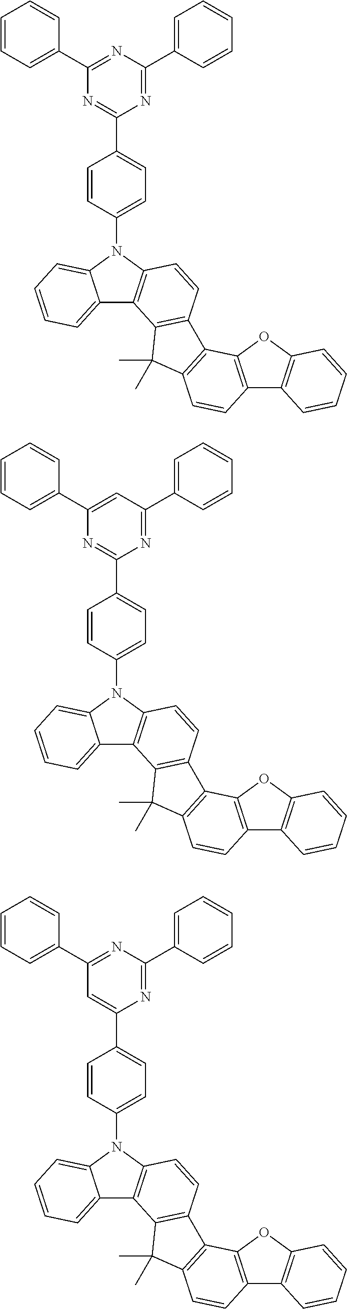

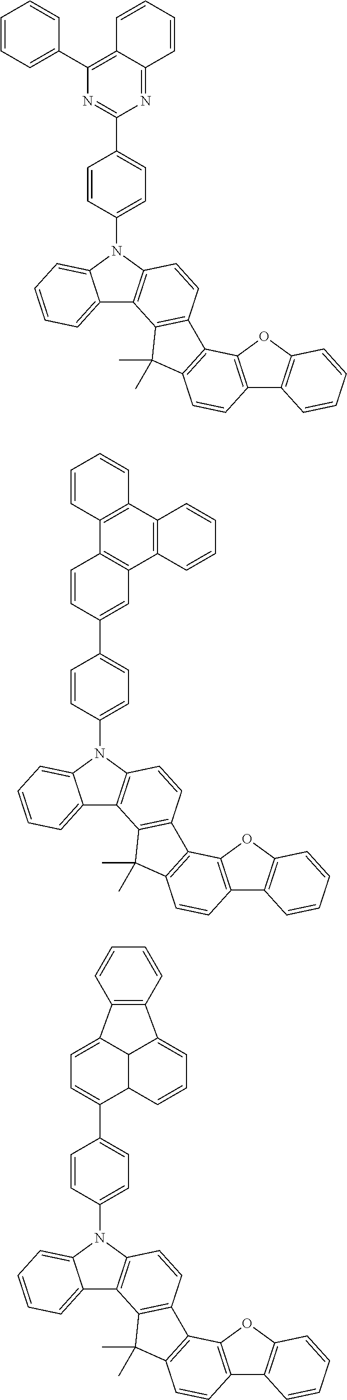 [SMILES: CC1(C)c2ccc3c(oc4ccccc43)c2-c2ccc3c(c21)c1ccccc1n3-c1ccc(-c2nc(-c3ccccc3)c3ccccc3n2)cc1 ; CC1(C)c2ccc3c(oc4ccccc43)c2-c2ccc3c(c21)c1ccccc1n3-c1ccc(-c2ccc3c4ccccc4c4ccccc4c3c2)cc1 ; CC1(C)c2ccc3c(oc4ccccc43)c2-c2ccc3c(c21)c1ccccc1n3-c1ccc(C2=CC=C3c4ccccc4C4=CC=CC2C43)cc1]

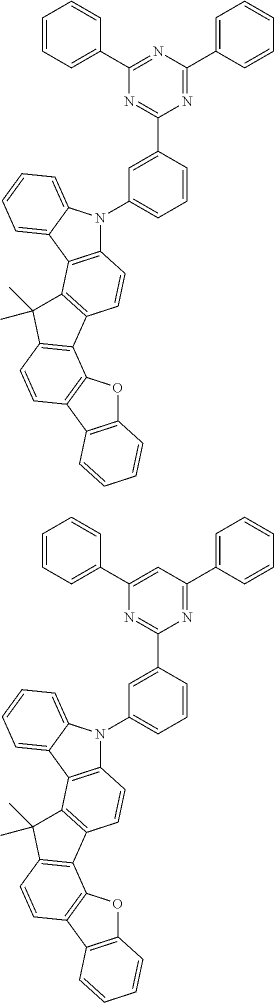

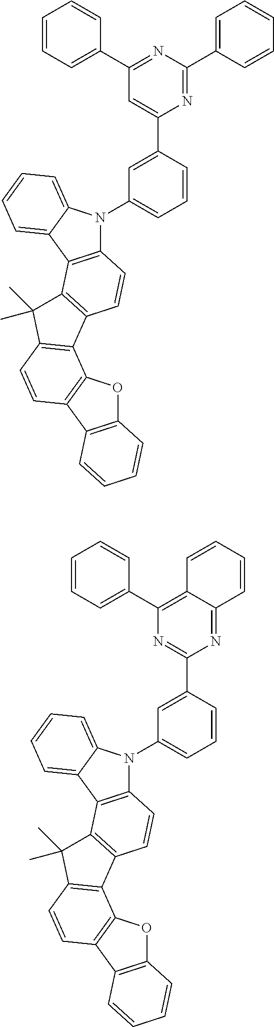 [SMILES: CC1(C)c2ccc3c(oc4ccccc43)c2-c2ccc3c(c21)c1ccccc1n3-c1cccc(-c2cc(-c3ccccc3)nc(-c3ccccc3)n2)c1 ; CC1(C)c2ccc3c(oc4ccccc43)c2-c2ccc3c(c21)c1ccccc1n3-c1cccc(-c2nc(-c3ccccc3)c3ccccc3n2)c1]

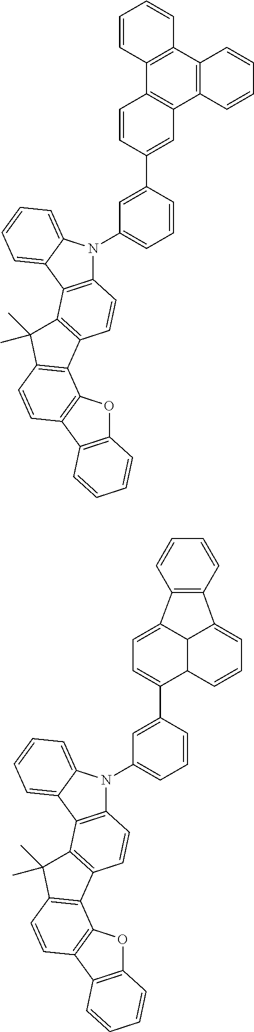 [SMILES: CC1(C)c2ccc3c(oc4ccccc43)c2-c2ccc3c(c21)c1ccccc1n3-c1cccc(-c2ccc3c4ccccc4c4ccccc4c3c2)c1 ; CC1(C)c2ccc3c(oc4ccccc43)c2-c2ccc3c(c21)c1ccccc1n3-c1cccc(C2=CC=C3c4ccccc4C4=CC=CC2C43)c1]

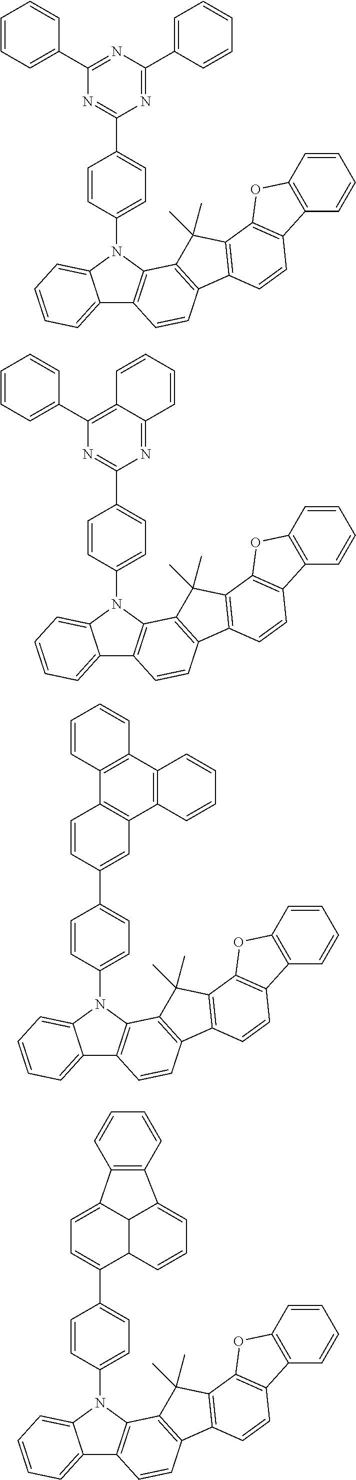

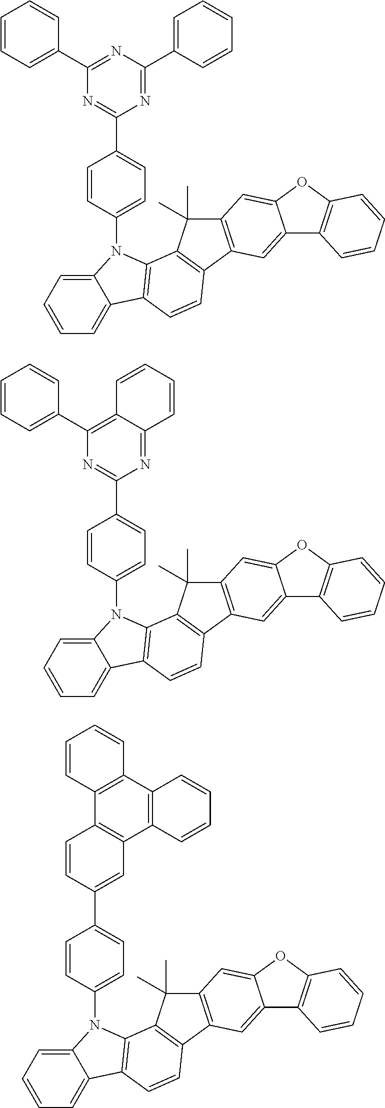

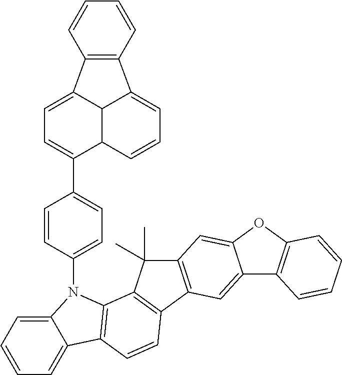 [SMILES: CC1(C)c2cc3oc4ccccc4c3cc2-c2ccc3c4ccccc4n(-c4ccc(C5=CC=C6c7ccccc7C7=CC=CC5C76)cc4)c3c21]

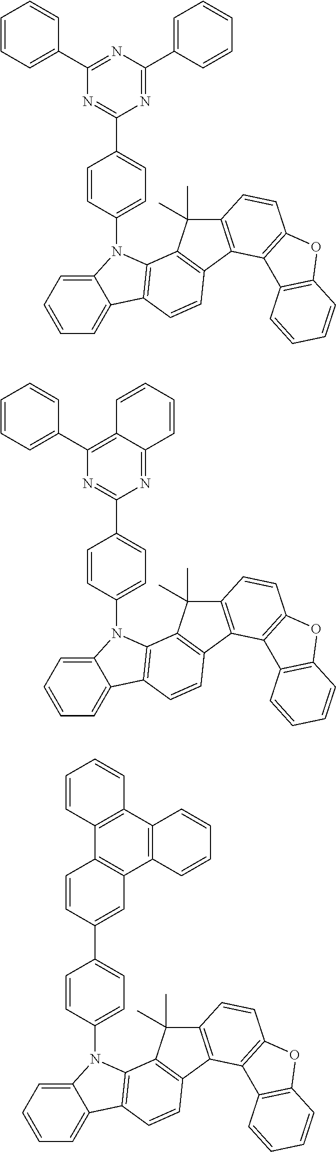

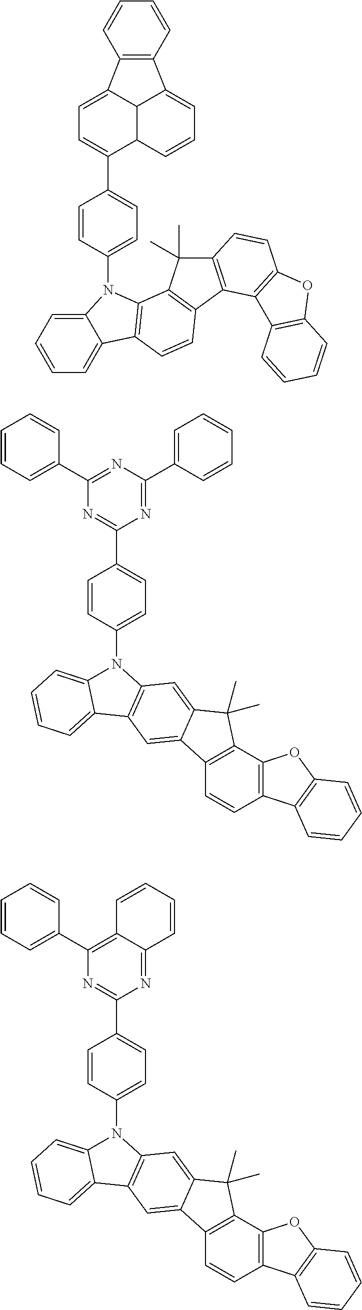 [SMILES: CC1(C)c2ccc3oc4ccccc4c3c2-c2ccc3c4ccccc4n(-c4ccc(C5=CC=C6c7ccccc7C7=CC=CC5C76)cc4)c3c21 ; CC1(C)c2cc3c(cc2-c2ccc4c(oc5ccccc54)c21)c1ccccc1n3-c1ccc(-c2nc(-c3ccccc3)nc(-c3ccccc3)n2)cc1 ; CC1(C)c2cc3c(cc2-c2ccc4c(oc5ccccc54)c21)c1ccccc1n3-c1ccc(-c2nc(-c3ccccc3)c3ccccc3n2)cc1]

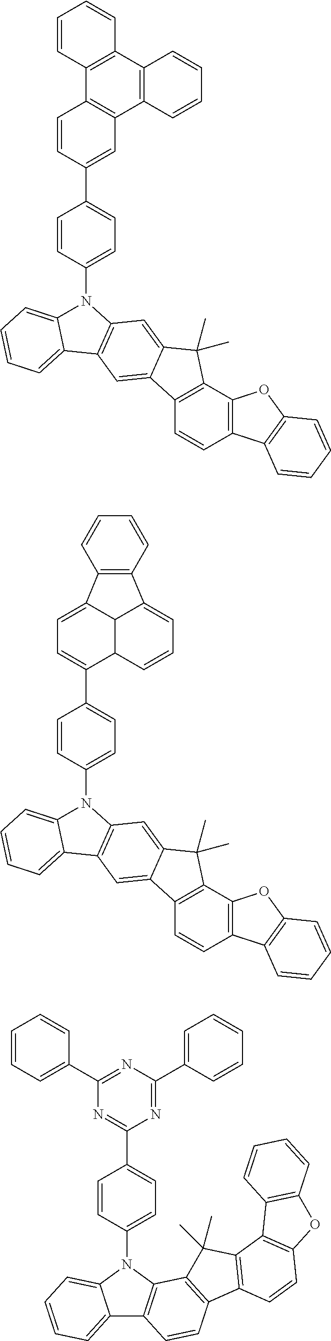 [SMILES: CC1(C)c2cc3c(cc2-c2ccc4c(oc5ccccc54)c21)c1ccccc1n3-c1ccc(-c2ccc3c4ccccc4c4ccccc4c3c2)cc1 ; CC1(C)c2cc3c(cc2-c2ccc4c(oc5ccccc54)c21)c1ccccc1n3-c1ccc(C2=CC=C3c4ccccc4C4=CC=CC2C43)cc1 ; CC1(C)c2c(ccc3oc4ccccc4c23)-c2ccc3c4ccccc4n(-c4ccc(-c5nc(-c6ccccc6)nc(-c6ccccc6)n5)cc4)c3c21]

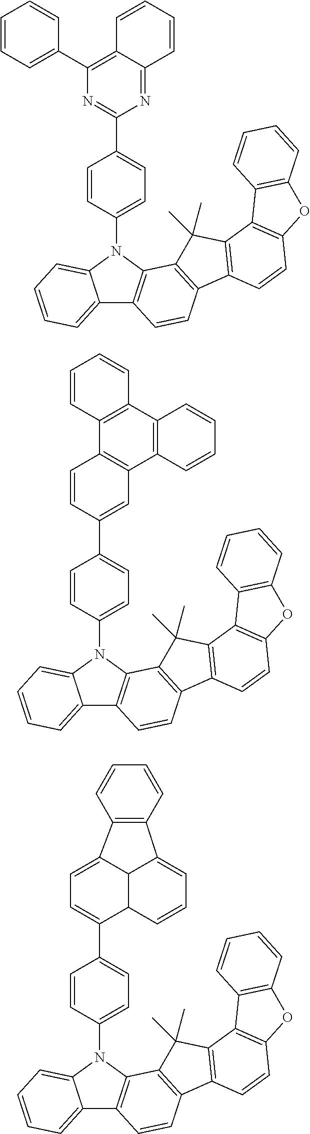

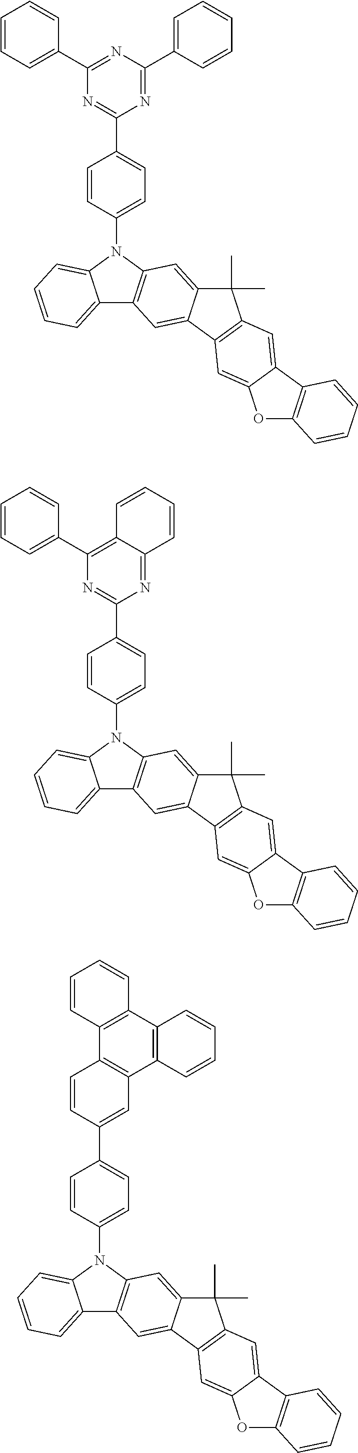 [SMILES: CC1(C)c2cc3c(cc2-c2cc4c5ccccc5n(-c5ccc(-c6nc(-c7ccccc7)nc(-c7ccccc7)n6)cc5)c4cc21)oc1ccccc13 ; CC1(C)c2cc3c(cc2-c2cc4c5ccccc5n(-c5ccc(-c6nc(-c7ccccc7)c7ccccc7n6)cc5)c4cc21)oc1ccccc13 ; CC1(C)c2cc3c(cc2-c2cc4c5ccccc5n(-c5ccc(-c6ccc7c8ccccc8c8ccccc8c7c6)cc5)c4cc21)oc1ccccc13]

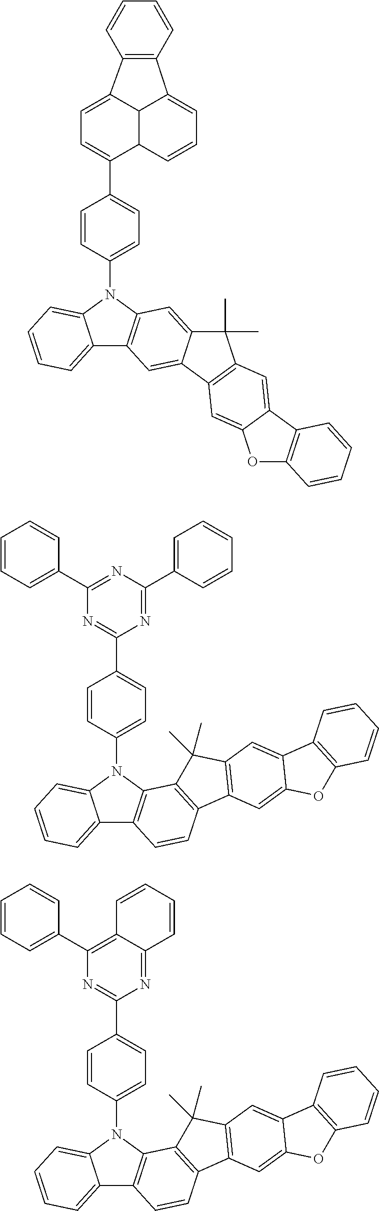

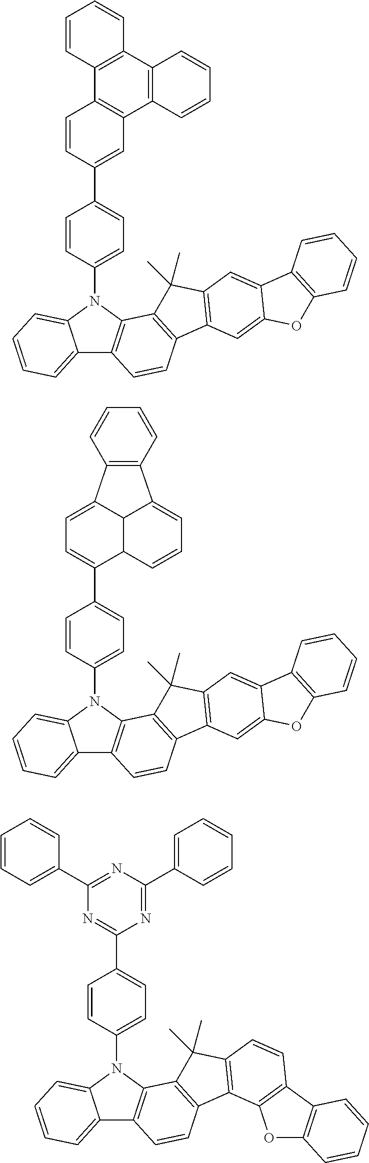

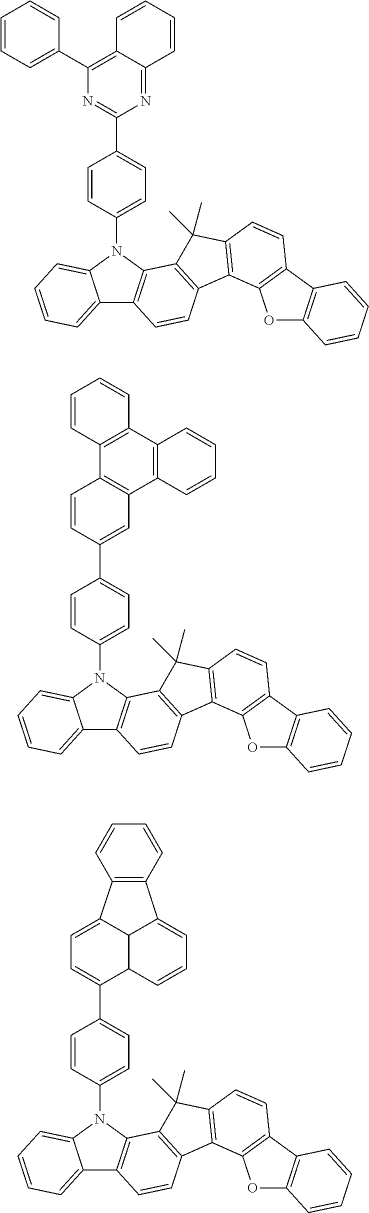 [SMILES: CC1(C)c2ccc3c(oc4ccccc43)c2-c2ccc3c4ccccc4n(-c4ccc(-c5nc(-c6ccccc6)c6ccccc6n5)cc4)c3c21 ; CC1(C)c2ccc3c(oc4ccccc43)c2-c2ccc3c4ccccc4n(-c4ccc(-c5ccc6c7ccccc7c7ccccc7c6c5)cc4)c3c21 ; CC1(C)c2ccc3c(oc4ccccc43)c2-c2ccc3c4ccccc4n(-c4ccc(C5=CC=C6c7ccccc7C7=CC=CC5C76)cc4)c3c21]

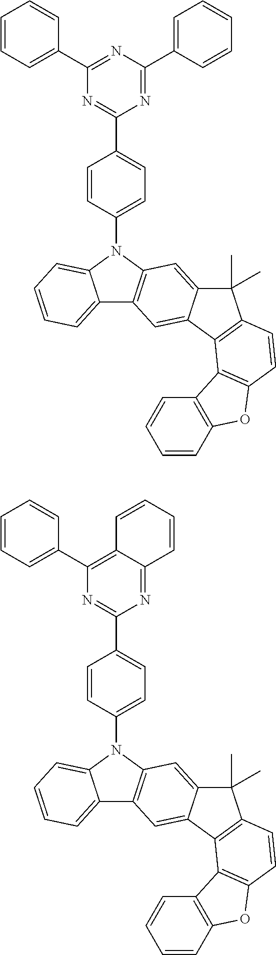 [SMILES: CC1(C)c2cc3c(cc2-c2c1ccc1oc4ccccc4c21)c1ccccc1n3-c1ccc(-c2nc(-c3ccccc3)nc(-c3ccccc3)n2)cc1 ; CC1(C)c2cc3c(cc2-c2c1ccc1oc4ccccc4c21)c1ccccc1n3-c1ccc(-c2nc(-c3ccccc3)c3ccccc3n2)cc1]

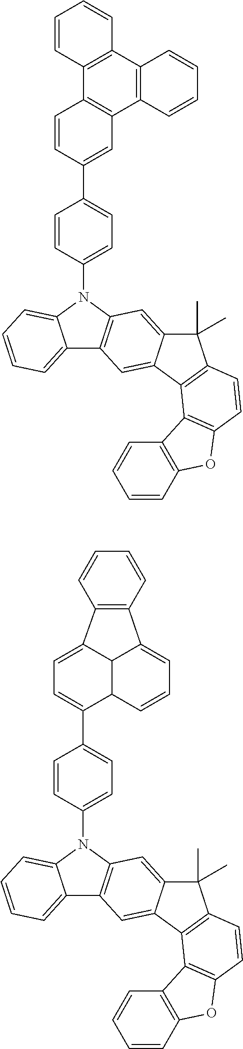 [SMILES: CC1(C)c2cc3c(cc2-c2c1ccc1oc4ccccc4c21)c1ccccc1n3-c1ccc(-c2ccc3c4ccccc4c4ccccc4c3c2)cc1 ; CC1(C)c2cc3c(cc2-c2c1ccc1oc4ccccc4c21)c1ccccc1n3-c1ccc(C2=CC=C3c4ccccc4C4=CC=CC2C43)cc1]

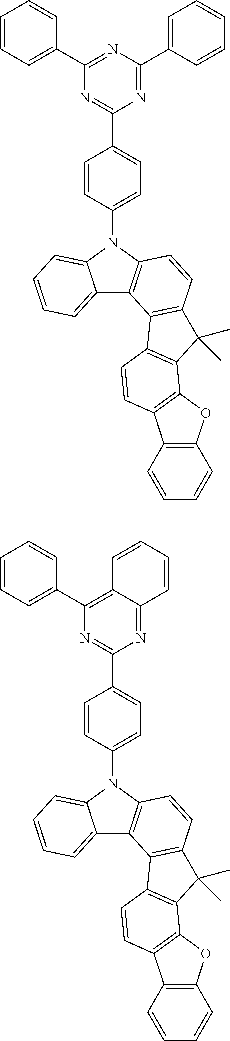 [SMILES: CC1(C)c2ccc3c(c2-c2ccc4c(oc5ccccc54)c21)c1ccccc1n3-c1ccc(-c2nc(-c3ccccc3)nc(-c3ccccc3)n2)cc1 ; CC1(C)c2ccc3c(c2-c2ccc4c(oc5ccccc54)c21)c1ccccc1n3-c1ccc(-c2nc(-c3ccccc3)c3ccccc3n2)cc1]

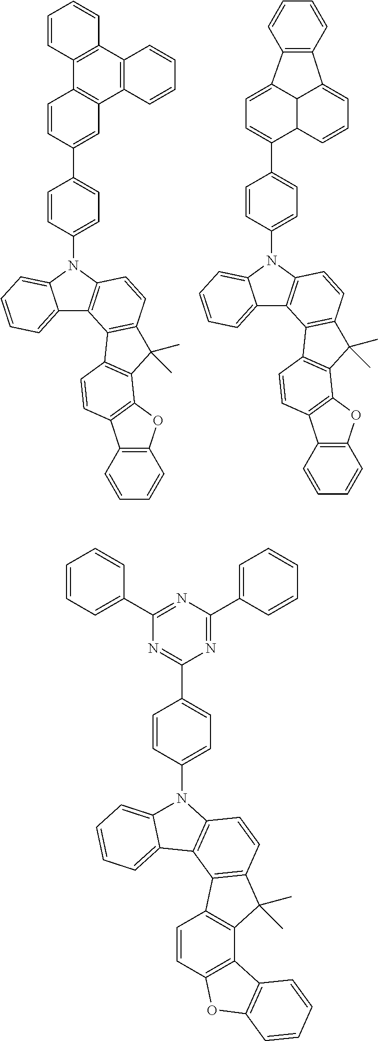

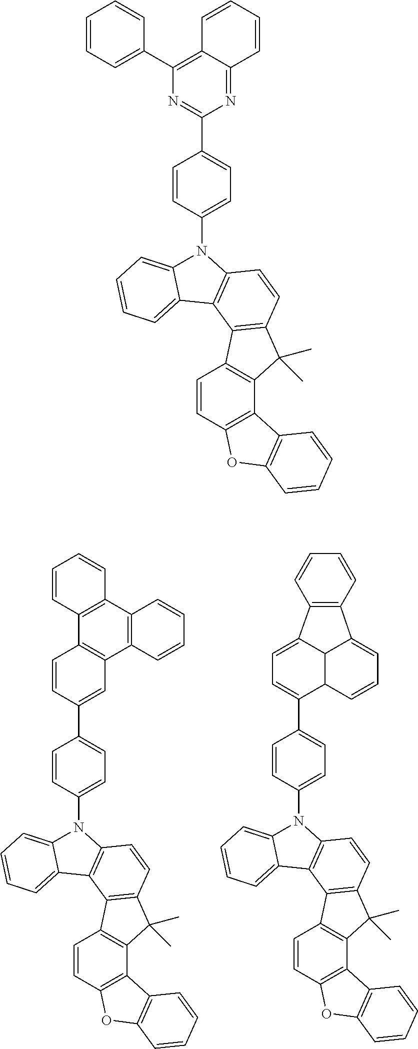 [SMILES: CC1(C)c2ccc3c(c2-c2ccc4oc5ccccc5c4c21)c1ccccc1n3-c1ccc(-c2nc(-c3ccccc3)c3ccccc3n2)cc1 ; CC1(C)c2ccc3c(c2-c2ccc4oc5ccccc5c4c21)c1ccccc1n3-c1ccc(-c2ccc3c4ccccc4c4ccccc4c3c2)cc1 ; CC1(C)c2ccc3c(c2-c2ccc4oc5ccccc5c4c21)c1ccccc1n3-c1ccc(C2=CC=C3c4ccccc4C4=CC=CC2C43)cc1]

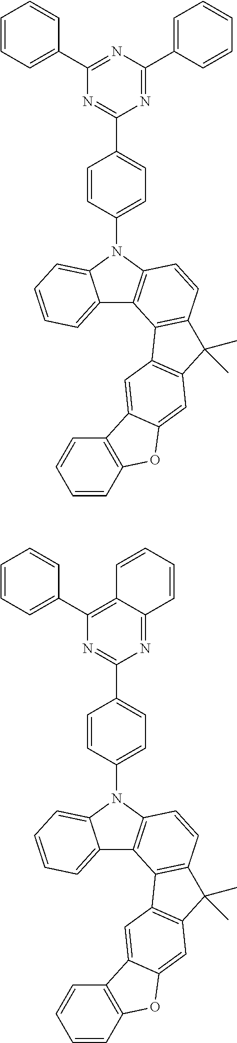 [SMILES: CC1(C)c2cc3oc4ccccc4c3cc2-c2c1ccc1c2c2ccccc2n1-c1ccc(-c2nc(-c3ccccc3)nc(-c3ccccc3)n2)cc1 ; CC1(C)c2cc3oc4ccccc4c3cc2-c2c1ccc1c2c2ccccc2n1-c1ccc(-c2nc(-c3ccccc3)c3ccccc3n2)cc1]

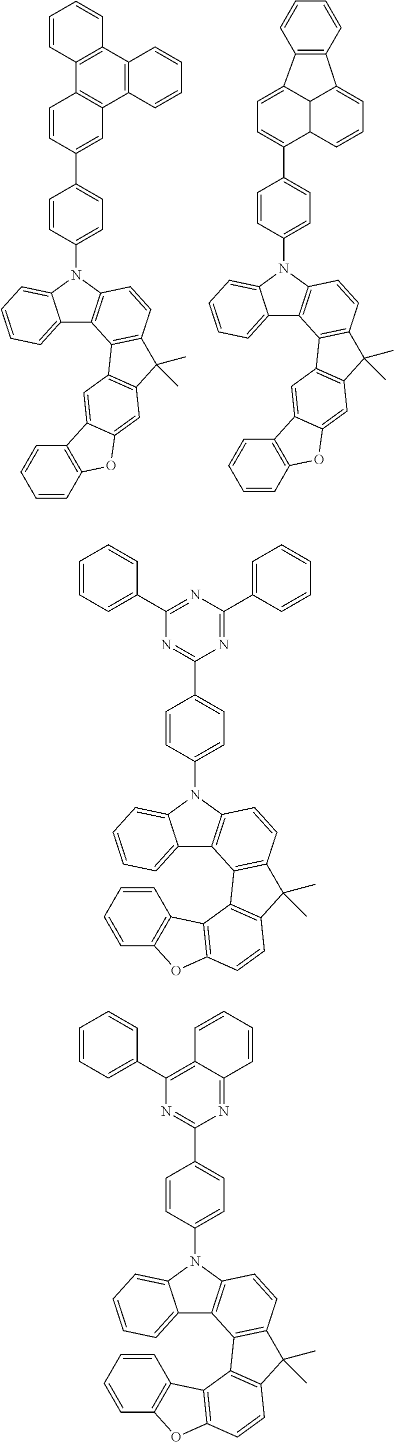

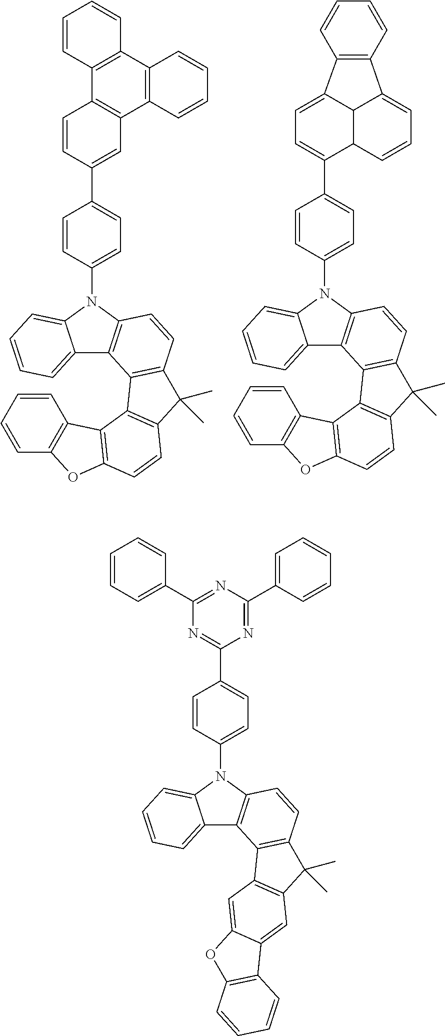

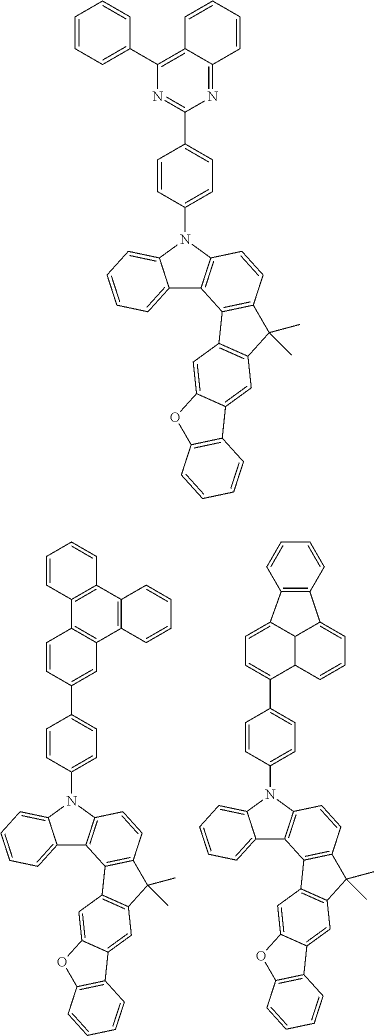 [SMILES: CC1(C)c2cc3c(cc2-c2c1ccc1c2c2ccccc2n1-c1ccc(-c2nc(-c4ccccc4)c4ccccc4n2)cc1)oc1ccccc13 ; CC1(C)c2cc3c(cc2-c2c1ccc1c2c2ccccc2n1-c1ccc(-c2ccc4c5ccccc5c5ccccc5c4c2)cc1)oc1ccccc13 ; CC1(C)c2cc3c(cc2-c2c1ccc1c2c2ccccc2n1-c1ccc(C2=CC=C4c5ccccc5C5=CC=CC2C54)cc1)oc1ccccc13]

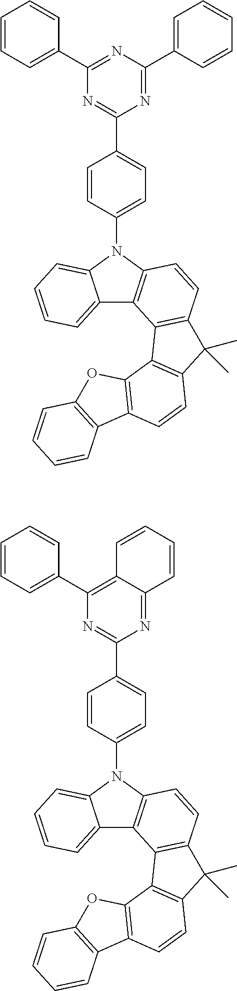 [SMILES: CC1(C)c2ccc3c(oc4ccccc43)c2-c2c1ccc1c2c2ccccc2n1-c1ccc(-c2nc(-c3ccccc3)nc(-c3ccccc3)n2)cc1 ; CC1(C)c2ccc3c(oc4ccccc43)c2-c2c1ccc1c2c2ccccc2n1-c1ccc(-c2nc(-c3ccccc3)c3ccccc3n2)cc1]

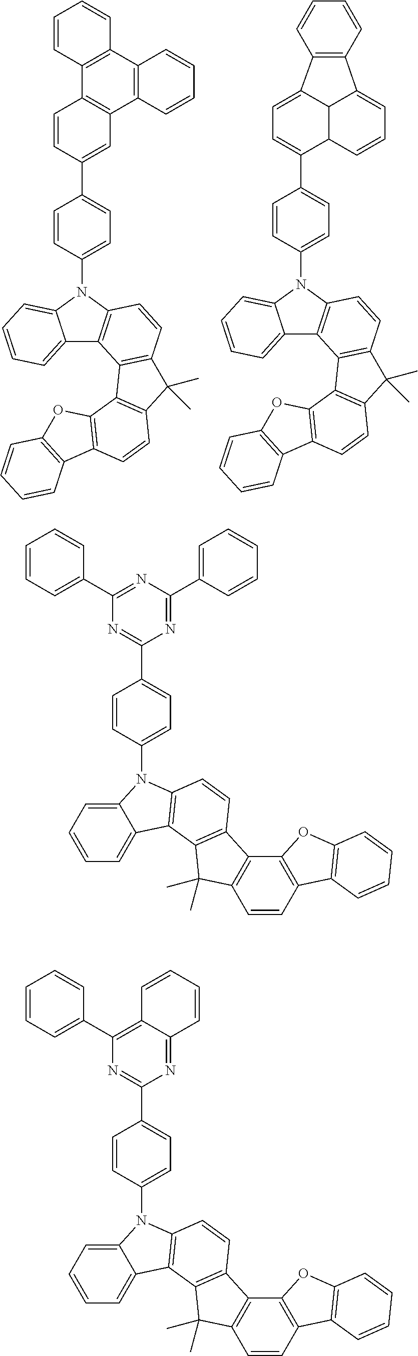

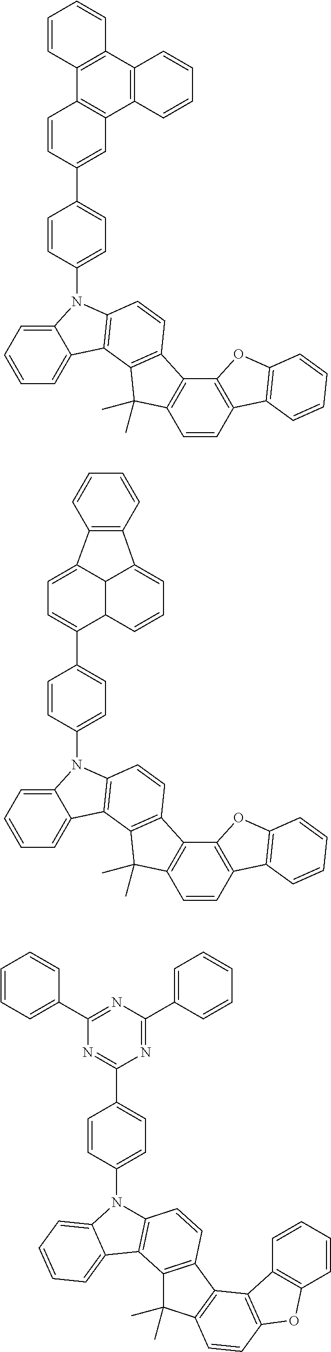

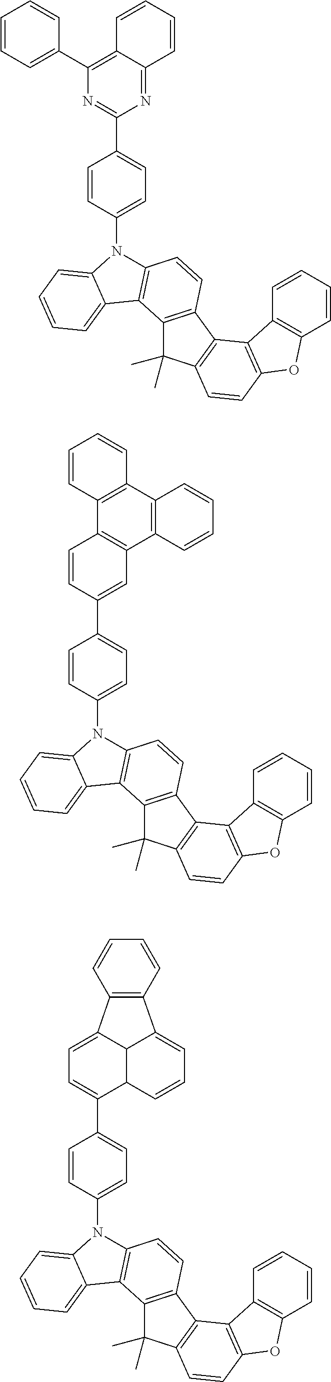 [SMILES: CC1(C)c2ccc3oc4ccccc4c3c2-c2ccc3c(c21)c1ccccc1n3-c1ccc(-c2nc(-c3ccccc3)c3ccccc3n2)cc1 ; CC1(C)c2ccc3oc4ccccc4c3c2-c2ccc3c(c21)c1ccccc1n3-c1ccc(-c2ccc3c4ccccc4c4ccccc4c3c2)cc1 ; CC1(C)c2ccc3oc4ccccc4c3c2-c2ccc3c(c21)c1ccccc1n3-c1ccc(C2=CC=C3c4ccccc4C4=CC=CC2C43)cc1]

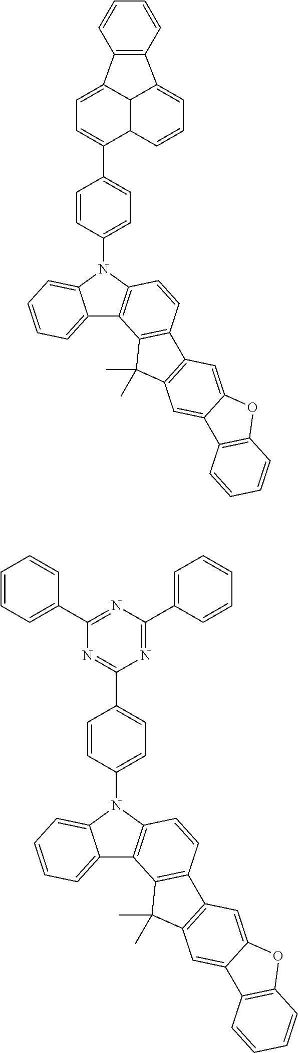 [SMILES: CC1(C)c2cc3c(cc2-c2ccc4c(c21)c1ccccc1n4-c1ccc(C2=CC=C4c5ccccc5C5=CC=CC2C54)cc1)oc1ccccc13 ; CC1(C)c2cc3c(cc2-c2ccc4c(c21)c1ccccc1n4-c1ccc(-c2nc(-c4ccccc4)nc(-c4ccccc4)n2)cc1)oc1ccccc13]

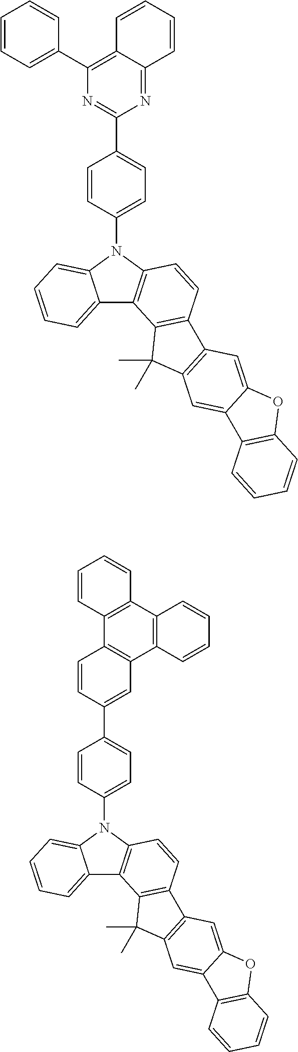 [SMILES: CC1(C)c2cc3c(cc2-c2ccc4c(c21)c1ccccc1n4-c1ccc(-c2nc(-c4ccccc4)c4ccccc4n2)cc1)oc1ccccc13 ; CC1(C)c2cc3c(cc2-c2ccc4c(c21)c1ccccc1n4-c1ccc(-c2ccc4c5ccccc5c5ccccc5c4c2)cc1)oc1ccccc13]

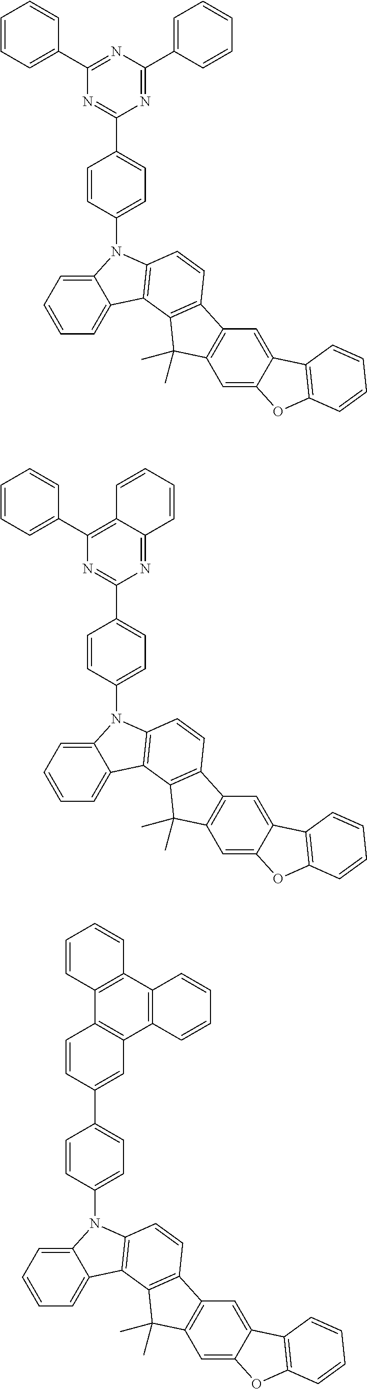 [SMILES: CC1(C)c2cc3oc4ccccc4c3cc2-c2ccc3c(c21)c1ccccc1n3-c1ccc(-c2nc(-c3ccccc3)nc(-c3ccccc3)n2)cc1 ; CC1(C)c2cc3oc4ccccc4c3cc2-c2ccc3c(c21)c1ccccc1n3-c1ccc(-c2nc(-c3ccccc3)c3ccccc3n2)cc1 ; CC1(C)c2cc3oc4ccccc4c3cc2-c2ccc3c(c21)c1ccccc1n3-c1ccc(-c2ccc3c4ccccc4c4ccccc4c3c2)cc1]

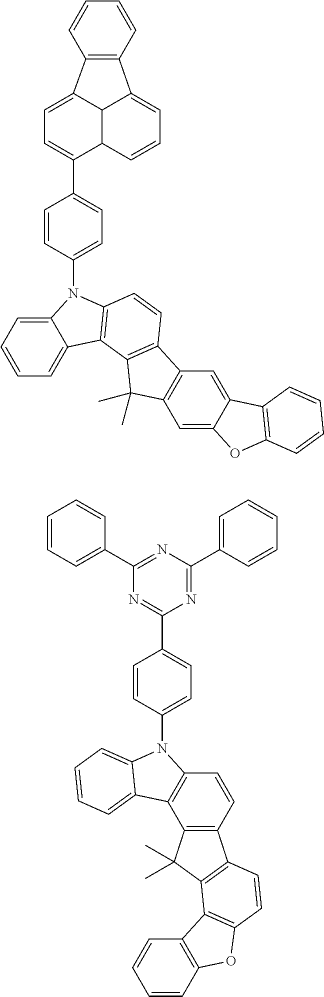 [SMILES: CC1(C)c2cc3oc4ccccc4c3cc2-c2ccc3c(c21)c1ccccc1n3-c1ccc(C2=CC=C3c4ccccc4C4=CC=CC2C43)cc1 ; CC1(C)c2c(ccc3oc4ccccc4c23)-c2ccc3c(c21)c1ccccc1n3-c1ccc(-c2nc(-c3ccccc3)nc(-c3ccccc3)n2)cc1]

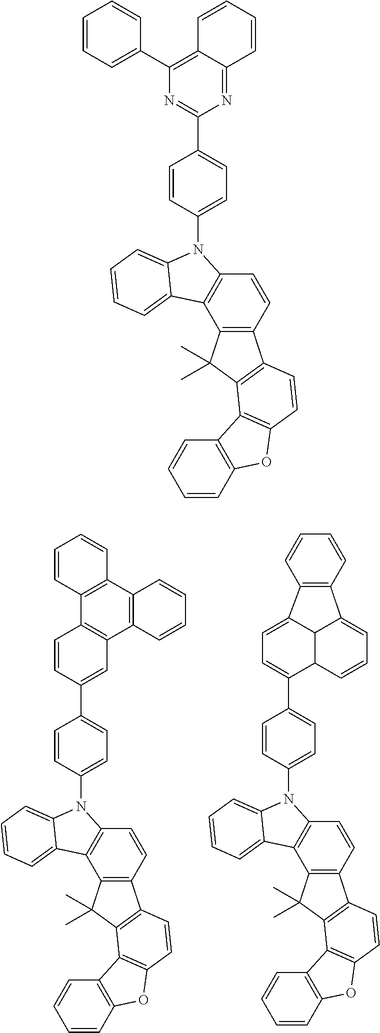 [SMILES: CC1(C)c2c(ccc3oc4ccccc4c23)-c2ccc3c(c21)c1ccccc1n3-c1ccc(-c2nc(-c3ccccc3)c3ccccc3n2)cc1 ; CC1(C)c2c(ccc3oc4ccccc4c23)-c2ccc3c(c21)c1ccccc1n3-c1ccc(-c2ccc3c4ccccc4c4ccccc4c3c2)cc1 ; CC1(C)c2c(ccc3oc4ccccc4c23)-c2ccc3c(c21)c1ccccc1n3-c1ccc(C2=CC=C3c4ccccc4C4=CC=CC2C43)cc1]

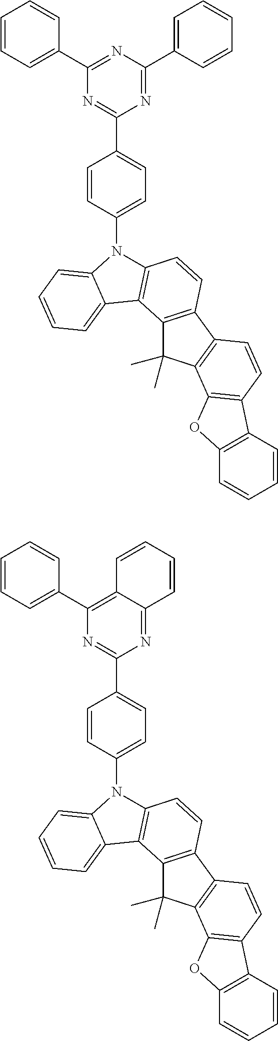 [SMILES: CC1(C)c2c(ccc3c2oc2ccccc23)-c2ccc3c(c21)c1ccccc1n3-c1ccc(-c2nc(-c3ccccc3)nc(-c3ccccc3)n2)cc1 ; CC1(C)c2c(ccc3c2oc2ccccc23)-c2ccc3c(c21)c1ccccc1n3-c1ccc(-c2nc(-c3ccccc3)c3ccccc3n2)cc1]

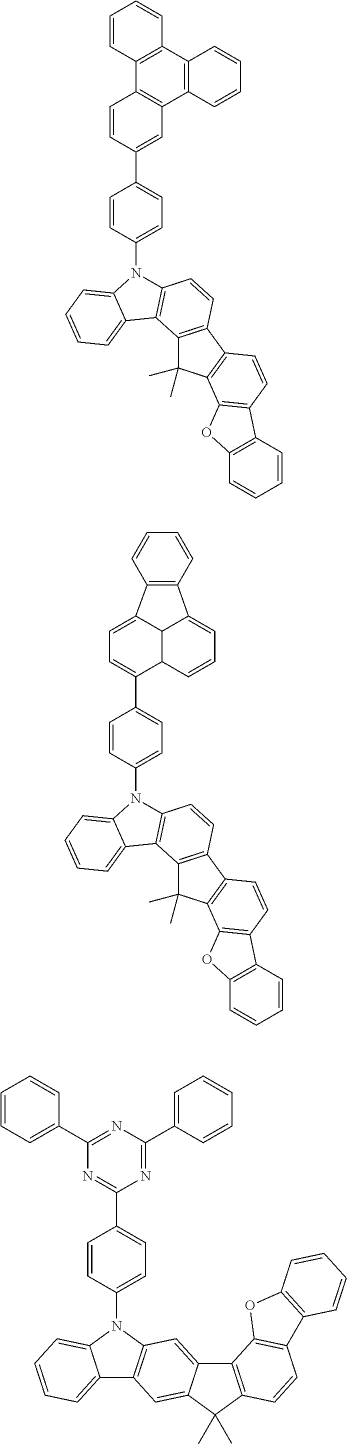 [SMILES: CC1(C)c2c(ccc3c2oc2ccccc23)-c2ccc3c(c21)c1ccccc1n3-c1ccc(-c2ccc3c4ccccc4c4ccccc4c3c2)cc1 ; CC1(C)c2c(ccc3c2oc2ccccc23)-c2ccc3c(c21)c1ccccc1n3-c1ccc(C2=CC=C3c4ccccc4C4=CC=CC2C43)cc1 ; CC1(C)c2cc3c4ccccc4n(-c4ccc(-c5nc(-c6ccccc6)nc(-c6ccccc6)n5)cc4)c3cc2-c2c1ccc1c2oc2ccccc21]

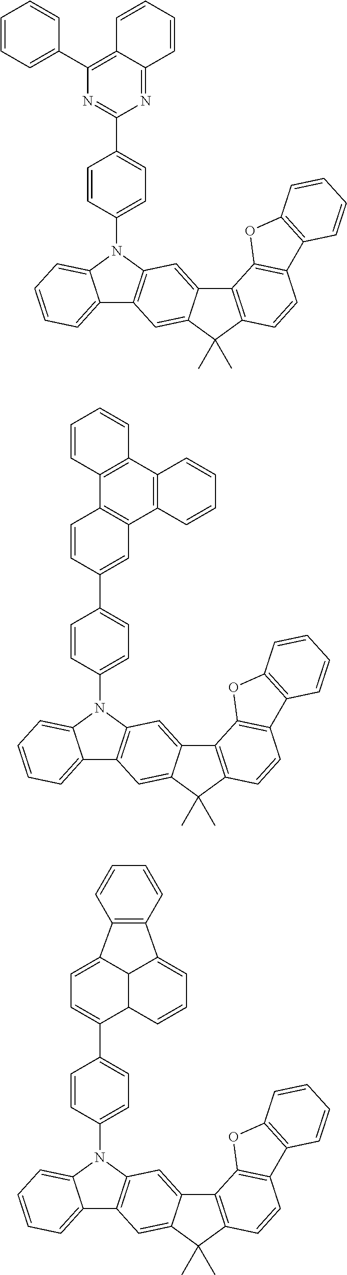 [SMILES: CC1(C)c2cc3c4ccccc4n(-c4ccc(-c5nc(-c6ccccc6)c6ccccc6n5)cc4)c3cc2-c2c1ccc1c2oc2ccccc21 ; CC1(C)c2cc3c4ccccc4n(-c4ccc(-c5ccc6c7ccccc7c7ccccc7c6c5)cc4)c3cc2-c2c1ccc1c2oc2ccccc21 ; CC1(C)c2cc3c4ccccc4n(-c4ccc(C5=CC=C6c7ccccc7C7=CC=CC5C76)cc4)c3cc2-c2c1ccc1c2oc2ccccc21]

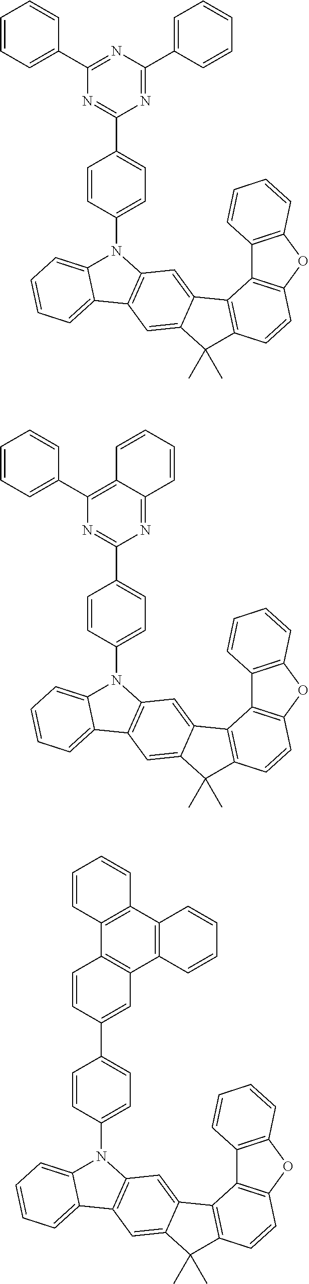 [SMILES: CC1(C)c2cc3c4ccccc4n(-c4ccc(-c5nc(-c6ccccc6)nc(-c6ccccc6)n5)cc4)c3cc2-c2c1ccc1oc3ccccc3c21 ; CC1(C)c2cc3c4ccccc4n(-c4ccc(-c5nc(-c6ccccc6)c6ccccc6n5)cc4)c3cc2-c2c1ccc1oc3ccccc3c21 ; CC1(C)c2cc3c4ccccc4n(-c4ccc(-c5ccc6c7ccccc7c7ccccc7c6c5)cc4)c3cc2-c2c1ccc1oc3ccccc3c21]

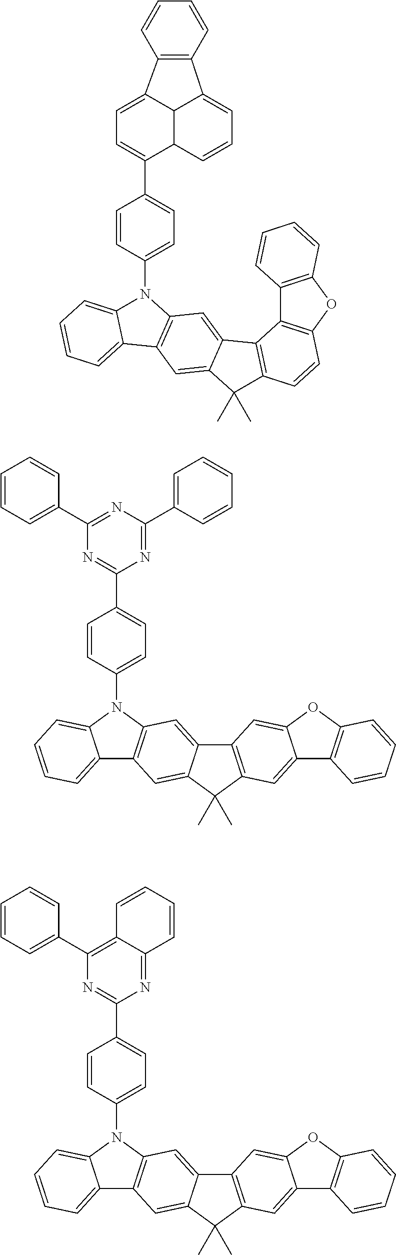

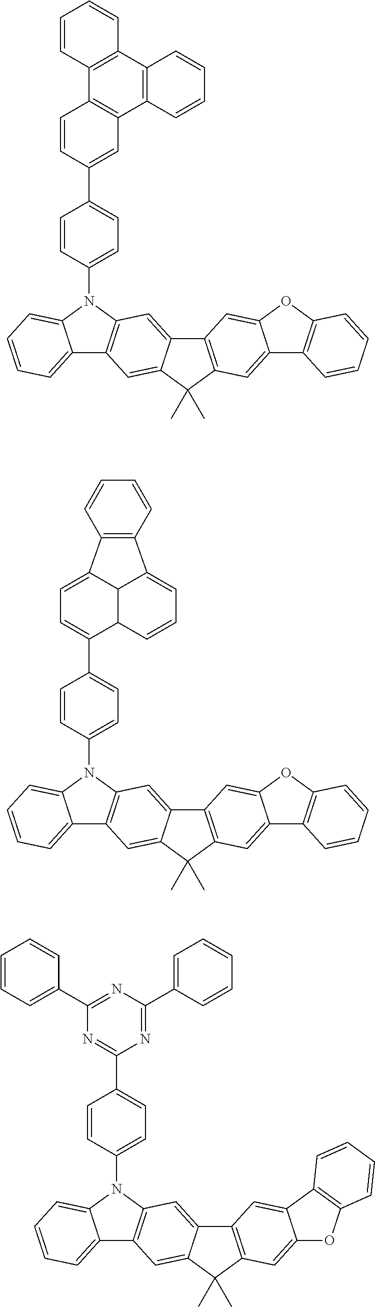 [SMILES: CC1(C)c2cc3c(cc2-c2cc4c(cc21)c1ccccc1n4-c1ccc(-c2ccc4c5ccccc5c5ccccc5c4c2)cc1)oc1ccccc13 ; CC1(C)c2cc3c(cc2-c2cc4c(cc21)c1ccccc1n4-c1ccc(C2=CC=C4c5ccccc5C5=CC=CC2C54)cc1)oc1ccccc13 ; CC1(C)c2cc3oc4ccccc4c3cc2-c2cc3c(cc21)c1ccccc1n3-c1ccc(-c2nc(-c3ccccc3)nc(-c3ccccc3)n2)cc1]

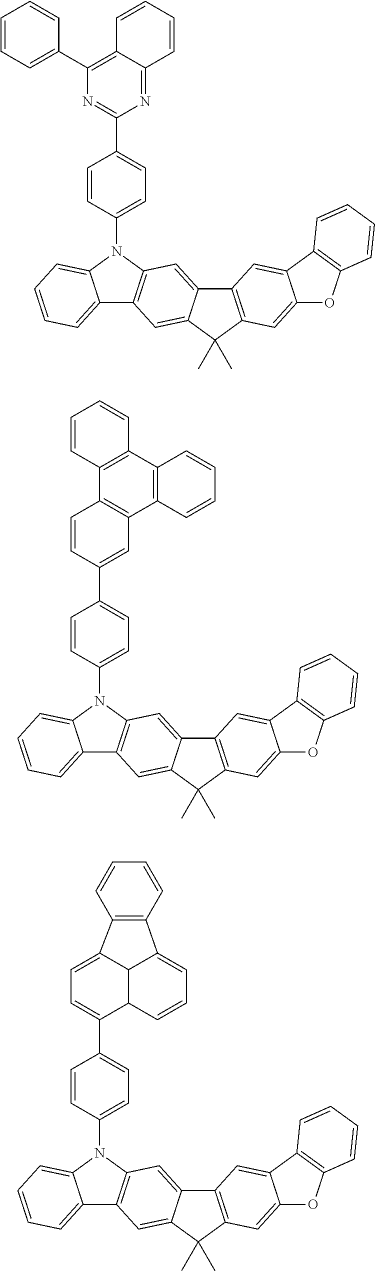 [SMILES: CC1(C)c2cc3oc4ccccc4c3cc2-c2cc3c(cc21)c1ccccc1n3-c1ccc(-c2nc(-c3ccccc3)c3ccccc3n2)cc1 ; CC1(C)c2cc3oc4ccccc4c3cc2-c2cc3c(cc21)c1ccccc1n3-c1ccc(-c2ccc3c4ccccc4c4ccccc4c3c2)cc1 ; CC1(C)c2cc3oc4ccccc4c3cc2-c2cc3c(cc21)c1ccccc1n3-c1ccc(C2=CC=C3c4ccccc4C4=CC=CC2C43)cc1]

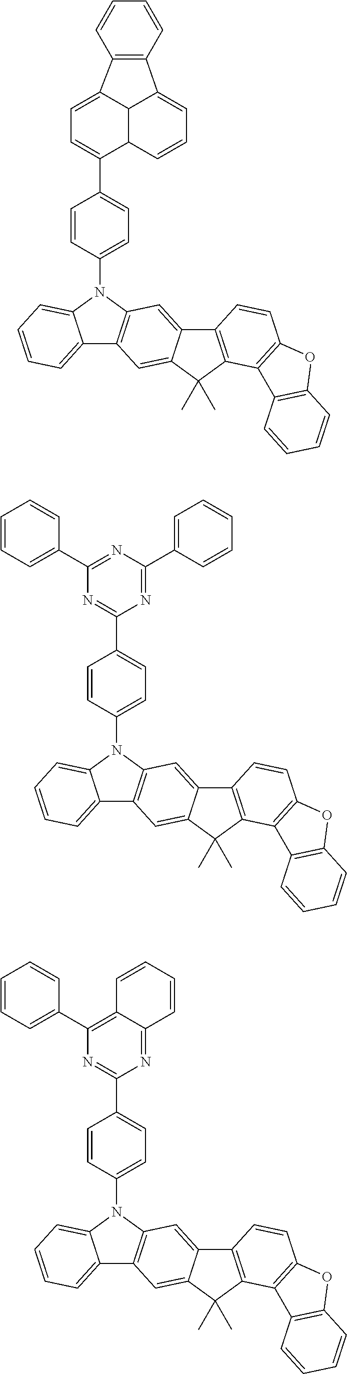 [SMILES: CC1(C)c2cc3c4ccccc4n(-c4ccc(C5=CC=C6c7ccccc7C7=CC=CC5C76)cc4)c3cc2-c2ccc3oc4ccccc4c3c21 ; CC1(C)c2cc3c4ccccc4n(-c4ccc(-c5nc(-c6ccccc6)nc(-c6ccccc6)n5)cc4)c3cc2-c2ccc3oc4ccccc4c3c21 ; CC1(C)c2cc3c4ccccc4n(-c4ccc(-c5nc(-c6ccccc6)c6ccccc6n5)cc4)c3cc2-c2ccc3oc4ccccc4c3c21]

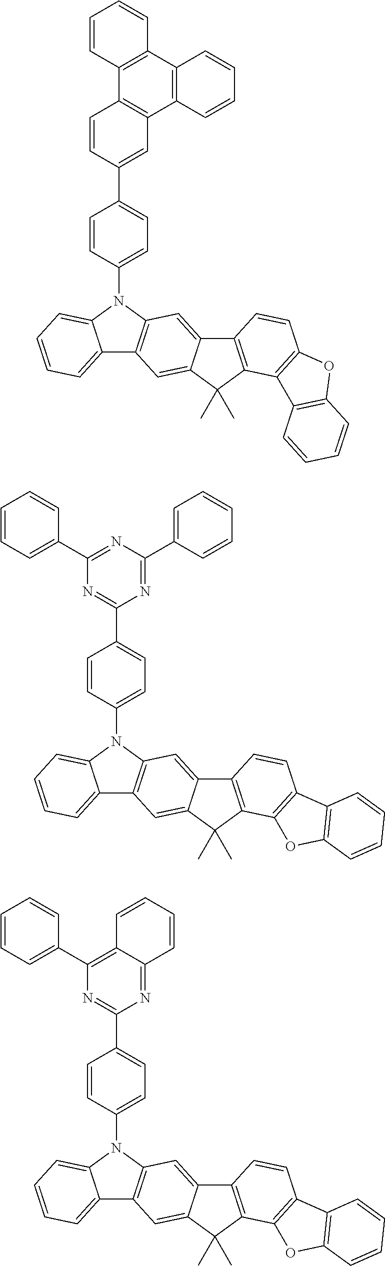 [SMILES: CC1(C)c2cc3c4ccccc4n(-c4ccc(-c5ccc6c7ccccc7c7ccccc7c6c5)cc4)c3cc2-c2ccc3oc4ccccc4c3c21 ; CC1(C)c2cc3c4ccccc4n(-c4ccc(-c5nc(-c6ccccc6)nc(-c6ccccc6)n5)cc4)c3cc2-c2ccc3c(oc4ccccc43)c21 ; CC1(C)c2cc3c4ccccc4n(-c4ccc(-c5nc(-c6ccccc6)c6ccccc6n5)cc4)c3cc2-c2ccc3c(oc4ccccc43)c21]

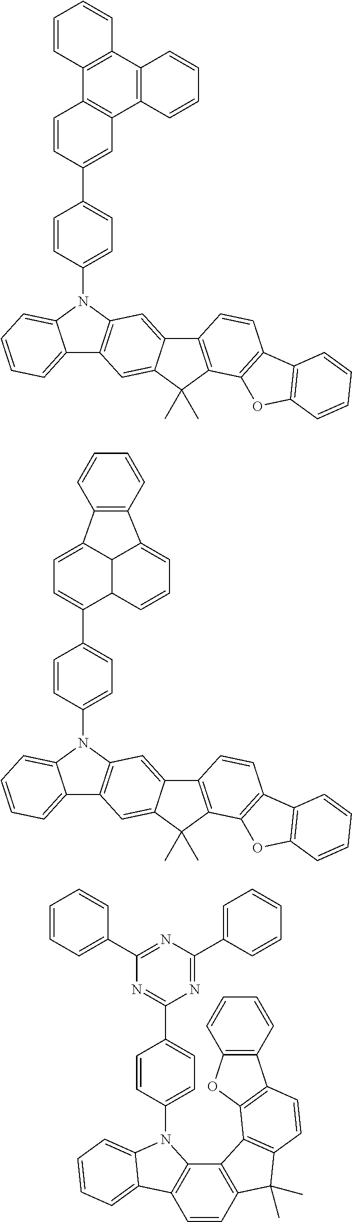

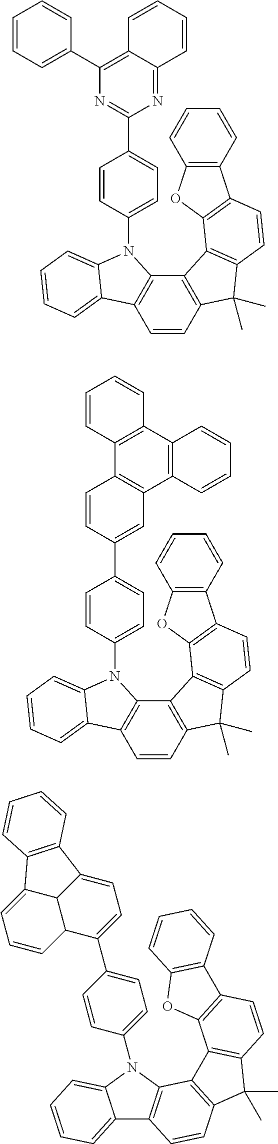 [SMILES: CC1(C)c2ccc3c(oc4ccccc43)c2-c2c1ccc1c3ccccc3n(-c3ccc(-c4nc(-c5ccccc5)c5ccccc5n4)cc3)c21 ; CC1(C)c2ccc3c(oc4ccccc43)c2-c2c1ccc1c3ccccc3n(-c3ccc(-c4ccc5c6ccccc6c6ccccc6c5c4)cc3)c21 ; CC1(C)c2ccc3c(oc4ccccc43)c2-c2c1ccc1c3ccccc3n(-c3ccc(C4=CC=C5c6ccccc6C6=CC=CC4C65)cc3)c21]

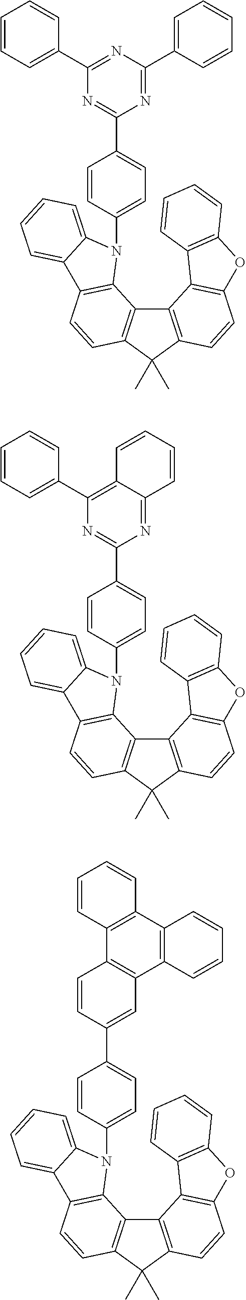

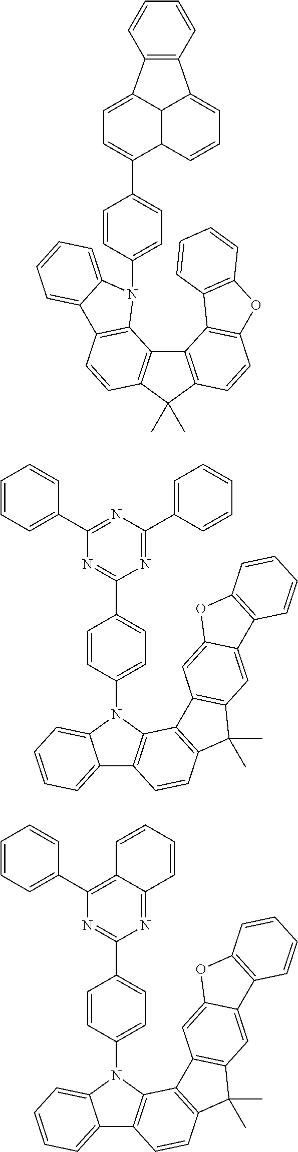

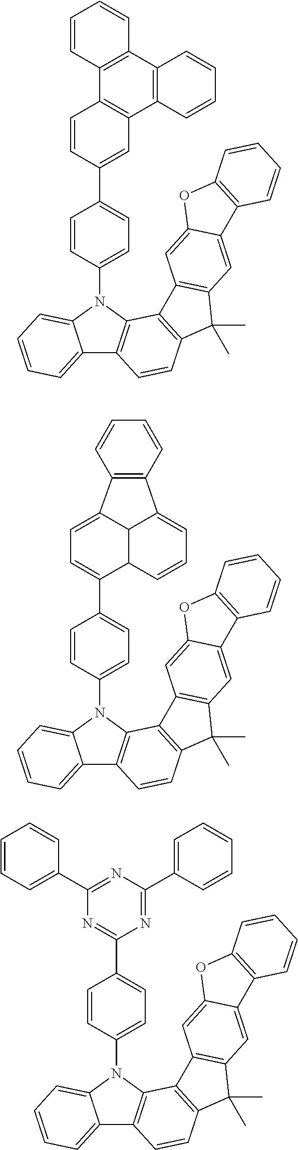

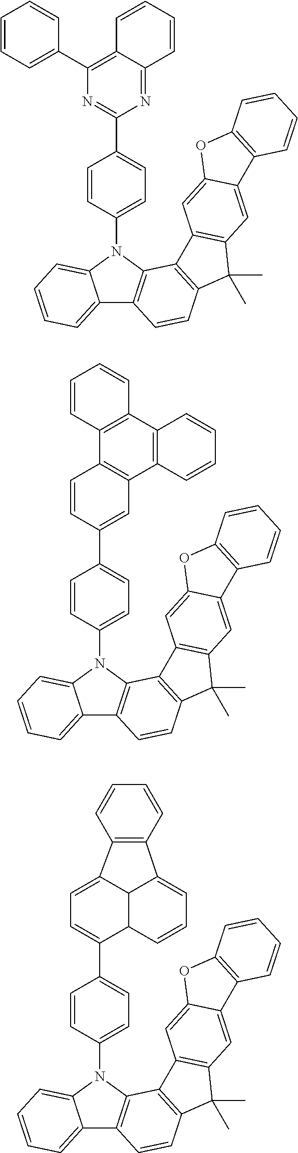

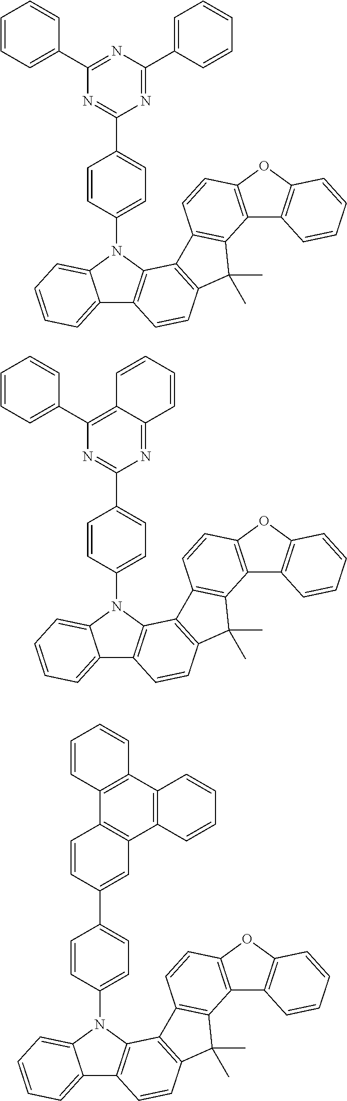

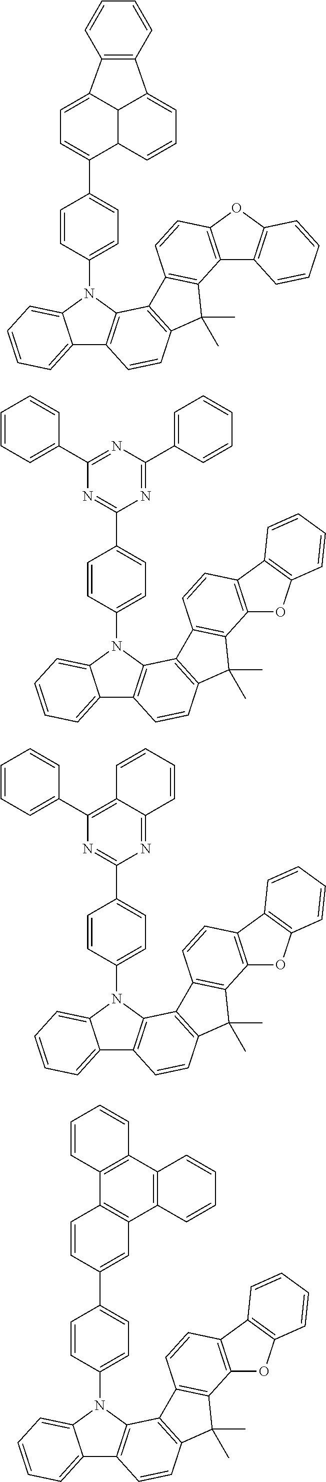

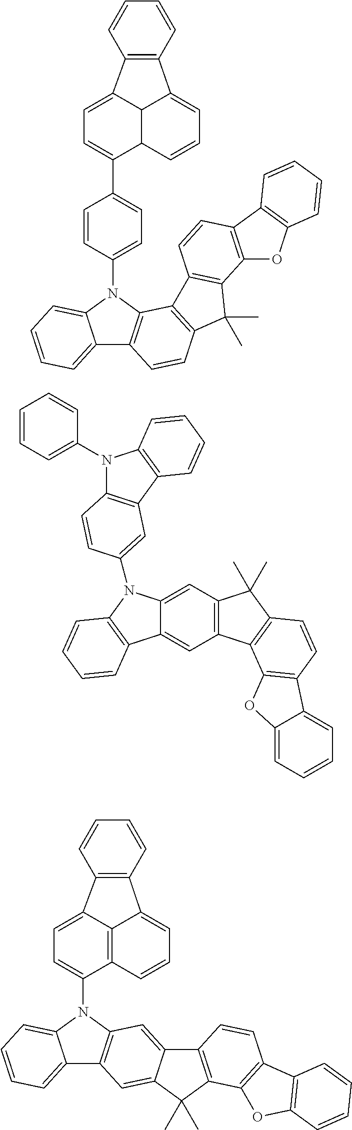

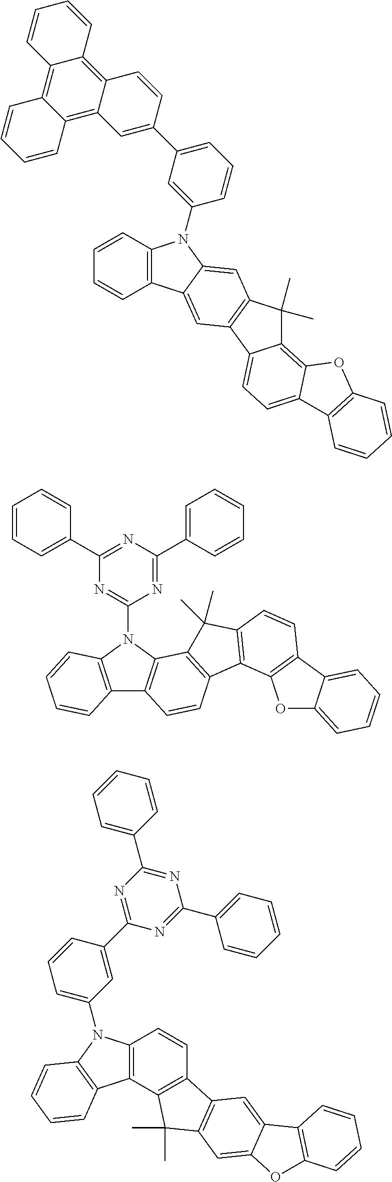 [SMILES: CC1(C)c2cc3c(cc2-c2ccc4c(oc5ccccc54)c21)c1ccccc1n3-c1cccc(-c2ccc3c4ccccc4c4ccccc4c3c2)c1 ; CC1(C)c2ccc3c(oc4ccccc43)c2-c2ccc3c4ccccc4n(-c4nc(-c5ccccc5)nc(-c5ccccc5)n4)c3c21 ; CC1(C)c2cc3oc4ccccc4c3cc2-c2ccc3c(c21)c1ccccc1n3-c1cccc(-c2nc(-c3ccccc3)nc(-c3ccccc3)n2)c1]

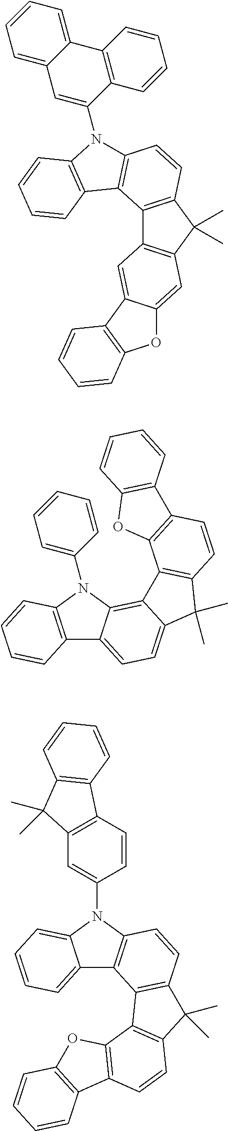

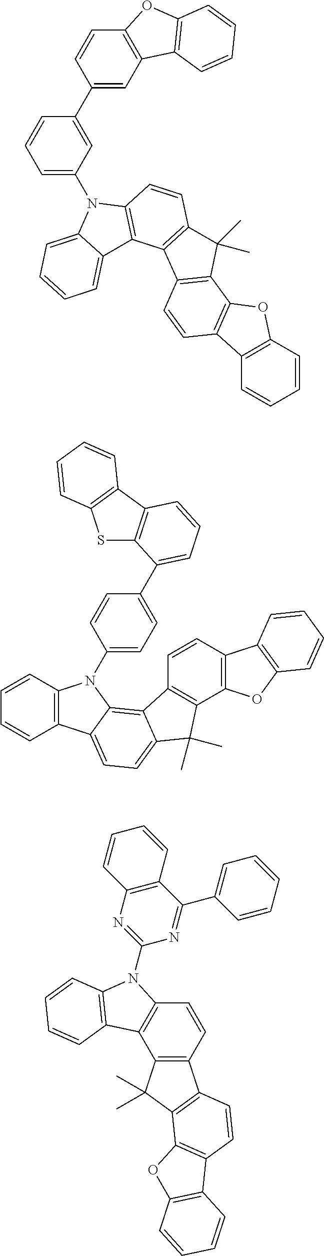 [SMILES: CC1(C)c2ccc3c(c2-c2ccc4c(oc5ccccc54)c21)c1ccccc1n3-c1cccc(-c2ccc3oc4ccccc4c3c2)c1 ; CC1(C)c2ccc3c4ccccc4n(-c4ccc(-c5cccc6c5sc5ccccc56)cc4)c3c2-c2ccc3c(oc4ccccc43)c21 ; CC1(C)c2c(ccc3c2oc2ccccc23)-c2ccc3c(c21)c1ccccc1n3-c1nc(-c2ccccc2)c2ccccc2n1]

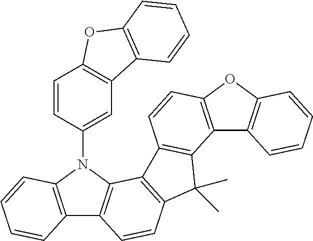 [SMILES: CC1(C)c2ccc3c4ccccc4n(-c4ccc5oc6ccccc6c5c4)c3c2-c2ccc3oc4ccccc4c3c21]

### (Material for Organic EL Devices)

The material for organic EL devices of the embodiment of the present invention includes the aforementioned compound (1) and preferably includes a compound selected from the compounds (2) to (5) and (2-1) to (2-36).

The material for organic EL devices of the embodiment of the present invention is useful as a material in organic EL devices, and for example, it is useful as a material of one or more organic thin film layers disposed between an anode and a cathode of an organic EL device, and especially useful as a host material of a light emitting layer.

### [Organic EL Device]

Next, the organic EL device of the embodiment of the present invention is described.

The organic EL device of the embodiment of the present invention includes an anode, a cathode, and one or more organic thin film layers between the cathode and the anode. The one or more organic thin film layers include a light emitting layer, and at least one layer of the one or more organic thin film layers is a layer containing the compound represented by the formula (1) (compound (1)).

Examples of the organic thin film layer in which the compound (1) is contained include an anode-side organic thin film layer provided between an anode and a light emitting layer (e.g., a hole transporting layer, a hole injecting layer, etc.), a light emitting layer, a cathode-side organic thin film layer provided between a cathode and a light emitting layer (e.g., an electron transporting layer, an electron injecting layer, etc.), a space layer, a blocking layer, and the like. However, it should not be construed that the present invention is limited thereto.

The compound (1) may be contained in any of the aforementioned layers, and for example, it may be used as a host material or a dopant material (fluorescent light emitting material) in a light emitting layer of a fluorescent light emitting unit, a host material in a light emitting layer of a phosphorescent light emitting unit, a hole transporting layer material or an electron transporting layer material of a light emitting unit, or the like. The compound (1) is preferably used as a host material of a light emitting layer and more preferably used as a host material of a light emitting layer of a phosphorescent light emitting unit.

In the embodiment of the present invention, a content of the compound (1) in the organic thin film layer (preferably the light emitting layer) is preferably 30 mol % or more, more preferably 50 mol % or more, still more preferably 70 mol % or more, and yet still more preferably 90 mol % or more relative to the whole molar number (100 mol %) of all of the components constituting the organic thin film layer.

In the embodiment of the present invention, the organic EL device may be any of a single color emitting device of a fluorescent or phosphorescent type, a white emitting device of a fluorescent/phosphorescent hybrid type, an emitting device of a simple type having a single light emitting unit, and an emitting device of a tandem type having two or more light emitting units, with the phosphorescent device being preferred. The “light emitting unit” referred to herein is the smallest unit for emitting light by the recombination of injected holes and injected electrons, which includes one or more organic layers, wherein at least one layer is a light emitting layer.

Accordingly, the following device constitutions may be exemplified as a representative device constitution of the simplr-type organic EL device.

### (1) Anode/Light Emitting Unit/Cathode

The light emitting unit may be a laminate having plural layers, such as a phosphorescent light emitting layer and a fluorescent light emitting layer. In that case, for the purpose of preventing diffusion of excitons generated in the phosphorescent light emitting layer into the fluorescent light emitting layer, a space layer may be disposed between the respective light emitting layers. Representative layer constitutions of the light emitting unit are shown below.

(a) Hole transporting layer/light emitting layer (/electron transporting layer)

(b) Hole transporting layer/first phosphorescent light emitting layer/second phosphorescent light emitting layer (/electron transporting layer)

(c) Hole transporting layer/phosphorescent light emitting layer/space layer/fluorescent light emitting layer (/electron transporting layer)

(d) Hole transporting layer/first phosphorescent light emitting layer/second phosphorescent light emitting layer/space layer/fluorescent light emitting layer (/electron transporting layer)

(e) Hole transporting layer/first phosphorescent light emitting layer/space layer/second phosphorescent light emitting layer/space layer/fluorescent light emitting layer (/electron transporting layer)

(f) Hole transporting layer/phosphorescent light emitting layer/space layer/first fluorescent light emitting layer/second fluorescent light emitting layer (/electron transporting layer)

(g) Hole transporting layer/electron blocking layer/light emitting layer (/electron transporting layer)

(h) Hole transporting layer/light emitting layer/hole blocking layer (/electron transporting layer)

(i) Hole transporting layer/fluorescent light emitting layer/triplet blocking layer (/electron transporting layer)

The emission color of the phosphorescent light emitting layer and that of the fluorescent light emitting layer may be different from each other. Specifically, examples of the laminated light emitting layer (d) include a layer constitution of hole transporting layer/first phosphorescent light emitting layer (red emission)/second phosphorescent light emitting layer (green emission)/space layer/fluorescent light emitting layer (blue emission)/electron transporting layer; and the like.

It is to be noted that an electron blocking layer may be properly provided between the each light emitting layer and the hole transporting layer or the space layer. In addition, a hole blocking layer may be properly provided between the each light emitting layer and the electron transporting layer. By providing the electron blocking layer or the hole blocking layer, electrons or holes are confined in the light emitting layer to increase a probability of charge recombination in the light emitting layer, whereby the emission efficiency may be improved.

The following device constitution may be exemplified as a representative device constitution of the tandem-type organic EL device.

### (2) Anode/First Light Emitting Unit/Intermediate Layer/Second Light Emitting Unit/Cathode

Here, the first light emitting unit and the second light emitting unit may be each independently selected from those described above with respect to the light emitting unit.

Generally, the intermediate layer is also called an intermediate electrode, an intermediate conductive layer, a charge generation layer, an electron withdrawing layer, a connecting layer, or an intermediate insulating layer. Known material constitutions capable of supplying electrons to the first light emitting unit and holes to the second light emitting unit may be adopted.

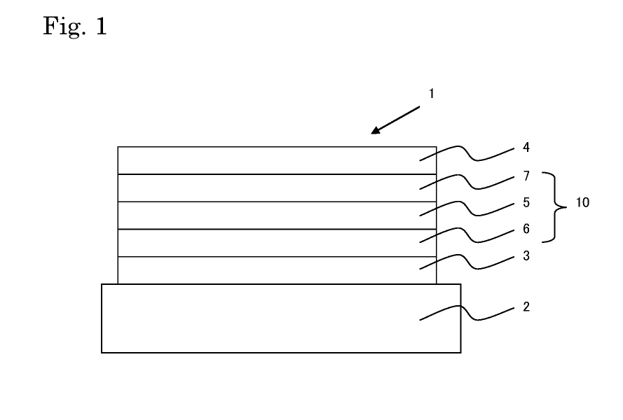

A diagrammatic constitution of an example of the organic EL device is shown in FIG. 1. An organic EL device 1 includes a substrate 2, an anode 3, a cathode 4, and a light emitting unit 10 disposed between the anode 3 and the cathode 4. The light emitting unit 10 includes at least a light emitting layer 5 containing a host material and a dopant (light emitting material). A hole injecting/transporting layer (an anode-side organic thin film layer) 6 or the like may be formed between the light emitting layer 5 and the anode 3, and an electron injecting/transporting layer (a cathode-side organic thin film layer) 7 or the like may be formed between the light emitting layer 5 and the cathode 4. In addition, an electron blocking layer (not shown) may be provided on the side of the anode 3 of the light emitting layer 5, and a hole blocking layer (not shown) may also be provided on the side of the cathode 4 of the light emitting layer 5. According to this, electrons or holes are confined in the light emitting layer 5, whereby a probability of exciton generation in the light emitting layer 5 may be more enhanced.

It is to be noted that in the present invention, a host combined with a fluorescent dopant (fluorescent emitting material) is referred to as a fluorescent host, and a host combined with a phosphorescent dopant (phosphorescent emitting material) is referred to as a phosphorescent host. The fluorescent host and the phosphorescent host are not distinguished from each other merely by a difference in molecular structure. That is, the term “phosphorescent host” means a material for forming a phosphorescent emitting layer containing a phosphorescent dopant and does not mean a material that cannot be utilized as a material for forming a fluorescent emitting layer. The same applies to the fluorescent host.

### (Substrate)

The substrate is used as a support of a light emitting device. As the substrate, for example, glass, quartz, a plastic, and the like may be used. In addition, a flexible substrate may also be used. The flexible substrate refers to a bendable (flexible) substrate, and examples thereof include a plastic substrate made of polycarbonate or polyvinyl chloride, and the like.

### (Anode)

For the anode which is formed on the substrate, it is preferred to use a metal, an alloy, an electroconductive compound, or a mixture thereof, each having a large work function (specifically 4.0 eV or more), or the like. Specifically, examples thereof include indium oxide-tin oxide (ITO: indium tin oxide), indium oxide-tin oxide containing silicon or silicon oxide, indium oxide-zinc oxide, tungsten oxide, indium oxide or graphene each containing zinc oxide, and the like. Besides, gold (Au), platinum (Pt), a nitride of a metal material (for example, titanium nitride), and the like are exemplified.

### (Cathode)

For the cathode, it is preferred to use a metal, an alloy, an electroconductive compound, or a mixture thereof, each having a small work function (specifically 3.8 eV or less), or the like. Specific examples of such a cathode material include elements belonging to the group 1 or group 2 of the periodic table of elements, namely, alkali metals, such as lithium (Li), cesium (Cs), etc., alkaline earth metals, such as magnesium (Mg), etc., rare earth metals, such as alloys containing the same (for example, MgAg and AlLi), alloys thereof, and the like.

### (Guest Material of Light Emitting Layer)

The light emitting layer is a layer containing a substance with high luminescence, and various materials may be used.

With respect to the guest material of the light emitting layer, for example, a fluorescent light emitting material that emits fluorescence and a phosphorescent light emitting material that emits phosphorescence may be used as the substance with high luminescence. The fluorescent light emitting material is a compound capable of emitting light from a singlet excited state, and the phosphorescent light emitting material is a compound capable of emitting light from a triplet excited state.

In the organic EL device of the embodiment of the present invention, it is preferred that the light emitting layer further contains one or more selected from a fluorescent light emitting material and a phosphorescent light emitting material.

As a blue fluorescent light emitting material which may be used in the light emitting layer, a pyrene derivative, a styrylamine derivative, a chrysene derivative, a fluoranthene derivative, a fluorene derivative, a diamine derivative, a triarylamine derivative, and the like may be used. Specific examples thereof include N,N′-bis[4-(9H-carbazol-9-yl)phenyl]-N,N′-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(10-phenyl-9-anthryl)-4′-(9-phenyl-9H-carbazol-3-yl)triphenylamine (abbreviation: PCBAPA), and the like.

As a green fluorescent light emitting material which may be used in the light emitting layer, an aromatic amine derivative and the like may be used. Specific examples thereof include N-(9,10-diphenyl-2-anthryl)-N,9-diphenyl-9H-carbazol-3-amine (abbreviation: 2PCAPA), N-[9,10-bis(1,1′-biphenyl-2-yl)-2-anthryl]-N,9-diphenyl-9H-carbazol-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(1,1′-biphenyl-2-yl)-2-anthryl]-N,N′,N′-triphenyl-1,4-phenylenediamine (abbreviation: 2DPABPhA), N-[9,10-bis(1,1′-biphenyl-2-yl)]-N-[4-(9H-carbazol-9-yl)phenyl]-N-phenylanthrace n-2-amine (abbreviation: 2YGABPhA), N,N,9-triphenylanthracen-9-amine (abbreviation: DPhAPhA), and the like.

As a red fluorescent light emitting material which may be used in the light emitting layer, a tetracene derivative, a diamine derivative, and the like may be used. Specific examples thereof include N,N,N′,N′-tetrakis(4-methylphenyl)tetracene-5,11-diamine (abbreviation: p-mPhTD), 7,14-diphenyl-N,N,N′,N′-tetrakis(4-methylphenyl)acenaphtho[1,2-a]fluoranthene-3,10-diamine (abbreviation: p-mPhAFD), and the like.

As a blue phosphorescent light emitting material which may be used in the light emitting layer, a metal complex, such as an iridium complex, an osmium complex, a platinum complex, etc., preferably an ortho-metalated complex of iridium, osmium, or platinum metal, is used. Specific examples thereof include bis[2-(4′,6′-difluorophenyl)pyridinato-N,C2′]iridium(III) tetrakis(1-pyrazolyl)borate (abbreviation: FIr6), bis[2-(4′,6′-difluorophenyl)pyridinato-N,C2′]iridium(III) picolinate (abbreviation: FIrpic), bis[2-(3′,5′-bistrifluoromethylphenyl)pyridinato-N,C2′]iridium(III) picolinate (abbreviation: Ir(CF₃ppy)₂(pic)), bis[2-(4′,6′-difluorophenyl)pyridinato-N,C2′]iridium(III) acetylacetonate (abbreviation: FIracac), and the like.

As a green phosphorescent light emitting material which may be used in the light emitting layer, an iridium complex and the like are used. Examples thereof include tris(2-phenylpyridinato-N,C2′)iridium(III) (abbreviation: Ir(ppy)₃), bis(2-phenylpyridinato-N,C2′)iridium(III) acetylacetonate (abbreviation: Ir(ppy)₂(acac)), bis(1,2-diphenyl-1H-benzoimidazolato)iridium(III) acetylacetonate (abbreviation: Ir(pbi)₂(acac)), bis(benzo[h]quinolinato)iridium(III) acetylacetonate (abbreviation: Ir(bzq)₂(acac)), and the like.

As a red phosphorescent light emitting material which may be used in the light emitting layer, an iridium complex, a platinum complex, a terbium complex, a europium complex, and the like are used. Specific examples thereof include organic metal complexes, such as bis[2-(2′-benzo[4,5-a]thienyl)pyridinato-N,C3′]iridium(III) acetylacetonate (abbreviation: Ir(btp)₂(acac)), bis(1-phenylisoquinalinato-N,C2′)iridium(III) acetylacetonate (abbreviation: Ir(piq)₂(acac)), (acetylacetonato)bis[2,3-bis(4-fluorophenyl)quinoxalinato]iridium(III) (abbreviation: Ir(Fdpq)₂(acac)), 2,3,7,8,12,13,17,18-octaethyl-21H,23H-porphyrin platinum(II) (abbreviation: PtOEP), etc.

In addition, a rare earth metal complex, such as tris(acetylacetonato)(monophenanthroline)terbium(III) (abbreviation: Tb(acac)₃(Phen)), tris(1,3-diphenyl-1,3-propanedionato)(monophenanthroline)europium(III) (abbreviation: Eu(DBM)₃(Phen)), tris[1-(2-thenoyl)-3,3,3-trifluoroacetonato)(monophenanthroline) europ ium(III) (abbreviation: Eu(TTA)₃(Phen)), etc., may be used as the phosphorescent light emitting material because it is concerned with emission from a rare earth metal ion (electronic transition between different multiplicities).

### (Host Material of Light Emitting Layer)

The light emitting layer may be constituted such that the aforementioned substance with high luminescence (guest material) is dispersed in other substance (host material).

With respect to the host material of the light emitting layer, various substances may be used so long as they are a substance for dispersing the substance with high luminescence. It is preferred to use a substance having a higher lowest unoccupied molecular orbital level (LUMO level) and a lower highest occupied molecular orbital level (HOMO level) than the substance with high luminescence.

As the substance (host material) for dispersing the substance with high luminescence, the compound (1) that is the embodiment of the present invention is preferred.

Besides the compound that is the embodiment of the present invention, for example, 1) a metal complex, such as an aluminum complex, a beryllium complex, a zinc complex, etc.; 2) a heterocyclic compound, such as an oxadiazole derivative, a benzimidazole derivative, a phenanthroline derivative, etc.; 3) a fused aromatic compound, such as a carbazole derivative, an anthracene derivative, a phenanthrene derivative, a pyrene derivative, a chrysene derivative, etc.; and 4) an aromatic amine compound, such as a triarylamine derivative, a fused polycyclic aromatic amine derivative, etc., may be used. More specifically, a metal complex, such as tris(8-quinolinolato)aluminum(III) (abbreviation: Alq), tris(4-methyl-8-quinolinolato)aluminum(III) (abbreviation: Almq₃), bis(10-hydroxybenzo[h]quinolinato)beryllium(II) (abbreviation: BeBq₂), bis(2-methyl-8-quinolinolato) (4-phenylphenolato)aluminum(III) (abbreviation: BAlq), bis(8-quinolinolato) zinc(II) (abbreviation: Znq), bis[2-(2-benzoxazolyl)phenolate]zinc(II) (abbreviation: ZnPBO), bis[2-(2-benzothiazolyl)phenolate]zinc(II) (abbreviation: ZnBTZ), etc.; a heterocyclic compound, such as 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 3-(4-biphenylyl)-4-phenyl-5-(4-tert-butylphenyl)-1,2,4-triazole (abbreviation: TAZ), 2,2′,2″-(1,3,5-benzenetriyl)tris(1-phenyl-1H-benzimidazole) (abbreviation: TPBI), bathophenanthroline (abbreviation: BPhen), bathocuproine (abbreviation: BCP), etc.; a fused aromatic compound, such as 9-[4-(10-phenyl-9-anthryl)phenyl]-9H-carbazole (abbreviation: CzPA), 3,6-diphenyl-9-[4-(10-phenyl-9-anthryl)phenyl]-9H-carbazole (abbreviation: DPCzPA), 9,10-bis(3,5-diphenylphenyl)anthracene (abbreviation: DPPA), 9,10-di(2-naphthyl)anthracene (abbreviation: DNA), 2-tert-butyl-9,10-di(2-naphthyl)anthracene (abbreviation: t-BuDNA), 9,9′-bianthryl (abbreviation: BANT), 9,9′-(stilbene-3,3′-diyl)diphenanthrene (abbreviation: DPNS), 9,9′-(stilbene-4,4′-diyl)diphenanthrene (abbreviation: DPNS2), 3,3′,3″-(benzene-1,3,5-triyl)tripyrene (abbreviation: TPB3), 9,10-diphenylanthracene (abbreviation: DPAnth), 6,12-dimethoxy-5,11-diphenylchrysene, etc.; an aromatic amine compound, such as N,N-dipheyl-9-[4-(10-phenyl-9-anthryl)phenyl]-9H-carbazol-3-amine (abbreviation: CzA1PA), 4-(10-phenyl-9-anthryl)triphenylamine (abbreviation: DPhPA), N, 9-diphenyl-N-[4-(10-phenyl-9-anthryl)phenyl]-9H-carbazol-3-amine (abbreviation: PCAPA), N, 9-diphenyl-N-{4-[4-(10-phenyl-9-anthryl)phenyl]phenyl}-9H-carbazol-3-amine (abbreviation: PCAPBA), N-(9,10-diphenyl-2-anthryl)-N, 9-diphenyl-9H-carbazol-3-amine (abbreviation: 2PCAPA), NPB (or α-NPD), TPD, DFLDPBi, BSPB, etc.; and the like may be used. A plurality of substances (host materials) for dispersing the substance with high luminescence (guest material) may be used.

### (Electron Transporting Layer)

The electron transporting layer is a layer containing a substance with high electron transporting properties. For the electron transporting layer, 1) a metal complex, such as an aluminum complex, a beryllium complex, a zinc complex, etc.; 2) a heterocyclic aromatic compound, such as an imidazole derivative, a benzimidazole derivative, an azine derivative, a carbazole derivative, a phenanthroline derivative; and 3) a polymer compound may be used.

### (Electron Injecting Layer)

The electron injecting layer is a layer containing a substance with high electron injecting properties. For the electron injecting layer, an alkali metal, an alkaline earth metal, or a compound thereof, such as lithium (Li), lithium fluoride (LiF), cesium fluoride (CsF), potassium fluoride (CaF₂), lithium oxide (LiOₓ), etc., may be used.

### (Hole Injecting Layer)

The hole injecting layer is a layer containing a substance with high hole injecting properties. As the substance with high hole injecting properties, molybdenum oxide, titanium oxide, vanadium oxide, rhenium oxide, ruthenium oxide, chromium oxide, zirconium oxide, hafnium oxide, tantalum oxide, silver oxide, tungsten oxide, manganese oxide, an aromatic amine compound, a polymer compound (e.g., an oligomer, a dendrimer, a polymer, etc.), and the like may also be used.

### (Hole Transporting Layer)

The hole transporting layer is a layer containing a substance with high hole transporting properties. For the hole transporting layer, an aromatic amine compound, a carbazole derivative, an anthracene derivative, and the like may be used. A polymer compound, such as poly(N-vinylcarbazole) (abbreviation: PVK), poly(4-vinyltriphenylamine) (abbreviation: PVTPA), etc., may also be used. However, other substances may also be used so long as they have higher transporting properties of holes than those of electrons. It is to be noted that the layer containing a substance with high hole transporting properties may be not only a single layer but also a laminate of two or more layers composed of the aforementioned substance.

In the embodiment of the present invention, each of the layers of the organic EL device may be formed by a conventionally known method, such as a vacuum vapor deposition method, a spin coating method, etc. For example, the each layer may be formed by a known method, such as a vacuum vapor deposition method, a molecular beam deposition method (MBE method), or a coating method of using a solution of a compound for forming the layer, e.g., a dipping method, a spin coating method, a casting method, a bar coating method, a roll coating method, etc.

Though a film thickness of each of the organic layers is not particularly limited, in general, when the film thickness is too thin, a defect, such as pinholes, etc., is liable to be generated, and conversely, when the film thickness is too thick, a high drive voltage becomes necessary, resulting in deterioration of efficiency. For that reason, the film thickness of the each organic layer is generally 5 nm to 10 μm, and preferably 10 nm to 1 μm.

### [Electronic Equipment]

The electronic equipment of the embodiment of the present invention is electronic equipment including the aforementioned organic EL device of the embodiment of the present invention mounted thereon.

Examples of such electronic equipment include a display part of an organic EL panel module, etc., a display of a television set, a mobile phone, a personal computer, etc., a light emitting apparatus of a lighting element, a vehicle lighting element, etc., and the like.

### EXAMPLES

The present invention is hereunder described in more detail with reference to the following Examples and Comparative Examples, but it should be construed that the present invention is not limited to the contents described in these Examples at all.

It is to be noted that it is possible to synthesize compounds as prescribed in the scope of claims of the present application by using known alternative reactions or raw materials tailored to the desired product with reference to the following synthesis reactions.

### Synthesis of the Compound of the Embodiment of the Present Invention

### Synthesis 1 (Synthesis of Intermediate A)

A synthesis scheme of Intermediate A is shown below.

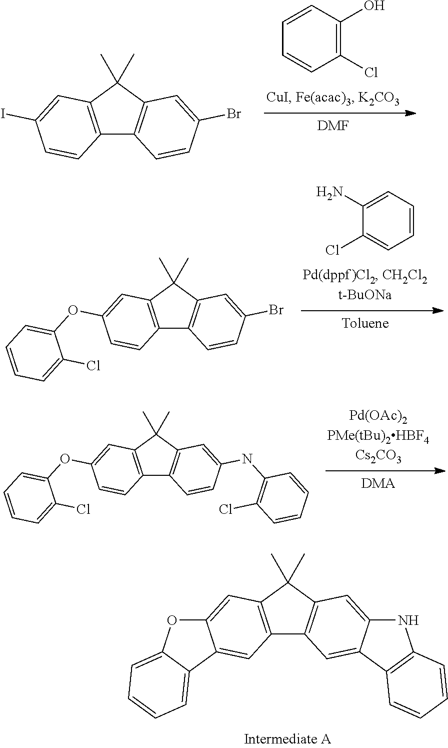

### (1) Synthesis of 2-bromo-7-(2-chlorophenoxy)-9,9-dimethyl-9H-fluorene

In a flask, 48.0 g of 2-bromo-7-iodo-9,9-dimethyl-9H-fluorene, 15 mL of 2-chlorophenol, 2.29 g of copper iodide, 8.47 g of tris(2,4-pentanedionato)iron(III), 33.2 g of potassium carbonate, and 240 mL of N,N-dimethylformamide were charged in an argon atmosphere and heated with stirring for 10 hours.

After cooling to room temperature (25° C.), a reaction solution was filtered, and the reaction solution was extracted with toluene. After removing an aqueous layer, an organic layer was washed with a saturated ammonium chloride aqueous solution. The organic layer was dried over sodium sulfate and then concentrated, and the solvent was distilled off under reduced pressure. A residue was purified by means of silica gel column chromatography, thereby obtaining 27.0 g (yield: 56%) of a white solid.

The resulting white solid was identified to be 2-bromo-7-(2-chlorophenoxy)-9,9-dimethyl-9H-fluorene through an LC-MS analysis.

### (2) Synthesis of 7-(2-chlorophenoxy)-N-(2-chlorophenyl)-9,9-dimethyl-9H-fluoren-3-amine

In a flask, 27.0 g of 2-bromo-7-(2-chlorophenoxy)-9,9-dimethyl-9H-fluorene, 10.5 mL of 2-chloroaniline, 0.98 g of a [1,1′-bis(diphenylphosphino)ferrocene]palladium(II) dichloride.dichloromethane adduct, 19.2 g of sodium tert-butoxide, and 100 mL of toluene were charged in an argon atmosphere and heated with stirring for 4 hours.

After cooling to room temperature (25° C.), a reaction solution was filtered, and the solvent was distilled off under reduced pressure. A residue was purified by means of silica gel column chromatography, thereby obtaining 17.9 g (yield: 59%) of a white solid.

The resulting white solid was identified to be 7-(2-chlorophenoxy)-N-(2-chlorophenyl)-9,9-dimethyl-9H-fluoren-3-amine through an LC-MS analysis.

### (3) Synthesis of Intermediate A

In a flask, 17.9 g of 7-(2-chlorophenoxy)-N-(2-chlorophenyl)-9,9-dimethyl-9H-fluoren-2-amine, 0.54 g of palladium acetate, 1.19 g of di-tert-butyl(methyl)phosphonium.tetrafluoroborate, 65.0 g of cesium carbonate, and 200 mL of N,N-dimethylacetamide were charged in an argon atmosphere and heated at 130° C. with stirring for 17 hours.

After cooling to room temperature (25° C.), a reaction solution was filtered, and the reaction solution was extracted with toluene. After removing an aqueous layer, an organic layer was washed with a saturated ammonium chloride aqueous solution. The organic layer was dried over sodium sulfate and then concentrated, and a residue was purified by means of silica gel column chromatography, thereby obtaining 5.0 g (yield: 33%) of a white solid.

The resulting white solid was identified to be Intermediate A through an LC-MS analysis.

### Synthesis Example 1

### Synthesis of Compound 1

A synthesis scheme of Compound 1 is shown below.

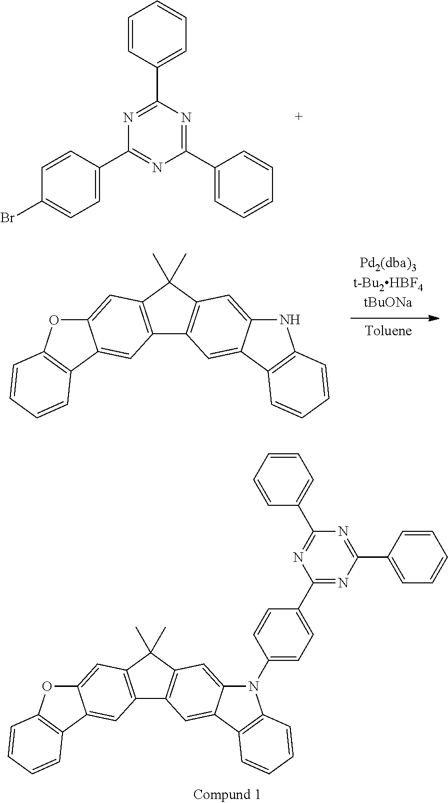

In a flask, 1.94 g of 2-(4-bromophenyl)-4,6-diphenyl-1,3,5-triazine, 1.86 g of Intermediate A obtained in Synthesis 1, 0.18 g of tris(dibenzylideneacetone)dipalladium(0), 0.12 g of tri-tert-butylphosphonium tetrafluoroborate, 0.67 g of sodium tert-butoxide, and 50 mL of toluene were charged in an argon atmosphere and heated at 50° C. with stirring for 5 hours.

After cooling to room temperature (25° C.), a reaction solution was filtered, and the solvent was distilled off under reduced pressure. The resulting sample was dissolved in toluene, methanol was then added for crystallization, and a solid was collected by filtration. The resulting solid was again dissolved in toluene, methanol was added for crystallization, and a solid was collected by filtration, thereby obtaining 1.6 g (yield: 47 g) of a white solid.

The resulting white solid was identified to be the titled Compound 1 through an LC-MS analysis.

### Synthesis Example 2

### Synthesis of Compound 2

A synthesis scheme of Compound 2 is shown below.

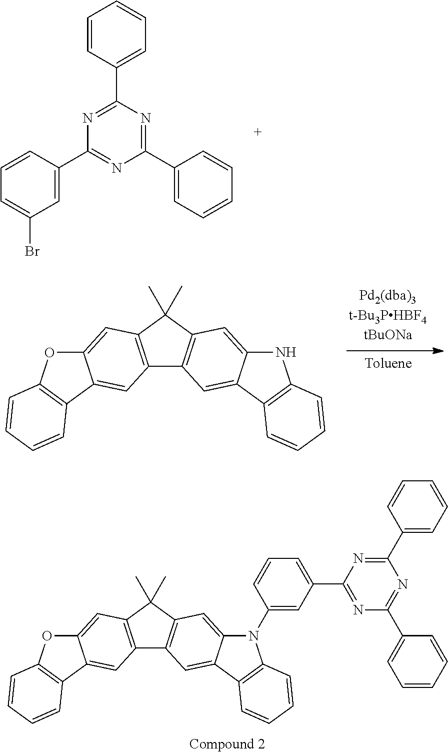 [SMILES: Brc1cccc(-c2nc(-c3ccccc3)nc(-c3ccccc3)n2)c1 ; CC1(C)c2cc3[nH]c4ccccc4c3cc2-c2cc3c(cc21)oc1ccccc13 ; CC1(C)c2cc3oc4ccccc4c3cc2-c2cc3c4ccccc4n(-c4cccc(-c5nc(-c6ccccc6)nc(-c6ccccc6)n5)c4)c3cc21]

Synthesis was carried out in the same method as in Synthesis Example 1, except that in the synthesis of Compound 1 of Synthesis Example 1, 2-(3-bromophenyl)-4,6-diphenyl-1,3,5-triazine (1.94 g) was used in place of the 2-(4-bromophenyl)-4,6-diphenyl-1,3,5-triazine (1.94 g), thereby obtaining 2.8 g (yield: 82%) of a white solid.

The resulting white solid was identified to be the titled Compound 2 through an LC-MS analysis.

### Synthesis Example 3

### Synthesis of Compound 3

A synthesis scheme of Compound 3 is shown below.

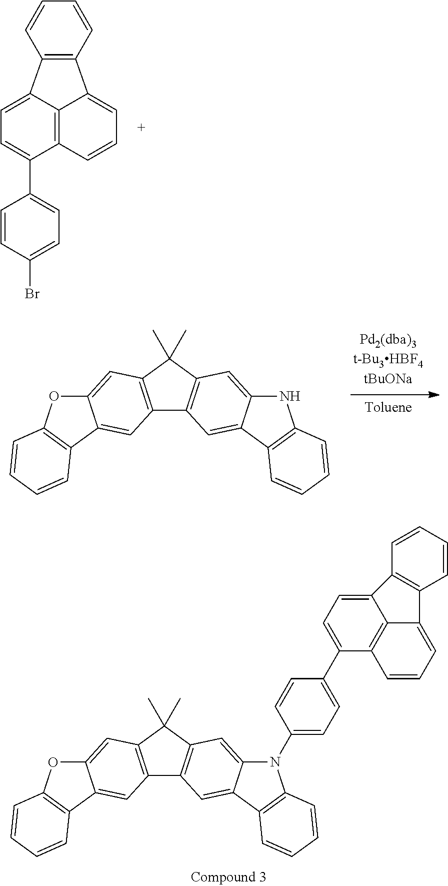 [SMILES: Brc1ccc(-c2ccc3c4c(cccc24)-c2ccccc2-3)cc1 ; CC1(C)c2cc3[nH]c4ccccc4c3cc2-c2cc3c(cc21)oc1ccccc13 ; CC1(C)c2cc3oc4ccccc4c3cc2-c2cc3c4ccccc4n(-c4ccc(-c5ccc6c7c(cccc57)-c5ccccc5-6)cc4)c3cc21]

Synthesis was carried out in the same method as in Synthesis Example 1, except that in the synthesis of Compound 1 of Synthesis Example 1, 3-(4-bromophenyl)fluoranthene (1.79 g) was used in place of the 2-(4-bromophenyl)-4,6-diphenyl-1,3,5-triazine (1.94 g), thereby obtaining 1.8 g (yield: 54%) of a white solid.

The resulting white solid was identified to be the titled Compound 3 through an LC-MS analysis.

### Synthesis Example 4

### Synthesis of Compound 4

A synthesis scheme of Compound 4 is shown below.

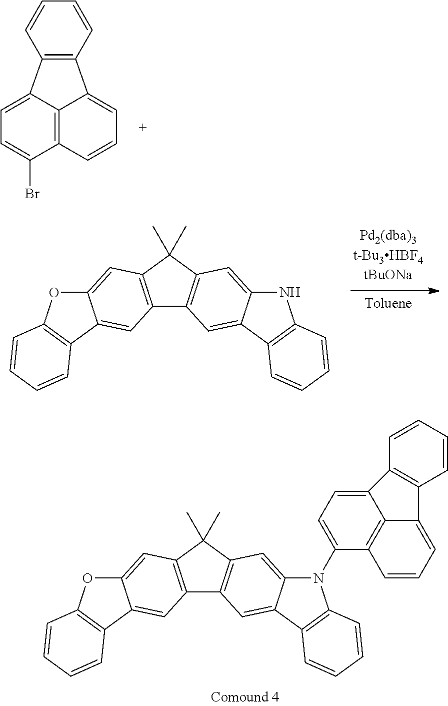 [SMILES: Brc1ccc2c3c(cccc13)-c1ccccc1-2 ; CC1(C)c2cc3[nH]c4ccccc4c3cc2-c2cc3c(cc21)oc1ccccc13 ; CC1(C)c2cc3oc4ccccc4c3cc2-c2cc3c4ccccc4n(-c4ccc5c6c(cccc46)-c4ccccc4-5)c3cc21]

Synthesis was carried out in the same method as in Synthesis Example 1, except that in the synthesis of Compound 1 of Synthesis Example 1, 3-bromofluoranthene (1.41 g) was used in place of the 2-(4-bromophenyl)-4,6-diphenyl-1,3,5-triazine (1.94 g), thereby obtaining 1.8 g (yield: 62%) of a white solid.

The resulting white solid was identified to be the titled Compound 4 through an LC-MS analysis.

### Synthesis Example 5

### Synthesis of Compound 5

A synthesis scheme of Compound 5 is shown below.

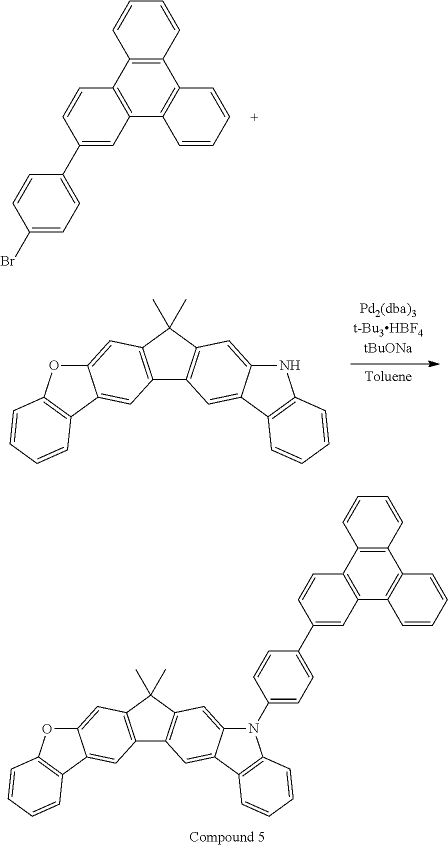 [SMILES: Brc1ccc(-c2ccc3c4ccccc4c4ccccc4c3c2)cc1 ; CC1(C)c2cc3[nH]c4ccccc4c3cc2-c2cc3c(cc21)oc1ccccc13 ; CC1(C)c2cc3oc4ccccc4c3cc2-c2cc3c4ccccc4n(-c4ccc(-c5ccc6c7ccccc7c7ccccc7c6c5)cc4)c3cc21]

Synthesis was carried out in the same method as in Synthesis Example 1, except that in the synthesis of Compound 1 of Synthesis Example 1, 2-(4-bromophenyl)triphenylene (1.92 g) was used in place of the 2-(4-bromophenyl)-4,6-diphenyl-1,3,5-triazine (1.94 g), thereby obtaining 1.9 g (yield: 55%) of a white solid.

The resulting white solid was identified to be the titled Compound 5 through an LC-MS analysis.

### Synthesis Example 6

### Synthesis of Compound 6

A synthesis scheme of Compound 6 is shown below.

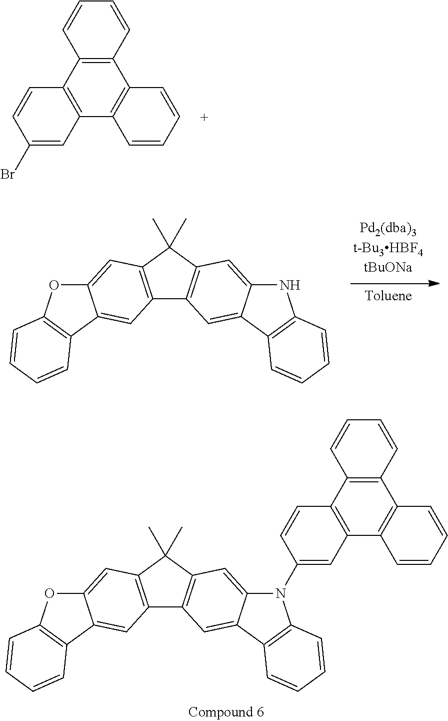 [SMILES: Brc1ccc2c3ccccc3c3ccccc3c2c1 ; CC1(C)c2cc3[nH]c4ccccc4c3cc2-c2cc3c(cc21)oc1ccccc13 ; CC1(C)c2cc3oc4ccccc4c3cc2-c2cc3c4ccccc4n(-c4ccc5c6ccccc6c6ccccc6c5c4)c3cc21]

Synthesis was carried out in the same method as in Synthesis Example 1, except that in the synthesis of Compound 1 of Synthesis Example 1, 2-bromotriphenylene (1.54 g) was used in place of the 2-(4-bromophenyl)-4,6-diphenyl-1,3,5-triazine (1.94 g), thereby obtaining 1.3 g (yield: 43%) of a white solid.

The resulting white solid was identified to be the titled Compound 6 through an LC-MS analysis.

### Synthesis Example 7

### Synthesis of Compound 7

A synthesis scheme of Compound 7 is shown below.

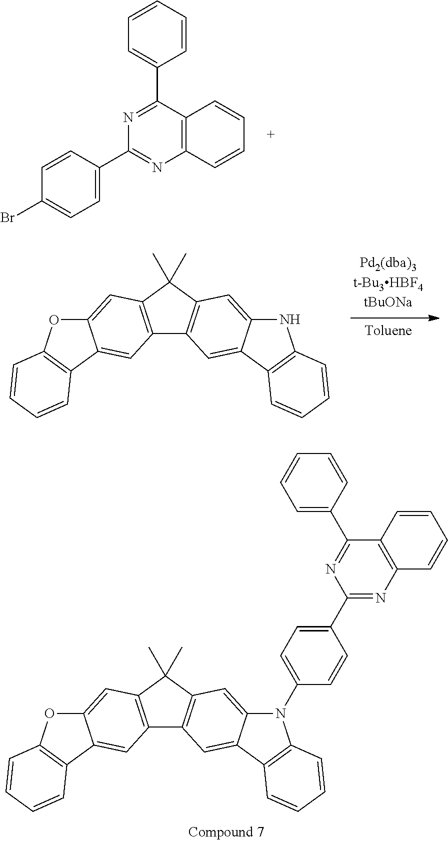

Synthesis was carried out in the same method as in Synthesis Example 1, except that in the synthesis of Compound 1 of Synthesis Example 1, 2-(4-bromophenyl)-4-phenylquinazoline (1.81 g) was used in place of the 2-(4-bromophenyl)-4,6-diphenyl-1,3,5-triazine (1.94 g), thereby obtaining 2.8 g (yield: 82%) of a white solid.

The resulting white solid was identified to be the titled Compound 7 through an LC-MS analysis.

### Synthesis Example 8

### Synthesis of Compound 8

A synthesis scheme of Compound 8 is shown below.

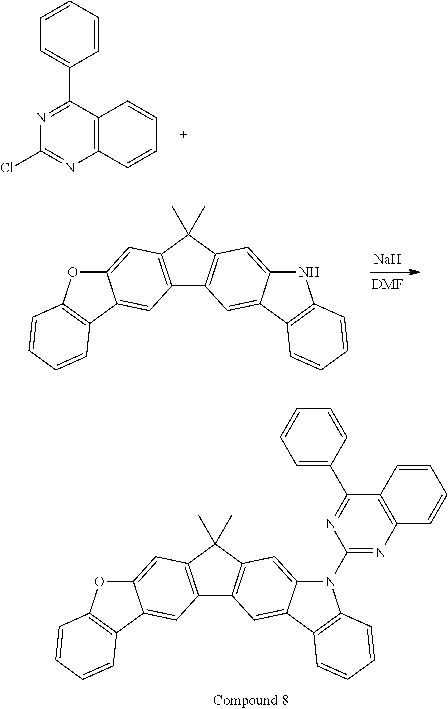

In a flask, 1.20 g of 2-chloro-4-phenylquinazoline, 1.86 g of Intermediate A obtained in Synthesis 1, and 5 mL of an N,N-dimethylformamide solution containing 0.33 g of sodium hydride were charged in an argon atmosphere and stirred at room temperature (25° C.) for 8 hours.

Water was added to a reaction solution, and a solid was collected by filtration. The resulting solid was purified by means of silica gel column chromatography, thereby obtaining 2.1 g (yield: 72%) of a white solid.

The resulting white solid was identified to be the titled Compound 8 through an LC-MS analysis.

### Synthesis Example 9

### Synthesis of Compound 9

A synthesis scheme of Compound 9 is shown below.

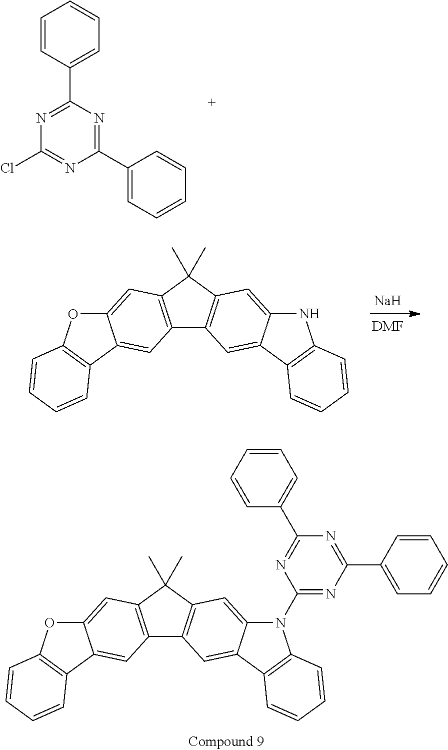

Synthesis was carried out in the same method as in Synthesis Example 8, except that in the synthesis of Compound 8 of Synthesis Example 8, 2-chloro-4,6-diphenyl-1,3,5-triazine was used in place of the 2-chloro-4-phenylquinazoline, thereby obtaining 2.3 g (yield: 77%) of a white solid.

The resulting white solid was identified to be the titled Compound 9 through an LC-MS analysis.

### Synthesis 2 (Synthesis of Intermediate 1-A)

A synthesis scheme of Intermediate 1-A is shown below.

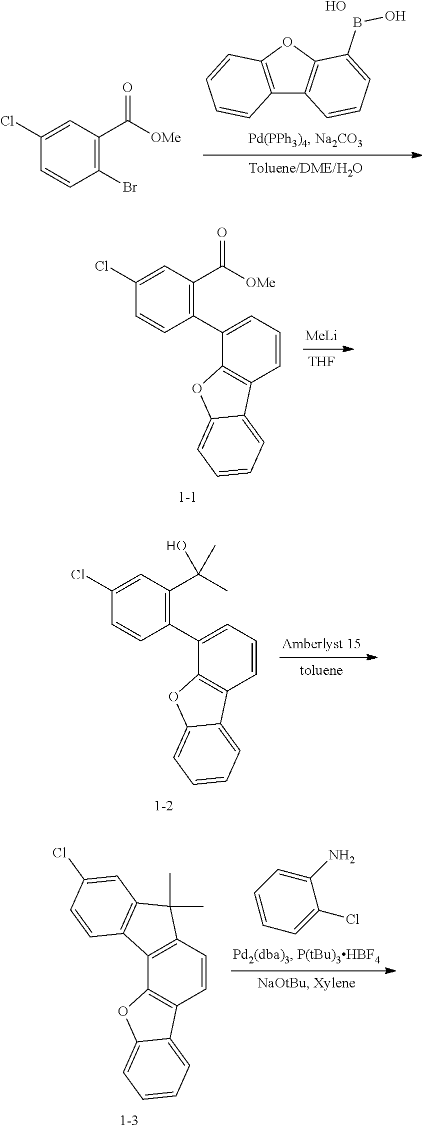

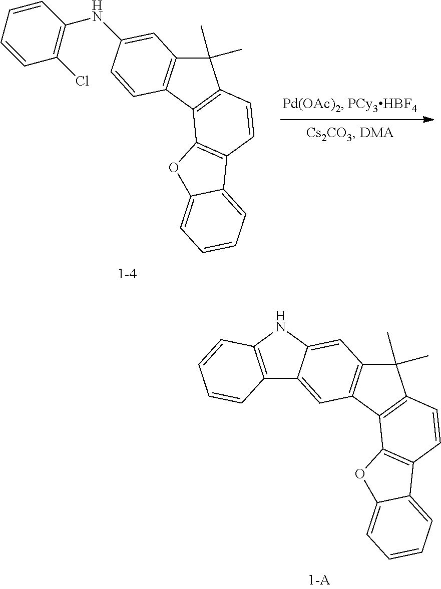 [SMILES: CC1(C)c2cc(Nc3ccccc3Cl)ccc2-c2c1ccc1c2oc2ccccc21 ; CC1(C)c2cc3[nH]c4ccccc4c3cc2-c2c1ccc1c2oc2ccccc21]

### Synthesis of Intermediate 1-1

In a flask, 3.0 g of dibenzofuran-4-boronic acid, 3.2 g of methyl 2-bromo-5-chlorobenzoate, 0.3 g of tetrakis(triphenylphosphine)palladium(0), 4.1 g of sodium carbonate, 30 mL of toluene, 10 mL of 1,2-dimethoxyethane, and 10 mL of water were charged in an argon atmosphere and heated under reflux with stirring for 8 hours.

After cooling to room temperature (25° C.), a reaction solution was transferred into a separatory funnel and extracted with toluene. Thereafter, an organic layer was dried over sodium sulfate, filtered, and then concentrated. A residue was purified by means of silica gel column chromatography, thereby obtaining 3.2 g (yield: 74%) of a white solid.

The resulting white solid was identified to be Intermediate 1-1 through an LC-MS analysis.

### Synthesis of Intermediate 1-2

In a flask, 3.2 g of Intermediate 1-1 and 40 mL of tetrahydrofuran were charged in an argon atmosphere. After cooling to −70° C., 15 mL of a methoxyethyllithium diethyl ether solution (1.6 M) was gradually added, and the contents were stirred for 30 hours as they were. Thereafter, the temperature was gradually increased to room temperature, followed by stirring for 6 hours.

Thereafter, an ammonium chloride aqueous solution was added to a reaction solution, and the resultant was transferred into a separatory funnel and extracted with toluene. Thereafter, an organic layer was dried over sodium sulfate, filtered, and then concentrated. A residue was purified by means of silica gel column chromatography, thereby obtaining 2.8 g (yield: 86%) of a white solid.

The resulting white solid was identified to be Intermediate 1-2 through an LC-MS analysis.

### Synthesis of Intermediate 1-3

In a flask, 2.8 g of Intermediate 1-2, 2.8 g of Amberlist 15, and 35 mL of toluene were charged in an argon atmosphere and heated under reflux with stirring for 12 hours.

After cooling to room temperature (25° C.), an unnecessary matter was filtered off, followed by concentration. A residue was purified by means of silica gel column chromatography, thereby obtaining 1.3 g (yield: 48%) of a white solid.

The resulting white solid was identified to be Intermediate 1-3 through an LC-MS analysis.

### Synthesis of Intermediate 1-4

In a flask, 1.3 g of Intermediate 1-3, 0.6 g of 2-chloroaniline, 73 mg of tris(dibenzylideneacetone)dipalladium(0), 93 mg of tri-tert-butylphosphonium tetrafluoroborate, 1.1 g of sodium tert-butoxide, and 12 mL of xylene were charged in an argon atmosphere and heated under reflux with stirring for 8 hours.

After cooling to room temperature (25° C.), a reaction solution was concentrated, and a residue was purified by means of silica gel column chromatography, thereby obtaining 1.3 g (yield: 78%) of a white solid.

The resulting white solid was identified to be Intermediate 1-4 through an LC-MS analysis.

### Synthesis of Intermediate 1-A

In a flask, 1.3 g of Intermediate 1-4, 35 mg of palladium(II) acetate, 114 mg of tricyclohexylphosphonium tetrafluoroborate, 3.0 g of cesium carbonate, and 30 mL of N,N-dimethylacetamide were charged in an argon atmosphere and heated at 130° C. with stirring for 12 hours.

After cooling to room temperature (25° C.), a reaction solution was transferred into a separatory funnel, and water was added, followed by extracting with a solution of hexane and ethyl acetate (3/1). Thereafter, an organic layer was dried over sodium sulfate, filtered, and then concentrated. A residue was purified by means of silica gel column chromatography, thereby obtaining 0.6 g (yield: 52%) of a white solid.

The resulting white solid was identified to be Intermediate 1-A through an LC-MS analysis.

### Synthesis 3 (Synthesis of Intermediate 2-A)

A synthesis scheme of Intermediate 2-A is shown below.

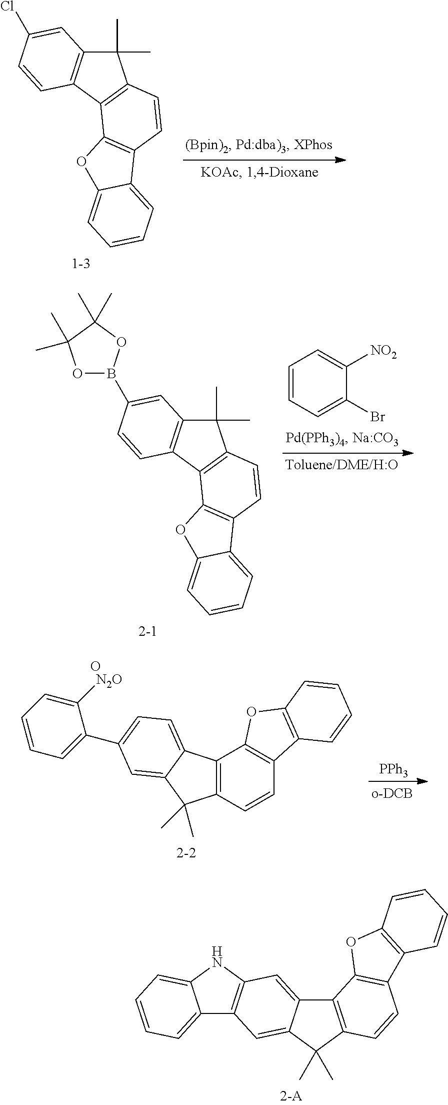

### Synthesis of Intermediate 2-1

In a flask, 0.6 g of Intermediate 1-3, 1.4 g of bis(pinacolato)diboron, 52 mg of tris(dibenzylideneacetone)dipalladium(0), 54 mg of 2-(dicyclohexylphosphino)-2′,4′6′-tri-isopropyl-1,1′-biphenyl, 0.6 g of potassium acetate, and 10 mL of 1,4-dioxane were charged in an argon atmosphere and heated under reflux with stirring for 8 hours.

After cooling to room temperature (25° C.), a reaction solution was transferred into a separatory funnel, and water was added, followed by extracting with toluene. Thereafter, an organic layer was dried over sodium sulfate, filtered, and then concentrated. A residue was purified by means of silica gel column chromatography, thereby obtaining 0.5 g (yield: 68%) of a white solid.

The resulting white solid was identified to be Intermediate 2-1 through an LC-MS analysis.

### Synthesis of Intermediate 2-2

Synthesis was carried out in the same method as in the synthesis of Intermediate 1-1, except for using Intermediate 2-1 and 1-bromo-2-nitrobenzene in place of the methyl 2-bromo-5-chlorobenzoate and the dibenzofuran-4-boronic acid, thereby obtaining 0.4 g (yield: 66%) of a white solid.

The resulting white solid was identified to be Intermediate 2-2 through an LC-MS analysis.

### Synthesis of Intermediate 2-A

In a flask, 0.4 g of Intermediate 2-2, 0.7 g of triphenylphosphine, and 9 mL of 1,3-dichlorobenzene were charged in an argon atmosphere and heated with stirring for 12 hours.

After cooling to room temperature (25° C.), a reaction solution was purified by means of silica gel column chromatography, thereby obtaining 187 mg (yield: 58%) of a white solid.

The resulting white solid was identified to be Intermediate 2-A through an LC-MS analysis.

### Synthesis 4 (Synthesis of Intermediate 3-A)

A synthesis scheme of Intermediate 3-A is shown below.

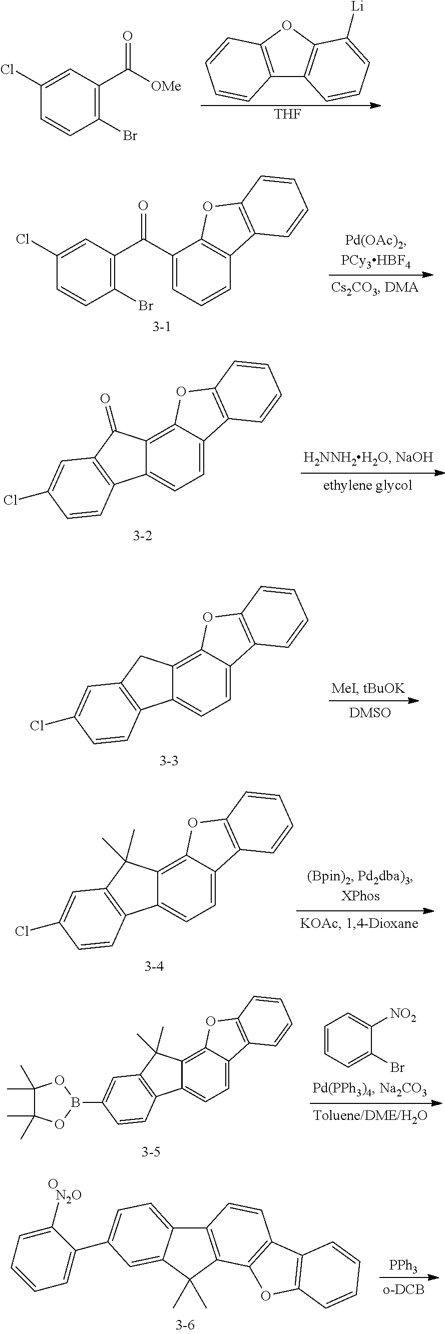

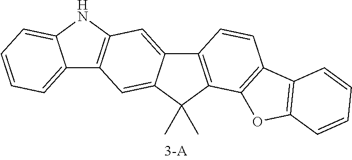 [SMILES: CC1(C)c2cc3c(cc2-c2ccc4c(oc5ccccc54)c21)[nH]c1ccccc13]

### Synthesis of Intermediate 3-1

In a flask, 8.6 g of 4-bromobenzofuran and 100 mL of tetrahydrofuran were charged in an argon atmosphere. After cooling to −30° C., 26 mL of a butyllithium hexane solution (1.6 M) was gradually added, and the contents were stirred for 30 minutes as they were. Thereafter, the temperature was increased to 0° C., followed by stirring for one hour. Thereafter, in a flask in which 17.5 g of methyl 2-bromo-5-chlorobenzoate and 40 mL of tetrahydrofuran had been separately charged in an argon atmosphere, the preceding prepared solution was added as it was at 0° C. After increasing the temperature to room temperature, the contents were stirred for 5 hours.

Thereafter, an ammonium chloride aqueous solution was added to a reaction solution, and the resultant was transferred into a separatory funnel and extracted with toluene. Thereafter, an organic layer was dried over sodium sulfate, filtered, and then concentrated. A residue was purified by means of silica gel column chromatography, thereby obtaining 7.3 g (yield: 55%) of a white solid.

The resulting white solid was identified to be Intermediate 3-1 through an LC-MS analysis.

### Synthesis of Intermediate 3-2

Synthesis was carried out in the same method as in the synthesis of Intermediate 1-A, except for using Intermediate 3-1 in place of the Intermediate 1-4, thereby obtaining 3.0 g (yield: 41%) of a white solid.

The resulting white solid was identified to be Intermediate 3-2 through an LC-MS analysis.

### Synthesis of Intermediate 3-3

In a flask, 3.0 g of Intermediate 3-2, 300 mg of hydrazine monohydrate, 1.6 g of sodium hydroxide, and 30 mL of ethylene glycol were charged in an argon atmosphere and heated at 160° C. with stirring for 4 hours.

After cooling to room temperature (25° C.), a reaction solution was transferred into a separatory funnel, and water was added, followed by extracting with a solution of hexane and ethyl acetate (3/1). Thereafter, an organic layer was dried over sodium sulfate, filtered, and then concentrated. A residue was purified by means of silica gel column chromatography, thereby obtaining 1.1 g (yield: 50%) of a white solid.

The resulting white solid was identified to be Intermediate 3-3 through an LC-MS analysis.

### Synthesis of Intermediate 3-4

In a flask, 1.1 g of Intermediate 3-3, 1.7 mL of iodomethane, 1.3 g of potassium tert-butoxide, and 15 mL of dimethyl sulfoxide were charged in an argon atmosphere and stirred at room temperature for 24 hours.

After cooling to room temperature (25° C.), a reaction solution was transferred into a separatory funnel, and water was added, followed by extracting with a solution of hexane and ethyl acetate (3/1). Thereafter, an organic layer was dried over sodium sulfate, filtered, and then concentrated. A residue was purified by means of silica gel column chromatography, thereby obtaining 1.1 g (yield: 92%) of a white solid.

The resulting white solid was identified to be Intermediate 3-4 through an LC-MS analysis.

### Synthesis of Intermediate 3-5

Synthesis was carried out in the same manner as in the synthesis of Intermediate 2-1, except for using Intermediate 3-4 in place of the Intermediate 1-3, thereby obtaining 1.0 g (yield: 69%) of a white solid.

The resulting white solid was identified to be Intermediate 3-5 through an LC-MS analysis.

### Synthesis of Intermediate 3-6

Synthesis was carried out in the same method as in the synthesis of Intermediate 1-1, except for using Intermediate 3-5 and 1-bromo-2-nitrobenzene in place of the methyl 2-bromo-5-chlorobenzoate and the dibenzofuran-4-boronic acid, thereby obtaining 730 mg (yield: 71%) of a white solid.

The resulting white solid was identified to be Intermediate 3-6 through an LC-MS analysis.

### Synthesis of Intermediate 3-A

Synthesis was carried out in the same method as in the synthesis of Intermediate 2-A, except for using Intermediate 3-6 in place of the Intermediate 2-2, thereby obtaining 450 g (yield: 64%) of a white solid.

The resulting white solid was identified to be Intermediate 3-A through an LC-MS analysis.

### Synthesis 5 (Synthesis of Intermediate 4-A)

A synthesis scheme of Intermediate 4-A is shown below.

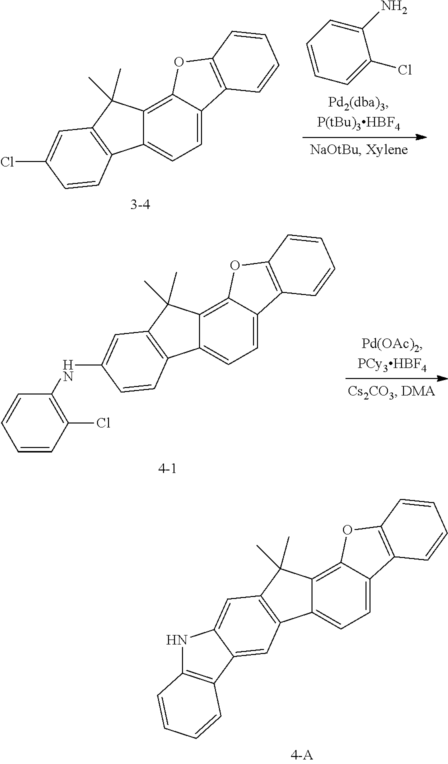

### Synthesis of Intermediate 4-1

Synthesis was carried out in the same method as in the synthesis of Intermediate 1-4, except for using Intermediate 3-4 in place of the Intermediate 1-3, thereby obtaining 1.0 g (yield: 82%) of a white solid.

The resulting white solid was identified to be Intermediate 4-1 through an LC-MS analysis.

### Synthesis of Intermediate 4-A

Synthesis was carried out in the same method as in the synthesis of Intermediate 1-A, except for using Intermediate 4-1 in place of the Intermediate 1-4, thereby obtaining 0.6 g (yield: 62%) of a white solid.

The resulting white solid was identified to be Intermediate 4-A through an LC-MS analysis.

### Synthesis 6 (Synthesis of Intermediate 5-A)

A synthesis scheme of Intermediate 5-A is shown below.

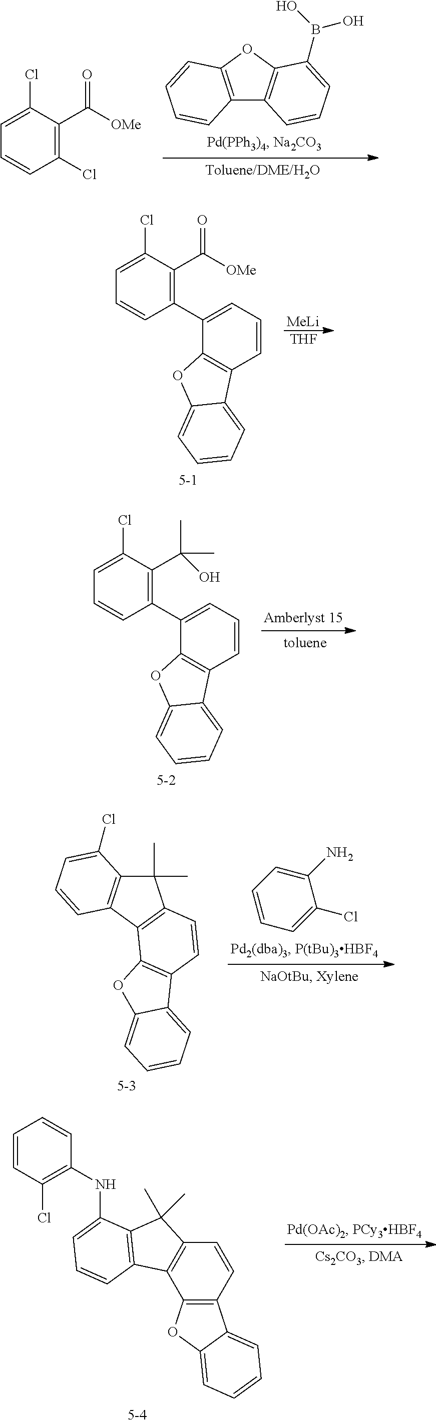

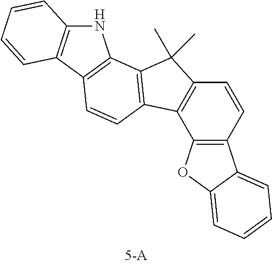 [SMILES: CC1(C)c2ccc3c(oc4ccccc43)c2-c2ccc3c([nH]c4ccccc43)c21]

### Synthesis of Intermediate 5-1

Synthesis was carried out in the same method as in the synthesis of Intermediate 1-1, except for using methyl 2,6-dichlorobenzoate in place of the methyl 2-bromo-5-chlorobenzoate, thereby obtaining 1.4 g (yield: 81%) of a white solid.

The resulting white solid was identified to be Intermediate 5-1 through an LC-MS analysis.

### Synthesis of Intermediate 5-2

Synthesis was carried out in the same method as in the synthesis of Intermediate 1-2, except for using Intermediate 5-1 in place of the Intermediate 1-1, thereby obtaining 0.8 g (yield: 56%) of a white solid.

The resulting white solid was identified to be Intermediate 5-2 through an LC-MS analysis.

### Synthesis of Intermediate 5-3

Synthesis was carried out in the same method as in the synthesis of Intermediate 1-3, except for using Intermediate 5-2 in place of the Intermediate 1-2, thereby obtaining 318 mg (yield: 44%) of a white solid.

The resulting white solid was identified to be Intermediate 5-3 through an LC-MS analysis.

### Synthesis of Intermediate 5-4

Synthesis was carried out in the same method as in the synthesis of Intermediate 1-4, except for using Intermediate 5-3 in place of the Intermediate 1-3, thereby obtaining 278 mg (yield: 68%) of a white solid.

The resulting white solid was identified to be Intermediate 5-4 through an LC-MS analysis.

### Synthesis of Intermediate 5-A

Synthesis was carried out in the same method as in the synthesis of Intermediate 1-A, except for using Intermediate 5-4 in place of the Intermediate 1-4, thereby obtaining 183 mg (yield: 72%) of a white solid.

The resulting white solid was identified to be Intermediate 5-A through an LC-MS analysis.

### Synthesis 7 (Synthesis of Intermediate 6-A)

A synthesis scheme of Intermediate 6-A is shown below.

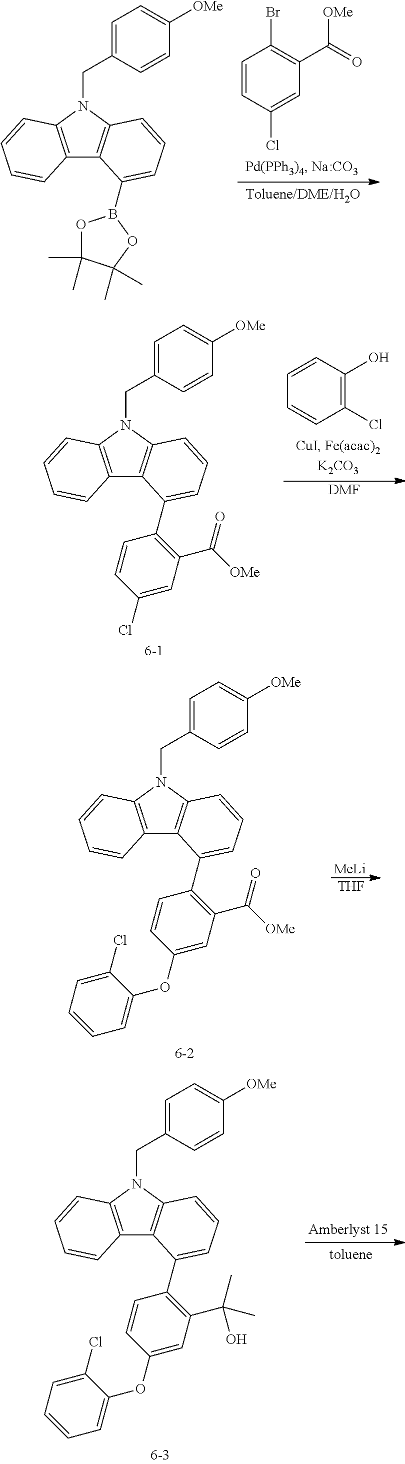

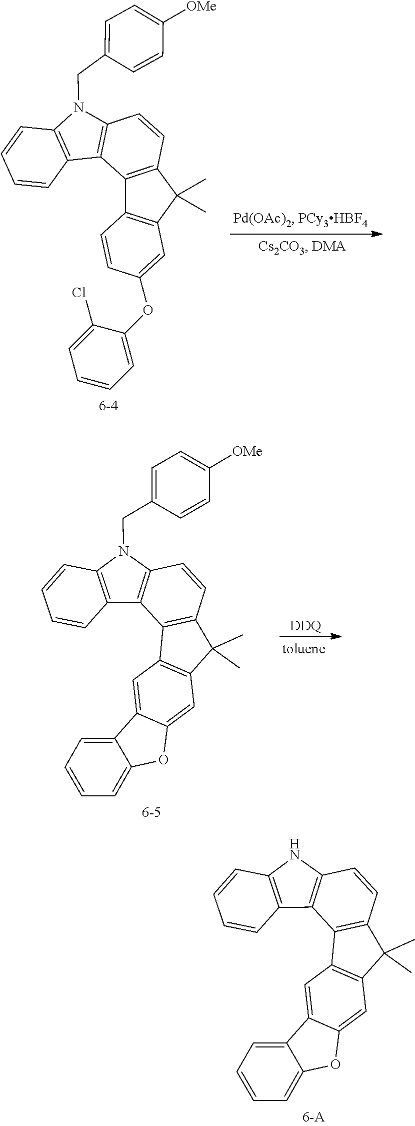

### Synthesis of Intermediate 6-1

Synthesis was carried out in the same method as in the synthesis of Intermediate 1-1, except for using 9-[(4-methoxyphenyl)methyl]-4-(4,4,5,5-tetramethyl)-1,3,2-dioxaborolanyl carbazole in place of the dibenzofuran-4-boronic acid, thereby obtaining 2.1 g (yield: 65%) of a white solid.

The resulting white solid was identified to be Intermediate 6-1 through an LC-MS analysis.

### Synthesis of Intermediate 6-2

In a flask, 2.1 g of Intermediate 6-1, 1.2 g of 2-chlorophenol, 45 mg of copper iodide, 166 mg of tris(2,4-pentanedionato)iron(III), 1.9 g of potassium carbonate, and 15 mL of N,N-dimethylformamide were charged in an argon atmosphere and heated at 130° C. with stirring for 12 hours.

After cooling to room temperature (25° C.), a reaction solution was filtered, a filtrate was transferred into a separatory funnel, and water was added, followed by extracting with a solution of hexane and ethyl acetate (3/1). Thereafter, an organic layer was dried over sodium sulfate, filtered, and then concentrated. A residue was purified by means of silica gel column chromatography, thereby obtaining 986 mg (yield: 38%) of a white solid.

The resulting white solid was identified to be Intermediate 6-2 through an LC-MS analysis.

### Synthesis of Intermediate 6-3

Synthesis was carried out in the same method as in the synthesis of Intermediate 1-2, except for using Intermediate 6-1 in place of the Intermediate 1-1, thereby obtaining 417 mg (yield: 42%) of a white solid.

The resulting white solid was identified to be Intermediate 6-3 through an LC-MS analysis.

### Synthesis of Intermediate 6-4

Synthesis was carried out in the same method as in the synthesis of Intermediate 1-3, except for using Intermediate 6-3 in place of the Intermediate 1-2, thereby obtaining 228 mg (yield: 56%) of a white solid.

The resulting white solid was identified to be Intermediate 6-4 through an LC-MS analysis.

### Synthesis of Intermediate 6-5

Synthesis was carried out in the same method as in the synthesis of Intermediate 1-A, except for using Intermediate 6-4 in place of the Intermediate 1-4, thereby obtaining 128 mg (yield: 61%) of a white solid.

The resulting white solid was identified to be Intermediate 6-5 through an LC-MS analysis.

### Synthesis of Intermediate 6-A

In a flask, 128 mg of Intermediate 6-5, 180 mg of 2,3-dichloro-5,6-dicyano-1,4-benzoquinone, and 2.0 mL of toluene were charged in an argon atmosphere and heated under reflux with stirring for 12 hours.

After cooling to room temperature (25° C.), a reaction solution was filtered, and a filtrate was concentrated. A residue was purified by means of silica gel column chromatography, thereby obtaining 76 mg (yield: 78%) of a white solid.

The resulting white solid was identified to be Intermediate 6-A through an LC-MS analysis.

### Synthesis 8 (Synthesis of Intermediate 7-A)

A synthesis scheme of Intermediate 7-A is shown below.

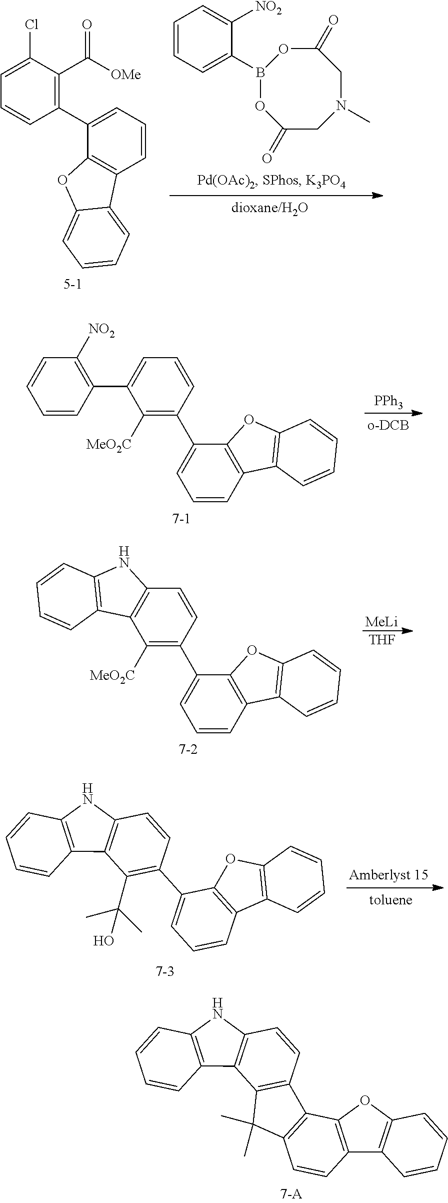

### Synthesis of Intermediate 7-1

In a flask, 640 mg of Intermediate 5-1, 792 g of 6-methyl-2-(2-nitrophenyl)-1,3,6,2-dioxazaborocane-4,8-dione, 34 mg of palladium(II) acetate, 124 mg of 2-dicyclohexylphosphino-2′,6′-dimethoxybiphenyl, 3.1 g of tripotassium phosphate, 15 mL of 1,4-dioxane, and 3.0 mL of water were charged in an argon atmosphere and heated under reflux with stirring for 12 hours.

After cooling to room temperature (25° C.), a reaction solution was transferred into a separatory funnel, and water was added, followed by extracting with toluene. Thereafter, an organic layer was dried over sodium sulfate, filtered, and then concentrated. A residue was purified by means of silica gel column chromatography, thereby obtaining 592 mg (yield: 75%) of a white solid.

The resulting white solid was identified to be Intermediate 7-1 through an LC-MS analysis.

### Synthesis of Intermediate 7-2

Synthesis was carried out in the same method as in the synthesis of Intermediate 2-A, except for using Intermediate 7-1 in place of the Intermediate 2-2, thereby obtaining 235 mg (yield: 41%) of a white solid.

The resulting white solid was identified to be Intermediate 7-2 through an LC-MS analysis.

### Synthesis of Intermediate 7-3

Synthesis was carried out in the same method as in the synthesis of Intermediate 1-2, except for using Intermediate 7-2 in place of the Intermediate 1-1, thereby obtaining 145 mg (yield: 62%) of a white solid.

The resulting white solid was identified to be Intermediate 7-3 through an LC-MS analysis.

### Synthesis of Intermediate 7-4

Synthesis was carried out in the same method as in the synthesis of Intermediate 1-3, except for using Intermediate 7-3 in place of the Intermediate 1-2, thereby obtaining 75 mg (yield: 54%) of a white solid.

The resulting white solid was identified to be Intermediate 7-4 through an LC-MS analysis.

### Synthesis 9 (Synthesis of Intermediate 8-A)

A synthesis scheme of Intermediate 8-A is shown below.

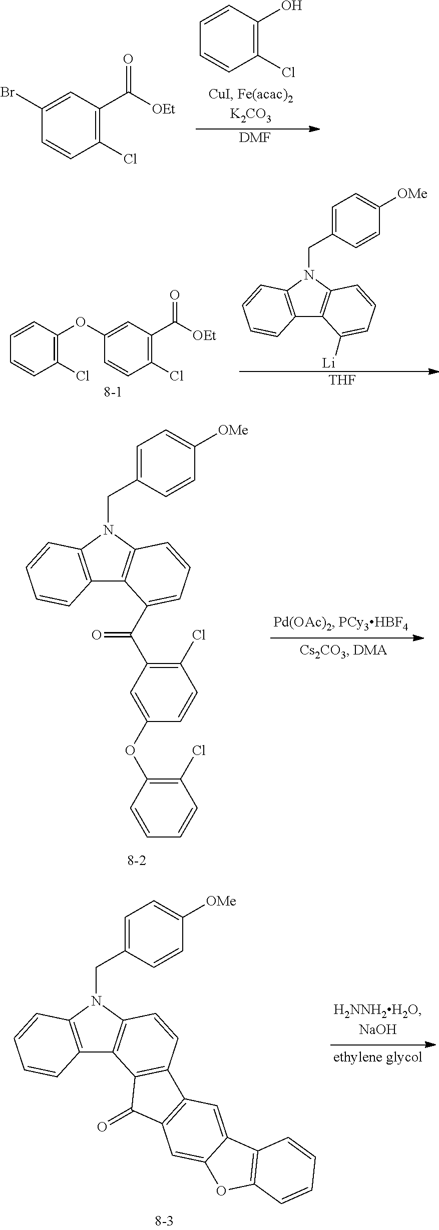

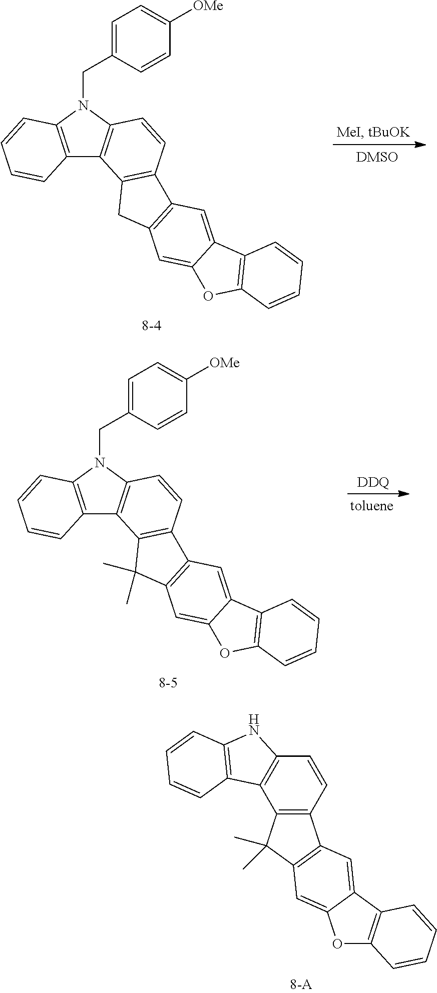

### Synthesis of Intermediate 8-1

Synthesis was carried out in the same method as in the synthesis of Intermediate 6-2, except for using methyl 2-bromo-5-chlorobenzoate in place of the Intermediate 6-1, thereby obtaining 10.0 g (yield: 42%) of a white solid.

The resulting white solid was identified to be Intermediate 8-1 through an LC-MS analysis.

### Synthesis of Intermediate 8-2

Synthesis was carried out in the same method as in the synthesis of Intermediate 3-1, except for using Intermediate 8-1 in place of the methyl 2-bromo-5-chlorobenzoate and using 4-bromo-9-[4-methoxyphenylmethyl]carbazole in place of the 4-bromodibenzofuran, thereby obtaining 7.6 g (yield: 43%) of a white solid.

The resulting white solid was identified to be Intermediate 8-2 through an LC-MS analysis.

### Synthesis of Intermediate 8-3

Synthesis was carried out in the same method as in the synthesis of Intermediate 1-A, except for using Intermediate 8-2 in place of the Intermediate 1-4, thereby obtaining 2.1 g (yield: 32%) of a white solid.

The resulting white solid was identified to be Intermediate 8-3 through an LC-MS analysis.

### Synthesis of Intermediate 8-4

Synthesis was carried out in the same method as in the synthesis of Intermediate 3-3, except for using Intermediate 8-3 in place of the Intermediate 3-2, thereby obtaining 1.4 g (yield: 66%) of a white solid.

The resulting white solid was identified to be Intermediate 8-4 through an LC-MS analysis.

### Synthesis of Intermediate 8-5

Synthesis was carried out in the same method as in the synthesis of Intermediate 8-A, except for using Intermediate 8-4 in place of the Intermediate 3-3, thereby obtaining 1.3 g (yield: 94%) of a white solid.

The resulting white solid was identified to be Intermediate 8-5 through an LC-MS analysis.

### Synthesis of Intermediate 8-A

Synthesis was carried out in the same method as in the synthesis of Intermediate 6-A, except for using Intermediate 8-5 in place of the Intermediate 6-5 thereby obtaining 747 mg (yield: 73%) of a white solid.

The resulting white solid was identified to be Intermediate 8-A through an LC-MS analysis.

### Synthesis 10 (Synthesis of Intermediate 9-A)

A synthesis scheme of Intermediate 9-A is shown below.

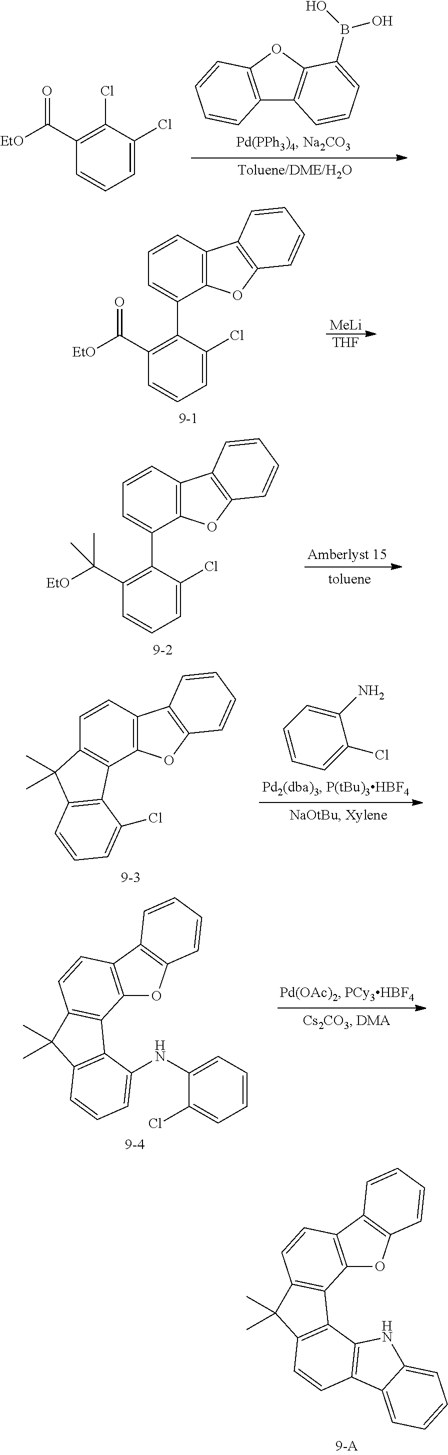

### Synthesis of Intermediate 9-1

Synthesis was carried out in the same method as in the synthesis of Intermediate 1-1, except for using ethyl 2,3-dichlorobenzoate in place of the methyl 2-bromo-5-chlorobenzoate, thereby obtaining 2.8 g (yield: 81%) of a white solid.

The resulting white solid was identified to be Intermediate 9-1 through an LC-MS analysis.

### Synthesis of Intermediate 9-2

Synthesis was carried out in the same method as in the synthesis of Intermediate 1-2, except for using Intermediate 9-1 in place of the Intermediate 1-1, thereby obtaining 1.6 g (yield: 62%) of a white solid.

The resulting white solid was identified to be Intermediate 9-2 through an LC-MS analysis.

### Synthesis of Intermediate 9-3

Synthesis was carried out in the same method as in the synthesis of Intermediate 1-3, except for using Intermediate 9-2 in place of the Intermediate 1-2, thereby obtaining 854 mg (yield: 55%) of a white solid.

The resulting white solid was identified to be Intermediate 9-3 through an LC-MS analysis.

### Synthesis of Intermediate 9-4

Synthesis was carried out in the same method as in the synthesis of Intermediate 1-4, except for using Intermediate 9-3 in place of the Intermediate 1-3, thereby obtaining 791 mg (yield: 72%) of a white solid.

The resulting white solid was identified to be Intermediate 9-4 through an LC-MS analysis.

### Synthesis of Intermediate 9-A

Synthesis was carried out in the same method as in the synthesis of Intermediate 1-A, except for using Intermediate 9-4 in place of the Intermediate 1-4, thereby obtaining 560 mg (yield: 78%) of a white solid.

The resulting white solid was identified to be Intermediate 9-A through an LC-MS analysis.

### Synthesis 11 (Synthesis of Intermediate 10-A)

A synthesis scheme of Intermediate 10-A is shown below.

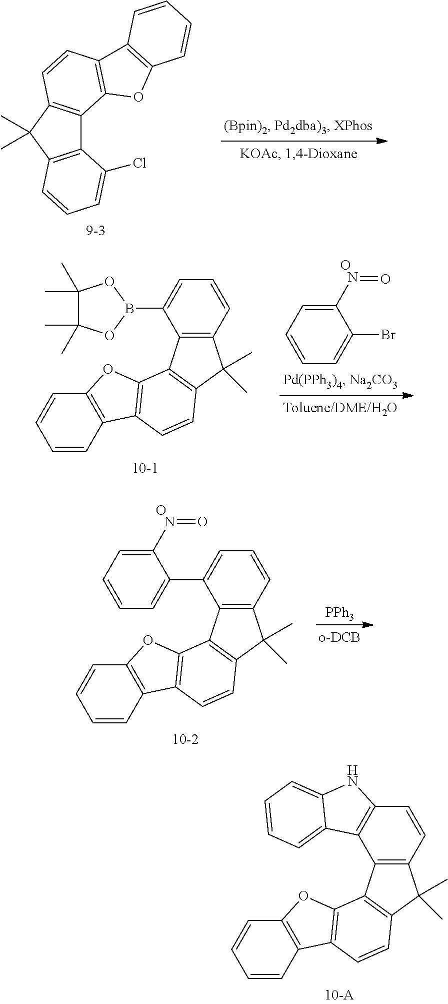

### Synthesis of Intermediate 10-1

Synthesis was carried out in the same manner as in the synthesis of Intermediate 2-1, except for using Intermediate 9-3 in place of the Intermediate 1-3, thereby obtaining 2.1 g (yield: 52%) of a white solid.

The resulting white solid was identified to be Intermediate 10-1 through an LC-MS analysis.

### Synthesis of Intermediate 10-2

Synthesis was carried out in the same method as in the synthesis of Intermediate 1-1, except for using Intermediate 10-1 and 1-bromo-2-nitrobenzene in place of the methyl 2-bromo-5-chlorobenzoate and the dibenzofuran-4-boronic acid, thereby obtaining 1.2 g (yield: 58%) of a white solid.

The resulting white solid was identified to be Intermediate 10-2 through an LC-MS analysis.

### Synthesis of Intermediate 10-A

Synthesis was carried out in the same method as in the synthesis of Intermediate 2-A, except for using Intermediate 10-2 in place of the Intermediate 2-2, thereby obtaining 659 mg (yield: 59%) of a white solid.

The resulting white solid was identified to be Intermediate 10-A through an LC-MS analysis.

### Synthesis 12 (Synthesis of Intermediate 11-A)

A synthesis scheme of Intermediate 11-A is shown below.

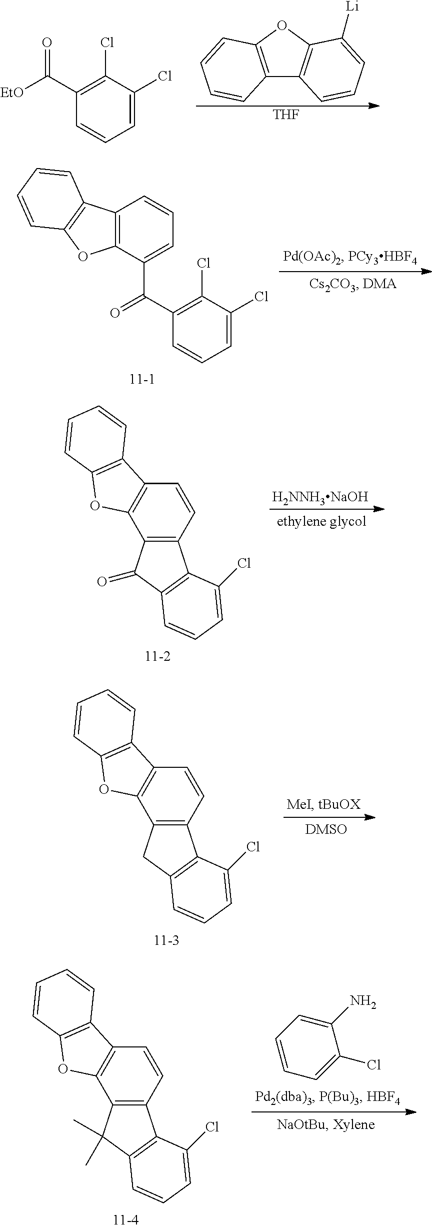

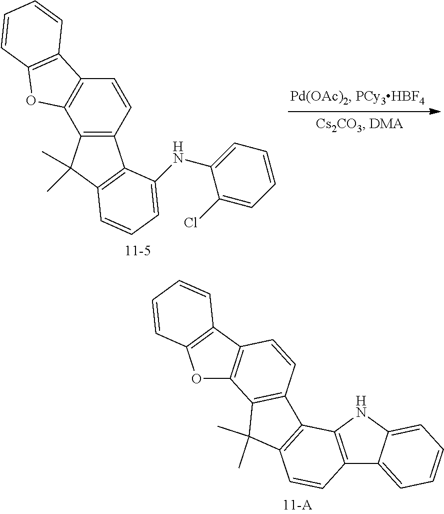 [SMILES: CC1(C)c2cccc(Nc3ccccc3Cl)c2-c2ccc3c(oc4ccccc43)c21 ; CC1(C)c2ccc3c([nH]c4ccccc43)c2-c2ccc3c(oc4ccccc43)c21]

### Synthesis of Intermediate 11-1

Synthesis was carried out in the same method as in the synthesis of Intermediate 3-1, except for using ethyl 2,3-dichlorobenzoate in place of the methyl 2-bromo-5-chlorobenzoate, thereby obtaining 2.8 g (yield: 76%) of a white solid.

The resulting white solid was identified to be Intermediate 11-1 through an LC-MS analysis.

### Synthesis of Intermediate 11-2

Synthesis was carried out in the same method as in the synthesis of Intermediate 1-A, except for using Intermediate 11-1 in place of the Intermediate 1-4, thereby obtaining 1.4 g (yield: 57%) of a white solid.

The resulting white solid was identified to be Intermediate 11-2 through an LC-MS analysis.

### Synthesis of Intermediate 11-3

Synthesis was carried out in the same method as in the synthesis of Intermediate 3-3, except for using Intermediate 11-2 in place of the Intermediate 3-2, thereby obtaining 927 mg (yield: 69%) of a white solid.

The resulting white solid was identified to be Intermediate 11-3 through an LC-MS analysis.

### Synthesis of Intermediate 11-4

Synthesis was carried out in the same method as in the synthesis of Intermediate 3-4, except for using Intermediate 11-3 in place of the Intermediate 3-3, thereby obtaining 892 mg (yield: 88%) of a white solid.

The resulting white solid was identified to be Intermediate 11-4 through an LC-MS analysis.

### Synthesis of Intermediate 11-5

Synthesis was carried out in the same method as in the synthesis of Intermediate 1-4, except for using Intermediate 11-4 in place of the Intermediate 1-3, thereby obtaining 951 mg (yield: 83%) of a white solid.

The resulting white solid was identified to be Intermediate 11-5 through an LC-MS analysis.

### Synthesis of Intermediate 11-A

Synthesis was carried out in the same method as in the synthesis of Intermediate 1-A, except for using Intermediate 11-5 in place of the Intermediate 1-4, thereby obtaining 506 mg (yield: 58%) of a white solid.

The resulting white solid was identified to be Intermediate 11-A through an LC-MS analysis.

### Synthesis 13 (Synthesis of Intermediate 12-A)

A synthesis scheme of Intermediate 12-A is shown below.

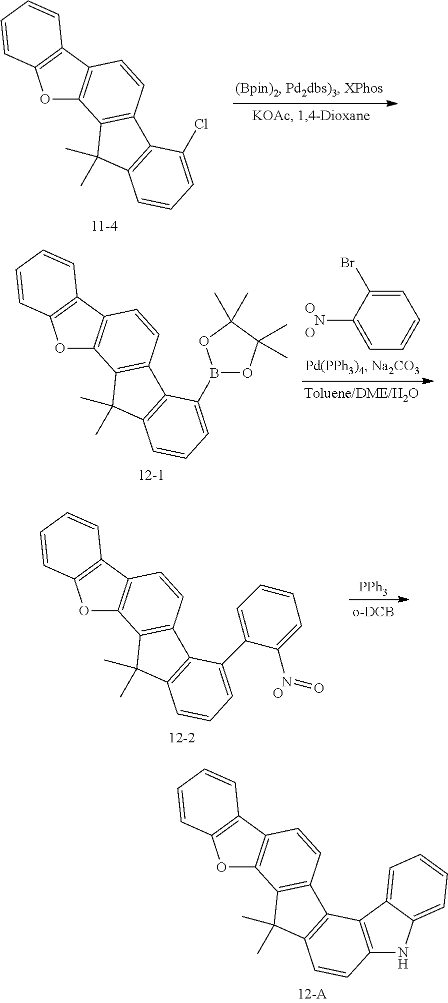

### Synthesis of Intermediate 12-1

Synthesis was carried out in the same manner as in the synthesis of Intermediate 2-1, except for using Intermediate 11-4 in place of the Intermediate 1-3, thereby obtaining 1.2 g (yield: 54%) of a white solid.

The resulting white solid was identified to be Intermediate 12-1 through an LC-MS analysis.

### Synthesis of Intermediate 12-2

Synthesis was carried out in the same method as in the synthesis of Intermediate 1-1, except for using Intermediate 12-1 and 1-bromo-2-nitrobenzene in place of the methyl 2-bromo-5-chlorobenzoate and the dibenzofuran-4-boronic acid, thereby obtaining 835 mg (yield: 72%) of a white solid.

The resulting white solid was identified to be Intermediate 12-2 through an LC-MS analysis.

### Synthesis of Intermediate 12-A

Synthesis was carried out in the same method as in the synthesis of Intermediate 2-A, except for using Intermediate 12-2 in place of the Intermediate 2-2, thereby obtaining 515 mg (yield: 67%) of a white solid.

The resulting white solid was identified to be Intermediate 12-A through an LC-MS analysis.

### Synthesis 14 (Synthesis of Intermediate 13-A)

A synthesis scheme of Intermediate 13-A is shown below.

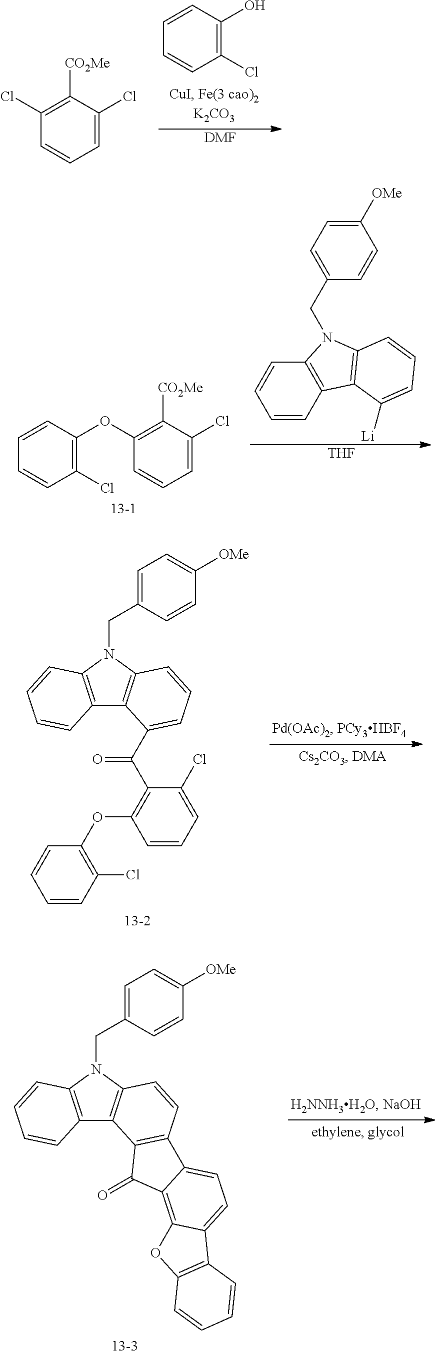

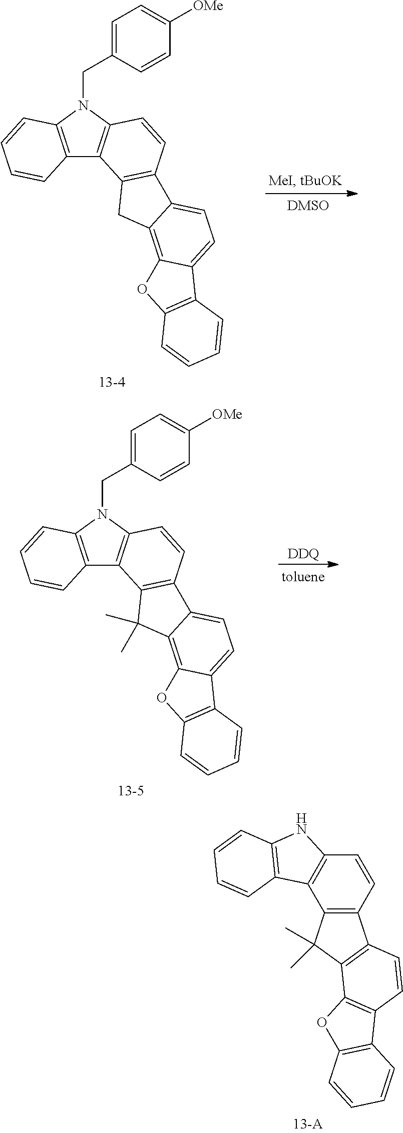

### Synthesis of Intermediate 13-1

Synthesis was carried out in the same method as in the synthesis of Intermediate 6-2, except for using methyl 2,6-dichlorobenzoate in place of the Intermediate 6-1, thereby obtaining 4.0 g (yield: 78%) of a white solid.

The resulting white solid was identified to be Intermediate 13-1 through an LC-MS analysis.

### Synthesis of Intermediate 13-2

Synthesis was carried out in the same method as in the synthesis of Intermediate 3-1, except for using Intermediate 13-1 in place of the methyl 2-bromo-5-chlorobenzoate and using 4-bromo-9-[4-methoxyphenylmethyl]carbazole in place of the 4-bromodibenzofuran, thereby obtaining 4.3 g (yield: 58%) of a white solid.

The resulting white solid was identified to be Intermediate 13-2 through an LC-MS analysis.

### Synthesis of Intermediate 13-3

Synthesis was carried out in the same method as in the synthesis of Intermediate 1-A, except for using Intermediate 13-2 in place of the Intermediate 1-4, thereby obtaining 1.3 g (yield: 35%) of a white solid.

The resulting white solid was identified to be Intermediate 13-3 through an LC-MS analysis.

### Synthesis of Intermediate 13-4

Synthesis was carried out in the same method as in the synthesis of Intermediate 3-3, except for using Intermediate 13-3 in place of the Intermediate 3-2, thereby obtaining 843 mg (yield: 67%) of a white solid.

The resulting white solid was identified to be Intermediate 13-4 through an LC-MS analysis.

### Synthesis of Intermediate 13-5

Synthesis was carried out in the same method as in the synthesis of Intermediate 3-4, except for using Intermediate 13-4 in place of the Intermediate 3-3, thereby obtaining 814 mg (yield: 91%) of a white solid.

The resulting white solid was identified to be Intermediate 13-5 through an LC-MS analysis.

### Synthesis of Intermediate 13-A

Synthesis was carried out in the same method as in the synthesis of Intermediate 6-A, except for using Intermediate 13-5 in place of the Intermediate 6-5 thereby obtaining 481 mg (yield: 78%) of a white solid.

The resulting white solid was identified to be Intermediate 13-A through an LC-MS analysis.

### Synthesis 15 (Synthesis of Intermediate 14-A)

A synthesis scheme of Intermediate 14-A is shown below.

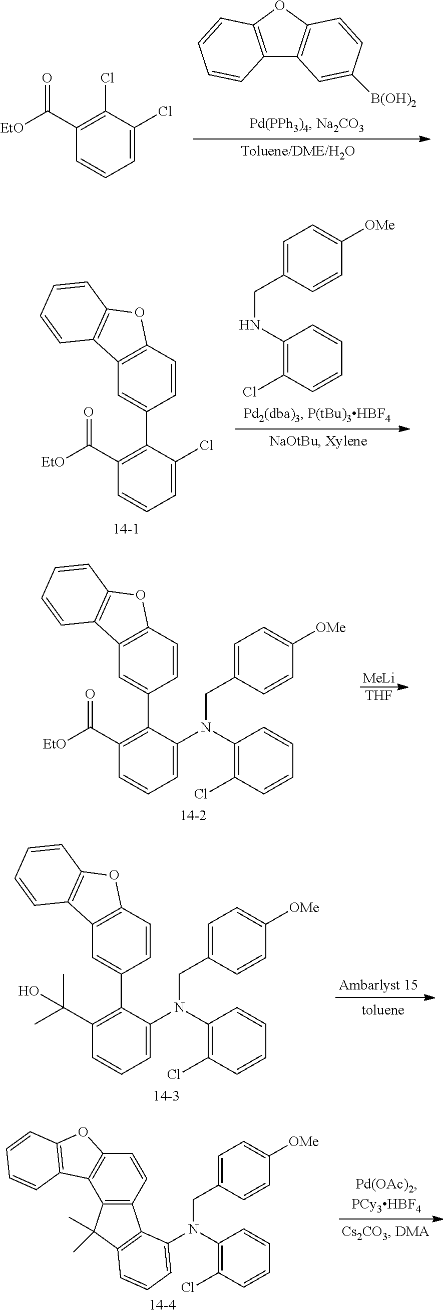

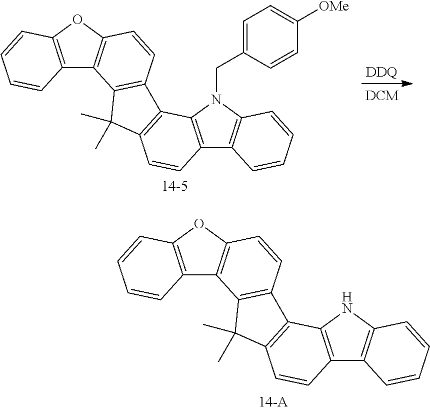

### Synthesis of Intermediate 14-1

Synthesis was carried out in the same method as in the synthesis of Intermediate 1-1, except for using ethyl 2,3-dichlorobenzoate and dibenzofuran-2-boronic acid in place of the methyl 2-bromo-5-chlorobenzoate and the dibenzofuran-4-boronic acid, thereby obtaining 6.6 g (yield: 74%) of a white solid.

The resulting white solid was identified to be Intermediate 14-1 through an LC-MS analysis.

### Synthesis of Intermediate 14-2

Synthesis was carried out in the same method as in the synthesis of Intermediate 1-4, except for using Intermediate 14-1 in place of the Intermediate 1-3, thereby obtaining 7.8 g (yield: 74%) of a white solid.

The resulting white solid was identified to be Intermediate 14-2 through an LC-MS analysis.

### Synthesis of Intermediate 6-1

Synthesis was carried out in the same method as in the synthesis of Intermediate 1-2, except for using Intermediate 14-2 in place of the Intermediate 1-1, thereby obtaining 4.4 g (yield: 58%) of a white solid.

The resulting white solid was identified to be Intermediate 14-3 through an LC-MS analysis.

### Synthesis of Intermediate 14-4

Synthesis was carried out in the same method as in the synthesis of Intermediate 1-3, except for using Intermediate 14-3 in place of the Intermediate 1-2, thereby obtaining 1.5 g (yield: 35%) of a white solid.

The resulting white solid was identified to be Intermediate 14-4 through an LC-MS analysis.

### Synthesis of Intermediate 14-5

Synthesis was carried out in the same method as in the synthesis of Intermediate 1-A, except for using Intermediate 14-4 in place of the Intermediate 1-4, thereby obtaining 1.0 g (yield: 72%) of a white solid.

The resulting white solid was identified to be Intermediate 14-5 through an LC-MS analysis.

### Synthesis of Intermediate 14-A

Synthesis was carried out in the same method as in the synthesis of Intermediate 6-A, except for using Intermediate 14-5 in place of the Intermediate 6-5 thereby obtaining 515 mg (yield: 68%) of a white solid.

The resulting white solid was identified to be Intermediate 14-A through an LC-MS analysis.

### Synthesis 16 (Synthesis of Intermediate 15-A)

A synthesis scheme of Intermediate 15-A is shown below.

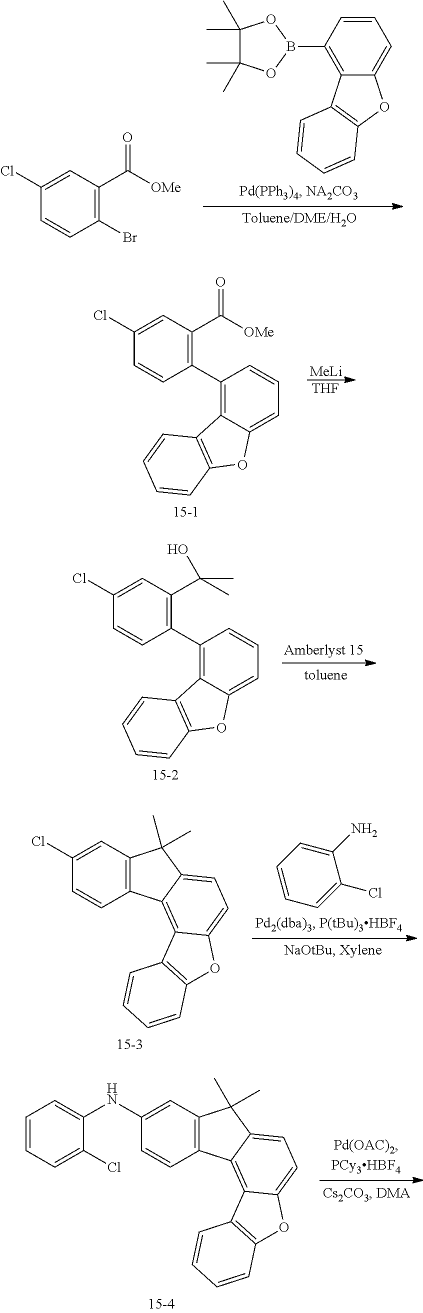

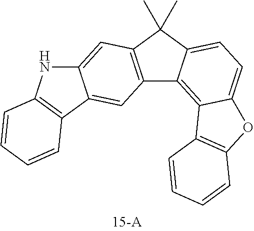 [SMILES: CC1(C)c2cc3[nH]c4ccccc4c3cc2-c2c1ccc1oc3ccccc3c21]

### Synthesis of Intermediate 15-1

Synthesis was carried out in the same method as in the synthesis of Intermediate 1-1, except for using 2-(1-dibenzofuranyl)-4,4,5,5-tetramethyl-1,3,2-dioxaborolane in place of the dibenzofuran-4-boronic acid, thereby obtaining 2.4 g (yield: 87%) of a white solid.

The resulting white solid was identified to be Intermediate 15-1 through an LC-MS analysis.

### Synthesis of Intermediate 15-2

Synthesis was carried out in the same method as in the synthesis of Intermediate 1-2, except for using Intermediate 15-1 in place of the Intermediate 1-1, thereby obtaining 1.3 g (yield: 5287%) of a white solid.

The resulting white solid was identified to be Intermediate 15-2 through an LC-MS analysis.

### Synthesis of Intermediate 15-3

Synthesis was carried out in the same method as in the synthesis of Intermediate 1-3, except for using Intermediate 15-2 in place of the Intermediate 1-2, thereby obtaining 800 mg (yield: 6152%) of a white solid.

The resulting white solid was identified to be Intermediate 15-3 through an LC-MS analysis.

### Synthesis of Intermediate 15-4

Synthesis was carried out in the same method as in the synthesis of Intermediate 1-4, except for using Intermediate 15-3 in place of the Intermediate 1-3, thereby obtaining 832 mg (yield: 81%) of a white solid.

The resulting white solid was identified to be Intermediate 15-4 through an LC-MS analysis.

### Synthesis of Intermediate 15-A

Synthesis was carried out in the same method as in the synthesis of Intermediate 1-A, except for using Intermediate 15-4 in place of the Intermediate 1-4, thereby obtaining 441 mg (yield: 58%) of a white solid.

The resulting white solid was identified to be Intermediate 15-A through an LC-MS analysis.

Next, synthesis of the following Compounds 10 to 24 is shown.

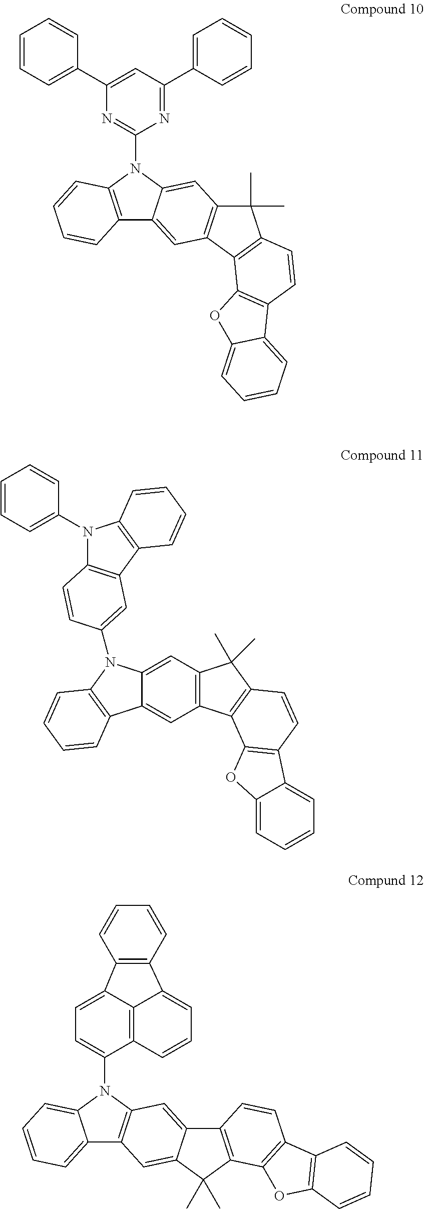 [SMILES: CC1(C)c2cc3c(cc2-c2c1ccc1c2oc2ccccc21)c1ccccc1n3-c1nc(-c2ccccc2)cc(-c2ccccc2)n1 ; CC1(C)c2cc3c(cc2-c2c1ccc1c2oc2ccccc21)c1ccccc1n3-c1ccc2c(c1)c1ccccc1n2-c1ccccc1 ; CC1(C)c2cc3c4ccccc4n(-c4ccc5c6c(cccc46)-c4ccccc4-5)c3cc2-c2ccc3c(oc4ccccc43)c21]

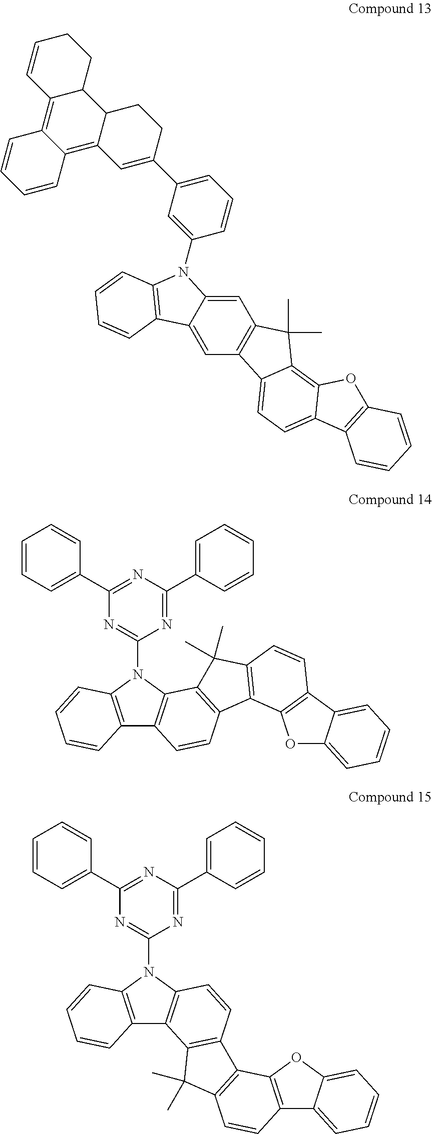 [SMILES: CC1(C)c2cc3c(cc2-c2ccc4c(oc5ccccc54)c21)c1ccccc1n3-c1cccc(C2=CC3=c4ccccc4=C4C=CCCC4C3CC2)c1 ; CC1(C)c2ccc3c(oc4ccccc43)c2-c2ccc3c4ccccc4n(-c4nc(-c5ccccc5)nc(-c5ccccc5)n4)c3c21 ; CC1(C)c2ccc3c(oc4ccccc43)c2-c2ccc3c(c21)c1ccccc1n3-c1nc(-c2ccccc2)nc(-c2ccccc2)n1]

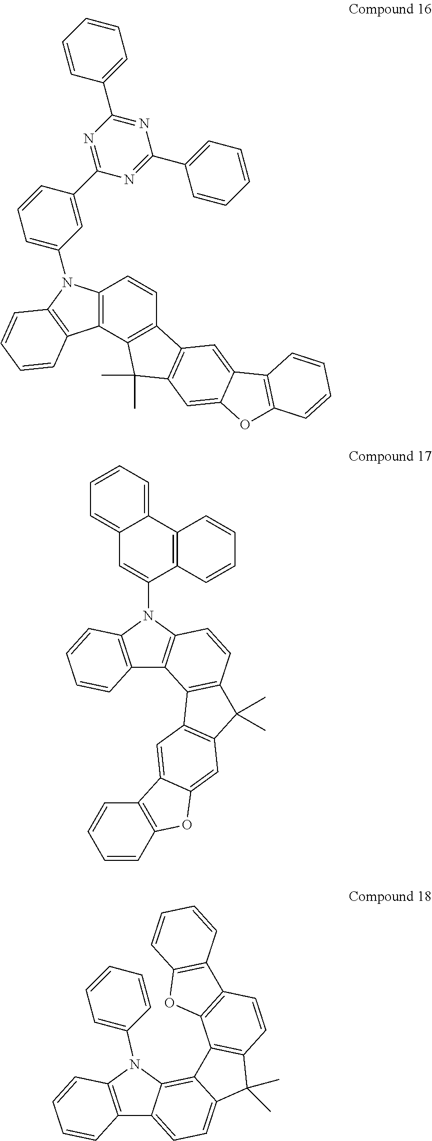 [SMILES: CC1(C)c2cc3oc4ccccc4c3cc2-c2ccc3c(c21)c1ccccc1n3-c1cccc(-c2nc(-c3ccccc3)nc(-c3ccccc3)n2)c1 ; CC1(C)c2cc3oc4ccccc4c3cc2-c2c1ccc1c2c2ccccc2n1-c1cc2ccccc2c2ccccc12 ; CC1(C)c2ccc3c(oc4ccccc43)c2-c2c1ccc1c3ccccc3n(-c3ccccc3)c21]

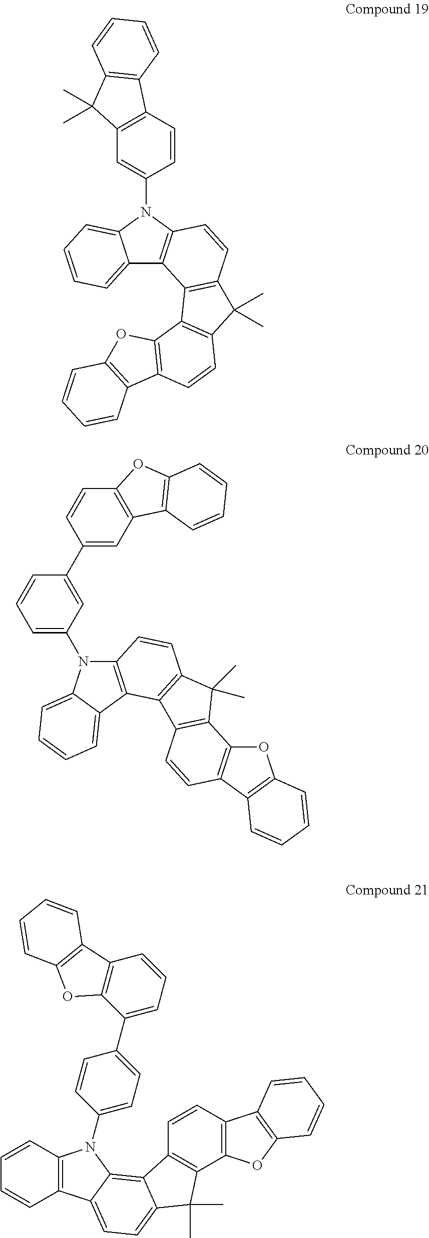 [SMILES: CC1(C)c2ccccc2-c2ccc(-n3c4ccccc4c4c5c(ccc43)C(C)(C)c3ccc4c(oc6ccccc64)c3-5)cc21 ; CC1(C)c2ccc3c(c2-c2ccc4c(oc5ccccc54)c21)c1ccccc1n3-c1cccc(-c2ccc3oc4ccccc4c3c2)c1 ; CC1(C)c2ccc3c4ccccc4n(-c4ccc(-c5cccc6c5oc5ccccc56)cc4)c3c2-c2ccc3c(oc4ccccc43)c21]

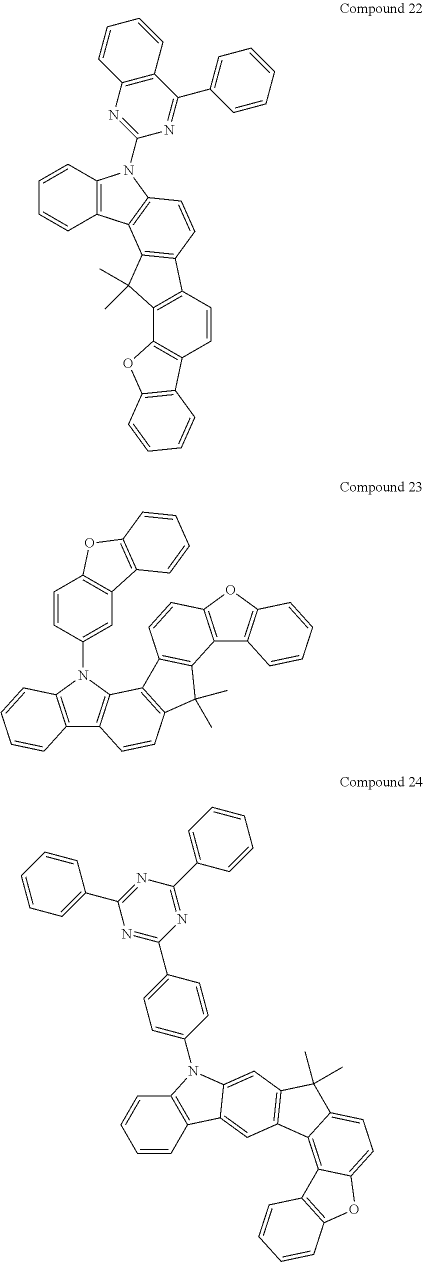 [SMILES: CC1(C)c2c(ccc3c2oc2ccccc23)-c2ccc3c(c21)c1ccccc1n3-c1nc(-c2ccccc2)c2ccccc2n1 ; CC1(C)c2ccc3c4ccccc4n(-c4ccc5oc6ccccc6c5c4)c3c2-c2ccc3oc4ccccc4c3c21 ; CC1(C)c2cc3c(cc2-c2c1ccc1oc4ccccc4c21)c1ccccc1n3-c1ccc(-c2nc(-c3ccccc3)nc(-c3ccccc3)n2)cc1]

### Synthesis Example 10

### Synthesis of Compound 10

Synthesis was carried out in the same method as in the synthesis of Intermediate 1-4, except for using Intermediate 1-A and 2-chloro-4,6-diphenyl-pyrimidine in place of the 2-chloroaniline and the Intermediate 1-3, thereby obtaining 485 mg (yield: 76%) of a white solid.

The resulting white solid was identified to be Compound 10 through an LC-MS analysis.

### Synthesis Example 11

### Synthesis of Compound 11

Synthesis was carried out in the same method as in the synthesis of Intermediate 1-4, except for using Intermediate 2-A and 3-bromo-9-phenyl-carbazole in place of the 2-chloroaniline and the Intermediate 1-3, thereby obtaining 391 mg (yield: 48%) of a white solid.

The resulting white solid was identified to be Compound 11 through an LC-MS analysis.

### Synthesis Example 12

### Synthesis of Compound 12

Synthesis was carried out in the same method as in the synthesis of Intermediate 1-4, except for using Intermediate 3-A and 3-bromofluoranthene in place of the 2-chloroaniline and the Intermediate 1-3, thereby obtaining 628 mg (yield: 68%) of a white solid.

The resulting white solid was identified to be Compound 12 through an LC-MS analysis.

### Synthesis Example 13

### Synthesis of Compound 13

Synthesis was carried out in the same method as in the synthesis of Intermediate 1-4, except for using Intermediate 4-A and 2-(3-bromophenyl)triphenylene in place of the 2-chloroaniline and the Intermediate 1-3, thereby obtaining 484 mg (yield: 54%) of a white solid.

The resulting white solid was identified to be Compound 13 through an LC-MS analysis.

### Synthesis Example 14

### Synthesis of Compound 14

Synthesis was carried out in the same method as in the synthesis of Intermediate 1-4, except for using Intermediate 5-A and 2-chloro-4,6-diphenyl-1,3,5-triazine in place of the 2-chloroaniline and the Intermediate 1-3, thereby obtaining 369 mg (yield: 59%) of a white solid.

The resulting white solid was identified to be Compound 14 through an LC-MS analysis.

### Synthesis Example 15

### Synthesis of Compound 15

Synthesis was carried out in the same method as in the synthesis of Intermediate 1-4, except for using Intermediate 6-A and 2-chloro-4,6-diphenyl-1,3,5-triazine in place of the 2-chloroaniline and the Intermediate 1-3, thereby obtaining 319 mg (yield: 56%) of a white solid.

The resulting white solid was identified to be Compound 15 through an LC-MS analysis.

### Synthesis Example 16

### Synthesis of Compound 16

Synthesis was carried out in the same method as in the synthesis of Intermediate 1-4, except for using Intermediate 7-A and 2-(3-bromophenyl)-4,6-diphenyl-1,3,5-triazine in place of the 2-chloroaniline and the Intermediate 1-3, thereby obtaining 299 mg (yield: 59%) of a white solid.

The resulting white solid was identified to be Compound 16 through an LC-MS analysis.

### Synthesis Example 17

### Synthesis of Compound 17

Synthesis was carried out in the same method as in the synthesis of Intermediate 1-4, except for using Intermediate 8-A and 9-bromophenanthrene in place of the 2-chloroaniline and the Intermediate 1-3, thereby obtaining 621 mg (yield: 71%) of a white solid.

The resulting white solid was identified to be Compound 17 through an LC-MS analysis.

### Synthesis Example 18

### Synthesis of Compound 18

Synthesis was carried out in the same method as in the synthesis of Intermediate 1-4, except for using Intermediate 9-A and bromobenzene in place of the 2-chloroaniline and the Intermediate 1-3, thereby obtaining 518 mg (yield: 71%) of a white solid.

The resulting white solid was identified to be Compound 18 through an LC-MS analysis.

### Synthesis Example 19

### Synthesis of Compound 19

Synthesis was carried out in the same method as in the synthesis of Intermediate 1-4, except for using Intermediate 10-A and 2-bromo-9,9-dimethyl-fluorene in place of the 2-chloroaniline and the Intermediate 1-3, thereby obtaining 481 mg (yield: 69%) of a white solid.

The resulting white solid was identified to be Compound 19 through an LC-MS analysis.

### Synthesis Example 20

### Synthesis of Compound 20

Synthesis was carried out in the same method as in the synthesis of Intermediate 1-4, except for using Intermediate 11-A and 2-(3-bromophenyl)dibenzofuran in place of the 2-chloroaniline and the Intermediate 1-3, thereby obtaining 374 mg (yield: 65%) of a white solid.

The resulting white solid was identified to be Compound 20 through an LC-MS analysis.

### Synthesis Example 21

### Synthesis of Compound 21

Synthesis was carried out in the same method as in the synthesis of Intermediate 1-4, except for using Intermediate 12-A and 4-(4-bromophenyl)dibenzothiophene in place of the 2-chloroaniline and the Intermediate 1-3, thereby obtaining 368 mg (yield: 69%) of a white solid.

The resulting white solid was identified to be Compound 21 through an LC-MS analysis.

### Synthesis Example 22

### Synthesis of Compound 22

Synthesis was carried out in the same method as in the synthesis of Intermediate 1-4, except for using Intermediate 13-A and 2-chloro-4-phenyl-quinazoline in place of the 2-chloroaniline and the Intermediate 1-3, thereby obtaining 351 mg (yield: 71%) of a white solid.

The resulting white solid was identified to be Compound 22 through an LC-MS analysis.

### Synthesis Example 23

### Synthesis of Compound 23

Synthesis was carried out in the same method as in the synthesis of Intermediate 1-4, except for using Intermediate 14-A and 2-bromodibenzofuran in place of the 2-chloroaniline and the Intermediate 1-3, thereby obtaining 396 mg (yield: 67%) of a white solid.

The resulting white solid was identified to be Compound 23 through an LC-MS analysis.

### Synthesis Example 24

### Synthesis of Compound 24

Synthesis was carried out in the same method as in the synthesis of Intermediate 1-4, except for using Intermediate 15-A and 2-(4-bromophenyl-4,6-diphenyl-1,3,5-triazine in place of the 2-chloroaniline and the Intermediate 1-3, thereby obtaining 466 mg (yield: 71%) of a white solid.

The resulting white solid was identified to be Compound 24 through an LC-MS analysis.

### Preparation of Organic EL Device of the Embodiment of the Present Invention

### Example 1

### Preparation of Organic EL Device Using Compound 1

A glass substrate provided with an ITO transparent electrode (anode) and having a size of 25 mm×75 mm×1.1 mm (manufactured by Geomatec Co., Ltd.) was subjected to ultrasonic cleaning in isopropyl alcohol for 5 minutes and further subjected to UV ozone cleaning for 30 minutes. It is to be noted that a thickness of ITO was set to 130 nm.

The cleaned glass substrate provided with a transparent electrode line was installed in a substrate holder of a vacuum vapor deposition apparatus. The following compound (HI1) was first vapor-deposited on the surface of the side on which the transparent electrode line was formed, so as to cover the transparent electrode, thereby fabricating a hole injecting layer having a film thickness of 5 nm.

On the hole injecting layer made of this compound (HI1), the following compound (HT1) was vapor-deposited to fabricate a first hole transporting layer having a film thickness of 90 nm. Furthermore, on the first hole transporting layer made of this compound (HT1), the following compound (HT2) was vapor-deposited to fabricate a second hole transporting layer having a film thickness of 60 nm.

Subsequently, on the second hole transporting layer made of this compound (HT2), the aforementioned Compound 1 obtained in Synthesis Example 1 as a host material was subjected to co-vapor deposition with the following compound (Ir(ppy)₃) as a dopant material, thereby fabricating an organic layer (light emitting layer) having a film thickness of 40 nm. It is to be noted that a concentration of the following compound (Ir(ppy)₃) in the organic layer (light emitting layer) was set to 5% by mass.

On this light emitting layer, the following compound (ET1) and the following compound (Liq) were subjected to co-vapor deposition, thereby fabricating an electron transporting layer having a film thickness of 30 nm. It is to be noted that a concentration of Liq in this organic layer was set to 50% by mass. This organic layer functions as an electron transporting layer.

Furthermore, on this electron transporting layer, the following compound (Liq) was vapor-deposited to fabricate an Liq film having a film thickness of 1 nm, and on this Liq film, metal Al was vapor-deposited to fabricate a metal cathode having a film thickness of 80 nm, thereby preparing an organic EL device.

A device constitution of the organic EL device prepared in the present Example 1 is schematically shown as follows.

ITO (130 nm)/HI1 (5 nm)/HT1 (90 nm)/HT2 (60 nm)/Compound 1+Ir(ppy)₃ (5 wt %) (40 nm)/ET1+Liq (50 wt %) (30 nm)/Liq (1 nm)/Al (80 nm)

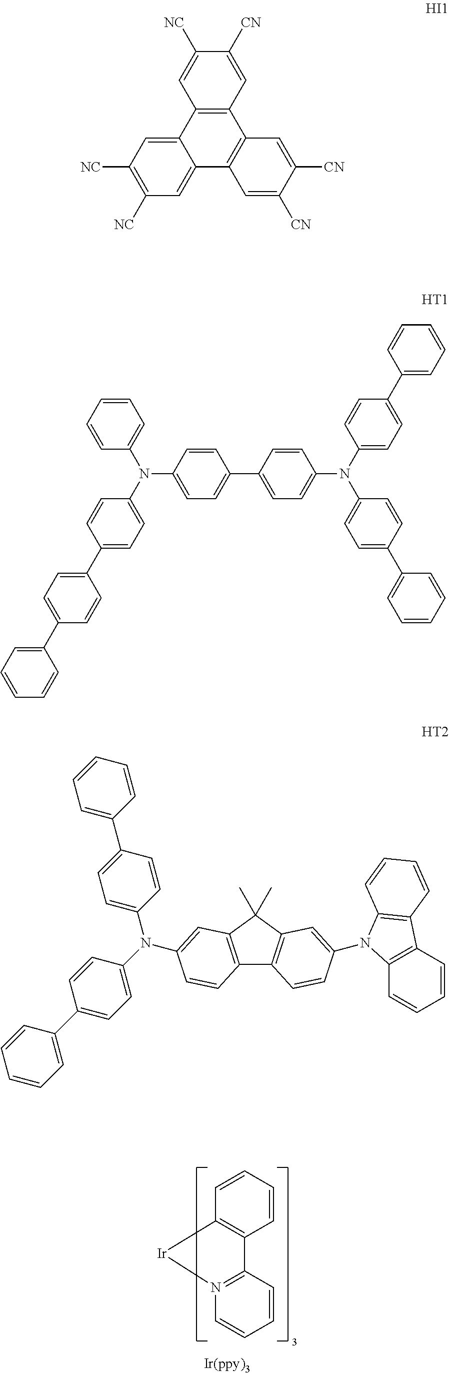

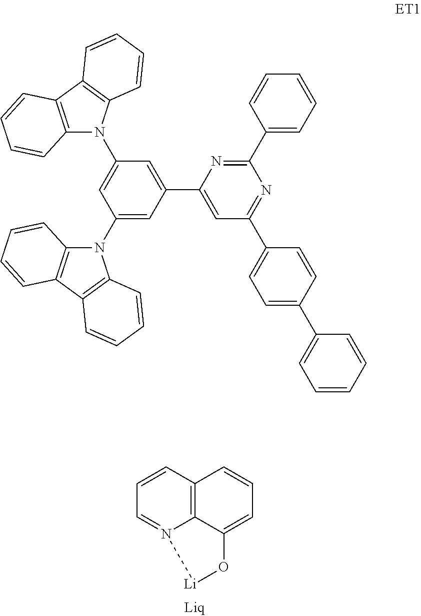

### Example 2

### Preparation of Organic EL Device Using Compound 2

An organic EL device was prepared in the same manner as in Example 1, except that in Example 1, an organic layer (light emitting layer) was formed by using the aforementioned Compound 2 obtained in Synthesis Example 2 in place of the Compound 1 used as the host material.

A device constitution of the organic EL device prepared in the present Example 2 is schematically shown as follows.

ITO (130 nm)/HI1 (5 nm)/HT1 (90 nm)/HT2 (60 nm)/Compound 2+Ir(ppy)₃ (5 wt %) (40 nm)/ET1+Liq (50 wt %) (30 nm)/Liq (1 nm)/Al (80 nm)

### Examples 3 to 5

### Preparation of Organic EL Devices Using Compounds 10, 15, and 16, Respectively

Organic EL devices were prepared in the same manner as in Example 1, except that in Example 1, an organic layer (light emitting layer) was formed by using the aforementioned Compound 10, Compound 15, and Compound 16 obtained in Synthesis Examples 10, 15, and 16, respectively in place of the Compound 1 used as the host material.

A device constitution of each of the organic EL devices prepared in the present Examples 3 to 5 is schematically shown as follows.

ITO (130 nm)/HI1 (5 nm)/HT1 (90 nm)/HT2 (60 nm)/Compound 10, 15, or 16+Ir(ppy)₃ (5 wt %) (40 nm)/ET1+Liq (50 wt %) (30 nm)/Liq (1 nm)/Al (80 nm)

### Comparative Example 1

### Preparation of Organic EL Device Using the Following Compound (Host1)

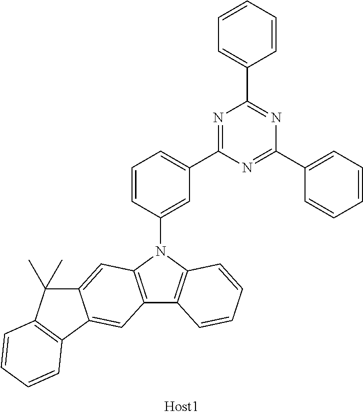 [SMILES: CC1(C)c2ccccc2-c2cc3c4ccccc4n(-c4cccc(-c5nc(-c6ccccc6)nc(-c6ccccc6)n5)c4)c3cc21]

An organic EL device was prepared in the same manner as in Example 1, except that in Example 1, an organic layer (light emitting layer) was formed by using the aforementioned compound (Host1) in place of the Compound 1 used as the host material.

A device constitution of the organic EL device prepared in the present Comparative Example 1 is schematically shown as follows.

ITO (130 nm)/HI1 (5 nm)/HT1 (90 nm)/HT2 (60 nm)/Host1+Ir(ppy)₃ (5 wt %) (40 nm)/ET1+Liq (50 wt %) (30 nm)/Liq (1 nm)/Al (80 nm)

### Comparative Examples 2 to 4

### Preparation of Organic EL Devices Using the Following Compounds 1′ to 3′, Respectively

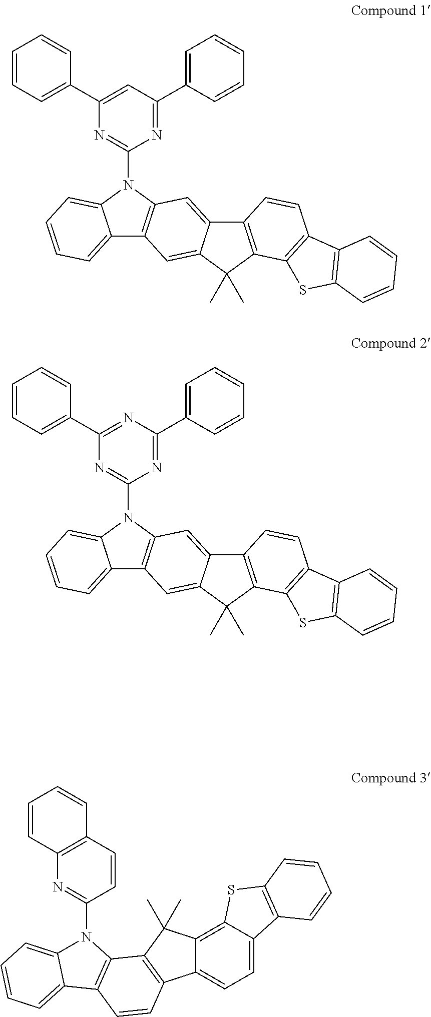 [SMILES: CC1(C)c2cc3c4ccccc4n(-c4nc(-c5ccccc5)cc(-c5ccccc5)n4)c3cc2-c2ccc3c(sc4ccccc43)c21 ; CC1(C)c2cc3c4ccccc4n(-c4nc(-c5ccccc5)nc(-c5ccccc5)n4)c3cc2-c2ccc3c(sc4ccccc43)c21 ; CC1(C)c2c(ccc3c2sc2ccccc23)-c2ccc3c4ccccc4n(-c4ccc5ccccc5n4)c3c21]

Organic EL devices were prepared in the same manner as in Example 1, except that in Example 1, an organic layer (light emitting layer) was formed by using the aforementioned compounds (Compounds 1′ to 3′), respectively as shown in Table 1 in place of the Compound 1 used as the host material.

A device constitution of each of the organic EL devices prepared in the present Comparative Examples 2 to 4 is schematically shown as follows.

ITO (130 nm)/HI1 (5 nm)/HT1 (90 nm)/HT2 (60 nm)/each of Compounds 1′ to 3′+Ir(ppy)₃ (5 wt %) (40 nm)/ET1+Liq (50 wt %) (30 nm)/Liq (1 nm)/Al (80 nm)

### <Evaluation of Organic EL Device>

With respect to the organic EL devices prepared in Examples 1 to 5 and Comparative Examples 1 to 4, the following evaluations were conducted. The evaluation results are shown in Table 1.

### (1) Luminance and CIE1931 Chromaticity

When a voltage was impressed on the device such that a current density was 10 mA/cm², a luminance (unit: cd/m²) and CIE1931 chromaticity coordinates (x,y) were measured using a spectral emission luminance meter “CS-1000” (a product name, manufactured by Konica Minolta Ltd.).

### (2) Main Peak Wavelength (λₚ)

When a voltage was impressed on the device such that a current density was 10 mA/cm², a spectral emission luminance spectrum was measured using a spectral emission luminance meter “CS-1000” (a product name, manufactured by Konica Minolta Ltd.). A main peak wavelength (λₚ) (unit: nm) was determined from the resulting spectral emission luminance spectrum.

### (3) External Quantum Efficiency (EQE)

When a voltage was impressed on the device such that a current density was 10 mA/cm², a spectral emission luminance spectrum was measured using a spectral emission luminance meter “CS-1000” (a product name, manufactured by Konica Minolta Ltd.).

On the assumption that Lambertian radiation was conducted, an external quantum efficiency (EQE) (unit: %) was calculated from the resulting spectral emission luminance spectrum.

TABLE 1

| | | CIE1931 | | | |
|---|---|---|---|---|---|
| | | chromaticity | | | |
| | Host material | x | y | λₚ (nm) | EQE (%) |
| Example 1 | Compound 1 | 0.319 | 0.631 | 520 | 14.90 |
| Example 2 | Compound 2 | 0.322 | 0.629 | 522 | 14.85 |
| Example 3 | Compound 10 | 0.323 | 0.628 | 522 | 14.62 |
| Example 4 | Compound 15 | 0.308 | 0.636 | 522 | 13.94 |
| Example 5 | Compound 16 | 0.318 | 0.631 | 522 | 15.32 |
| Comparative | Host1 | 0.319 | 0.631 | 520 | 11.99 |
| Example 1 | | | | | |
| Comparative | Compound 1′ | 0.325 | 0.626 | 521 | 10.60 |
| Example 2 | | | | | |
| Comparative | Compound 2′ | 0.326 | 0.626 | 522 | 9.20 |
| Example 3 | | | | | |
| Comparative | Compound 3′ | 0.315 | 0.629 | 519 | 9.60 |
| Example 4 | | | | | |

From Table 1, it may be considered that by using each of Compounds 1, 2, 10, 15, and 16 as the embodiment of the present invention as the host material to be contained in the light emitting layer of the organic EL device, the interaction with the dopant became strong, the efficiency of energy transfer was improved, and the external quantum efficiency (EQE) of the organic EL device was improved. Such effects are evident from comparison with the organic EL device of each of Comparative Examples 1 to 4 using the aforementioned compound (Host1) and Compounds 1′ to 3′, respectively.

### Preparation of Organic EL Device of Another Embodiment of the Present Invention

### Example 6

### Preparation of Organic EL Device Using Compound 1

A glass substrate provided with an ITO transparent electrode (anode) and having a size of 25 mm×75 mm×1.1 mm (manufactured by Geomatec Co., Ltd.) was subjected to ultrasonic cleaning in isopropyl alcohol for 5 minutes and further subjected to UV ozone cleaning for 30 minutes. It is to be noted that a thickness of ITO was set to 130 nm.

The cleaned glass substrate provided with a transparent electrode line was installed in a substrate holder of a vacuum vapor deposition apparatus. The following compound (HI1) was first vapor-deposited on the surface of the side on which the transparent electrode line was formed, so as to cover the transparent electrode, thereby fabricating a hole injecting layer having a film thickness of 5 nm.

On the hole injecting layer made of this compound (HI1), the following compound (HT1) was vapor-deposited to fabricate a first hole transporting layer having a film thickness of 200 nm. Furthermore, on the first hole transporting layer made of this compound (HT1), the following compound (HT2) was vapor-deposited to fabricate a second hole transporting layer having a film thickness of 10 nm.

Subsequently, on the second hole transporting layer made of this compound (HT2), the aforementioned Compound 1 obtained in Synthesis Example 1 as a host material was subjected to co-vapor deposition with the following compound (Ir(piq)₃) as a dopant material, thereby fabricating an organic layer (light emitting layer) having a film thickness of 40 nm. It is to be noted that a concentration of the following compound (Ir(piq)₃) in the organic layer (light emitting layer) was set to 2% by mass.

On this light emitting layer, the following compound (ET1) and the following compound (Liq) were subjected to co-vapor deposition, thereby fabricating an electron transporting layer having a film thickness of 30 nm. It is to be noted that a concentration of Liq in this organic layer was set to 50% by mass. This organic layer functions as an electron transporting layer.

Furthermore, on this electron transporting layer, the following compound (Liq) was vapor-deposited to fabricate an Liq film having a film thickness of 1 nm, and on this Liq film, metal Al was vapor-deposited to fabricate a metal cathode having a film thickness of 80 nm, thereby preparing an organic EL device.

A device constitution of the organic EL device prepared in the present Example 6 is schematically shown as follows.

ITO (130 nm)/HI1 (5 nm)/HT1 (200 nm)/HT2 (10 nm)/Compound 1+Ir (piq)₃ (2 wt %) (40 nm)/ET1+Liq (50 wt %) (30 nm)/Liq (1 nm)/Al (80 nm)

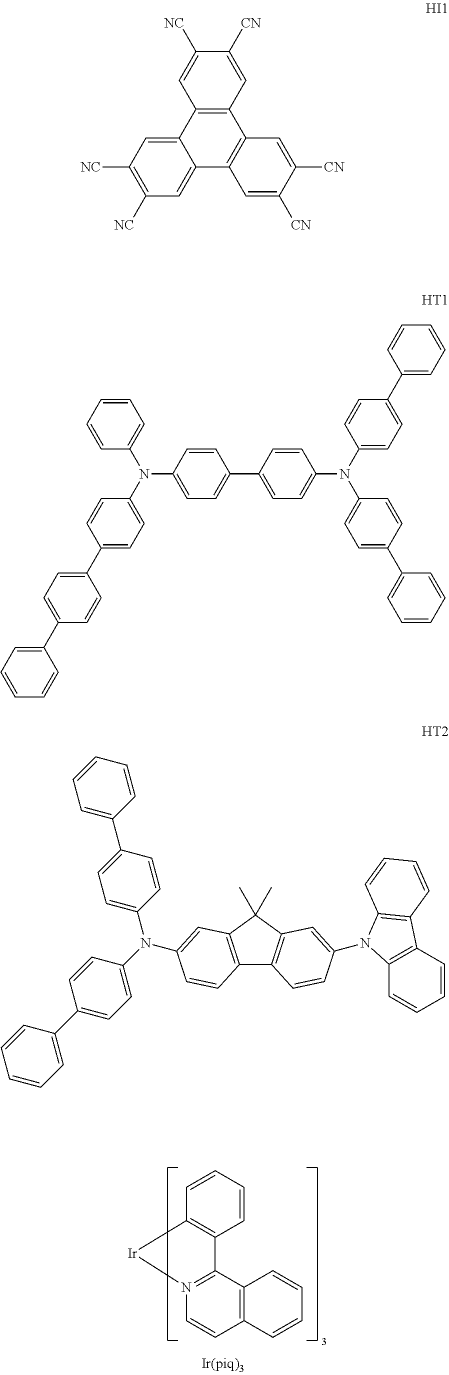

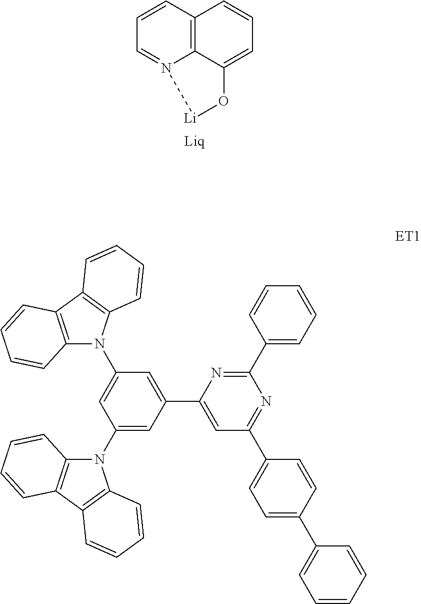

### Examples 7 to 29

### Preparation of Organic EL Devices Using Compounds 2 to 24, Respectively

Organic EL devices were prepared in the same manner as in Example 6, except that in Example 6, an organic layer (light emitting layer) was formed by using the aforementioned Compounds 2 to 24 obtained in Synthesis Examples 2 to 24, respectively as shown in Table 2 in place of the Compound 1 used as the host material.

A device constitution of each of the organic EL devices prepared in the present Examples 7 to 29 is schematically shown as follows.

ITO (130 nm)/HI1 (5 nm)/HT1 (200 nm)/HT2 (10 nm)/each of Compounds 2 to 24+Ir (piq)₃ (2 wt %) (40 nm)/ET1+Liq (50 wt %) (30 nm)/Liq (1 nm)/Al (80 nm)

### Comparative Examples 5 to 7

### Preparation of Organic EL Devices Using Compounds 1′ to 3′, Respectively

Organic EL devices were prepared in the same manner as in Example 6, except that in Example 6, an organic layer (light emitting layer) was formed by using the aforementioned compounds (Compounds 1′ to 3′), respectively as shown in Table 2 in place of the Compound 1 used as the host material.

A device constitution of each of the organic EL devices prepared in the present Comparative Examples 5 to 7 is schematically shown as follows.

ITO (130 nm)/HI1 (5 nm)/HT1 (200 nm)/HT2 (10 nm)/each of Compounds 1′ to 3′+Ir (piq)₃ (2 wt %) (40 nm)/ET1+Liq (50 wt %) (30 nm)/Liq (1 nm)/Al (80 nm)

### <Evaluation of Organic EL Device>

With respect to the organic EL devices prepared in Examples 6 to 29, the following evaluations were conducted. The evaluation results are shown in Table 2.

### (4) Main Peak Wavelength (λₚ)

When a voltage was impressed on the device such that a current density was 10 mA/cm², a spectral emission luminance spectrum was measured using a spectral emission luminance meter “CS-1000” (a product name, manufactured by Konica Minolta Ltd.). A main peak wavelength (λₚ) (unit: nm) was determined from the resulting spectral emission luminance spectrum.

### (5) Device Life (LT90)

A DC continuous-current test was carried out by setting an initial current density to 50 mA/cm², a time taken until the luminance was reduced to 90% of the luminance at the time of starting the test was measured, and its measurement time was defined as a life (LT90).

TABLE 2

| | | λₚ | LT90 |
|---|---|---|---|
| | Host material | (nm) | (hr) |
| Example 6 | Compound 1 | 619 | 216 |
| Example 7 | Compound 2 | 619 | 237 |
| Example 8 | Compound 3 | 621 | 285 |
| Example 9 | Compound 4 | 619 | 267 |
| Example 10 | Compound 5 | 621 | 346 |
| Example 11 | Compound 6 | 621 | 370 |
| Example 12 | Compound 7 | 621 | 215 |
| Example 13 | Compound 8 | 619 | 219 |
| Example 14 | Compound 9 | 621 | 245 |
| Example 15 | Compound 10 | 621 | 236 |
| Example 16 | Compound 11 | 621 | 388 |
| Example 17 | Compound 12 | 619 | 275 |
| Example 18 | Compound 13 | 619 | 358 |
| Example 19 | Compound 14 | 621 | 276 |
| Example 20 | Compound 15 | 620 | 252 |
| Example 21 | Compound 16 | 618 | 273 |
| Example 22 | Compound 17 | 619 | 222 |
| Example 23 | Compound 18 | 621 | 339 |
| Example 24 | Compound 19 | 621 | 230 |
| Example 25 | Compound 20 | 621 | 196 |
| Example 26 | Compound 21 | 619 | 285 |
| Example 27 | Compound 22 | 620 | 222 |
| Example 28 | Compound 23 | 619 | 305 |
| Example 29 | Compound 24 | 621 | 233 |
| Comparative | Compound 1′ | 619 | 109 |
| Example 5 | | | |
| Comparative | Compound 2′ | 618 | 131 |
| Example 6 | | | |
| Comparative | Compound 3′ | 618 | 153 |
| Example 7 | | | |

From Table 2, by using the compound of the embodiment of the present invention as the host material, the oxygen atom is more stable against the oxidation than the sulfur atom, and the durability is improved as compared with the case of using the comparative compound, and hence, it may be considered that the life was prolonged.

### REFERENCE SIGNS LIST

1: Organic EL device
2: Substrate
3: Anode
4: Cathode
5: Light emitting layer
6: Anode-side organic thin film layer
7: Cathode-side organic thin film layer
10: Light emitting unit

1. A compound represented by the following general formula (1): 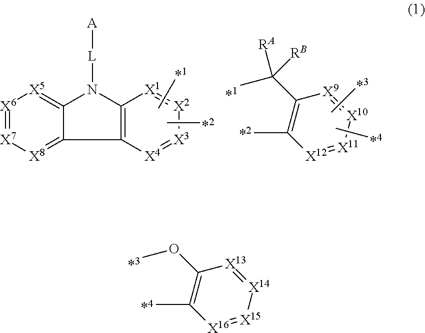
wherein,
in the general formula (1), A is a substituted or unsubstituted aryl group having 6 to 50 ring carbon atoms or a substituted or unsubstituted heteroaryl group having 3 to 50 ring atoms;
L is a single bond, a substituted or unsubstituted arylene group having 6 to 60 ring carbon atoms, or a substituted or unsubstituted heteroarylene group having 3 to 50 ring atoms;
two selected from X¹ to X⁴ are a carbon atom bonding to *¹ or *², respectively, and the other two of X¹ to X⁴ are each independently C(R) or a nitrogen atom;
two selected from X⁹ to X¹² are a carbon atom bonding to *3 or *4, respectively, and the other two of X⁹ to X¹² are each independently C(R) or a nitrogen atom;
X⁵ to X⁸ and X¹³ to X¹⁶ are each independently C(R) or a nitrogen atom,
Rs are each independently a hydrogen atom or a substituent, plural Rs may be the same as or different from every other R, and two selected from plural Rs may be bonded to each other to form a ring; and
R^{A} and R^{B} are each independently a hydrogen atom, a substituted or unsubstituted alkyl group having 1 to 20 carbon atoms, a substituted or unsubstituted aryl group having 6 to 50 ring carbon atoms, a substituted or unsubstituted heteroaryl group having 3 to 50 ring atoms, a halogen atom, a mono-substituted, di-substituted, or tri-substituted silyl group having a substituent selected from an alkyl group having 1 to 50 carbon atoms and an aryl group having 6 to 50 ring carbon atoms, a substituted or unsubstituted alkoxy group having 1 to 20 carbon atoms, a substituted or unsubstituted aryloxy group having 6 to 50 ring carbon atoms, or a cyano group, R^{A} and R^{B} may be the same as or different from each other, and R^{A} and R^{B} may be bonded to each other to form a ring.

2. The compound according to claim 1, which is represented by the following general formula (2): 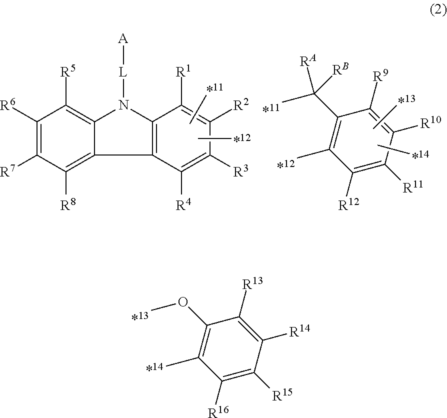
wherein,
in the general formula (2), A, L, R^{A}, and R^{B} are the same as those described regarding the general formula (1) of claim 1,
two groups selected from R¹ to R⁴ split off, the carbon atoms bonded to the subject groups bond to *¹¹ or *¹², and the other two of R¹ to R⁴ are each independently a hydrogen atom or a substituent;
two groups selected from R⁹ to R¹² split off, the carbon atoms bonded to the subject groups bond to *¹³ or *¹⁴, and the other two of R⁹ to R¹² are each independently a hydrogen atom or a substituent; and
R⁵ to R⁸ and R¹³ to R¹⁶ are each independently a hydrogen atom or a substituent and may be the same as or different from each other,
provided that two selected from R¹ to R¹⁶ which do not take part in the bonding to *¹¹ to *¹⁴ may be bonded to each other to form a ring.

3. The compound according to claim 1, which is represented by the following general formula (3): 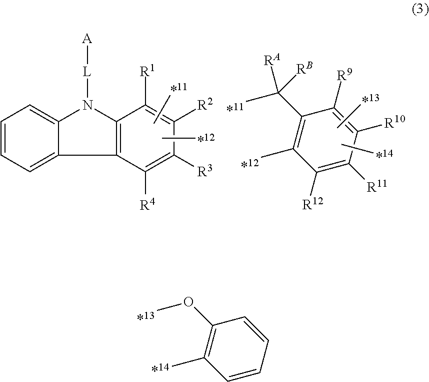
wherein,
in the general formula (3), A, L, R^{A}, and R^{B} are the same as those described regarding the general formula (1) of claim 1,
two groups selected from R¹ to R⁴ split off, the carbon atoms bonded to the subject groups bond to *¹¹ or *¹², and the other two of R¹ to R⁴ are a hydrogen atom; and
two groups selected from R⁹ to R¹² split off, the carbon atoms bonded to the subject groups bond to *¹³ or *¹⁴, and the other two of R⁹ to R¹² are a hydrogen atom.

4. The compound according to claim 2, wherein in the general formula (2) or (3), two groups selected from R¹ and R², R² and R³, and R³ and R⁴ split off, the carbon atoms bonded to the subject groups bond to *¹¹ or *¹²; and that two groups selected from R⁹ and R¹⁰, R¹⁰ and R¹¹, and R¹¹ and R¹² split off, the carbon atom bonded to the subject groups bond to *¹³ or *¹⁴.

5. The compound according to claim 2, which is represented by any one of the following general formulae (2-1) to (2-36): 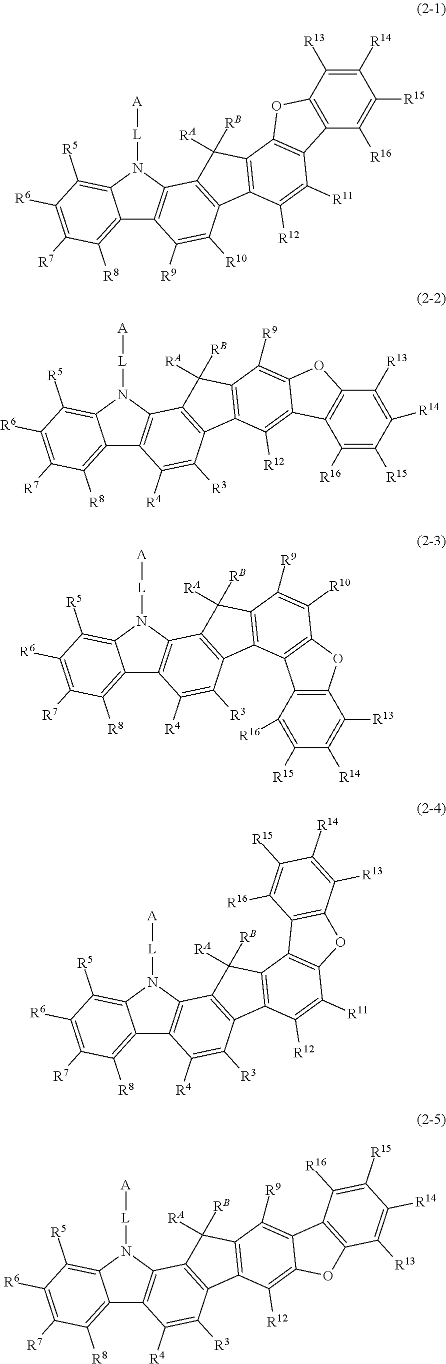 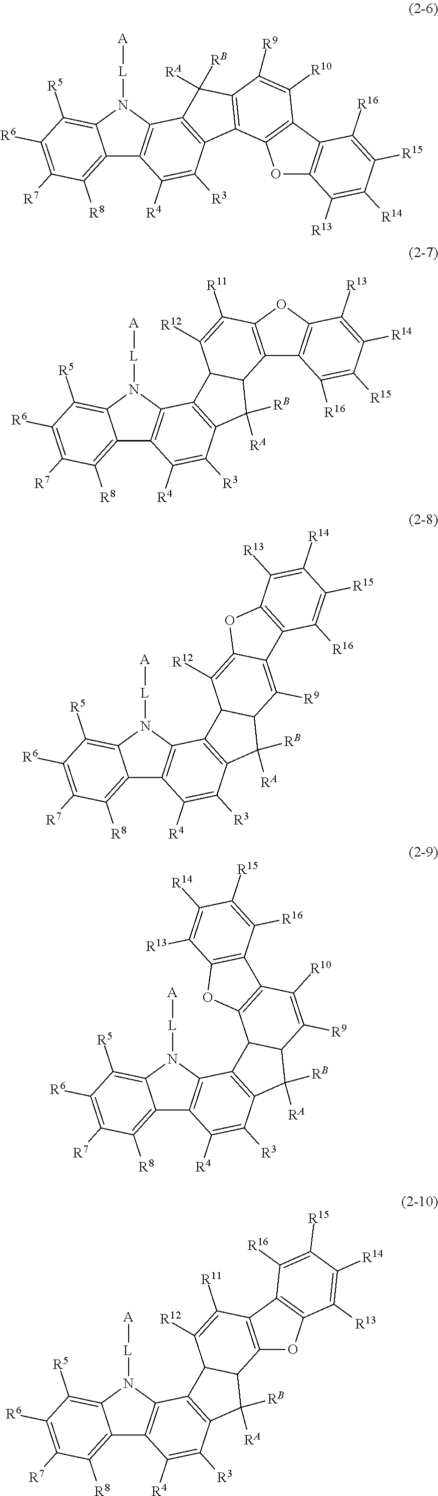 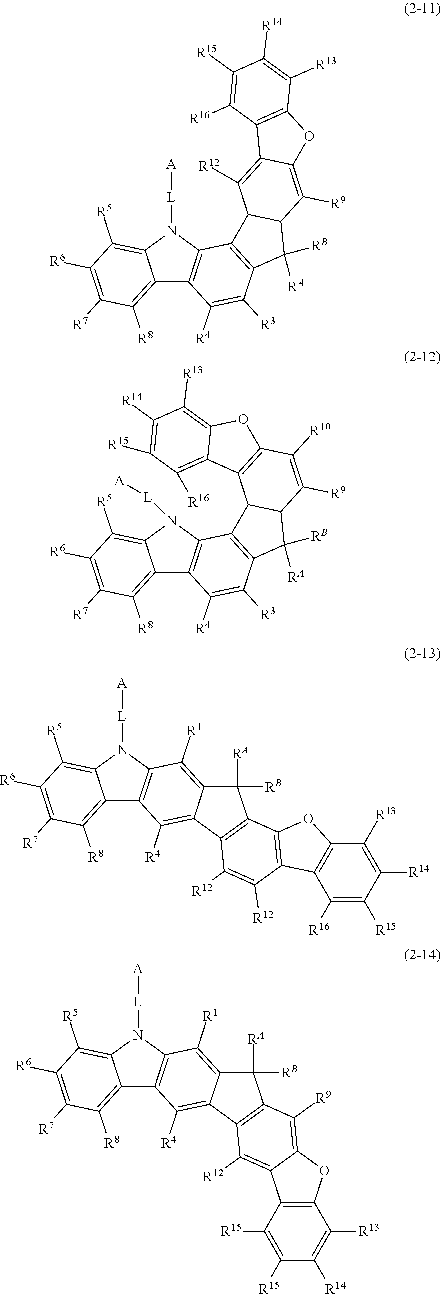 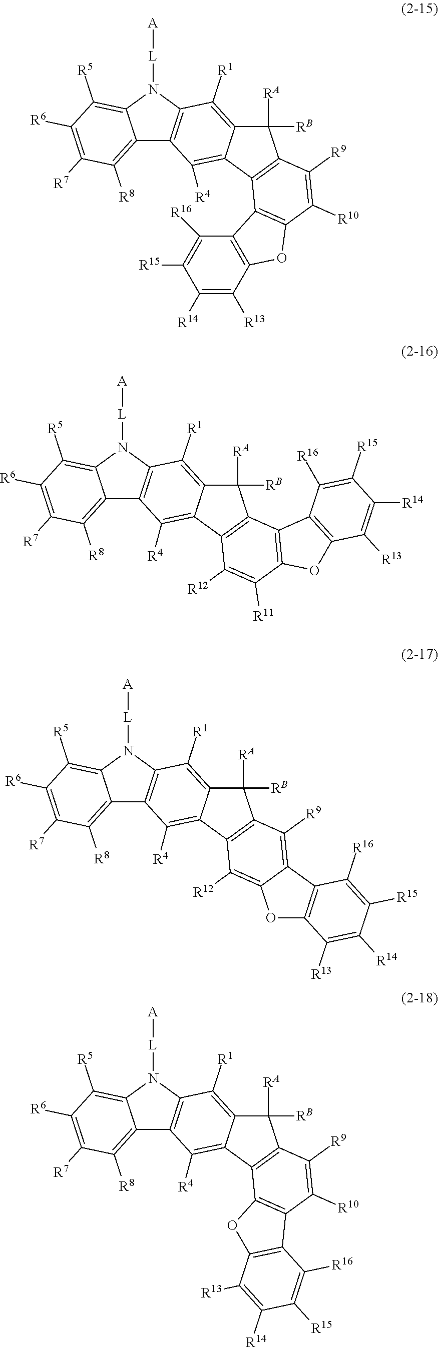 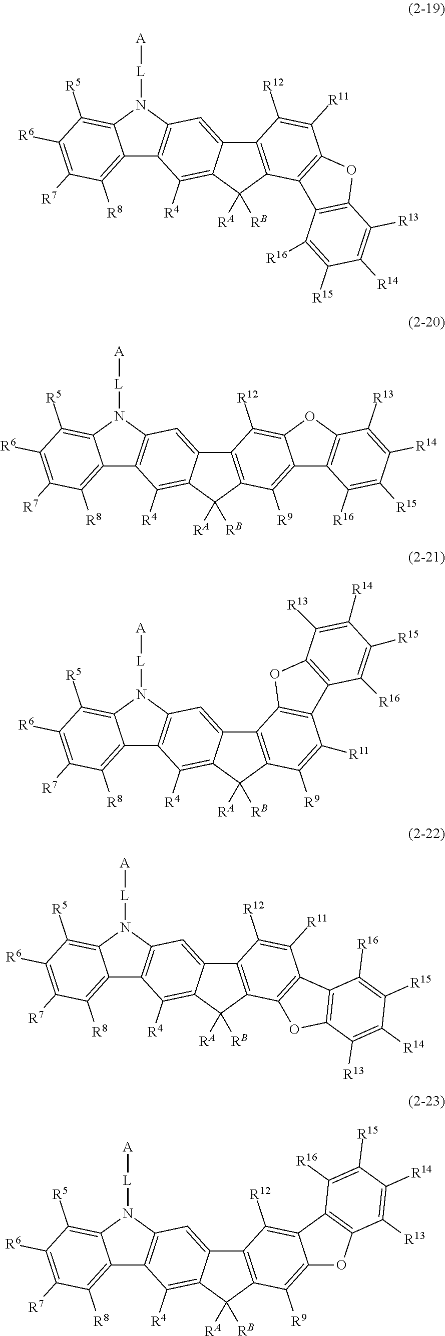 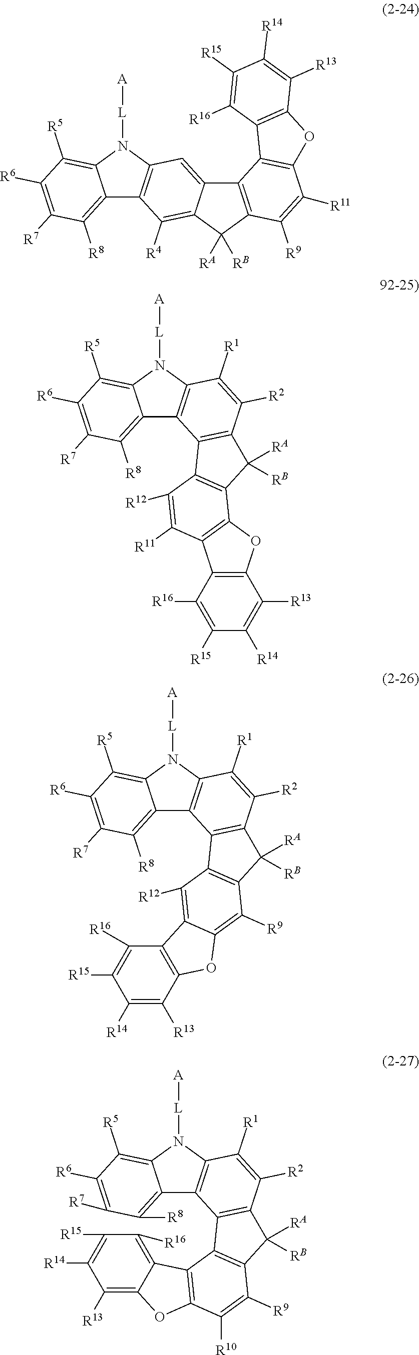 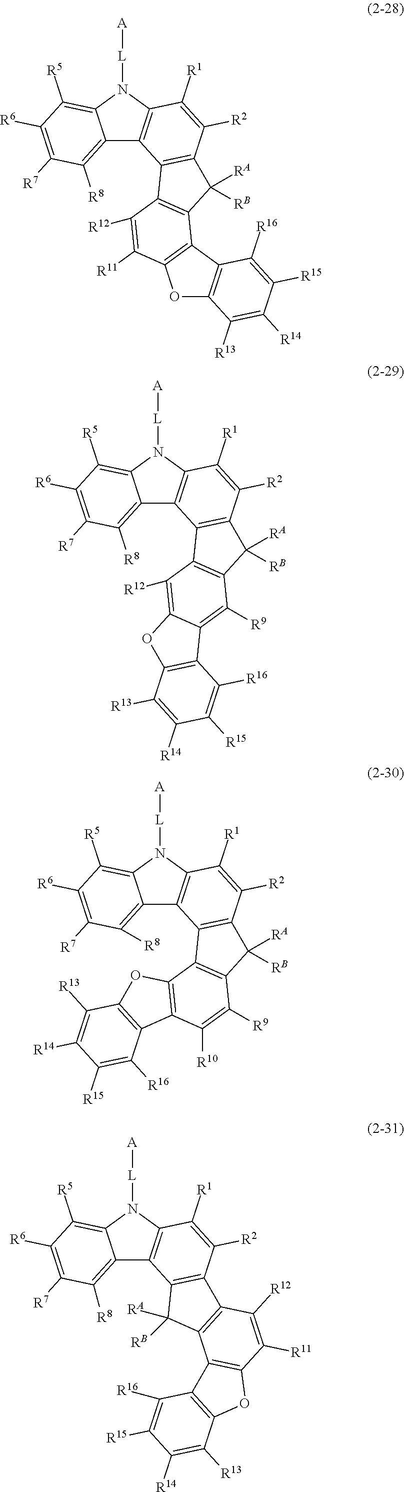 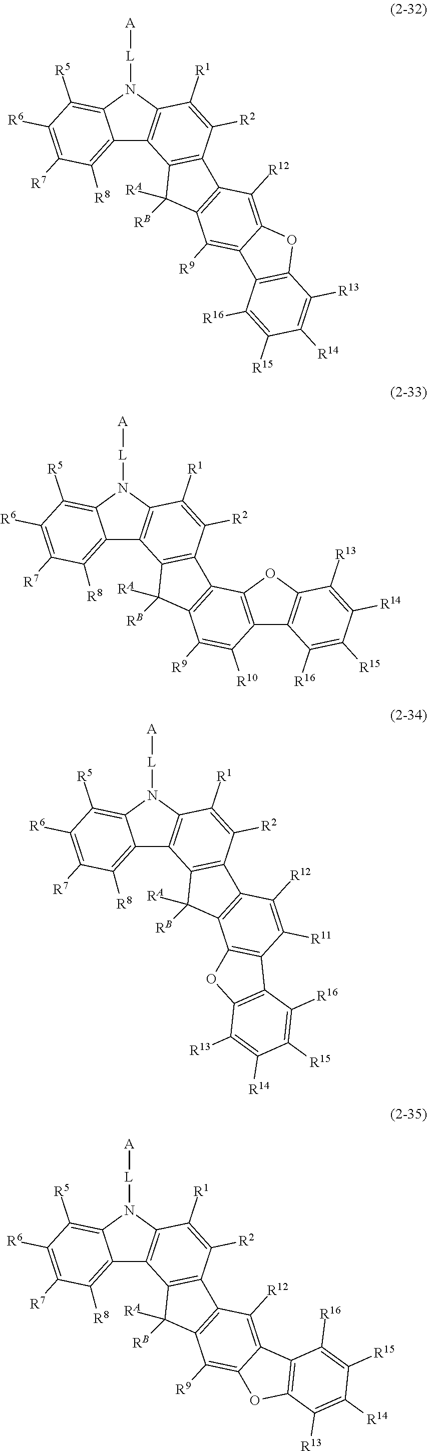 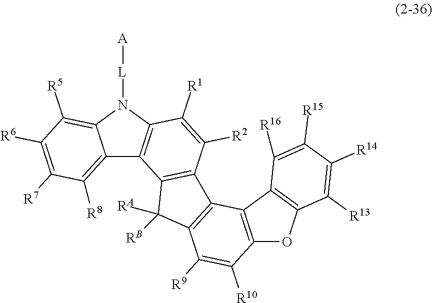
wherein,
in the general formulae (2-1) to (2-36), A, L, R^{A}, and R^{B} are the same as those described regarding the general formula (2) of claim 2; and
R¹ to R¹⁶ and *¹¹ to *¹⁴ are the same as those described regarding the general formula (2) of claim 2.

6. The compound according to claim 5, which is represented by any one of the general formulae (2-2), (2-3), (2-4), (2-6), (2-7), (2-8), (2-9), (2-10), (2-11), (2-12), (2-13), (2-14), (2-15), (2-16), (2-17), (2-18), (2-19), (2-21), (2-23), (2-24), (2-25), (2-26), (2-27), (2-28), (2-29), (2-30), (2-31), (2-32), (2-33), (2-34), (2-35), and (2-36).

7. The compound according to claim 5, which is represented by any one of the general formulae (2-7), (2-9), (2-11), (2-12), (2-14), (2-15), (2-16), (2-18), (2-26), (2-27), (2-28), (2-30), (2-31), (2-33), (2-35), and (2-36).

8. The compound according to claim 1, wherein A is a substituted or unsubstituted aryl group having 6 to 24 ring carbon atoms or a substituted or unsubstituted heteroaryl group having 3 to 24 ring atoms and containing a nitrogen atom, an oxygen atom, or a sulfur atom.

9. The compound according to claim 1, wherein A is a substituted or unsubstituted fused aryl group.

10. The compound according to claim 9, wherein the fused aryl group is a monovalent residual group of a compound represented by any one of the following general formulae (a1-1) to (a1-6): 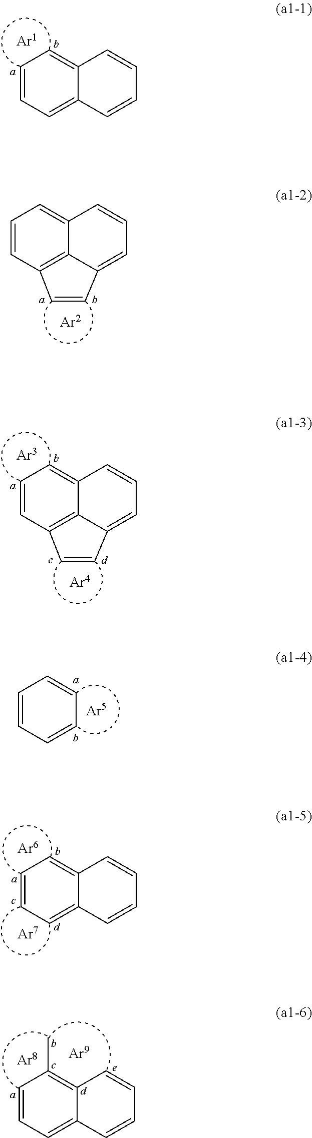
wherein,
in the general formulae (a1-1) to (a1-5), each of Ar¹ to Ar⁷ independently forms a ring structure having 6 to 18 ring carbon atoms together with two carbon atoms expressed by a and b, or c and d, in each of the formulae; and
in the general formula (a1-6), Ar⁸ forms a ring structure having 6 to 18 ring carbon atoms together with three carbon atoms expressed by a, b, and c in the formula, and Ar⁹ forms a ring structure having 6 to 18 ring carbon atoms together with four carbon atoms expressed by b, c, d, and e in the formula,
provided that the carbon atoms bonding to the hydrogen atoms in the structures in the general formula (a1-1) to (a1-6) may be substituted with the aforementioned substituents in place of the hydrogen atoms.

11. The compound according to claim 1, wherein A is a monovalent residual group of a compound represented by the following general formula (a2): 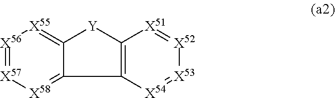
wherein,
in the general formula (a2), X⁵¹ to X⁵⁸ are each independently C(R) or a nitrogen atom, and Rs are each independently a hydrogen atom or a substituent, and two selected from plural Rs may be bonded to each other to form a ring; and
Y is an oxygen atom, a sulfur atom, —NR^{c}, or —C(R^{d})(R^{e})—, and R^{c}, R^{d}, and R^{e} are each independently a hydrogen atom or a substituent, and R^{d} and R^{e} may be bonded to each other to form a ring.

12. The compound according to claim 11, wherein A is a monovalent residual group of a compound represented by the following general formula (a2-1): 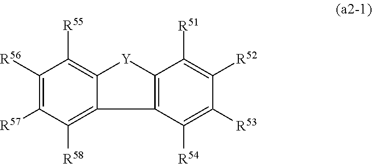
wherein,
in the general formula (a2-1), Y is the same as that described regarding the general formula (a2) of claim 11; and
R⁵¹ to R⁵⁸ are each independently a hydrogen atom or a substituent, and two selected from R⁵¹ to R⁵⁸ may be bonded to each other to form a ring.

13. The compound according to claim 1, wherein A is a monovalent residual group of a compound represented by the following general formula (a3): 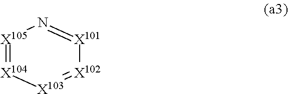
wherein,
in the general formula (a3), X¹⁰¹ to X¹⁰⁵ are each independently C(R^{Y}) or a nitrogen atom; and
R^{Y} is a hydrogen atom or a substituent, and plural R^{Y}s may be the same as or different from every other R^{Y}, and two selected from plural R^{Y}s may be bonded to each other to form a ring.

14. The compound according to claim 13, wherein A is a monovalent residual group of a compound represented by the following general formula (a3-2): 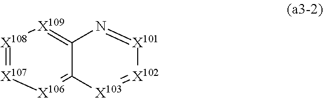
wherein,
in the general formula (a3-2), X¹⁰¹ to X¹⁰³ and X¹⁰⁶ to X¹⁰⁹ are each independently C(R^{Y}) or a nitrogen atom; and
R^{Y} is a hydrogen atom or a substituent, and plural R^{Y}s may be the same as or different from every other R^{Y}, and two selected from plural R^{Y}s may be bonded to each other to form a ring.

15. The compound according to claim 13, wherein A is a monovalent residual group of a compound represented by the following general formula (a3-1-i) or (a3-3-i): 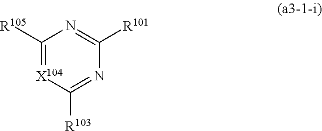
wherein,
in the general formula (a3-1-i), X¹⁰⁴ is C(R^{Y}) or a nitrogen atom; and
R¹⁰¹ and R¹⁰³ to R¹⁰⁵ are each independently a hydrogen atom or a substituent, and two selected from R¹⁰³ to R¹⁰⁵ may be bonded to each other to form a ring, and 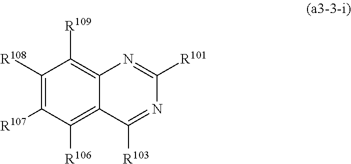
wherein,
in the general formula (a3-3-i), R¹⁰¹, R¹⁰³, and R¹⁰⁶ to R¹⁰⁹ are each independently a hydrogen atom or a substituent, and two selected from R¹⁰³ and R¹⁰⁶ to R¹⁰⁹ may be bonded to each other to form a ring.

16. The compound according to claim 2, which is represented by the following general formula (4): 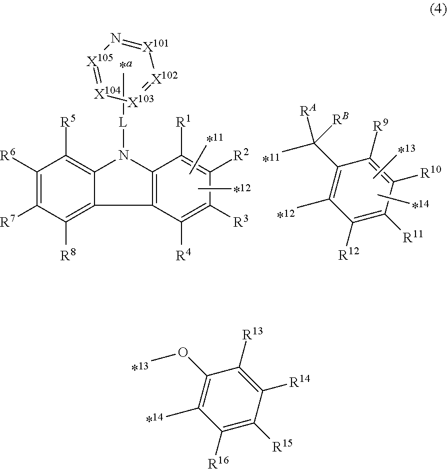
wherein,
in the general formula (4), L, R^{A}, and R^{B} are the same as those described regarding the general formula (2) of claim 2, and R¹ to R¹⁶ and *¹¹ to *¹⁴ are the same as those described regarding the general formula (2) of claim 2; and
X¹⁰¹ to X¹⁰⁵ are each independently a carbon atom bonding to *^{a}, C(R^{Y}), or a nitrogen atom, and R^{Y} is a hydrogen atom or a substituent, and plural R^{V}s may be the same as or different from every other R^{Y}, and two selected from plural R^{Y}s may be bonded to each other to form a ring.

17. The compound according to claim 2, which is represented by the following general formula (5): 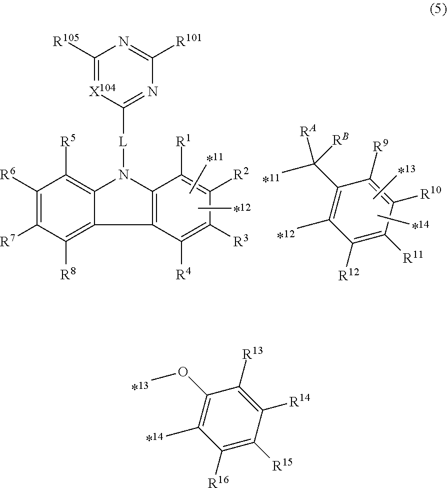
wherein,
in the general formula (5), L, R^{A}, and R^{B} are the same as those described regarding the general formula (2) of claim 2, and R¹ to R¹⁶ and *¹¹ to *¹⁴ are the same as those described regarding the general formula (2) of claim 2; and
X¹⁰⁴ is C(R¹⁰⁴) or a nitrogen atom, and R¹⁰¹, R¹⁰⁴, and R¹⁰⁵ are each independently a hydrogen atom or a substituent, and R¹⁰⁴ and R¹⁰⁵ may be bonded to each other to form a ring.

18. The compound according to claim 1, wherein L is a single bond or a group represented by any one of the following general formulae (i) to (vii): 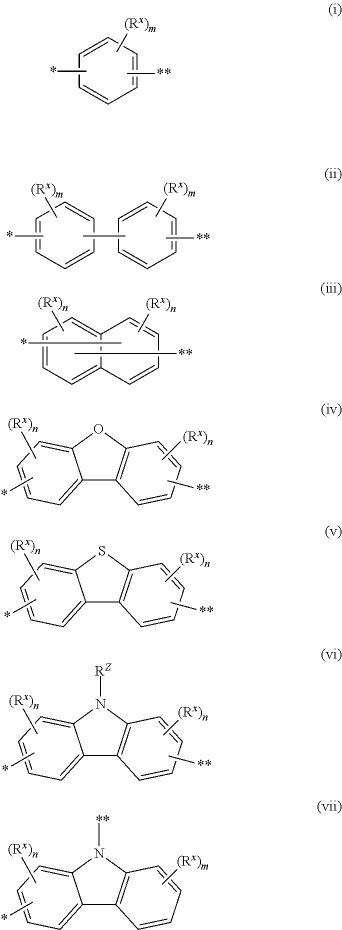
wherein,
R^{X}s are each independently a hydrogen atom or a substituent, and in the case where plural R^{X}s are present, plural R^{X}s may be the same as or different from every other R^{X}, and two selected from plural R^{X}s may be bonded to each other to form a ring structure;
in the formula (vi), R^{Z} is a hydrogen atom or a substituent;
each m is independently an integer of 0 to 4;
each n is independently an integer of 0 to 3; and
each of * and ** expresses a bonding position to the nitrogen atom or A in the general formula (1).

19. The compound according to claim 1, wherein the substituent or the substituent referred to by the description of “substituted or unsubstituted” is a group selected from the group consisting of an alkyl group having 1 to 50 carbon atoms; a cycloalkyl group having 3 to 50 ring carbon atoms; an aryl group having 6 to 50 ring carbon atoms; an aralkyl group having 7 to 51 carbon atoms having an aryl group having 6 to 50 ring carbon atoms; an amino group; a mono-substituted or di-substituted amino group having a substituent selected from an alkyl group having 1 to 50 carbon atoms and an aryl group having 6 to 50 ring carbon atoms; an alkoxy group having an alkyl group having 1 to 50 carbon atoms; an aryloxy group having an aryl group having 6 to 50 ring carbon atoms; a mono-substituted, di-substituted, or tri-substituted silyl group having a substituent selected from an alkyl group having 1 to 50 carbon atoms and an aryl group having 6 to 50 ring carbon atoms; a heteroaryl group having 5 to 50 ring atoms; a haloalkyl group having 1 to 50 carbon atoms; a halogen atom; a cyano group; a nitro group; a sulfonyl group having a substituent selected from an alkyl group having 1 to 50 carbon atoms and an aryl group having 6 to 50 ring carbon atoms; a di-substituted phosphoryl group having a substituent selected from an alkyl group having 1 to 50 carbon atoms and an aryl group having 6 to 50 ring carbon atoms; an alkylsulfonyloxy group; an arylsulfonyloxy group; an alkylcarbonyloxy group; an arylcarbonyloxy group; a boron-containing group; a zinc-containing group; a tin-containing group; a silicon-containing group; a magnesium-containing group; a lithium-containing group; a hydroxyl group; an alkyl-substituted or aryl-substituted carbonyl group; a carboxyl group; a vinyl group; a (meth)acryloyl group; an epoxy group; and an oxetanyl group.

20. A material for organic electroluminescence devices comprising the compound according to claim 1.

21. An organic electroluminescence device comprising a cathode, an anode, and one or more organic thin film layers between the cathode and the anode, wherein
the one or more organic thin film layers include a light emitting layer, and at least one of the one or more organic thin film layers is a layer containing the compound according to claim 1.

22. The organic electroluminescence device according to claim 21, wherein the light emitting layer comprises the compound according to claim 1.

23. The organic electroluminescence device according to claim 21, wherein the light emitting layer further comprises at least one selected from a fluorescent light emitting material and a phosphorescent light emitting material.

24. The organic electroluminescence device according to claim 21, wherein the one or more organic thin film layers further comprise a hole transporting layer.

25. The organic electroluminescence device according to claim 21, wherein the one or more organic thin film layers further comprise an electron transporting layer.

26. An electronic equipment comprising the organic electroluminescence device according to claim 1 mounted thereon.
